(19) United States
(12) Patent Application Publication
(10) Pub. No.: US 2012/0172361 A1
(43) Pub. Date: Jul. 5, 2012

(54) TRIAZINE DERIVATIVES AND THEIR THERAPEUTICAL APPLICATIONS

(75) Inventors: Chunlin Tao, Los Angeles, CA (US); Qinwei Wang, Alhambra, CA (US); Tulay Polat, Los Angeles, CA (US); Laxman Nallan, Alhambra, CA (US); David Ho, Monterey Park, CA (US); Neil Desai, Los Angeles, CA (US)

(73) Assignee: CALIFORNIA CAPITAL EQUITY, LLC, Los Angeles, CA (US)

(21) Appl. No.: 13/376,760

(22) PCT Filed: Jun. 7, 2010
(86) PCT No.: PCT/US10/37614

§ 371 Date: Mar. 26, 2012

Related U.S. Application Data
(60) Provisional application No. 61/185,056, filed on Jun. 8, 2009.

(51) Int. Cl. A61K 31/53; C07D 403/12; C07D 251/38; C07D 413/14; A61K 31/5377; A61P 11/00; A61P 9/04; A61P 9/10; A61P 19/02; A61P 29/00; A61P 37/06; A61P 17/02; C07D 403/14; A61P 35/00

(52) U.S. Cl. 514/236.2; 544/209; 514/245; 544/212; 544/218; 514/241; 544/113

(57) ABSTRACT

Compounds of the formula (I) and formula (II) and pharmaceutically acceptable salts thereof.

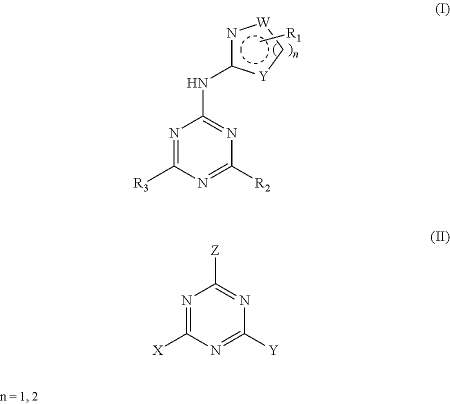

### FIELD OF THE INVENTION

The present invention relates generally to the use of compounds to treat a variety of disorders, diseases and pathologic conditions and more specifically to the use of triazine compounds to modulate protein kinases and for treating protein kinase-mediated diseases.

### BACKGROUND OF THE INVENTION

Protein kinases constitute a large family of structurally related enzymes that are responsible for the control of a variety of signal transduction processes within the cell. Protein kinases, containing a similar 250-300 amino acid catalytic domain, catalyze the phosphorylation of target protein substrates.

The kinases may be categorized into families by the substrates in the phosphorylate (e.g., protein-tyrosine, protein-serine/threonine, lipids, etc.). Tyrosine phosphorylation is a central event in the regulation of a variety of biological processes such as cell proliferation, migration, differentiation and survival. Several families of receptor and non-receptor tyrosine kinases control these events by catalyzing the transfer of phosphate from ATP to a tyrosine residue of specific cell protein targets. Sequence motifs have been identified that generally correspond to each of these kinase families [Hanks et al., FASEB J., (1995), 9, 576-596; Knighton et al., Science, (1991), 253, 407-414; Garcia-Bustos et al., EMBO J., (1994), 13:2352-2361). Examples of kinases in the protein kinase family include, without limitation, abl, Akt, bcr-abl, Blk, Brk, Btk, c-kit, c-Met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, Tie, Tie-2, TRK, Yes, and Zap70.

Studies indicated that protein kinases play a central role in the regulation and maintenance of a wide variety of cellular processes and cellular function. For example, kinase activity acts as molecular switches regulating cell proliferation, activation, and/or differentiation. Uncontrolled or excessive kinase activity has been observed in many disease states including benign and malignant proliferation disorders as well as diseases resulting from inappropriate activation of the immune system (autoimmune disorders), allograft rejection, and graft vs host disease.

It is reported that many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies and asthma, Alzheimer's disease and hormone-related diseases. In addition, endothelial cell specific receptor PTKs, such as VEGF-2 and Tie-2, mediate the angiogenic process and are involved in supporting the progression of cancers and other diseases involving uncontrolled vascularization. Accordingly, there has been a substantial effort in medicinal chemistry to find protein kinase inhibitors that are effective as therapeutic agents.

One kinase family of particular interest is the aurora kinases. The Aurora kinase family is a collection of highly related serine/threonine kinase that are key regulators of mitosis, essential for accurate and equal segtion of genomic material from parent to daught cells. Members of the Aurora kinase family include three related kinases kown as Aurora-A, Aurora-B, and Aurora-C. Despite significant sequence homology, the localization and functions of these kinases are largely distinct from one another (Richard D. Carvajal, et al. Clin Cancer Res 2006; 12(23): 6869-6875; Daruka Mahadevan, et al. Expert Opin. Drug Discov. 2007 2(7): 1011-1026).

Aurora-A is ubiquitously expressed and regulates cell cycle events occurring from late S phase through M phase, including centrosome maturation (Berdnik D, et al. Curr Biol 2002; 12:640-7), mitotic entry (Hirota T, et al. Cell 2003; 114:585-98; Dutertre S, et al. J Cell Sci 2004; 117:2523-31), centrosome separation (Marumoto T, et al. J Biol Chem 2003; 278:51786-95), bipolar-spindle assembly (Kufer T A, et al. J Cell Biol 2002; 158:617-23; Eyers P A, et al. Curr Biol 2003; 13:691-7.), chromosome alignment on the metaphase plate (Marumoto T, et al. J Biol Chem 2003; 278:51786-95; Kunitoku N, et al. Dev Cell 2003; 5:853-64.), cytokinesis (Marumoto T, et al. J Biol Chem 2003; 278:51786-95), and mitotic exit. Aurora-A protein levels and kinase activity both increase from late G2 through M phase, with peak activity in prometaphase. Once activated, Aurora-A mediates its multiple functions by interacting with various substrates including centrosomin, transforming acidic coiled-coil protein, cdc25b, Eg5, and centromere protein A.

Aurora-B is a chromosomal passenger protein critical for accurate chromosomal segregation, cytokinesis (Hauf S, et al. J Cell Biol 2003; 161:281-94; Ditchfield C, et al. J Cell Biol 2003; 161:267-80; Giet R, et al. J Cell Biol 2001; 152:669-82; Goto H, et al. J Biol Chem 2003; 278:8526-30), protein localization to the centromere and kinetochore, correct microtubule-kinetochore attachments (Murata-Hori M, et al. Curr Biol 2002; 12:894-9), and regulation of the mitotic checkpoint. Aurora-B localizes first to the chromosomes during prophase and then to the inner centromere region between sister chromatids during prometaphase and metaphase (Zeitlin S G, et al. J Cell Biol 2001; 155:1147-57). Aurora-B participates in the establishment of chromosomal biorientation, a condition where sister kinetochores are linked to opposite poles of the bipolar spindle via amphitelic attachments. Errors in this process, manifesting as a merotelic attachment state (one kinetochore attached to microtubules from both poles) or a syntelic attachment state (both sister kinetochores attached to microtubules from the same pole), lead to chromosomal instability and aneuploidy if not corrected before the onset of anaphase. The primary role of Aurora-B at this point of mitosis is to repair incorrect microtubule-kinetochore attachments (Hauf S, et al. J Cell Biol 2003; 161:281-94; Ditchfield C, et al. J Cell Biol 2003; 161:267-80; Lan W, et al. Curr Biol 2004; 14:273-86.). Without Aurora-B activity, the mitotic checkpoint is compromised, resulting in increased numbers of aneuploid cells, genetic instability, and tumorigenesis (Weaver B A, et al. Cancer Cell 2005; 8:7-12).

Aurora-A overexpression is a necessary feature of Aurora-A-induced tumorigenesis. In cells with Aurora-A overexpression, mitosis is characterized by the presence of multiple centrosomes and multipolar spindles (Meraldi P et al. EMBO J 2002; 21:483-92.). Despite the resulting aberrant microtubule-kinetochore attachments, cells abrogate the mitotic checkpoint and progress from metaphase to anaphase, resulting in numerous chromosomal separation defects. These cells fail to undergo cytokinesis, and, with additional cell cycles, polyploidy and progressive chromosomal instability develop (Anand S, et al. Cancer Cell 2003; 3:51-62).

The evidence linking Aurora overexpression and malignancy has stimulated interest in developing Aurora inhibitors for cancer therapy. In normal cells, Aurora-A inhibition results in delayed, but not blocked, mitotic entry, centrosome separation defects resulting in unipolar mitotic spindles, and failure of cytokinesis (Marumoto T, et al. J Biol Chem 2003; 278:51786-95). Encouraging antitumor effects with Aurora-A inhibition were shown in three human pancreatic cancer cell lines (Panc-1, MIA PaCa-2, and SU.86.86), with growth suppression in cell culture and near-total abrogation of tumorigenicity in mouse xenografts (Hata T, et al. Cancer Res 2005; 65:2899-905.).

Aurora-B inhibition results in abnormal kinetochore-microtubule attachments, failure to achieve chromosomal biorientation, and failure of cytokinesis (Goto H, et al. J Biol Chem 2003; 278:8526-30; Severson A F, et al. Curr Biol 2000; 10:1162-71). Recurrent cycles of aberrant itosis without cytokinesis result in massive polyploidy and, ultimately, to apoptosis (Hauf S, et al. J Cell Biol 2003; 161:281-94; Ditchfield C, et al. J Cell Biol 2003; 161:267-80; Giet R, et al. J Cell Biol 2001; 152:669-82; Murata-Hori M, Curr Biol 2002; 12:894-9; Kallio M J, et al. Curr Biol 2002; 12:900-5).

Inhibition of Aurora-A or Aurora-B activity in tumor cells results in impaired chromosome alignment, abrogation of the mitotic checkpoint, polyploidy, and subsequent cell death. These in vitro effects are greater in transformed cells than in either non-transformed or non-dividing cells (Ditchfield C, et al. J Cell Biol 2003; 161:267-80). Thus, targeting Aurora may achieve in vivo selectivity for cancer. Although toxicity to rapidly dividing cell of the hematopoietic and gastrointestinal system is expected, the activity and clinical tolerability shown in xenograft models indicates the presence of a reasonable therapeutic index.

Given the preclinical antitumor activity and potential for tumor selectivity, several Aurora kinase inhibitors have been developed. The first three small-molecule inhibitors of Aurora described include ZM447439 (Ditchfield C, et al. J Cell Biol 2003; 161:267-80), Hesperadin (Hauf S, et al. J Cell Biol 2003; 161:281-94), and MK0457 (VX680) (Harrington E A, et al. Nat Med 2004; 10:262-7). The following agents are nonspecific inhibitors: ZM447439 inhibits Aurora-A and Aurora-B; Herperadin inhibits primarily Aurora-B; MK0457 inhibits all three Aurora kinases. Each induces a similar phenotype in cell-based assays, characterized by inhibition of phosphorylation of histone H3 on Ser10, inhibition of cytokinesis, and the development of polyploidy. Selective inhibitors of Aurora have also been developed. A selective Aurora-A inhibitor is MLN8054 (Hoar H M, et al. [abstract C40]. Proc AACR-NCI-EORTC International Conference: Molecular Targets and Cancer Therapeutics 2005). A expmple of selective Aurora-B inhibitor is AZD1152 (Schellens J, et al. [abstract 3008]. Proc Am Soc Clin Oncol 2006; 24:122s). The next generation of Aurora inhibitors is currently being developed, including agents by Nerviano Medical Sciences (PHA-680632 and PHA-739358), Rigel (R763), Sunesis (SNS-314), NCE Discovery Ltd. (NCED#17), Astex Therapeutics (AT9283), and Montigen Pharmaceuticals (MP-235 and MP-529). Several of these agents are undergoing evaluation in clinical trials.

Considering the lack of currently available treatment options for the majority of the conditions associated with protein kinases, there is still a great need for new therapeutic agents for these conditions.

### BRIEF SUMMARY OF THE INVENTION

Accordingly, one aspect of the present invention provides an antitumor agent comprising a triazine derivative as described in formula (I) or formula (II), pharmaceutically-acceptable formulations thereof, methods for making novel compounds and compositions for using the compounds. The compounds and compositions comprising the compounds of formula (I) or formula (II) have utility in treatment of a variety of diseases.

The combination therapy described herein may be provided by the preparation of the triazine derivative of formula (I) or formula (II) and the other therapeutic agent as separate pharmaceutical formulations followed by the administration thereof to a patient simultaneously, semi-simultaneously, separately or over regular intervals.

The present invention provides methods of use for certain chemical compounds such as kinase inhibitors for treatment of various diseases, disorders, and pathologies, for example, cancer, and vascular disorders, such as myocardial infarction (MI), stroke, or ischemia. The triazine compounds described in this invention may block the enzymatic activity of some or many of the members of the Aurora kinase family, in addition to blocking the activity of other receptor and non-receptor kinase. Such compounds may be beneficial for treatment of the diseases where disorders affect cell motility, adhesion, and cell cycle progression, and in addition, diseases with related hypoxic conditions, osteoporosis and conditions, which result from or are related to increases in vascular permeability, inflammation or respiratory distress, tumor growth, invasion, angiogenesis, metastases and apoptosis.

### DETAILED DESCRIPTION OF THE INVENTION

The present invention comprises compounds as shown in formula (I)

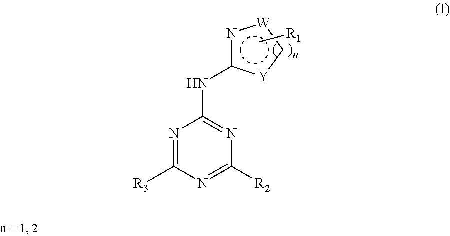

or a pharmaceutically acceptable salt thereof, wherein:

W and Y are independently selected from S, O, NR₄, CR₄ or CR₁;

R₄ is independently selected from hydrogen or an optionally substituted C₁₋₄ aliphatic group.

R₁ represents hydrogen, halogen, hydroxy, amino, cyano, alkyl, cycloalkyl, alkenyl, alkynyl, alkylthio, aryl, arylalkyl, heterocyclic, heteroaryl, heterocycloalkyl, alkylsulfonyl, alkoxycarbonyl and alkylcarbonyl.

R₂ is selected from:
(i) amino, alkyl amino, aryl amino, heteroaryl amino;
(ii) C₁-C₆ alkyl, C₂-C₆ alkenyl, C₂-C₆ alkynyl;
(iii) aryl, heterocyclic, heteroaryl; and
(iv) groups of the formula (Ia):

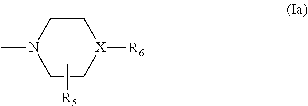

wherein:

R₅ represents hydrogen, C₁-C₄ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl)sulfonamido, and mono- and di-(C₁-C₆alkyl)aminocarbonyl, each of which is substituted with from 0 to 4 substituents independently chosen from halogen, hydroxy, cyano, amino, —COOH and oxo;

R₃ is selected from:

(i) C₁-C₆ alkyl, C₂-C₆ alkenyl, C₂-C₆ alkynyl;

(ii) heterocyclic,

(iii) K—Ar.

Ar represents heteroaryl or aryl, each of which is substituted with from 0 to 4 substituents independently chosen from:
(1) halogen, hydroxy, amino, amide, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl)sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

K is selected from

i) absence;

ii) O, S, SO, SO₂;

iii) (CH₂)ₘ, m=0-3, —O(CH₂)ₚ, p=1-3, —S(CH₂)ₚ, p=1-3, —N(CH₂)ₚ, p=1-3, —(CH₂)ₚO, p=1-3;

iv) NR₇

R₇ represents hydrogen, alkyl, cycloalkyl, alkenyl, alkynyl, alkylthio, aryl, arylalkyl.

The present invention also comprises compounds as shown in formula (II)

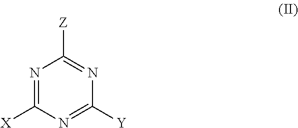

or a pharmaceutically acceptable salt thereof, wherein:

Y is selected from C₁-C₆ alkyl, C₂-C₆ alkenyl, C₂-C₆ alkynyl, —NR⁴R⁵, and -Q-R³;

Q is selected from aryl, heteroaryl, cycloalkyl, and heterocycloalkyl, each of which is optionally substituted with C₁-C₆ alkyl or oxo;

R³ is selected from H, C₁-C₆ alkyl, C₂-C₆ alkenyl, C₂-C₆ alkynyl, C₁-C₆ alkyl-R⁶, aryl, and heteroaryl;

R⁴ and R⁵ are each independently selected from H, C₁-C₆ alkyl, and C₁-C₆ alkyl-R⁶;

R⁶ is selected from hydroxy, —NH₂, mono(C₁-C₆ alkyl)amino, di(C₁-C₆ alkyl)amino, cycloalkyl, and heterocycloalkyl;

X is selected from —K—Ar¹—R¹, C₁-C₆ alkyl, cycloalkyl, and heterocycloalkyl, each of which is optionally substituted with C₁-C₆ alkyl, halogen, hydroxy, amino, cyano, —COOH, or oxo;

K is selected from O and S;

Ar¹ is selected from aryl and heteroaryl;

R¹ is selected from H, —NHC(O)W, —C(O)NHW, and —NH₂;

W is selected from C₁-C₆ alkyl, aryl, heteroaryl, and aryl(C₁-C₆)alkyl, each of which is optionally substituted with C₁-C₆ alkyl, halogen, hydroxy, amino, cyano, —COOH, or oxo;

Z is —(NH)ₙ—Ar²—R²;

n=0, 1;

Ar² is selected from aryl and heteroaryl, each of which is optionally substituted with C₁-C₆ alkyl, halogen, hydroxy, amino, cyano, —COOH, or oxo;

R² is selected from H, C₁-C₆ alkyl, —NH₂, ═NH, C₁-C₆ alkoxycarbonyl, halo, and cycloalkyl.

The invention further comprises compounds as shown in formula (II)

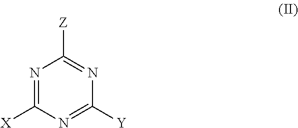

or a pharmaceutically acceptable salt thereof, wherein:

Y is selected from C₁-C₆ alkyl, phenyl, morpholinyl, piperidinyl, pyrrolidinyl, —NR⁴R⁵, and -Q-R³;

Q is piperazinyl;

R³ is selected from C₁-C₆ alkyl, hydroxy(C₁-C₆)alkyl, and pyridinyl;

R⁴ and R⁵ are each independently selected from H, C₁-C₆ alkyl, and C₁-C₆ alkyl-R⁶;

R⁶ is selected from morpholinyl and di(C₁-C₆ alkyl)amino;

X is selected from C₁-C₆ alkyl, methylpiperazinyl, and —K—Ar¹—R¹;

K is selected from O and S;

Ar¹ is phenyl;

R¹ is selected from —NHC(O)W, —C(O)NHW, and —NH₂;

W is selected from C₁-C₆ alkyl, phenyl, and halobenzyl;

Z is —(NH)ₙ—Ar²—R²;

n=0, 1;

Ar² is selected from methylthiazolyl, pyrazolyl, imidazolyl, triazolyl, benzimidazolyl, thiadiazolyl, thiazolyl, isoxazolyl, isothiazolyl, pyrimidinyl, and pyridinyl;

R² is selected from C₁-C₆ alkyl, —NH₂, ═NH, C₁-C₆ alkoxycarbonyl, and halo.

The following definitions refer to the various terms used above and throughout the disclosure.

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that include, variables (e.g. X, Ar.). Unless otherwise specified, each variable within such a formula is defined independently of any other variable, and any variable that occurs more than one time in a formula is defined independently at each occurrence.

The term “halo” or “halogen” refers to fluorine, chlorine, bromine or iodine.

The term “alkyl” herein alone or as part of another group refers to a monovalent alkane (hydrocarbon) derived radical containing from 1 to 12 carbon atoms unless otherwise defined. Alkyl groups may be substituted at any available point of attachment. An alkyl group substituted with another alkyl group is also referred to as a “branched alkyl group”. Exemplary alkyl groups include methyl, ethyl, propyl, isopropyl, n-butyl, t-butyl, isobutyl, pentyl, hexyl, isohexyl, heptyl, dimethylpentyl, octyl, 2,2,4-trimethylpentyl, nonyl, decyl, undecyl, dodecyl, and the like. Exemplary substituents include but are not limited to one or more of the following groups: alkyl, aryl, halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). In some preferred embodiments of the present invention, alkyl groups are substituted with, for example, amino, heterocycloalkyl, such as morpholine, piperazine, piperidine, azetidine, hydroxyl, methoxy, or heteroaryl groups such as pyrrolidine. “Alkyl” also includes cycloalkyl.

The term “cycloalkyl” herein alone or as part of another group refers to fully saturated and partially unsaturated hydrocarbon rings of 3 to 9, preferably 3 to 7 carbon atoms. The examples include cyclopropyl, cyclobutyl, cyclopentyl and cyclohexyl, and like. Further, a cycloalkyl may be substituted. A substituted cycloalkyl refers to such rings having one, two, or three substituents, selected from the group consisting of halo, alkyl, substituted alkyl, alkenyl, alkynyl, nitro, cyano, oxo (═O), hydroxy, alkoxy, thioalkyl, —CO₂H, —C(═O)H, CO₂-alkyl, —C(═O)alkyl, keto, ═N—OH, ═N—O-alkyl, aryl, heteroaryl, heterocyclo, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ are independently selected from hydrogen, alkyl, substituted alkyl, and cycloalkyl, or R′ and R″ together form a heterocyclo or heteroaryl ring.

The term “alkenyl” herein alone or as part of another group refers to a hydrocarbon radical straight, branched or cyclic containing from 2 to 12 carbon atoms and at least one carbon to carbon double bond. Examples of such groups include the vinyl, allyl, 1-propenyl, isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, and like. Alkenyl groups may also be substituted at any available point of attachment. Exemplary substituents for alkenyl groups include those listed above for alkyl groups, and especially include C₃ to C₇ cycloalkyl groups such as cyclopropyl, cyclopentyl and cyclohexyl, which may be further substituted with, for example, amino, oxo, hydroxyl, etc.

The term “alkynyl” refers to straight or branched chain alkyne groups, which have one or more unsaturated carbon-carbon bonds, at least one of which is a triple bond. Alkynyl groups include C₂-C₈ alkynyl, C₂-C₆ alkynyl and C₂-C₄ alkynyl groups, which have from 2 to 8, 2 to 6 or 2 to 4 carbon atoms, respectively. Illustrative of the alkynyl group include ethenyl, propenyl, isopropenyl, butenyl, isobutenyl, pentenyl, and hexenyl. Alkynyl groups may also be substituted at any available point of attachment. Exemplary substituents for alkynyl groups include those listed above for alkyl groups such as amino, alkylamino, etc. The numbers in the subscript after the symbol “C” define the number of carbon atoms a particular group can contain.

The term “alkoxy” alone or as part of another group denotes an alkyl group as described above bonded through an oxygen linkage (—O—). Preferred alkoxy groups have from 1 to 8 carbon atoms. Examples of such groups include the methoxy, ethoxy, n-propoxy, isopropoxy, n-butoxy, isobutoxy, sec-butoxy, tert-butoxy, n-pentyloxy, isopentyloxy, n-hexyloxy, cyclohexyloxy, n-heptyloxy, n-octyloxy and 2-ethylhexyloxy.

The term “alkylthio” refers to an alkyl group as described above attached via a sulfur bridge. Preferred alkoxy and alkylthio groups are those in which an alkyl group is attached via the heteroatom bridge. Preferred alkylthio groups have from 1 to 8 carbon atoms. Examples of such groups include the methylthio, ethylthio, n-propythiol, n-butylthiol, and like.

The term “oxo,” as used herein, refers to a keto (C═O) group. An oxo group that is a substituent of a nonaromatic carbon atom results in a conversion of —CH₂— to —C(═O)—.

The term “alkoxycarbonyl” herein alone or as part of another group denotes an alkoxy group bonded through a carbonyl group. An alkoxycarbonyl radical is represented by the formula: —C(O)OR, where the R group is a straight or branched C₁-C₆ alkyl group, cycloalkyl, aryl, or heteroaryl.

The term “alkylcarbonyl” herein alone or as part of another group refers to an alkyl group bonded through a carbonyl group or —C(O)R.

The term “arylalkyl” herein alone or as part of another group denotes an aromatic ring bonded through an alkyl group (such as benzyl) as described above.

The term “aryl” herein alone or as part of another group refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 20 atoms therein, with alternating (resonating) double bonds between adjacent carbon atoms or suitable heteroatoms. Aryl groups may optionally be substituted with one or more groups including, but not limited to halogen such as I, Br, F, or Cl; alkyl, such as methyl, ethyl, propyl, alkoxy, such as methoxy or ethoxy, hydroxy, carboxy, carbamoyl, alkyloxycarbonyl, nitro, alkenyloxy, trifluoromethyl, amino, cycloalkyl, aryl, heteroaryl, cyano, alkyl S(O)ₙ, (m=0, 1, 2), or thiol.

The term “aromatic” refers to a cyclically conjugated molecular entity with a stability, due to delocalization, significantly greater than that of a hypothetical localized structure, such as the Kekule structure.

The term “amino” herein alone or as part of another group refers to —NH₂. An “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl, thioalkyl, carbonyl or carboxyl. These substituents may be further substituted with a carboxylic acid, any of the alkyl or aryl substituents set out herein. In some embodiments, the amino groups are substituted with carboxyl or carbonyl to form N-acyl or N-carbamoyl derivatives.

The term “alkylsulfonyl” refers to groups of the formula (SO₂)-alkyl, in which the sulfur atom is the point of attachment. Preferably, alkylsulfonyl groups include C₁-C₆ alkylsulfonyl groups, which have from 1 to 6 carbon atoms. Methylsulfonyl is one representative alkylsulfonyl group.

The term “heteroatom” refers to any atom other than carbon, for example, N, O, or S.

The term “heteroaryl” herein alone or as part of another group refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom.

The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero, one, two or three substituents selected from the group consisting of halo, alkyl, substituted alkyl, alkenyl, alkynyl, aryl, nitro, cyano, hydroxy, alkoxy, thioalkyl, —CO₂H, —C(═O)H, —CO₂-alkyl, —C(═O)alkyl, phenyl, benzyl, phenylethyl, phenyloxy, phenylthio, cycloalkyl, substituted cycloalkyl, heterocyclo, heteroaryl, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ is independently selected from hydrogen, alkyl, substituted alkyl, and cycloalkyl, or R′ and R″ together form a heterocyclo or heteroaryl ring.

Preferably monocyclic heteroaryl groups include pyrrolyl, pyrazolyl, pyrazolinyl, imidazolyl, oxazolyl, diazolyl, isoxazolyl, thiazolyl, thiadiazolyl, S isothiazolyl, furanyl, thienyl, oxadiazolyl, pyridyl, pyrazinyl, pyrimidinyl, pyridazinyl, triazinyl and the like.

Preferably bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, dihydroisoindolyl, tetrahydroquinolinyl and the like.

Preferably tricyclic heteroaryl groups include carbazolyl, benzidolyl, phenanthrollinyl, acridinyl, phenanthridinyl, xanthenyl and the like.

The term “heterocycle” or “heterocycloalkyl” herein alone or as part of another group refers to a cycloalkyl group (nonaromatic) in which one of the carbon atoms in the ring is replaced by a heteroatom selected from O, S or N. The “heterocycle” has from 1 to 3 fused, pendant or spiro rings, at least one of which is a heterocyclic ring (i.e., one or more ring atoms is a heteroatom, with the remaining ring atoms being carbon). The heterocyclic ring may be optionally substituted which means that the heterocyclic ring may be substituted at one or more substitutable ring positions by one or more groups independently selected from alkyl (preferably lower alkyl), heterocycloalkyl, heteroaryl, alkoxy (preferably lower alkoxy), nitro, monoalkylamino (preferably a lower alkylamino), dialkylamino (preferably a alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy; lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. A heterocyclic group may generally be linked via any ring or substituent atom, provided that a stable compound results. N-linked heterocyclic groups are linked via a component nitrogen atom.

Typically, a heterocyclic ring comprises 1-4 heteroatoms; within certain embodiments each heterocyclic ring has 1 or 2 heteroatoms per ring. Each heterocyclic ring generally contains from 3 to 8 ring members (rings having from to 7 ring members are recited in certain embodiments), and heterocycles comprising fused, pendant or spiro rings typically contain from 9 to 14 ring members which consists of carbon atoms and contains one, two, or three heteroatoms selected from nitrogen, oxygen and/or sulfur.

Examples of “heterocycle” or “heterocycloalkyl groups include piperazine, piperidine, morpholine, thiomorpholine, pyrrolidine, imidazolidine and thiazolide.

The term “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member.

The term “optionally substituted” as it refers that the aryl or heterocyclyl or other group may be substituted at one or more substitutable positions by one or more groups independently selected from alkyl (preferably lower alkyl), alkoxy (preferably lower alkoxy), nitro, monoalkylamino (preferably with one to six carbons), dialkylamino (preferably with one to six carbons), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy and lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. Optional substitution is also indicated by the phrase “substituted with from 0 to X substituents,” where X is the maximum number of possible substituents. Certain optionally substituted groups are substituted with from 0 to 2, 3 or 4 independently selected substituents.

A dash (“-”) that is not between two letters or symbols is used to indicate a point of t attachment for a substituent. For example, —CONH₂ is attached through the carbon atom.

A dashed cycle that locates inside of a heterocyle ring is used to indicate a conjugated system. The bonds between two atomes may be single bond or double bond.

The term “anticancer” agent includes any known agent that is useful for the treatment of cancer including, but is not limited, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

The term “kinase” refers to any enzyme that catalyzes the addition of phosphate groups to a protein residue; for example, serine and threonine kineses catalyze the addition of phosphate groups to serine and threonine residues.

The terms “Src kinase,” “Src kinase family,” and “Src family” refer to the related homologs or analogs belonging to the mammalian family of Src kineses, including, for example, c-Src, Fyn, Yes and Lyn kineses and the hematopoietic-restricted kineses Hck, Fgr, Lck and Blk.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

The term “protected” refers that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, John Wiley & Sons, New York (1999).

The term “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC— (CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred. It will be apparent that each compound of formula (I) or formula (II) may, but need not, be formulated as a hydrate, solvate or non-covalent complex. In addition, the various crystal forms and polymorphs are within the scope of the present invention. Also provided herein are prodrugs of the compounds of formula (I) or formula (II).

The term of “prodrug” refers a compound that may not fully satisfy the structural requirements of the compounds provided herein, but is modified in vivo, following administration to a patient, to produce a compound of formula (I) or formula (II), or other formula provided herein. For example, a prodrug may be an acylated derivative of a compound as provided herein. Prodrugs include compounds wherein hydroxy, amine or thiol groups are bonded to any group that, when administered to a mammalian subject, cleaves to form a free hydroxy, amino, or thiol group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

Groups that are “optionally substituted” are unsubstituted or are substituted by other than hydrogen at one or more available positions. Such optional substituents include, for example, hydroxy, halogen, cyano, nitro, C₁-C₆ alkyl, C₂-C₆ alkenyl, C₂-C₆ alkynyl, C₁-C₆ alkoxy, C₂-C₆ alkyl ether, C₃-C₆ alkanone, C₂-C₆ alkylthio, amino, mono- or di-(C₁-C₆ alkyl)amino, C₁-C₆ haloalkyl, —COOH, —CONH₂, mono- or di-(C₁-C₆ alkyl)aminocarbonyl, —SO₂NH₂, and/or mono or di(C₁-C₆ alkyl)sulfonamido, as well as carbocyclic and heterocyclic groups.

Optional substitution is also indicated by the phrase “substituted with from 0 to X substituents,” where X is the maximum number of possible substituents. Certain optionally substituted groups are substituted with from 0 to 2, 3 or 4 independently selected substituents.

Preferred R₁ groups of formula (I) are listed below:

Hydrogen, halogen, hydroxy, amino, cyano, alkyl, cycloalkyl, alkenyl, alkynyl, alkylthio, aryl, arylalkyl, heterocyclic, heteroaryl, heterocycloalkyl, alkylsulfonyl, alkoxycarbonyl and alkylcarbonyl.

Preferred R₂ groups of formula (I) are listed below:

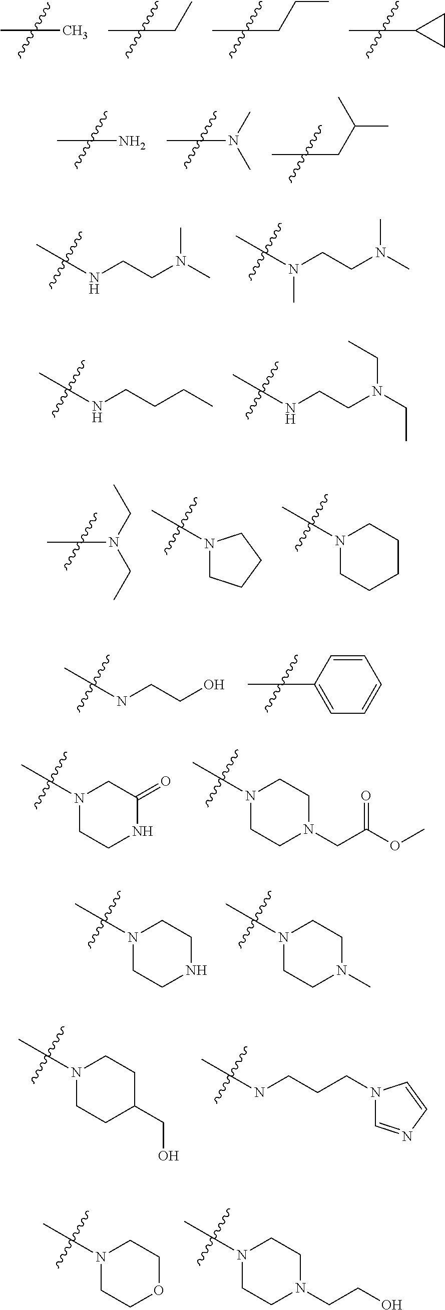

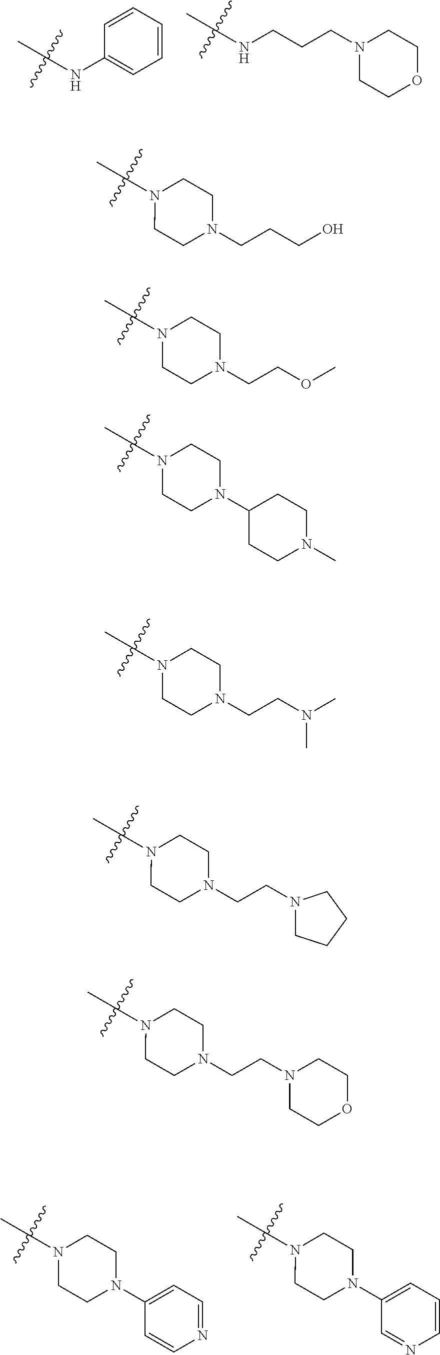

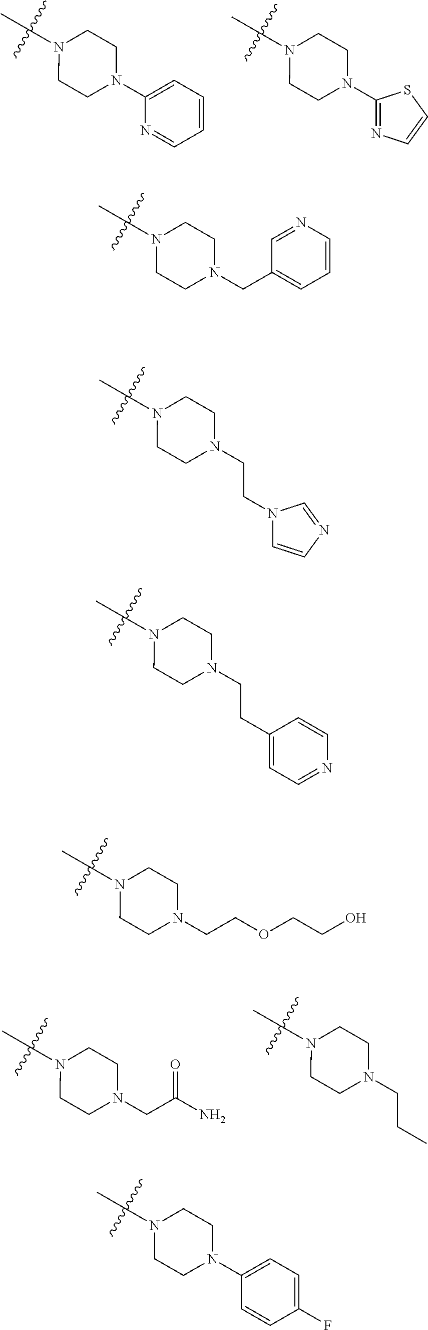

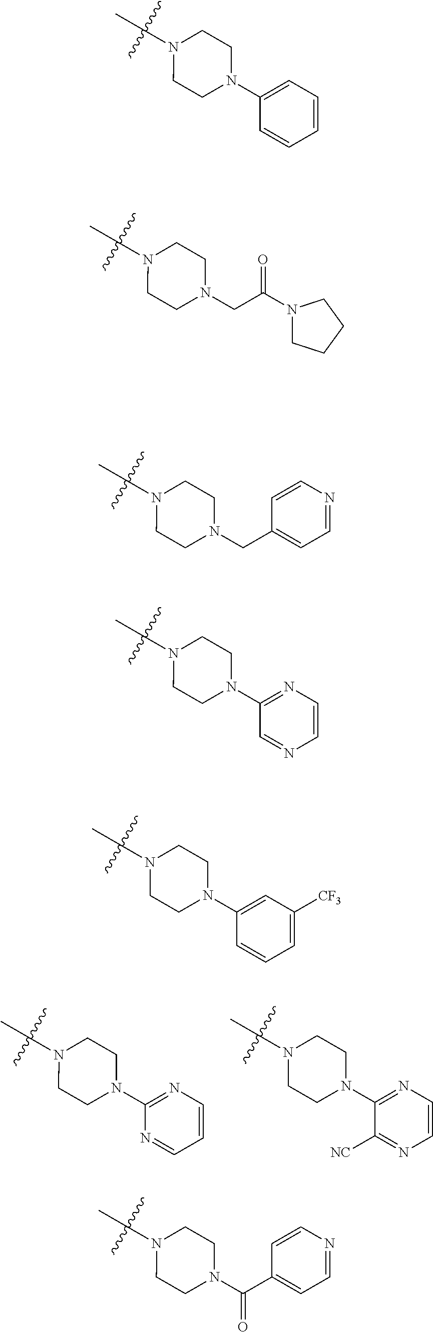

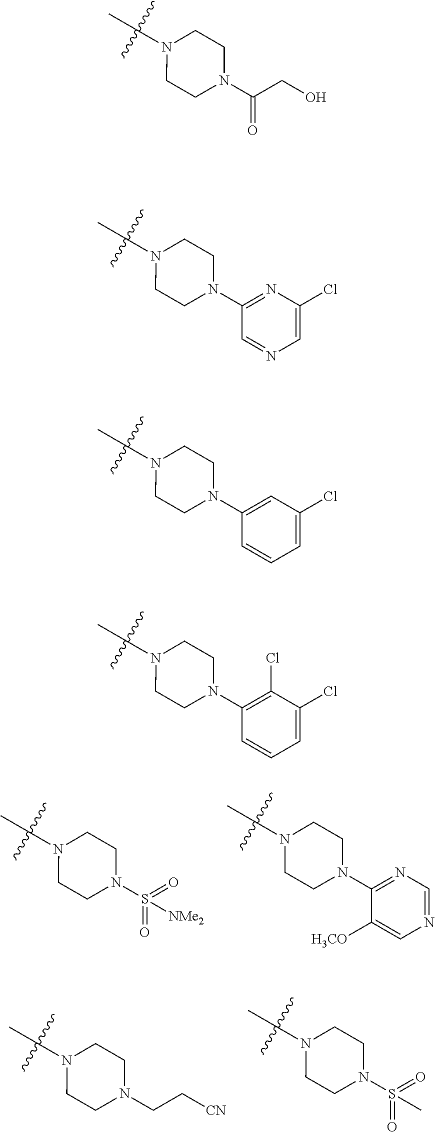

Preferred R₃ groups of formula (I) are listed below, wherein the substitute may be the specific ones as defined here or may be one or multiple substitutes as defined above:

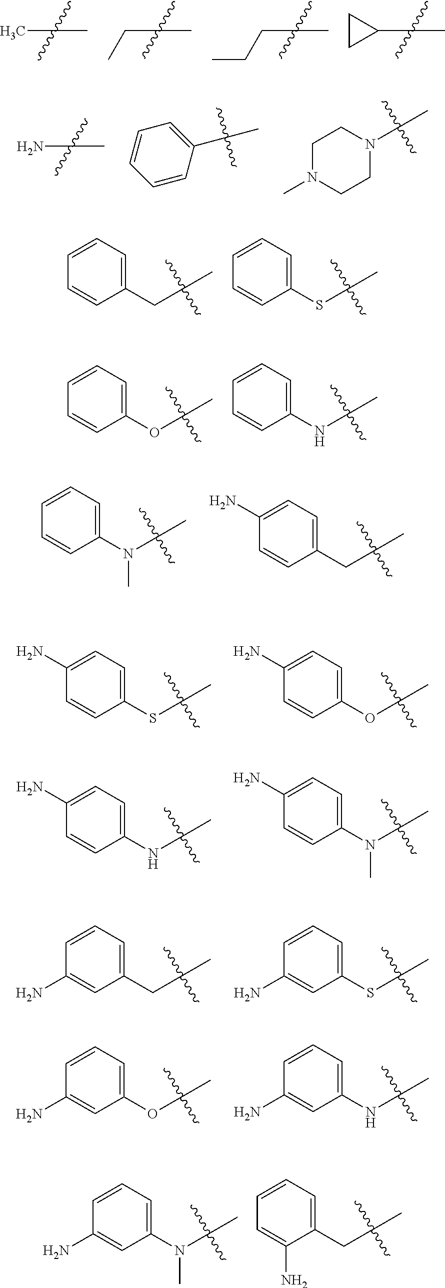

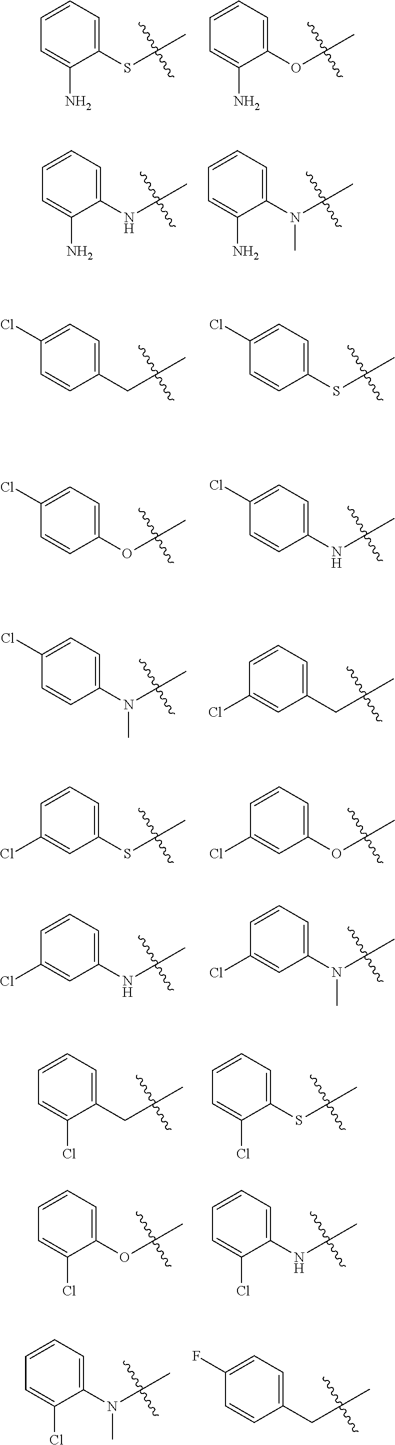

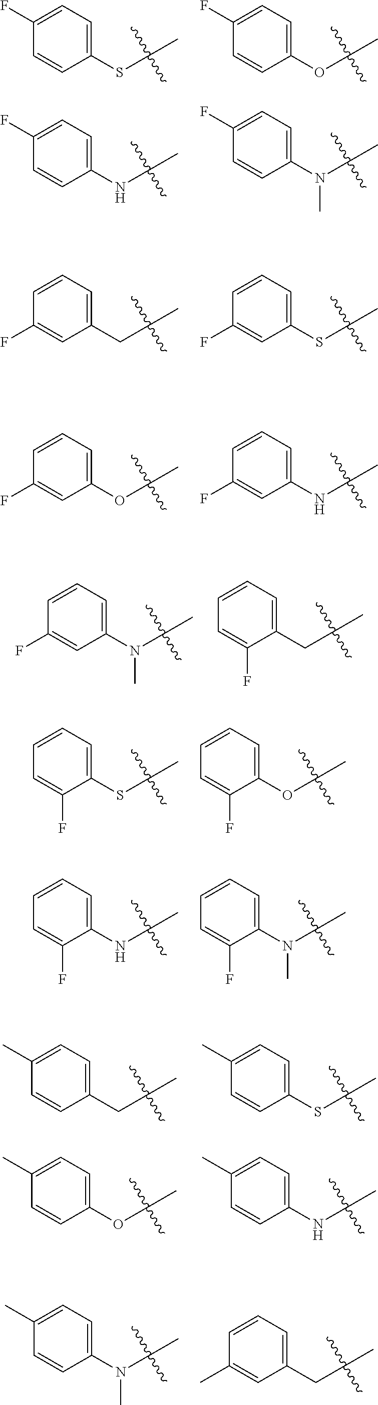

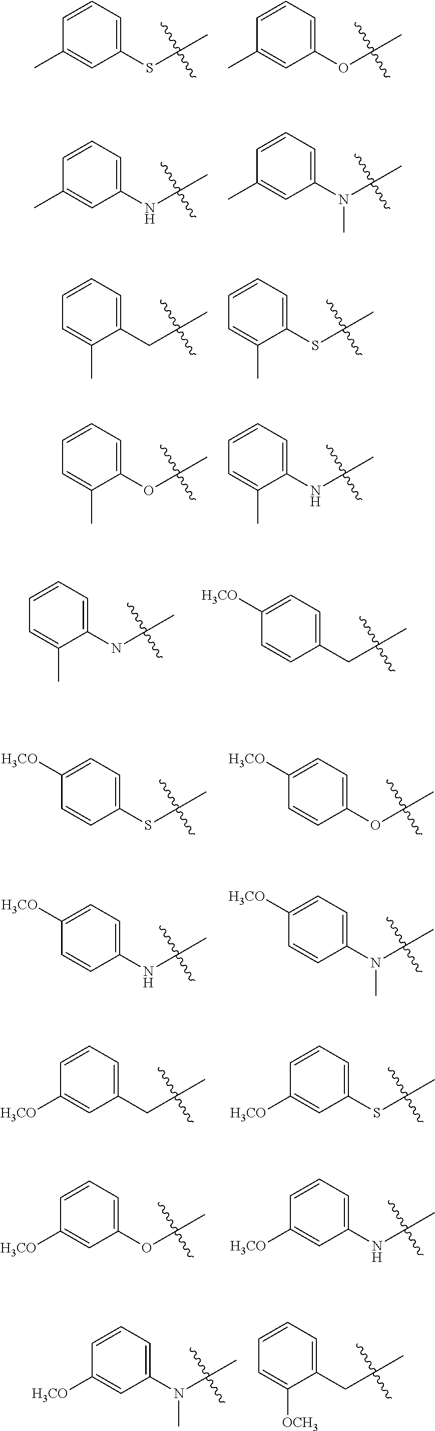

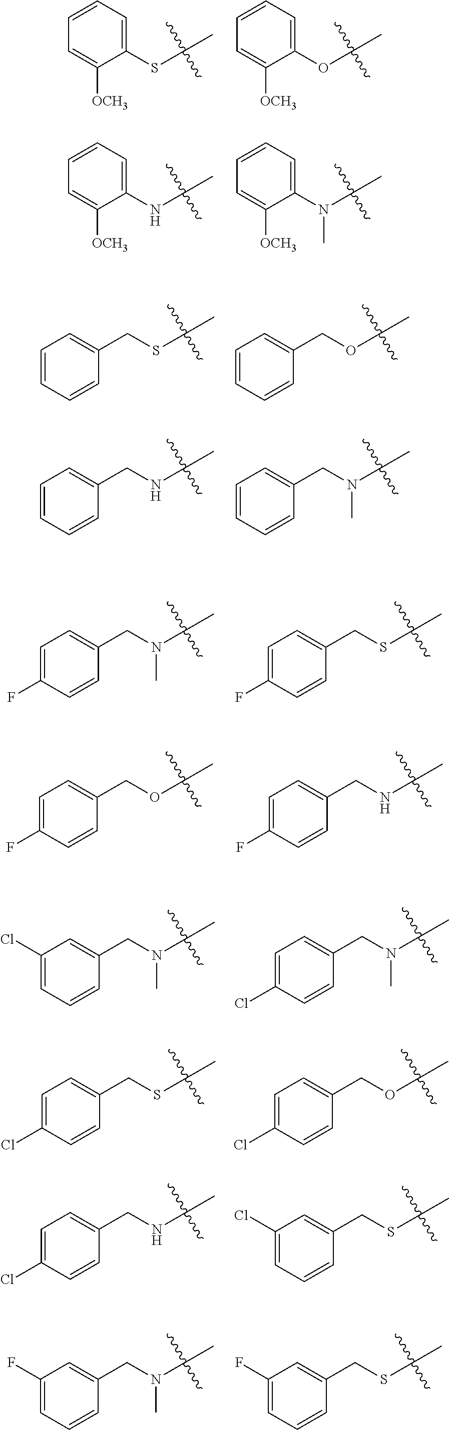

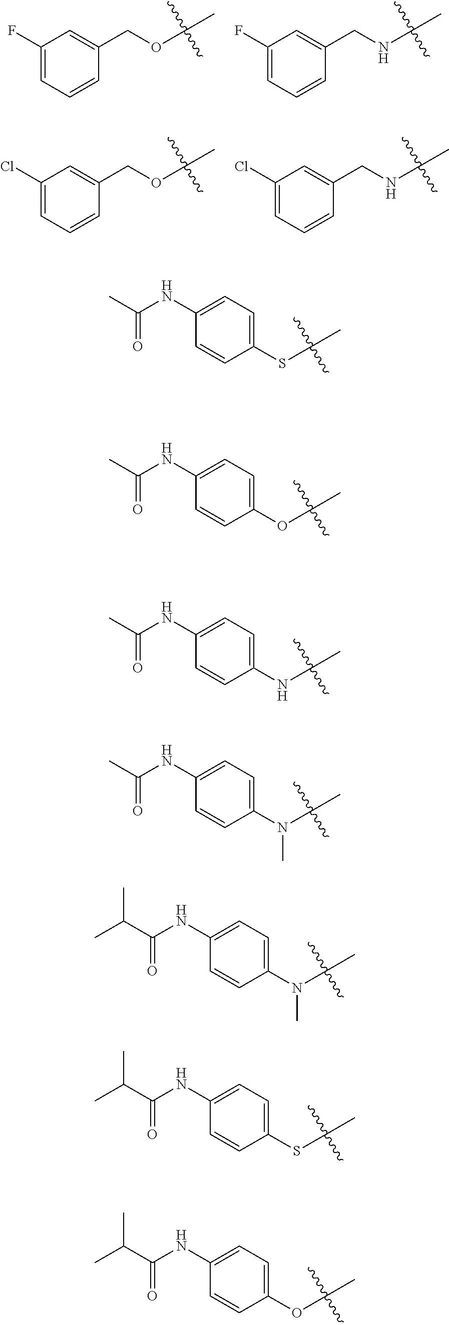

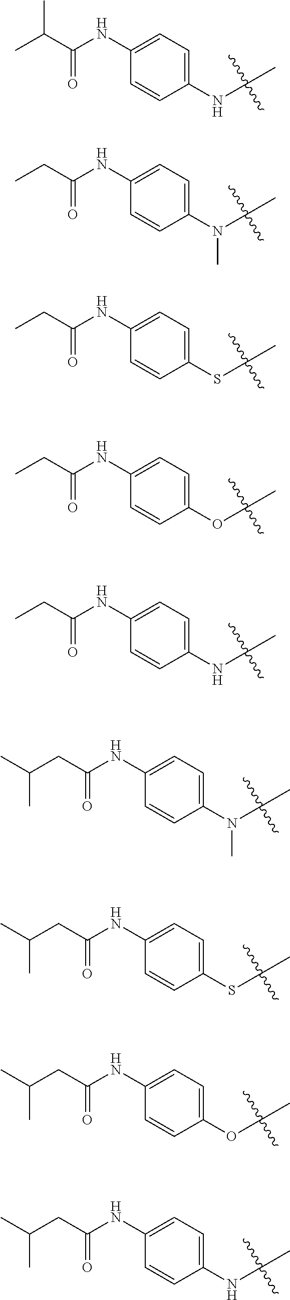

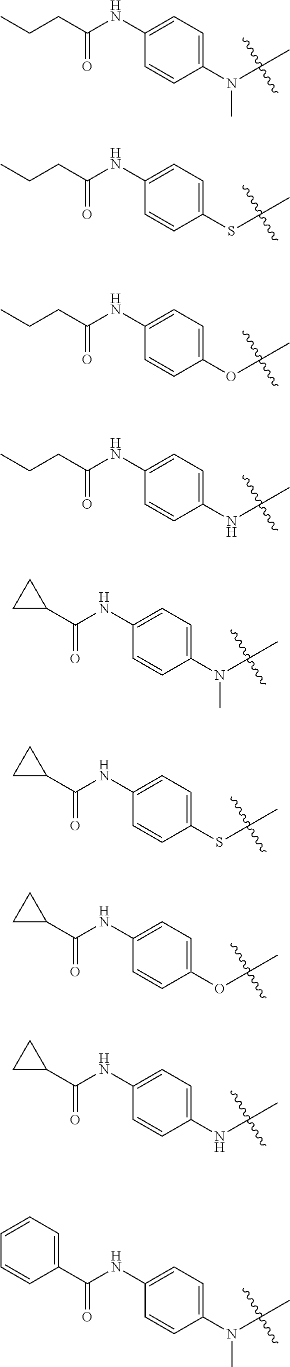

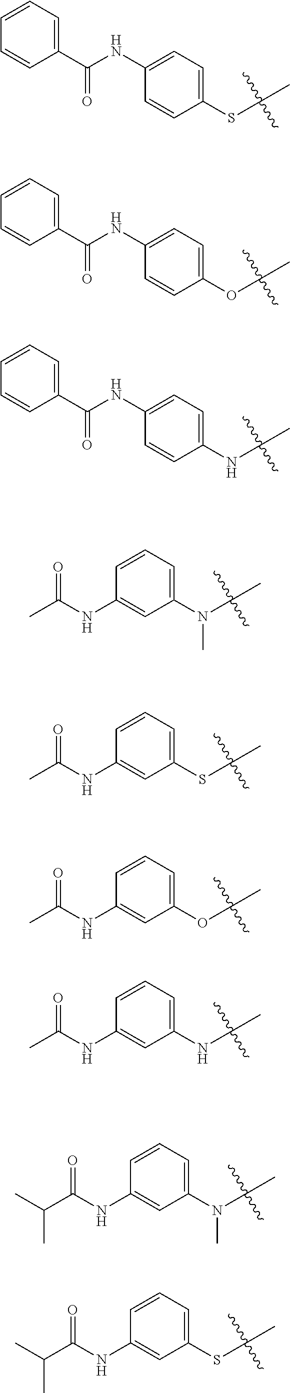

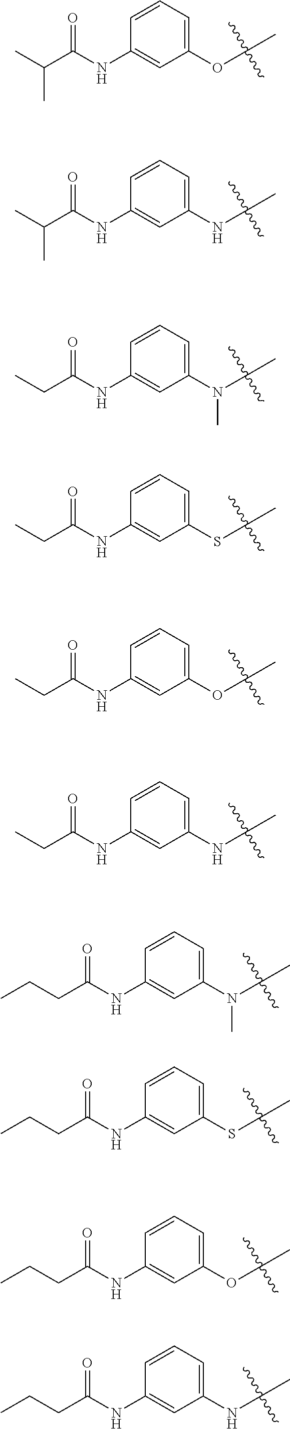

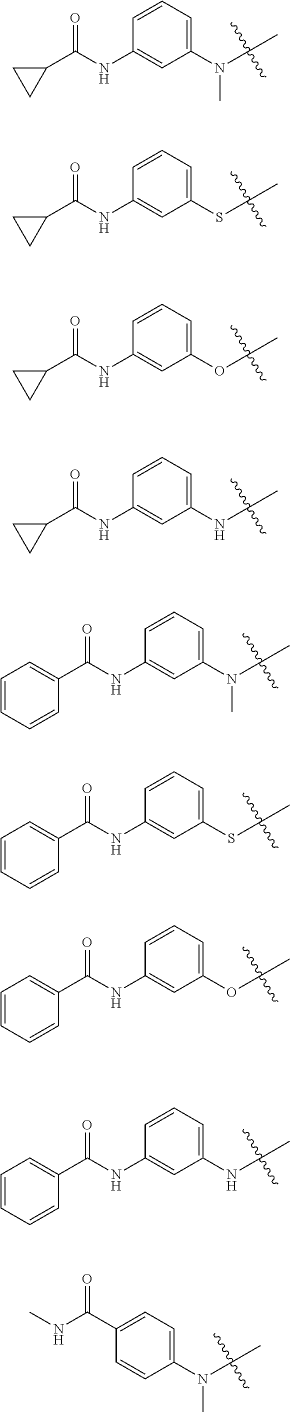

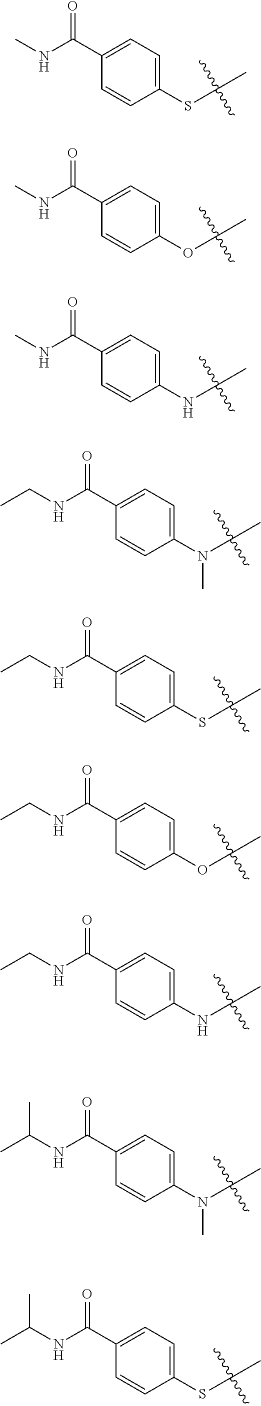

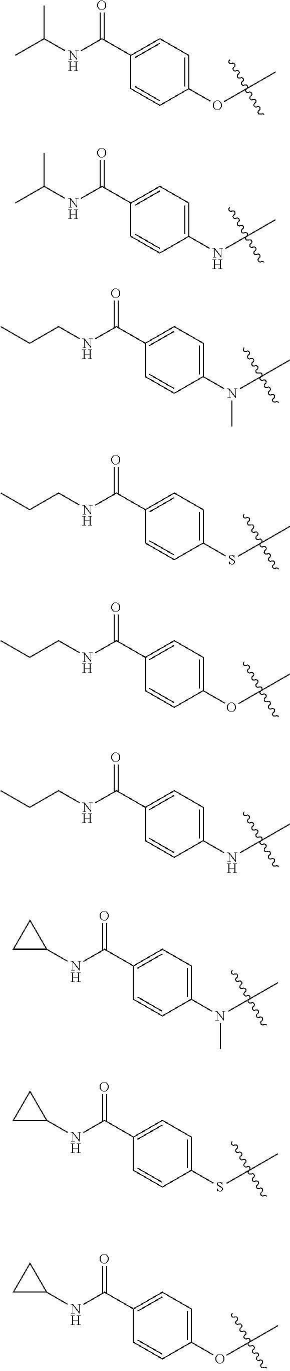

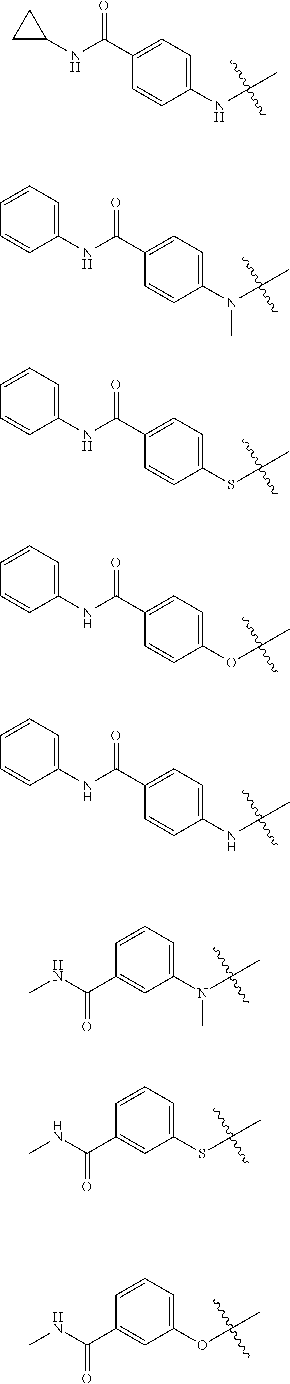

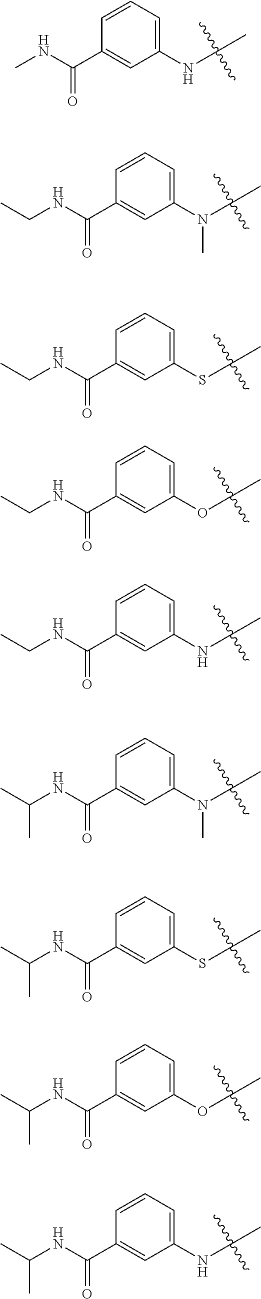

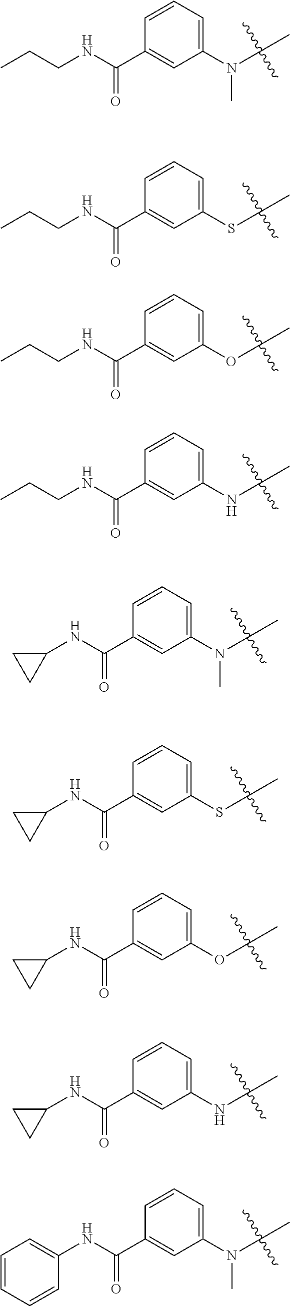

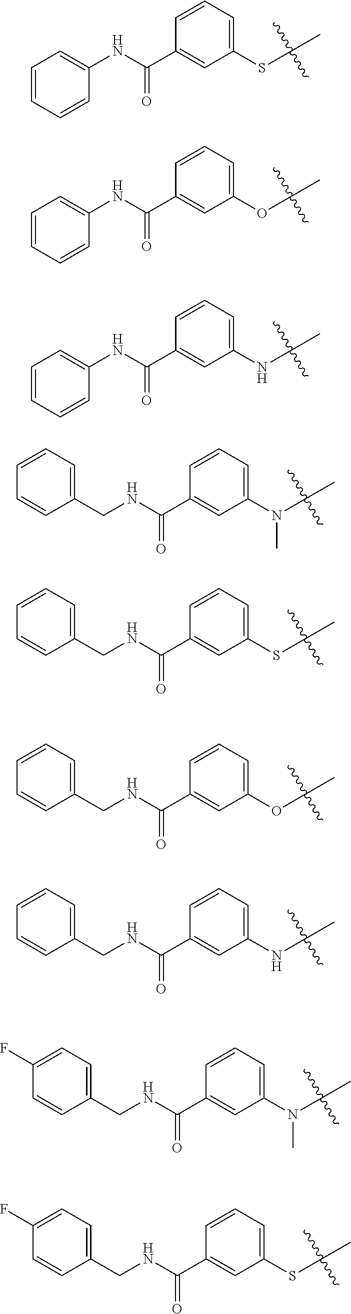

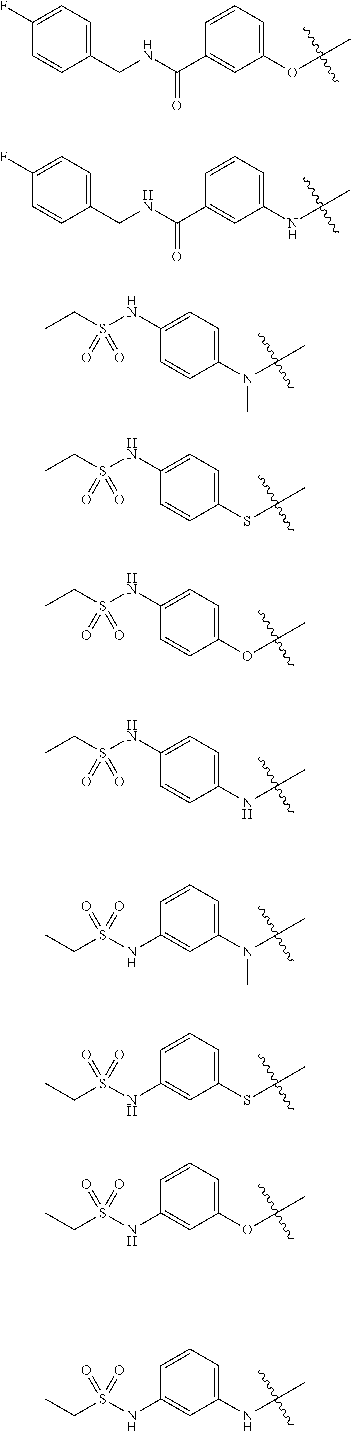

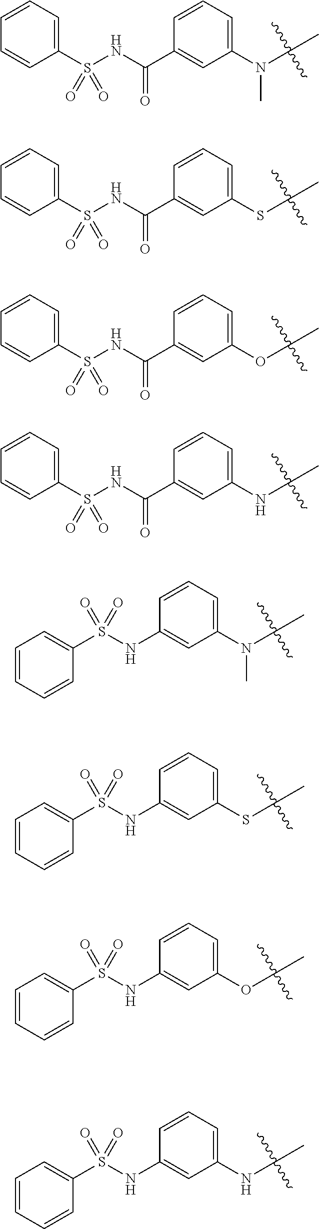

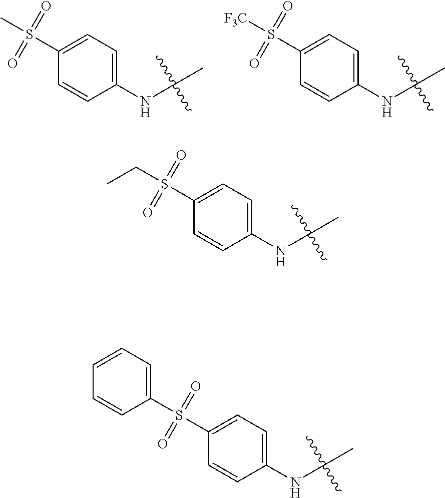

R₄ is independently selected from hydrogen or an optionally substituted C₁₋₄ aliphatic group.

Preferably, the compounds of the invention may be compounds of formula (I) wherein

R₁ groups of formula (I) are listed below:

—H, —CH₃, —CH₂CH₃, —CH₂CH₂CH₃, —CH₂CH₂CH₂CH₃, iso-propyl, cyclopropyl, cyclobutyl, tert-butyl, —CH₂OH, —COOCH₂CH₃, —Cl, —F, —Br.

W and Y are independently selected from S, O, NR₄, CR₄ or CR₁;

R₄ is independently selected from hydrogen or an optionally substituted C₁₋₄ aliphatic group.

n is 1 or 2.

R₂ is selected from:
(i) amino, alkyl amino, aryl amino, heteroaryl amino;
(ii) C₁-C₆ alkyl, C₂-C₆ alkenyl, C₂-C₆ alkynyl;
(iii) aryl, heterocyclic, heteroaryl; and
(iv) groups of the formula (Ia):

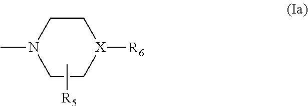

wherein:

R₅ represents hydrogen, C₁-C₄ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl)sulfonamido, and mono- and di-(C₁-C₆alkyl)aminocarbonyl, each of which is substituted with from 0 to 4 substituents independently chosen from halogen, hydroxy, cyano, amino, —COOH and oxo;

R₃ is selected from:

(i) C₁-C₆ alkyl, C₂-C₆ alkenyl, C₂-C₆ alkynyl;

(ii) heterocyclic,

(iii) K—Ar.

Ar represents heteroaryl or aryl, each of which is substituted with from 0 to 4 substituents independently chosen from:
(1) halogen, hydroxy, amino, amide, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl)sulfonamido and mono- and di-(C₁-C₆alkyl)aminocarbonyl; phenylC₀-C₄alkyl and (4- to 7-membered heterocycle)-(C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

K is selected from

i) absence;

ii) O, S, SO, SO₂;

iii) (CH₂)ₘ, m=0-3, —O(CH₂)ₚ, p=1-3, —S(CH₂)ₚ, p=1-3, —N(CH₂)ₚ, p=1-3, —(CH₂)ₚO, p=1-3;

iv) NR₇

R₇ represents hydrogen, alkyl, cycloalkyl, alkenyl, alkynyl, alkylthio, aryl, arylalkyl.

More preferably, the compounds of the invention may be compounds of formula (I) wherein

R₁ represents —H, —Cl, —CH₃, —CH₂CH₃, —CH₂CH₂CH₃, cyclopropyl, cyclobutyl, —CH₂CH(CH₃)₂, —CH(CH₃)₃, Ph.

W and Y are independently selected from S, O, NR₄, or CR₄;

R₄ is independently selected from hydrogen or an optionally substituted C₁₋₄ aliphatic group.

n is 1;

R₂ is selected from:

(i) amino, alkyl amino, aryl amino, heteroaryl amino;

(ii) C₁-C₆ alkyl, C₂-C₆ alkenyl, C₂-C₆ alkynyl;

(iii) aryl, heterocyclic, heteroaryl; and

(iv) groups of the formula (Ia):

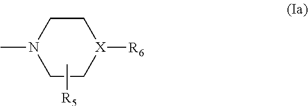

wherein:

R₅ represents hydrogen, C₁-C₄ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl)sulfonamido, and mono- and di-(C₁-C₆alkyl)aminocarbonyl, each of which is substituted with from 0 to 4 substituents independently chosen from halogen, hydroxy, cyano, amino, —COOH and oxo;

R₃ is selected from:

(i) C₁-C₀ alkyl, C₂-C₆ alkenyl, C₂-C₆ alkynyl;

(ii) heterocyclic,

(iii) K—Ar.

Ar represents heteroaryl or aryl, each of which is substituted with from 0 to 4 substituents independently chosen from:
(1) halogen, hydroxy, amino, amide, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl)sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

K is selected from

i) absence;

ii) O, S,

(iii) ((CH₂)ₘ, m=0-3, —O(CH₂)ₚ, p=1-3, —S(CH₂)ₚ, p=1-3, —N(CH₂)ₚ, p=1-3, —(CH₂)ₚ0, p=1-3;

iv) NR₇

R₇ represents hydrogen, alkyl, cycloalkyl, alkenyl, alkynyl, alkylthio, aryl, arylalkyl.

Most preferably, the compounds of the invention may be compounds of formula (I) wherein

R₁ represents —Cl, —CH₃, —CH₂CH₃, —CH₂CH₂CH₃, cyclopropanyl, cyclobutyl, —CH₂CH(CH₃)₂, —CH(CH₃)₃.

W and Y are independently selected from S, NR₄, or CR₄;

R₄ is independently selected from hydrogen or an optionally substituted C₁₋₄ aliphatic group.

n is 1;

R₂ is selected from:

(i) amino, alkyl amino, aryl amino, heteroaryl amino;

(ii) C₁-C₆ alkyl, C₂-C₆ alkenyl, C₂-C₆ alkynyl;

(iii) groups of the formula (Ia):

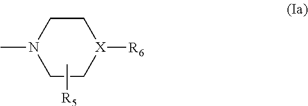

wherein:

R₅ represents hydrogen, C₁-C₁ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl)sulfonamido, and mono- and di-(C₁-C₆alkyl)aminocarbonyl, each of which is substituted with from 0 to 4 substituents independently chosen from halogen, hydroxy, cyano, amino, —COOH and oxo;

R₃ is selected from:

K—Ar.

Ar represents heteroaryl or aryl, each of which is substituted with from 0 to 4 substituents independently chosen from:
(1) halogen, hydroxy, amino, amide, cyano, and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl)sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

K is selected from

(i) O, S,

(ii) —O(CH₂)ₚ, p=1-3, —S(CH₂)ₚ, p=1-3, —N(CH₂)ₚ, p=1-3;

(iii) NR₇

R₇ represents hydrogen, alkyl.

Preferred heterocyclic groups in compounds of formula (I) include

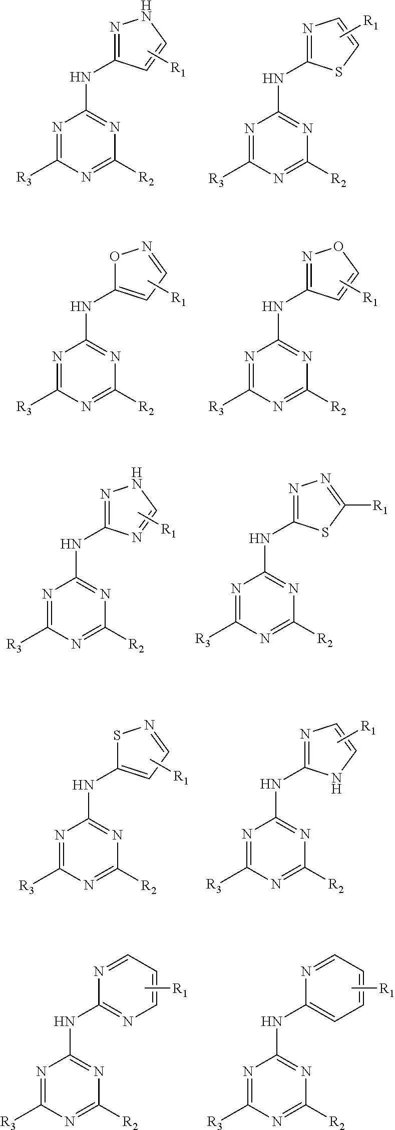

Which optionally may be substituted.

According to another embodiment, the present invention relates to a compound of formula (I) wherein R₁ is hydrogen.

According to another embodiment, the present invention relates to a compound of formula (I) wherein R₁ is chloro.

According to another embodiment, the present invention relates to a compound of formula (I) wherein R₁ is methyl.

According to another embodiment, the present invention relates to a compound of formula (I) wherein R₁ is ethyl.

According to another embodiment, the present invention relates to a compound of formula (I) wherein R₁ is propyl.

According to another embodiment, the present invention relates to a compound of formula (I) wherein R₁ is isopropyl.

According to another embodiment, the present invention relates to a compound of formula (I) wherein R₁ is isobutyl.

According to another embodiment, the present invention relates to a compound of formula (I) wherein R₁ is tert-butyl.

According to another embodiment, the present invention relates to a compound of formula (I) wherein R₁ is cyclopropyl.

According to another embodiment, the present invention relates to a compound of formula (I) wherein R₁ is cyclobutyl.

According to another embodiment, the present invention relates to a compound of formula (I) wherein R₂ is methyl-piperazinyl.

According to another embodiment, the present invention relates to a compound of formula (I) wherein R₂ is (2-hydroxylethyl)-piperazinyl.

According to another embodiment, the present invention relates to a compound of formula (I) wherein R₂ is (4-pyridinyl)-piperazinyl.

According to another embodiment, the present invention relates to a compound of formula (I) wherein R₂ is methyl.

According to another embodiment, the present invention relates to a compound of formula (I) wherein R₂ is ethyl.

According to another embodiment, the present invention relates to a compound of formula (I) wherein R₂ is cyclopropyl.

Examples of specific compounds of the present invention are those compounds defined in the following:

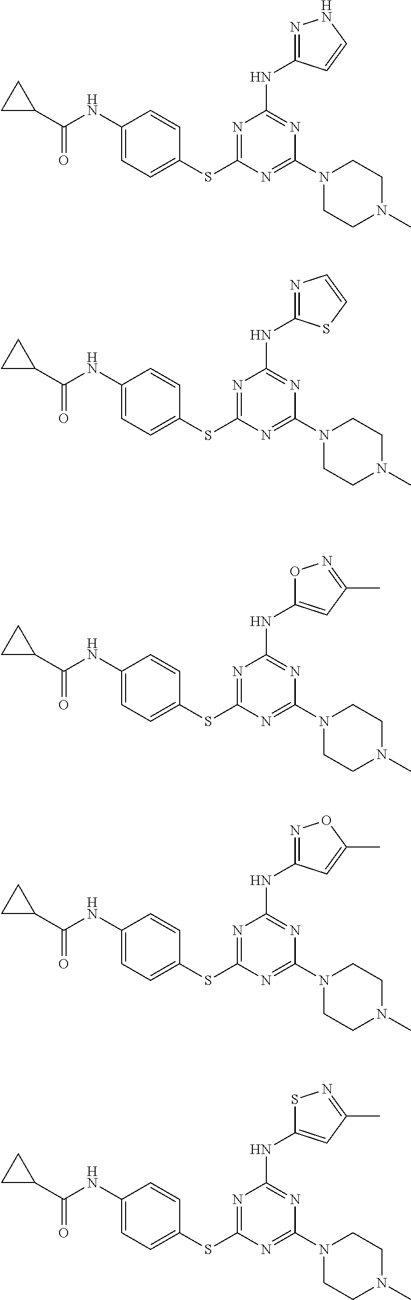

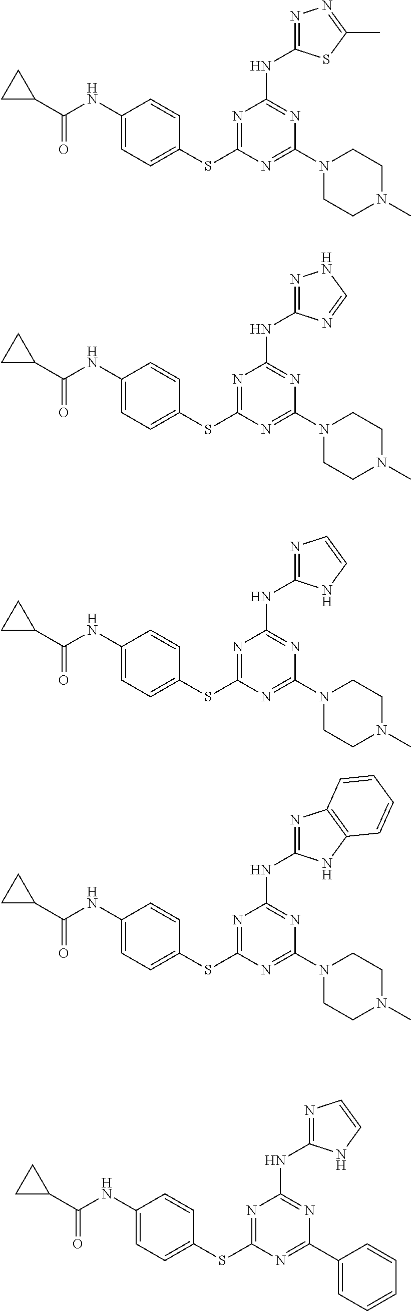

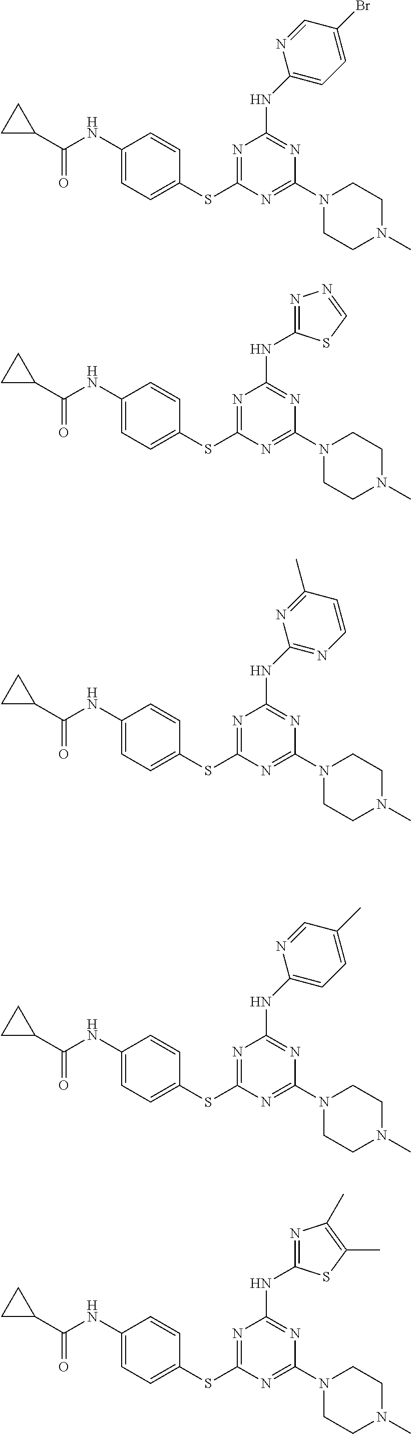

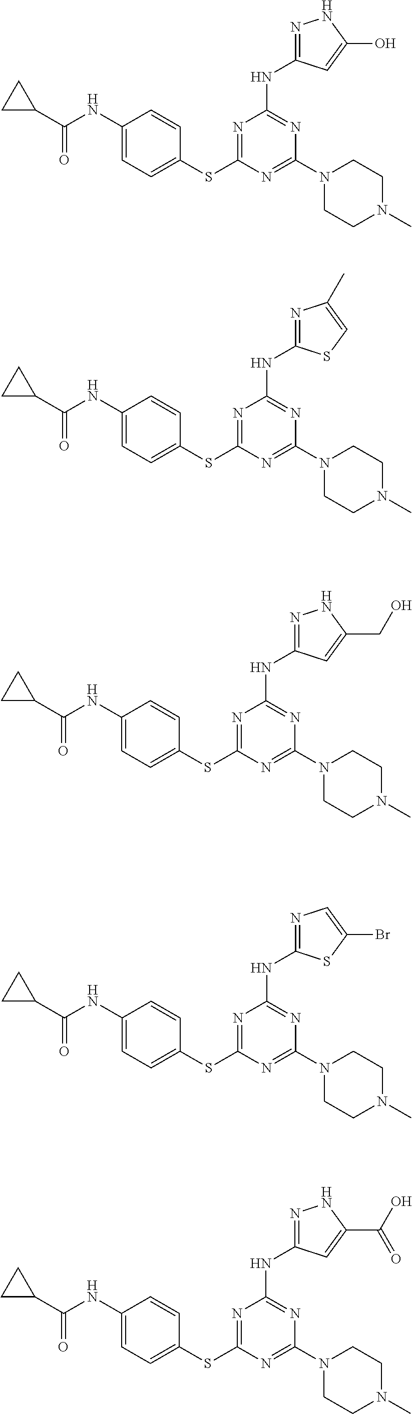

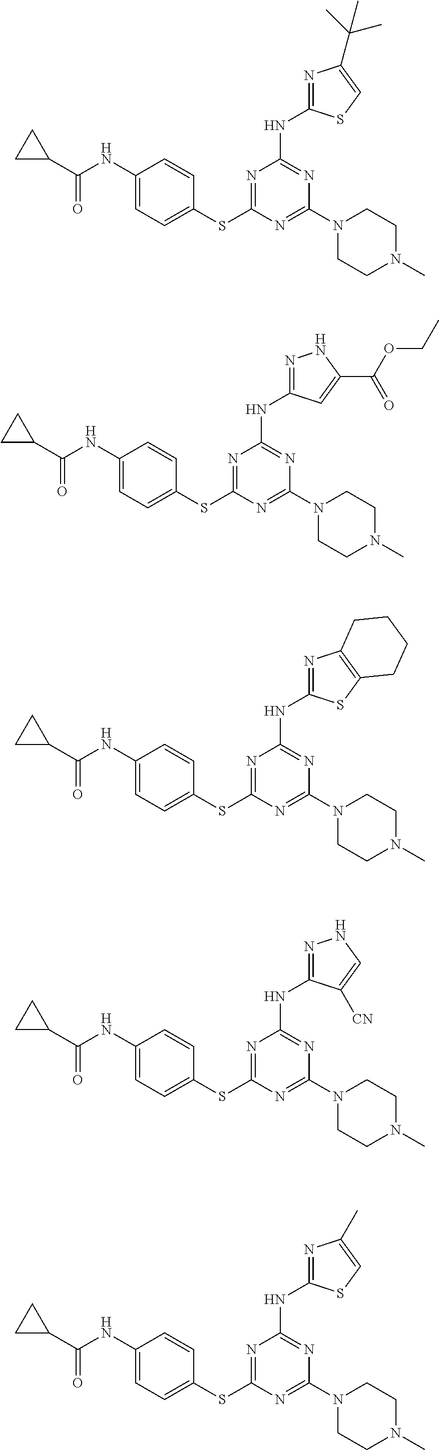

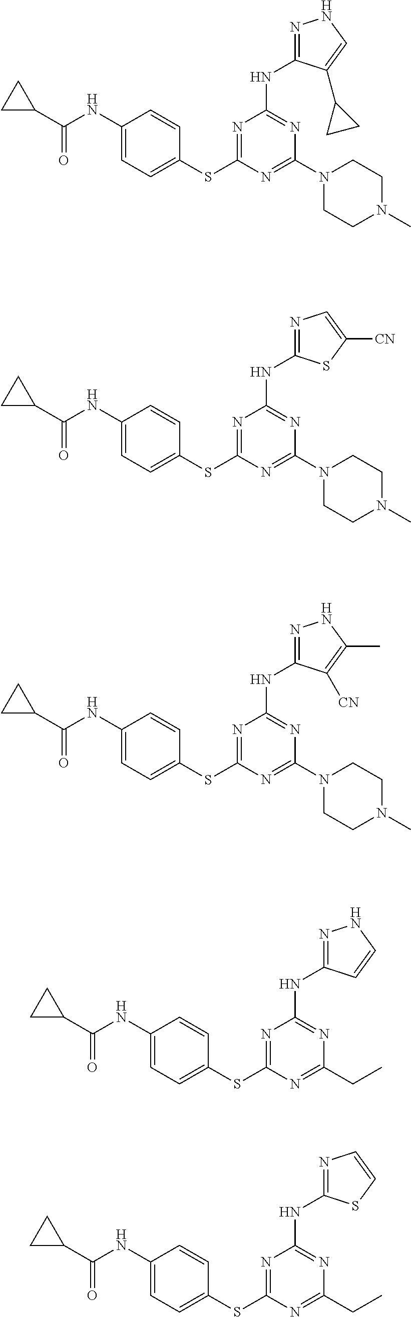

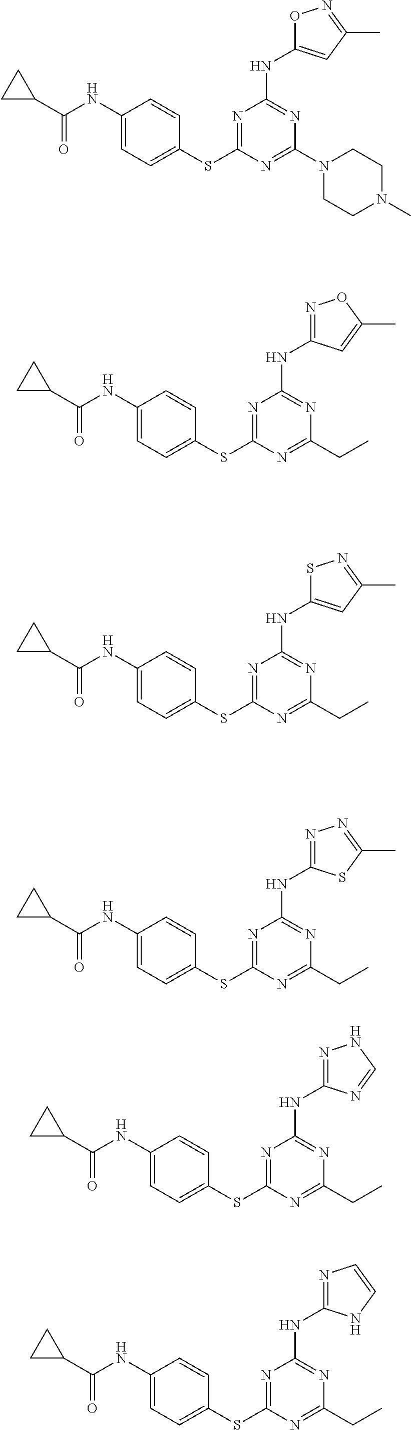

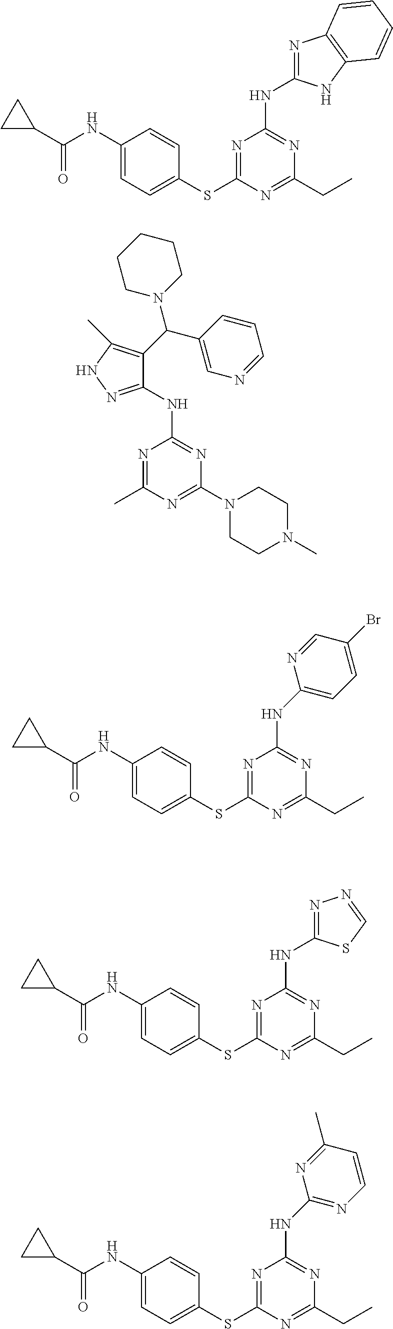

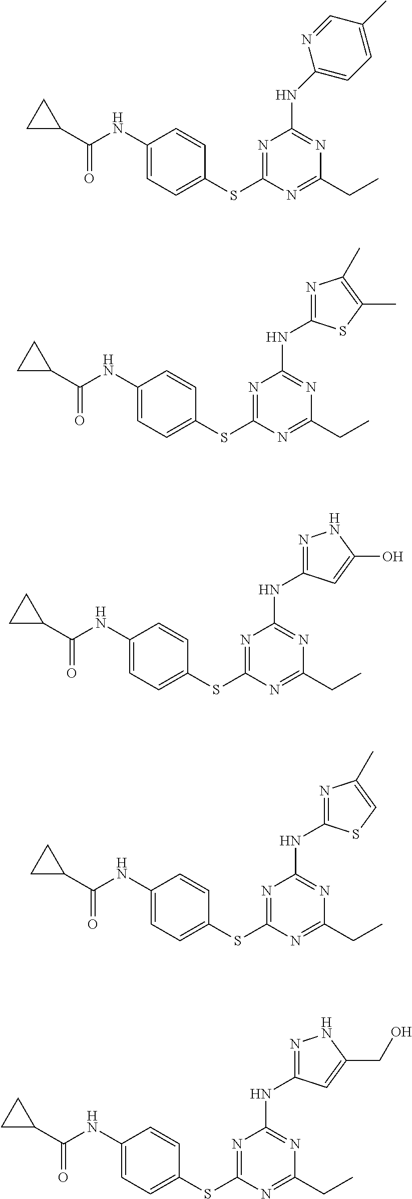

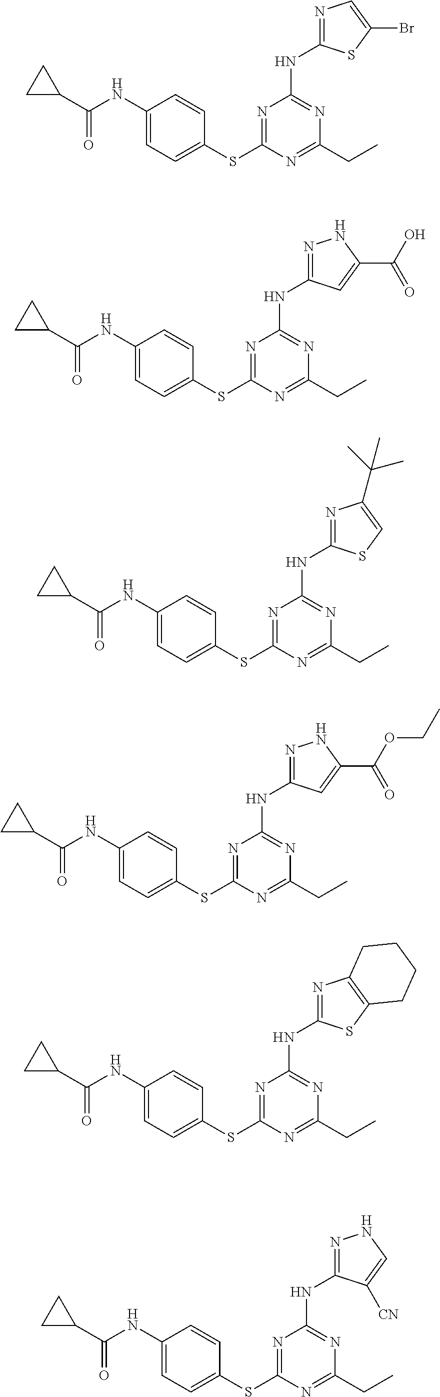

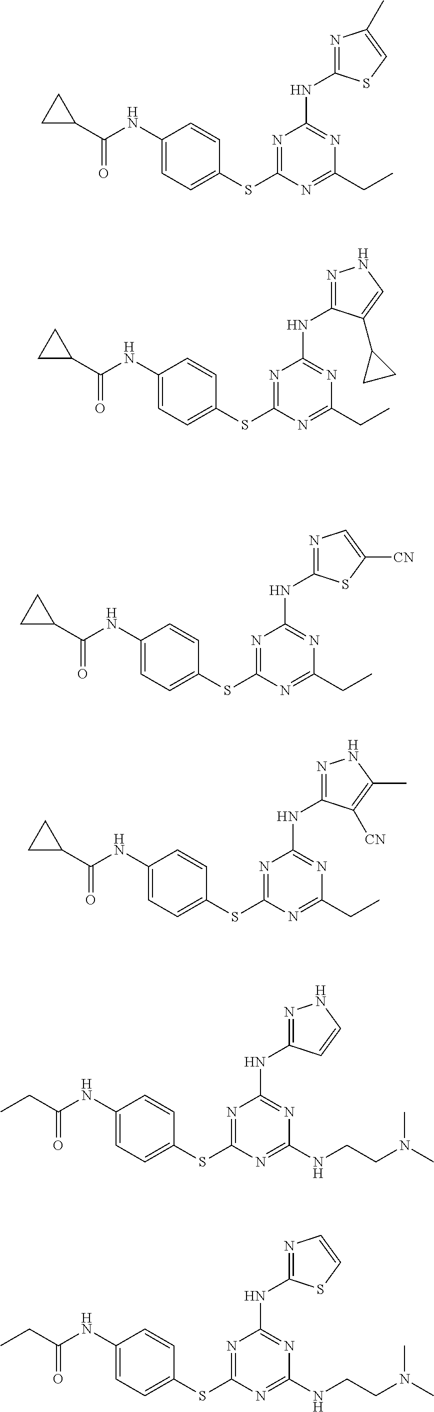

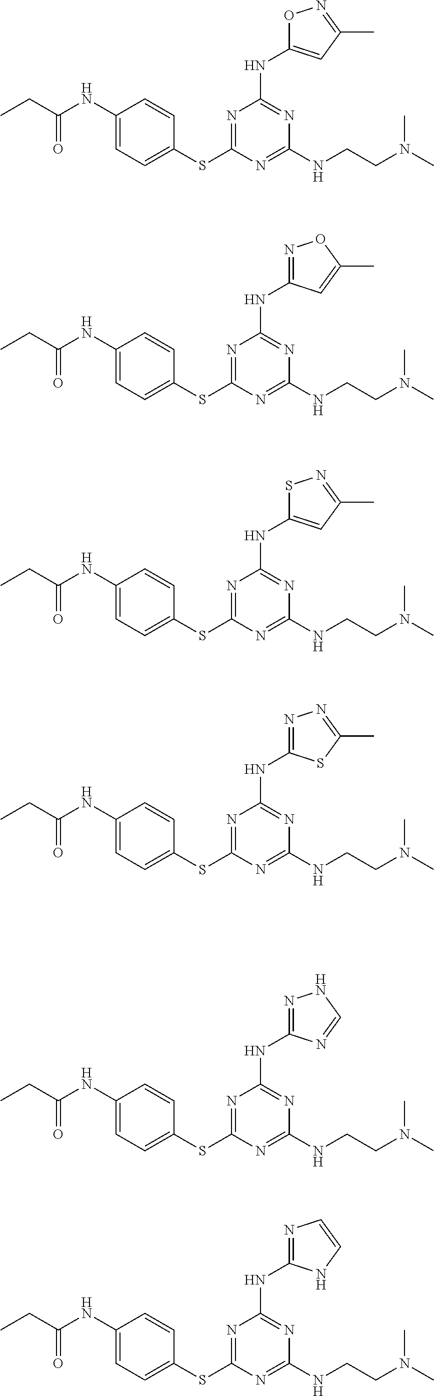

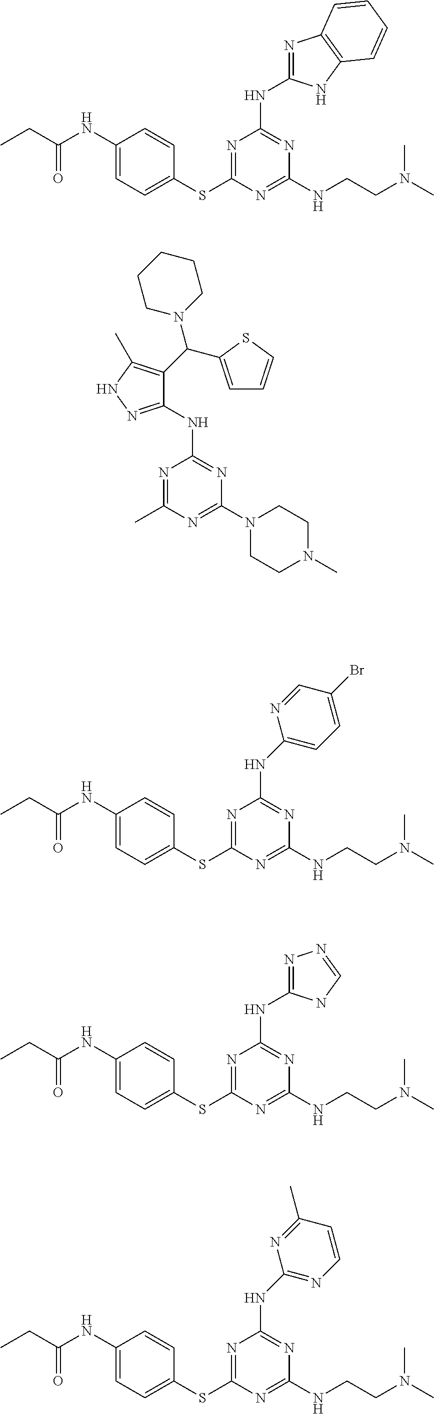

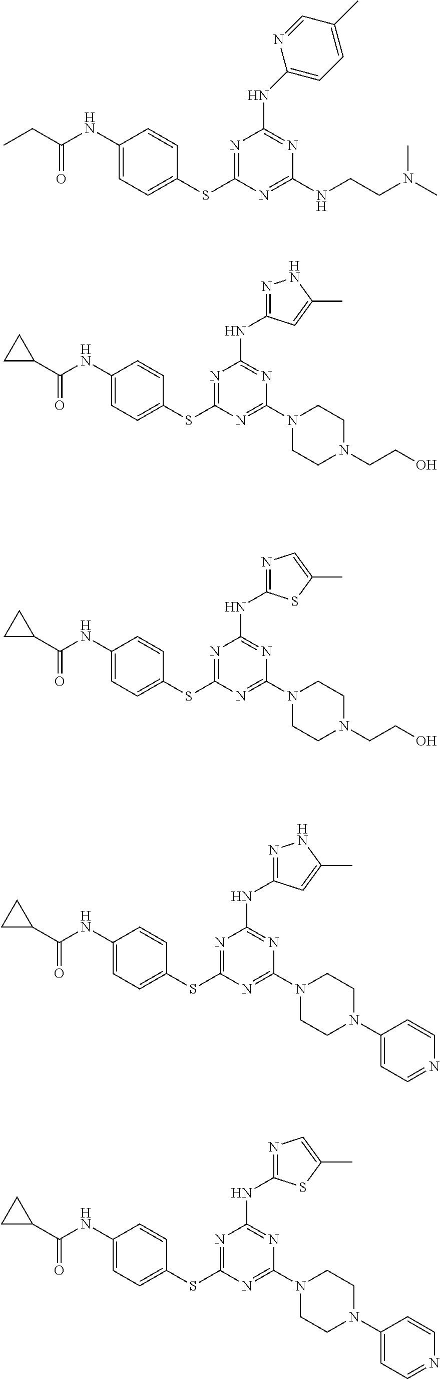

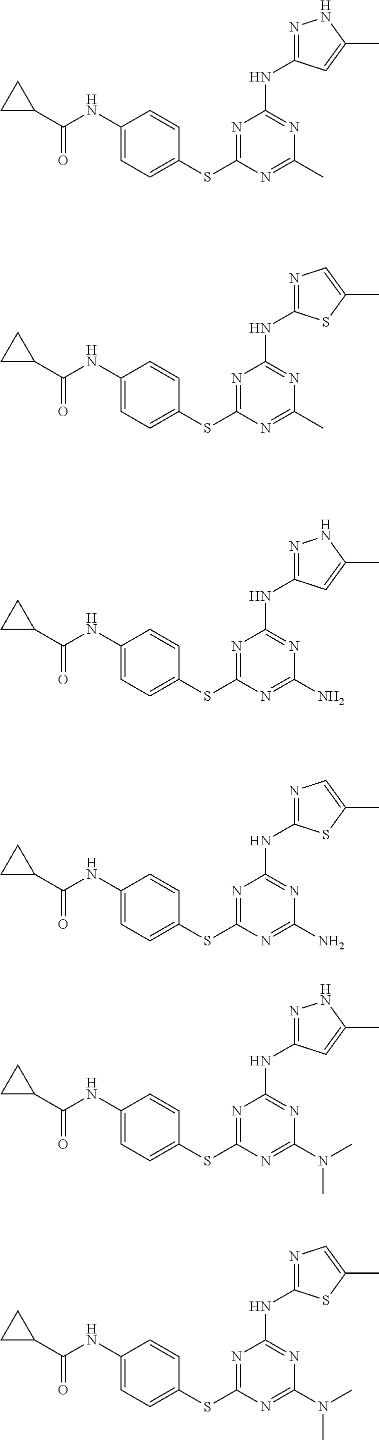

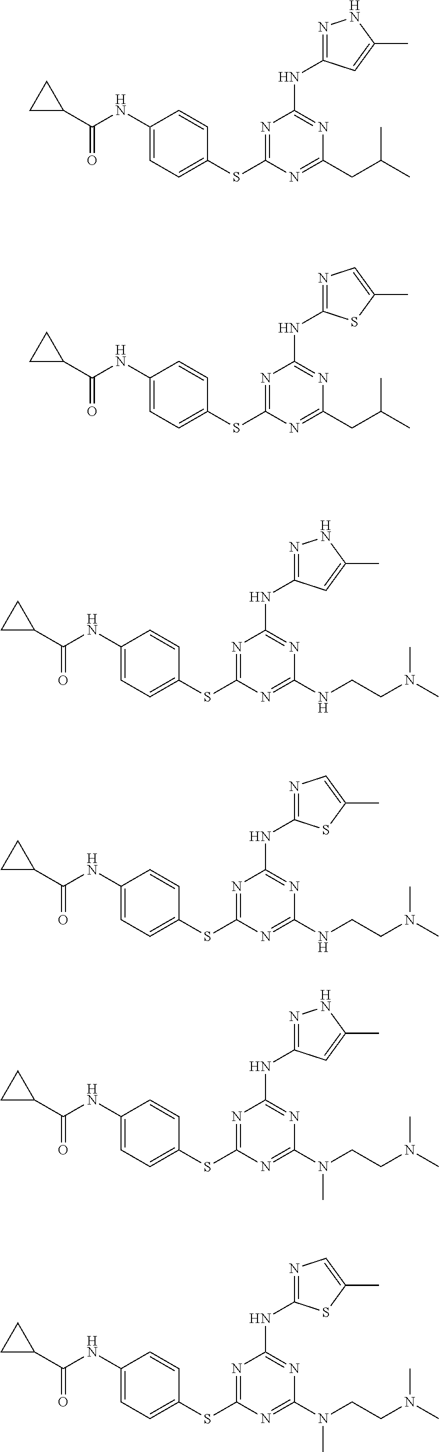

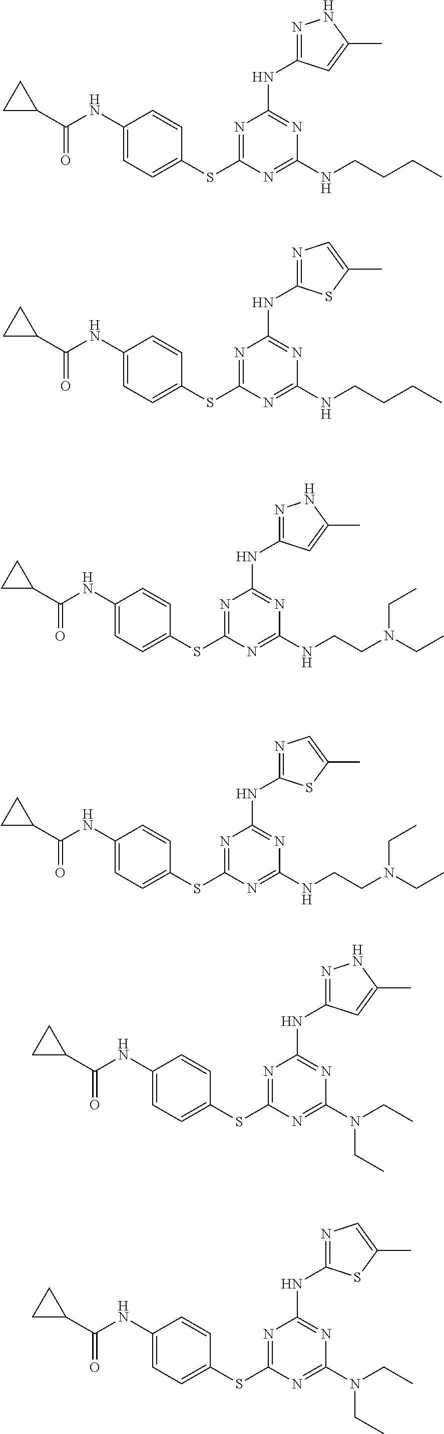

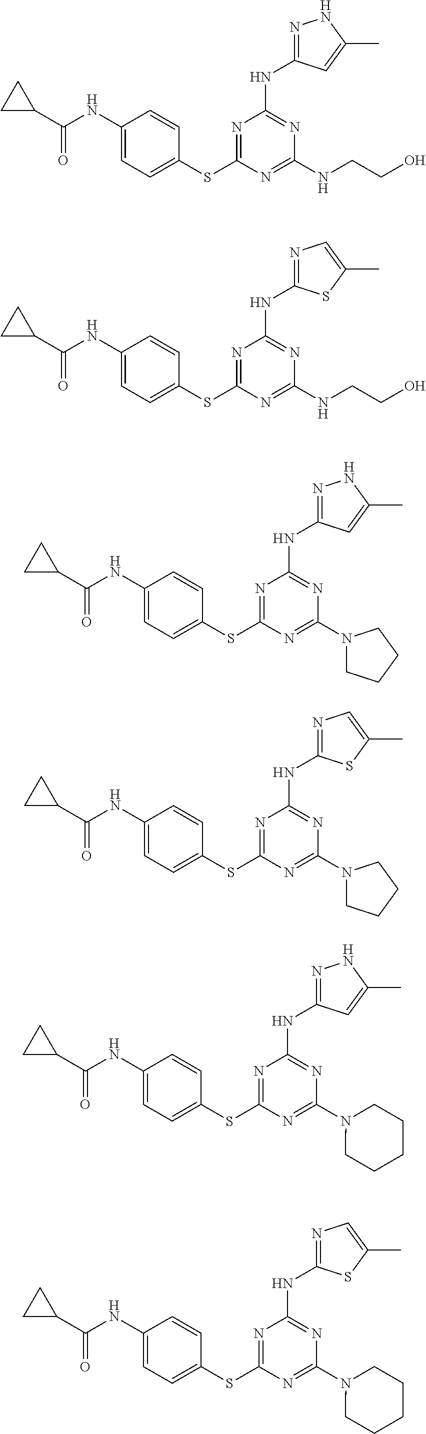

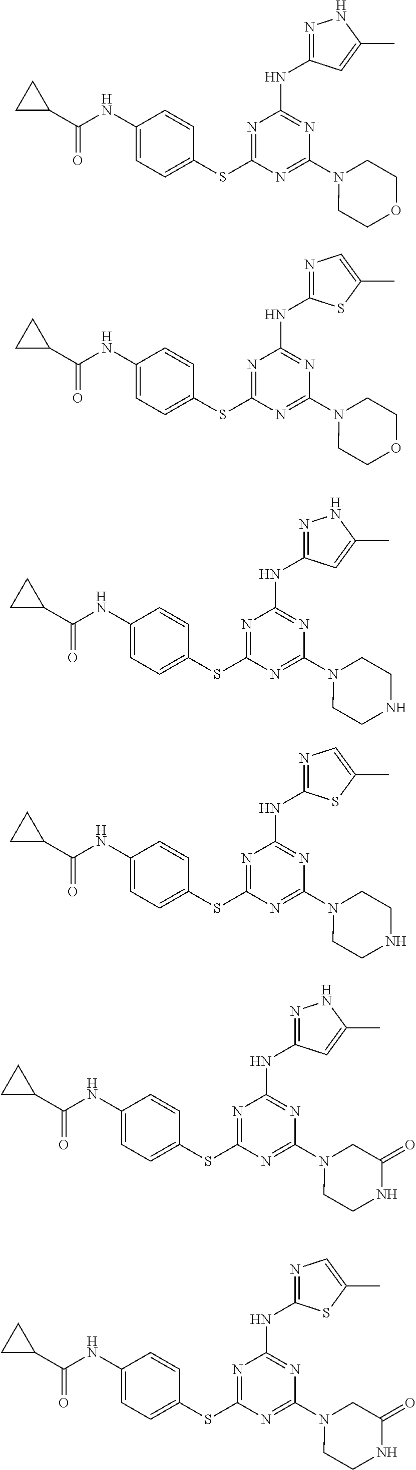

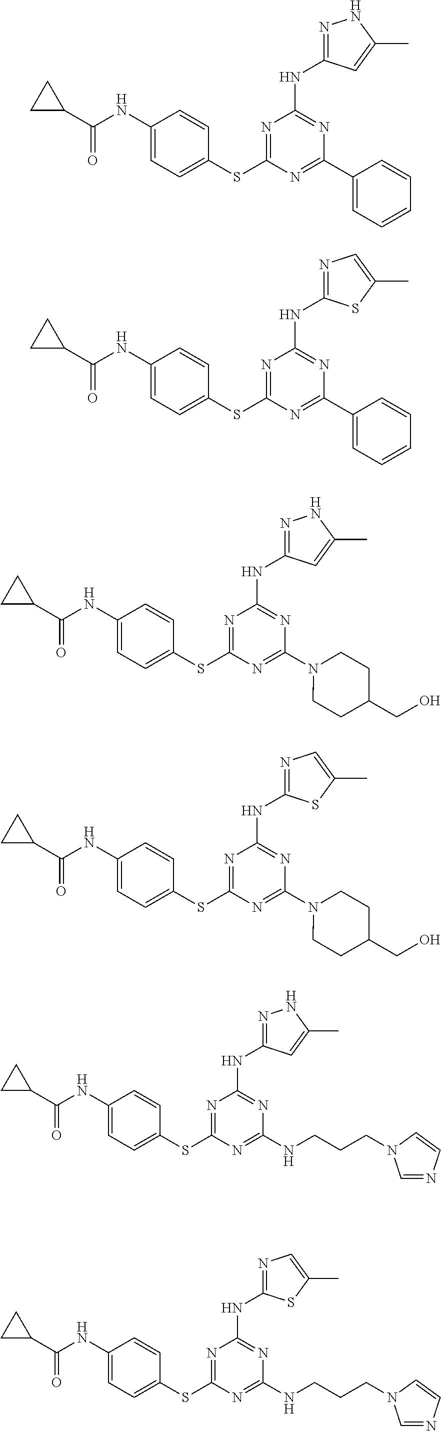

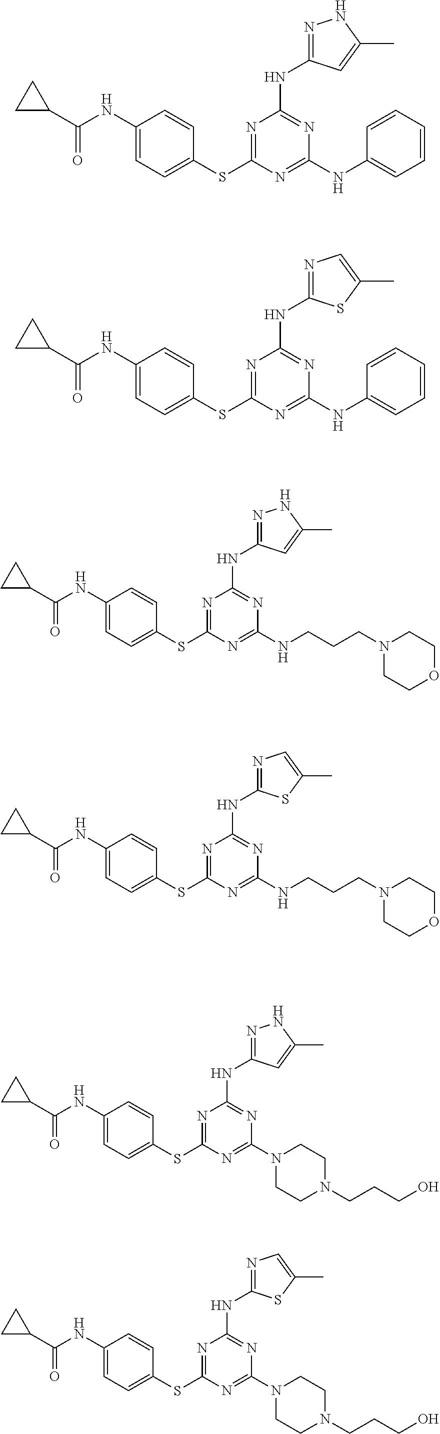

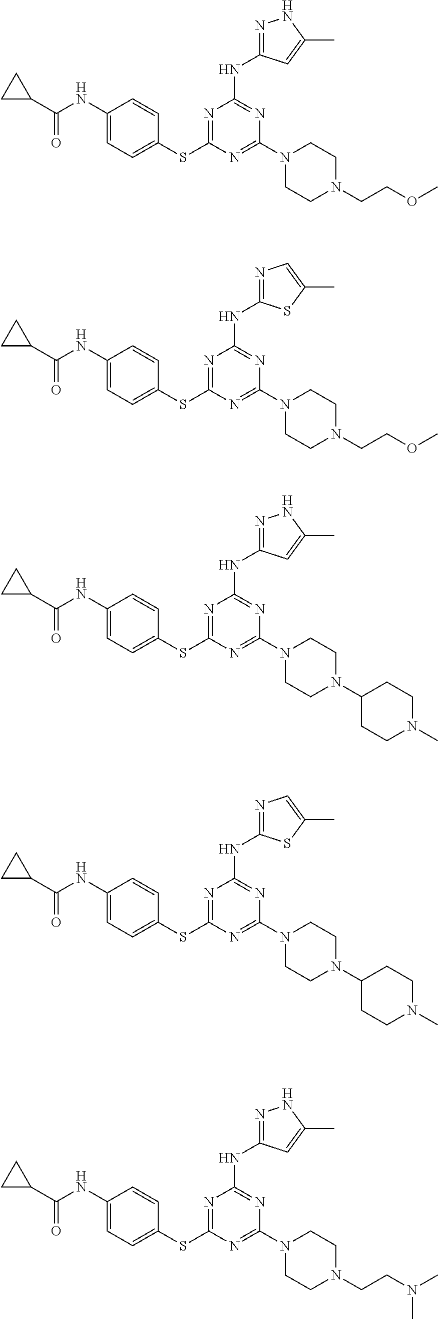

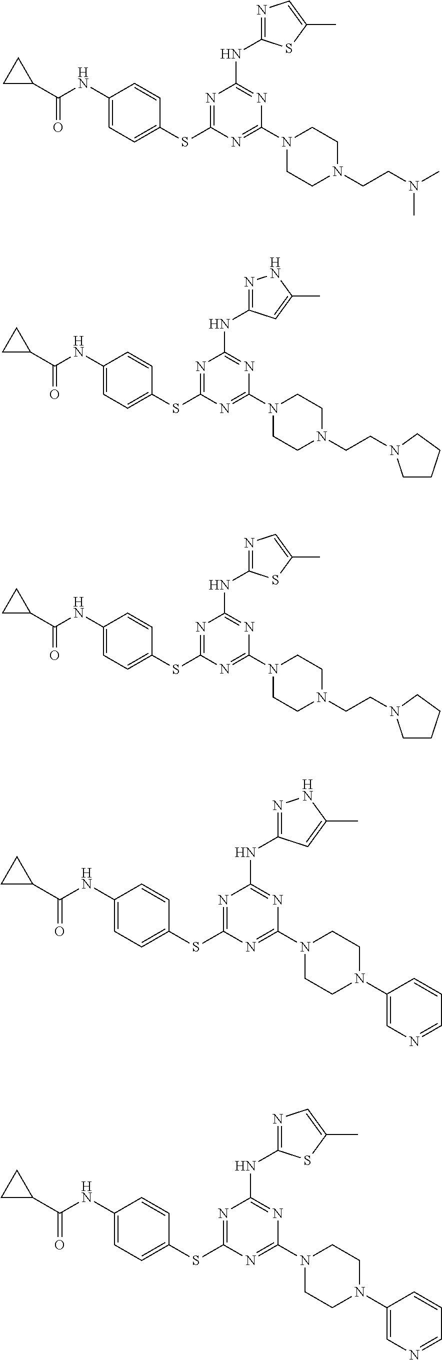

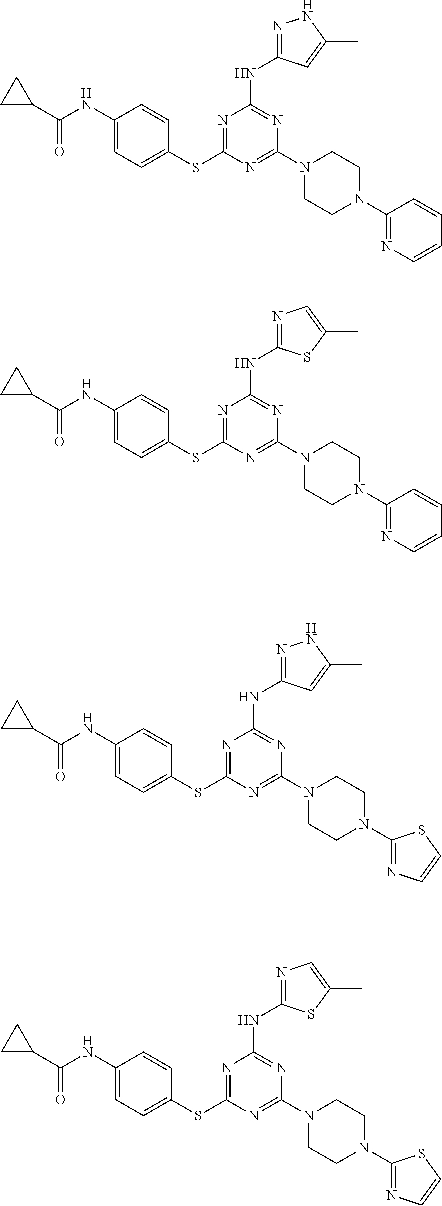

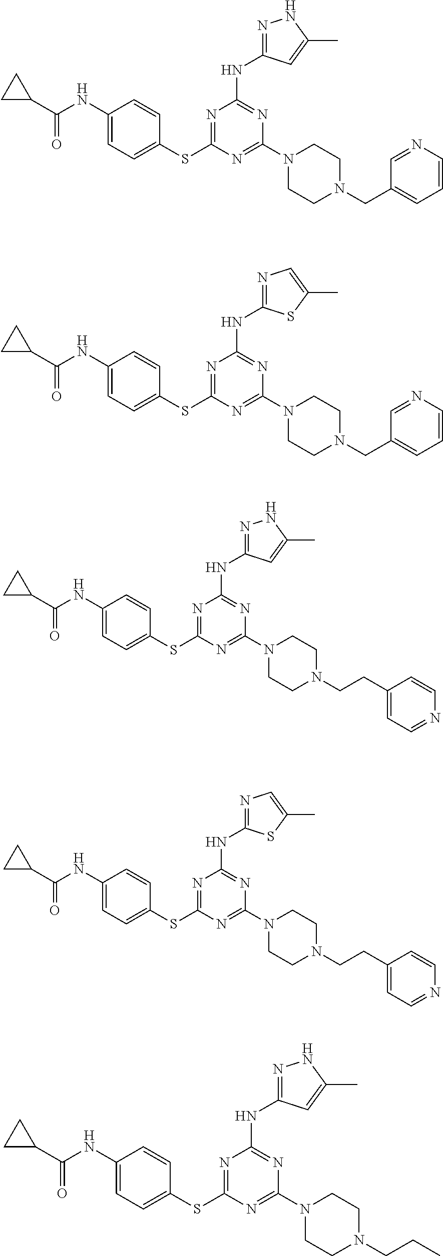

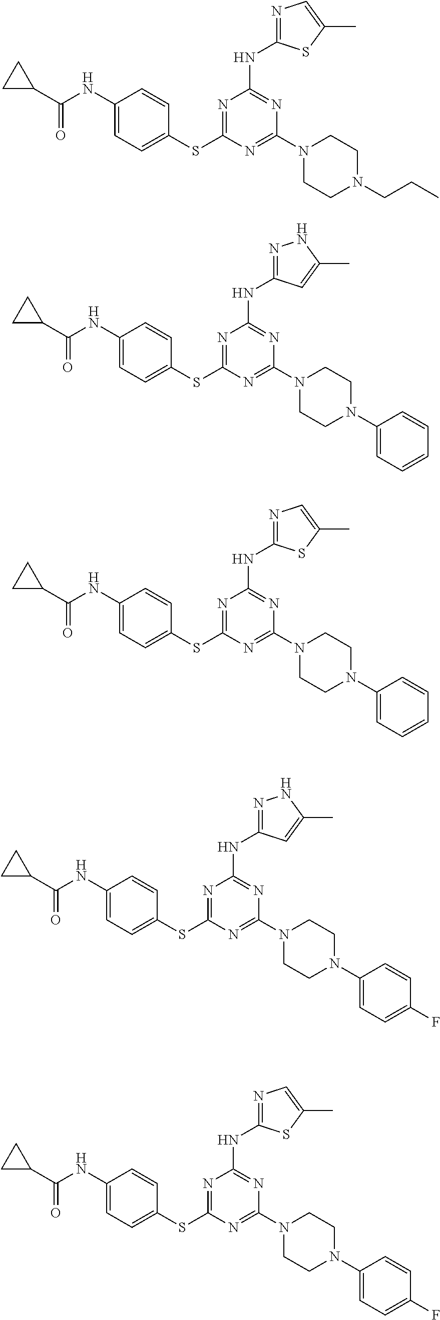

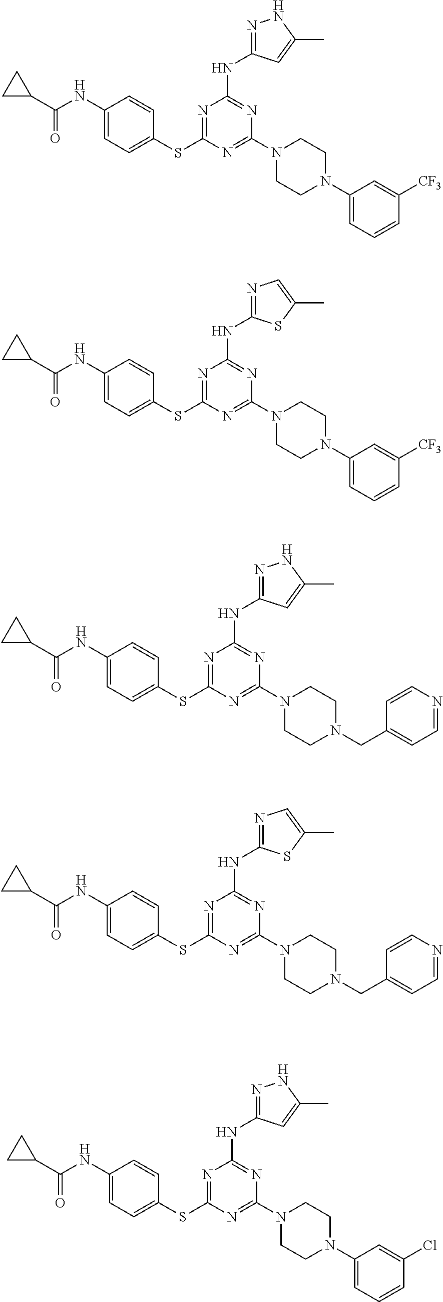

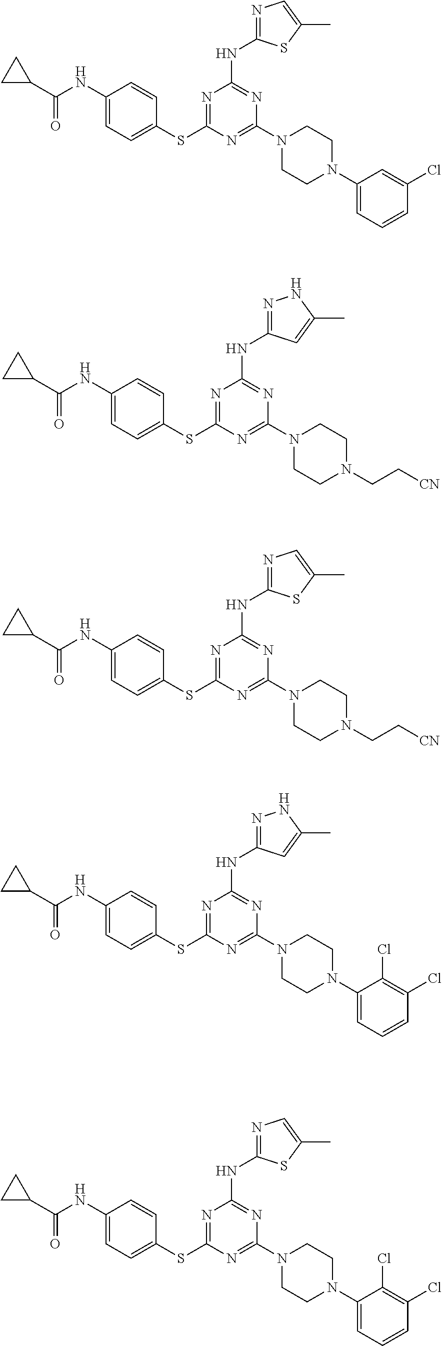

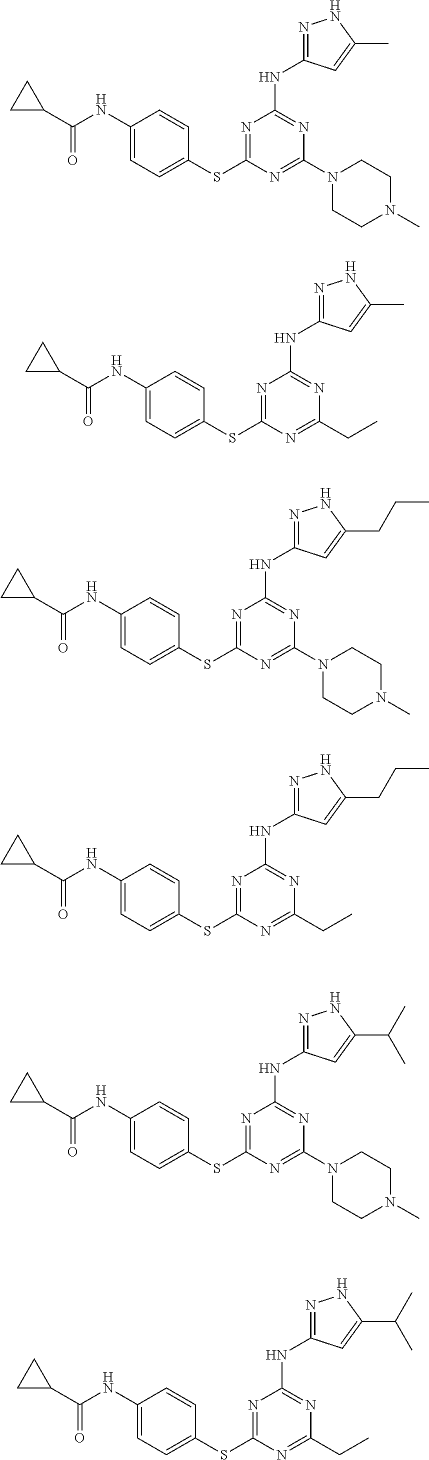

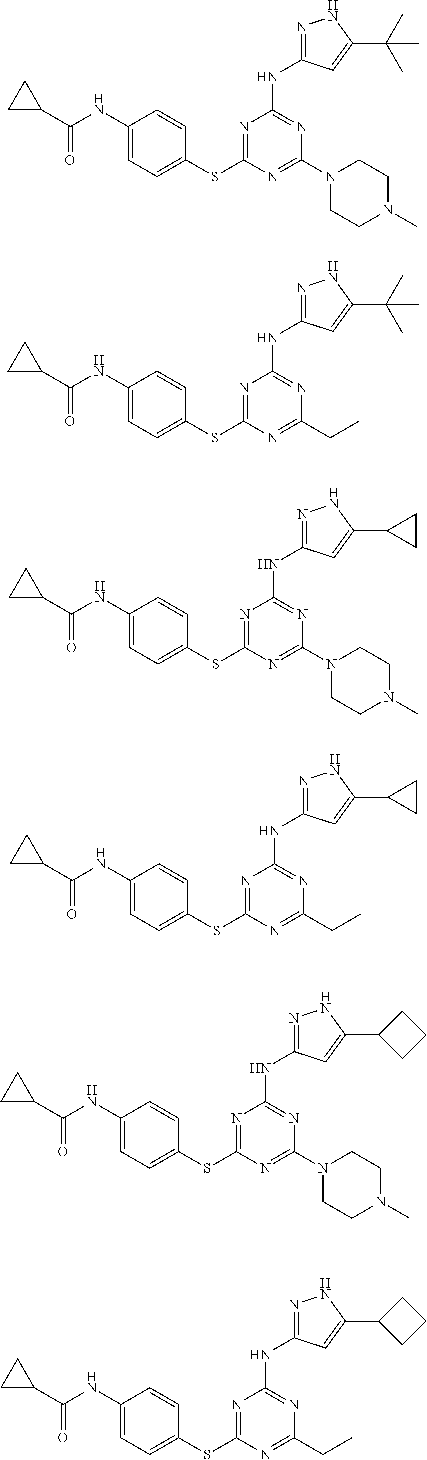

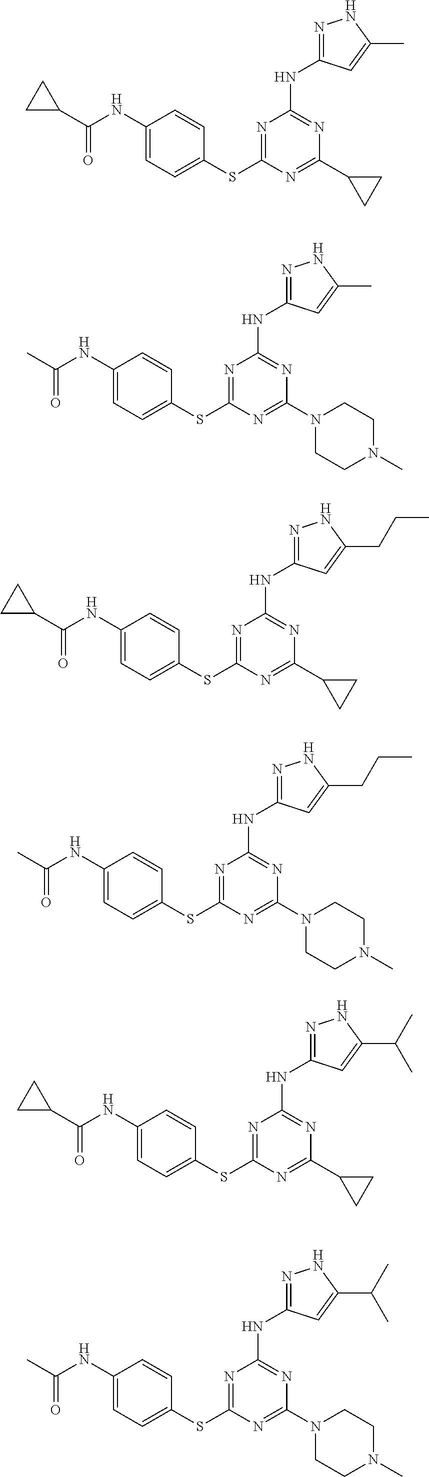

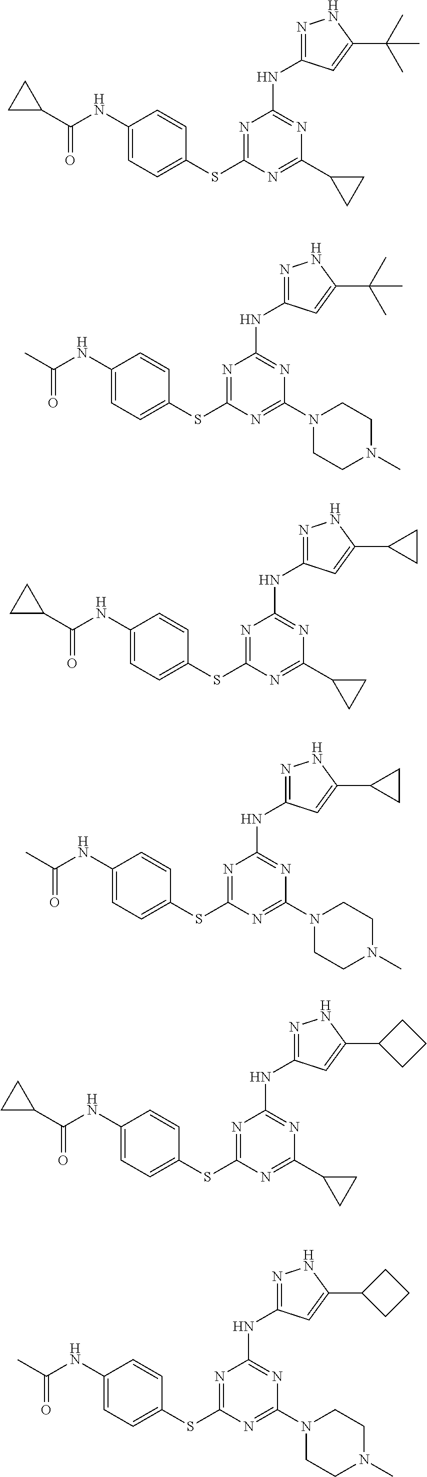

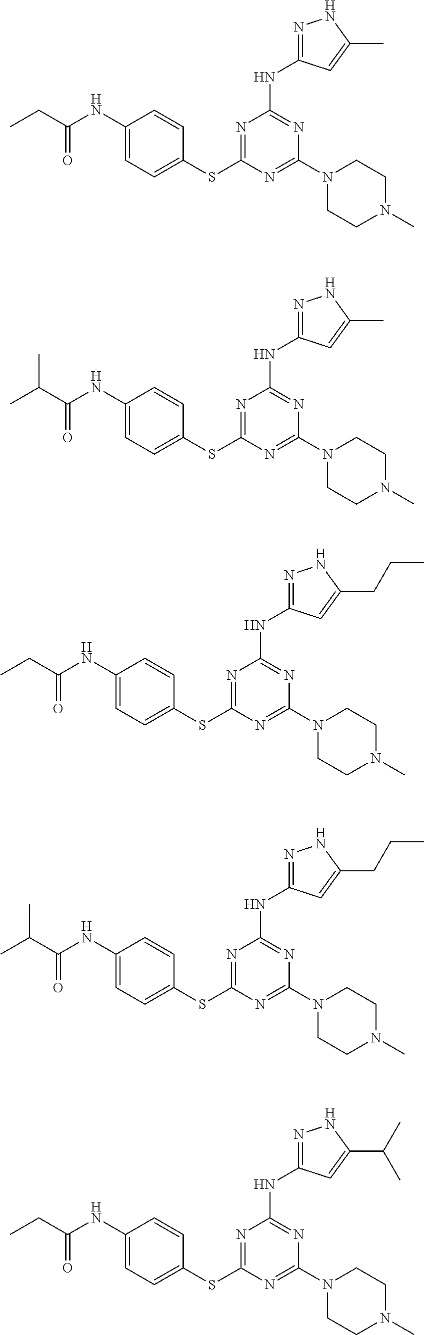

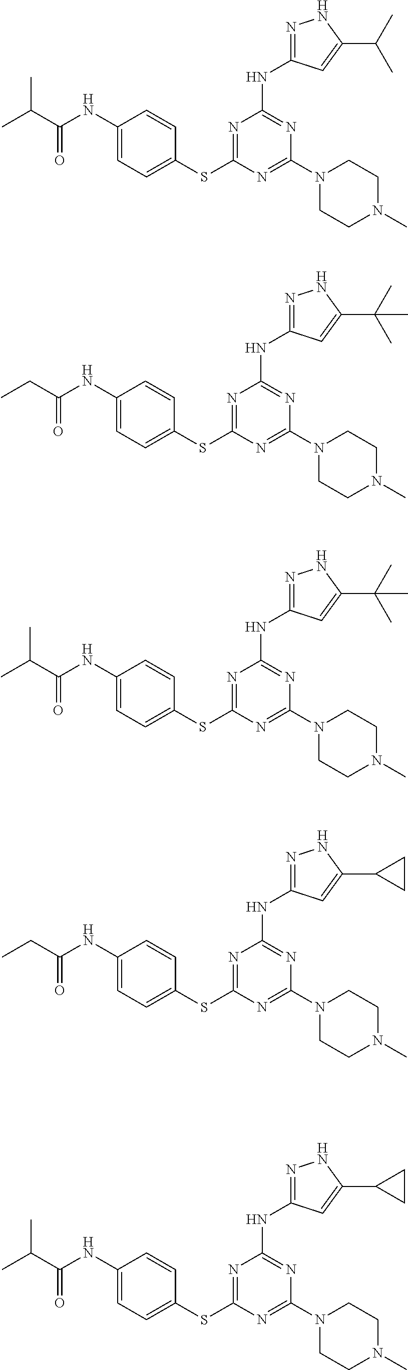

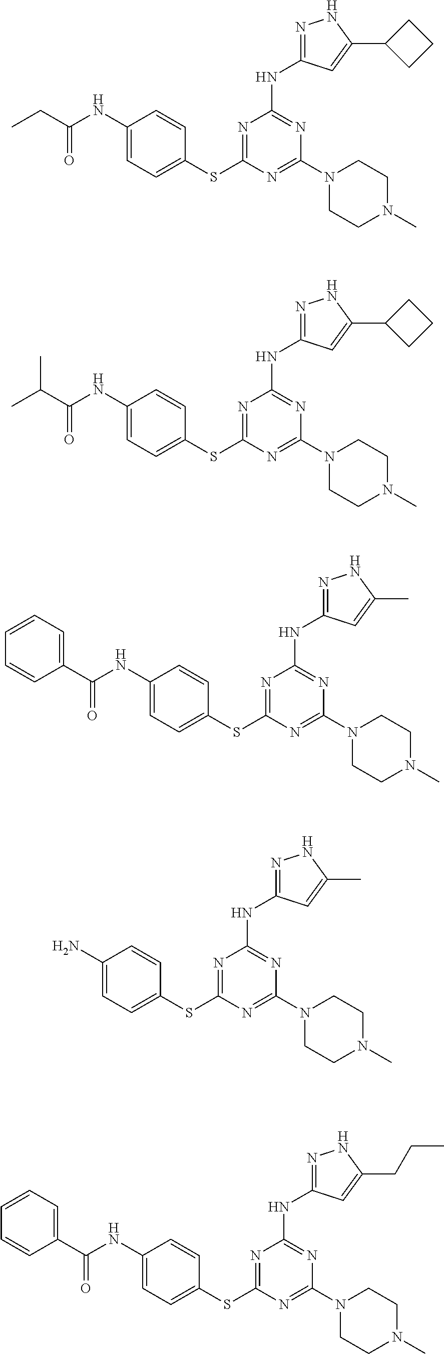

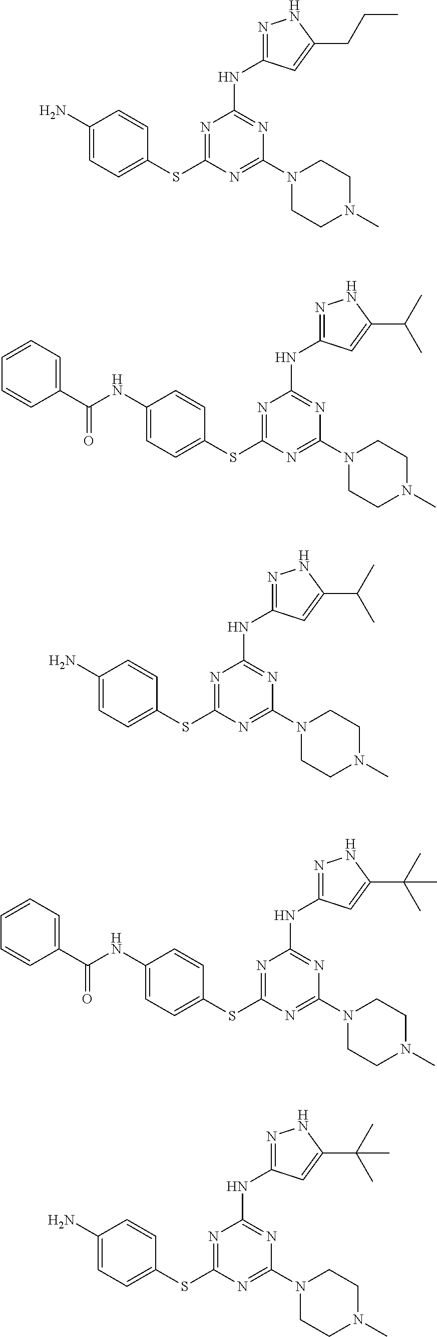

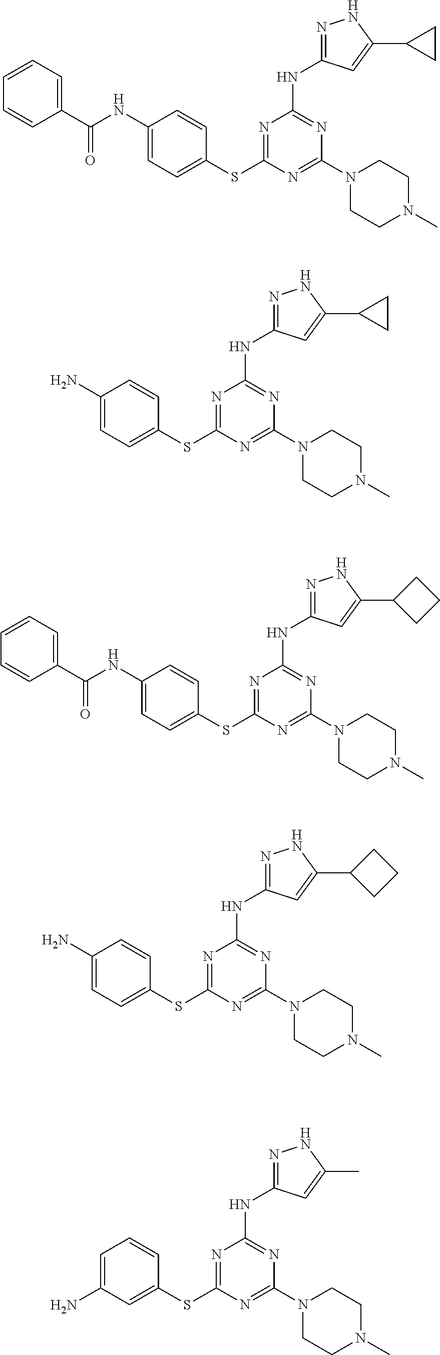

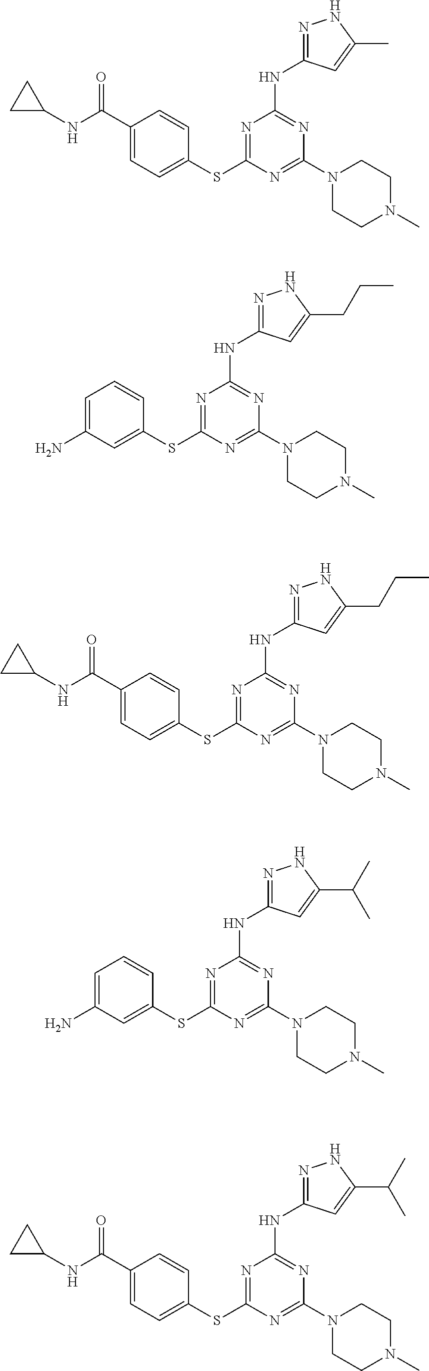

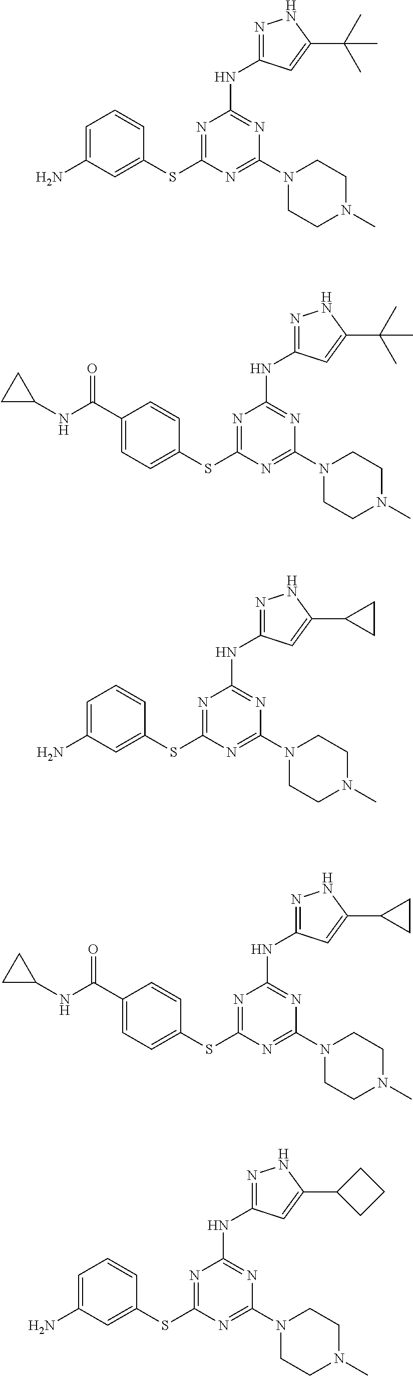

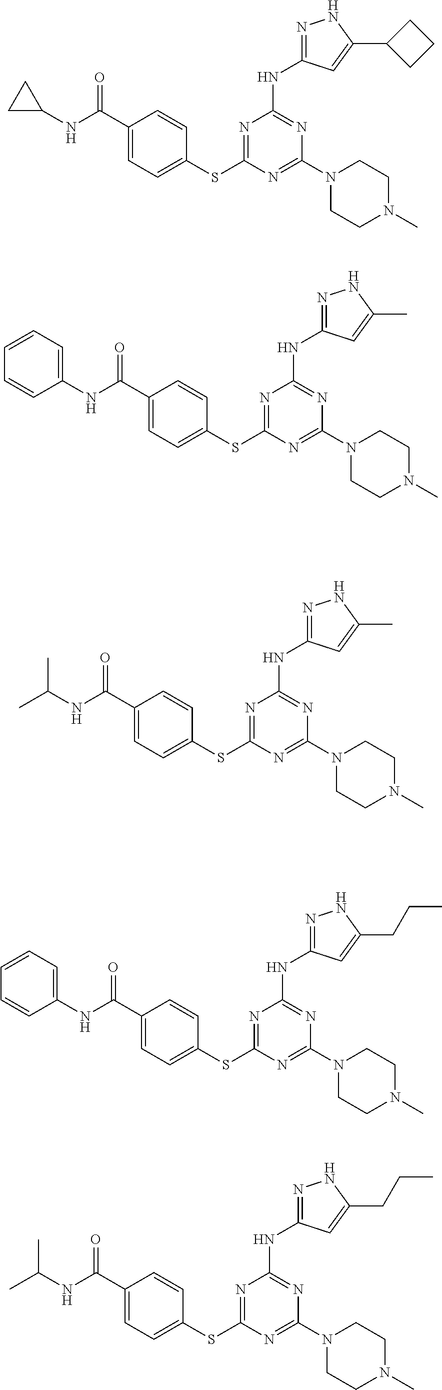

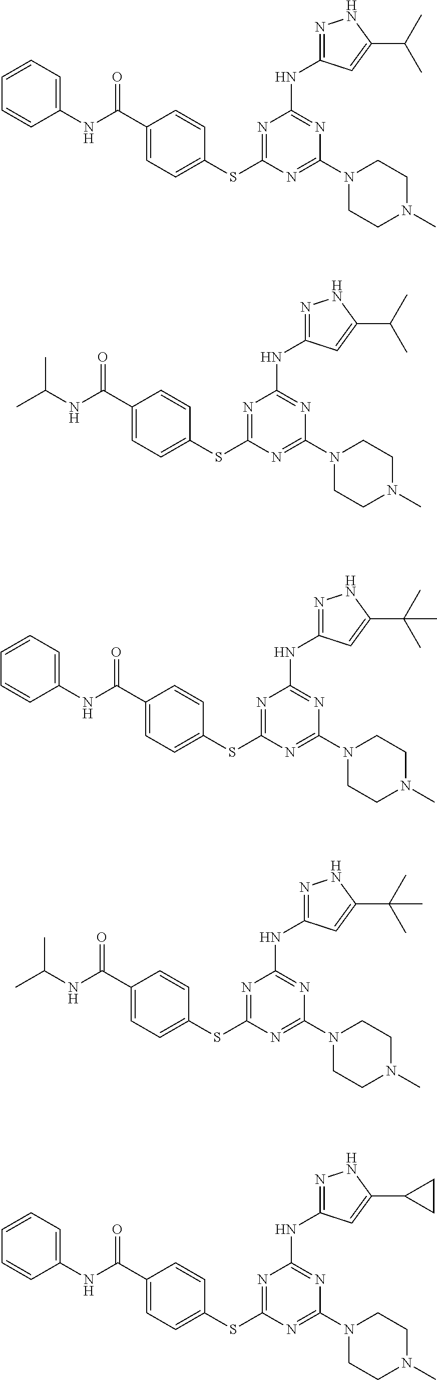

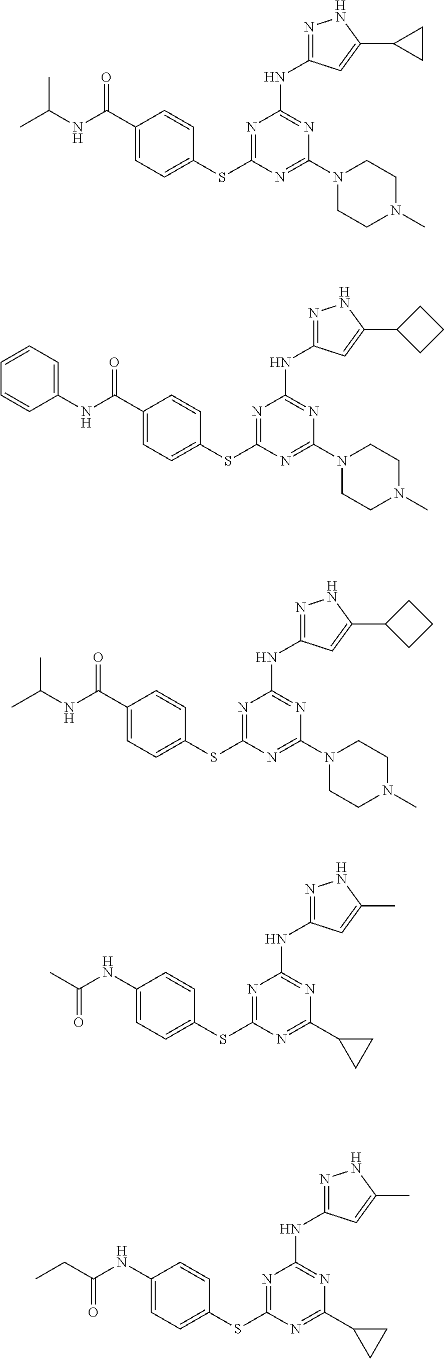

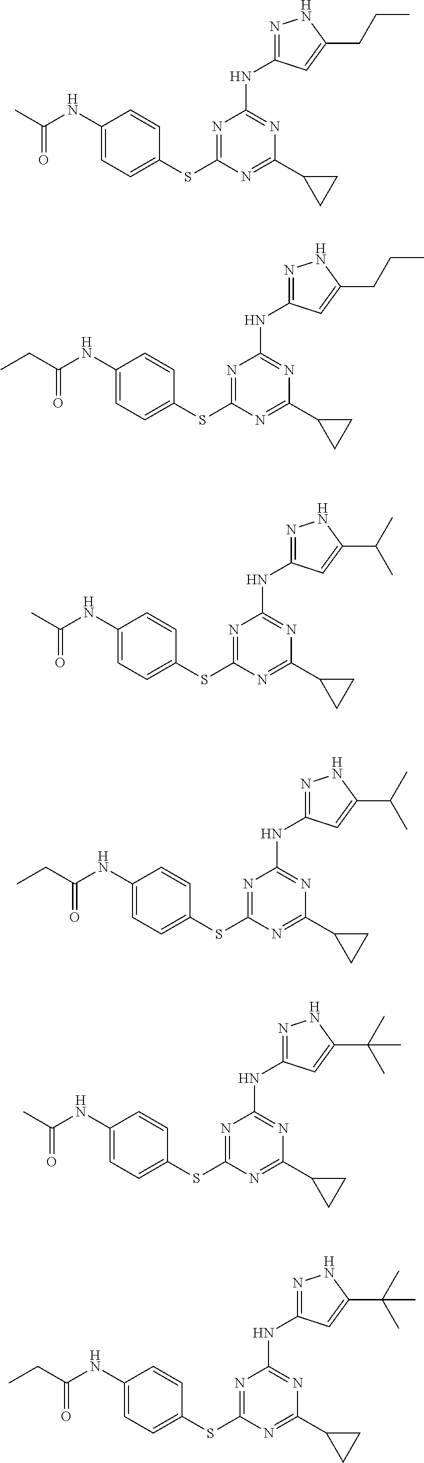

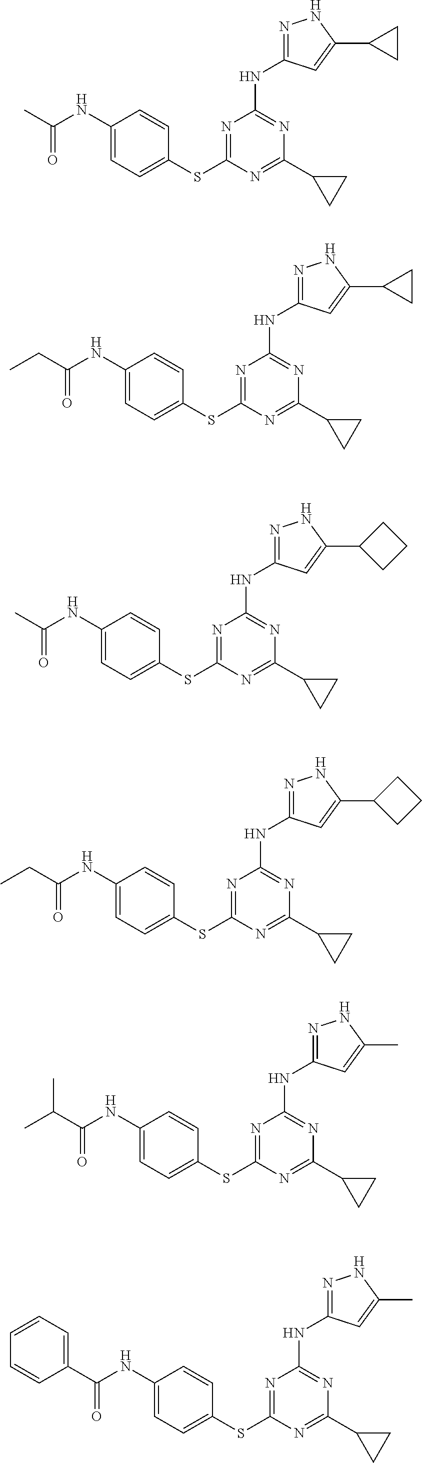

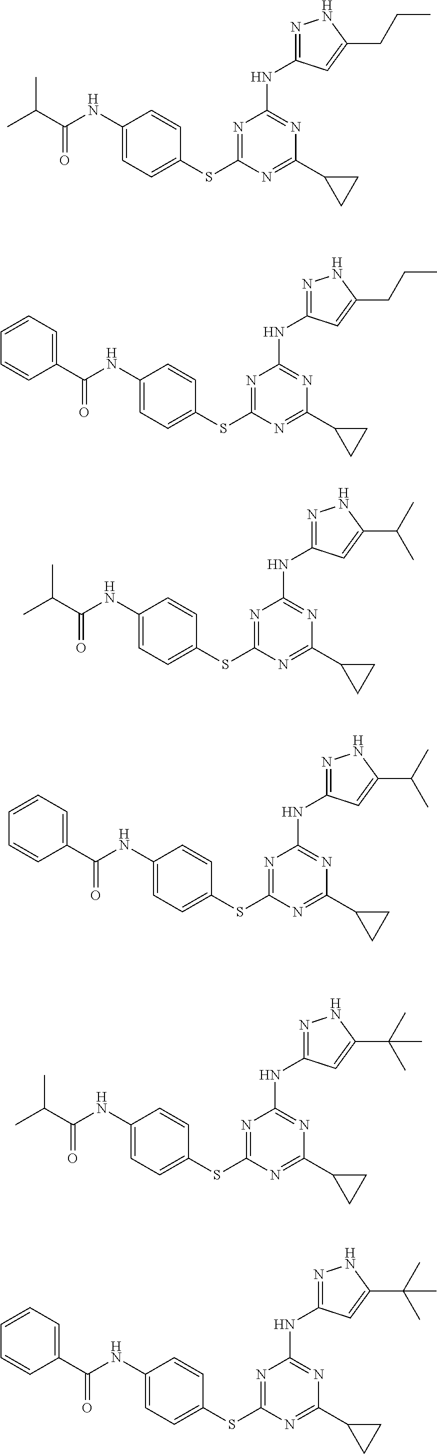

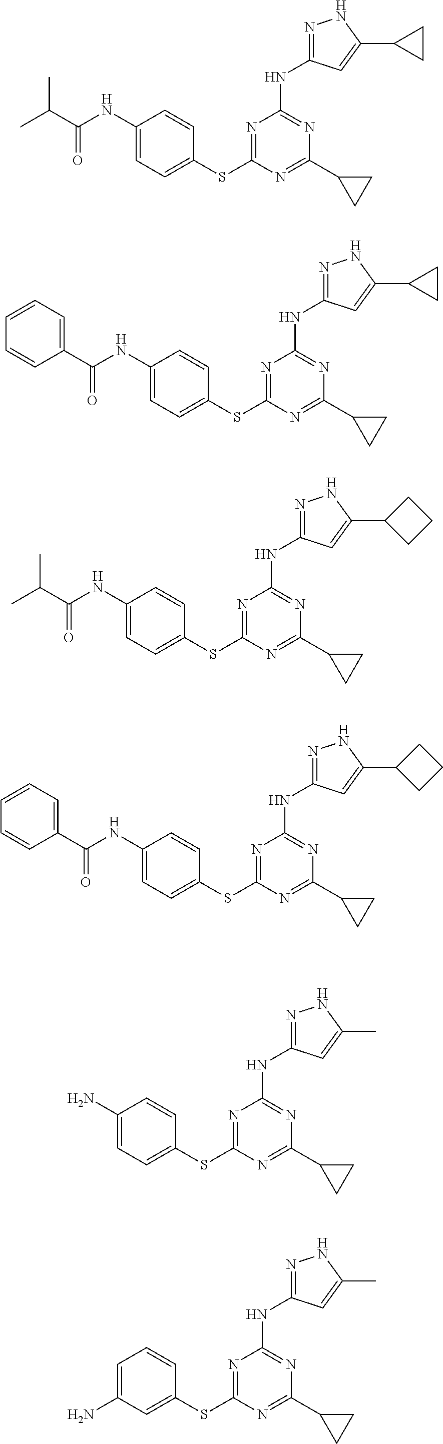

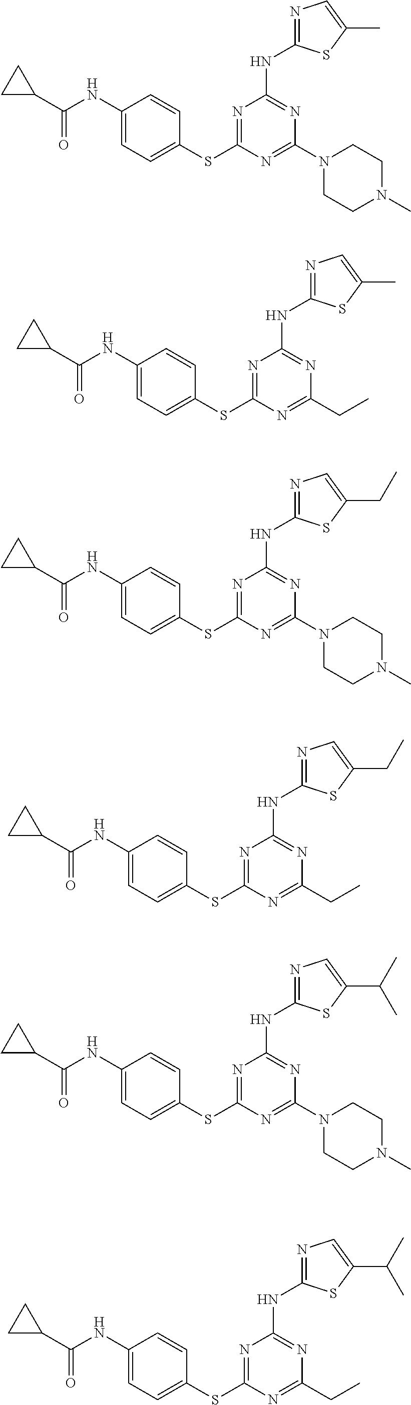

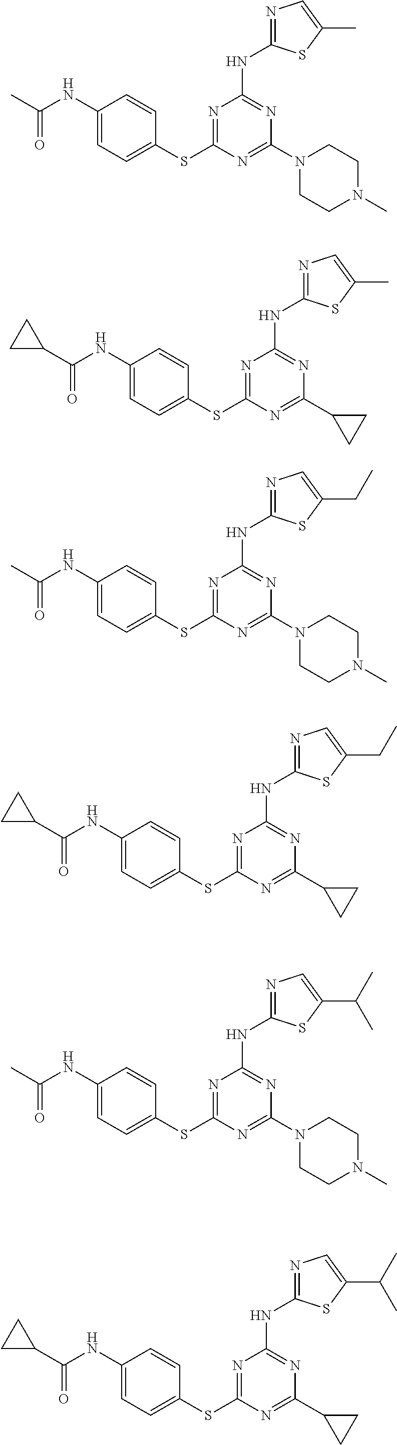

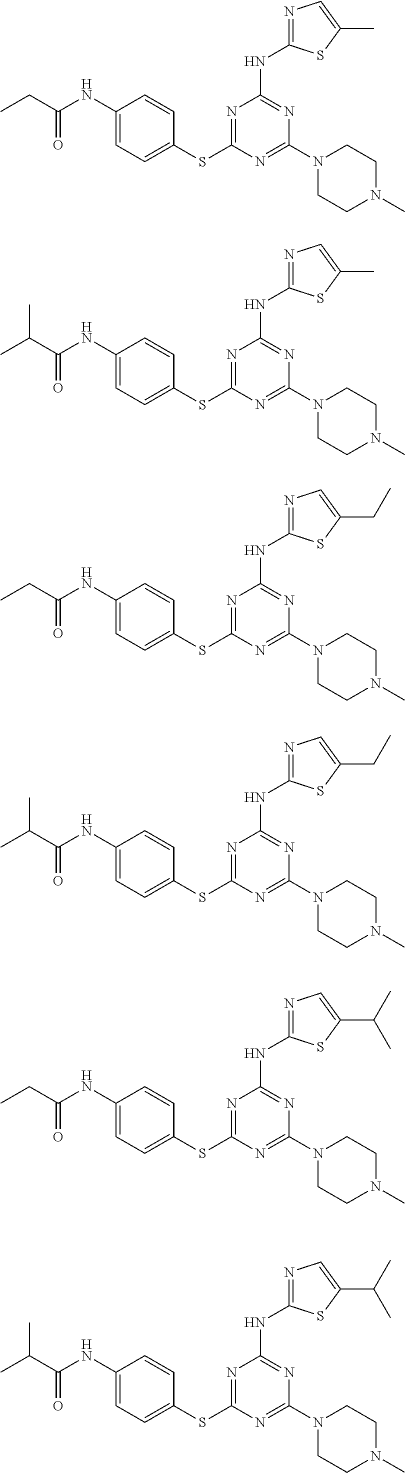

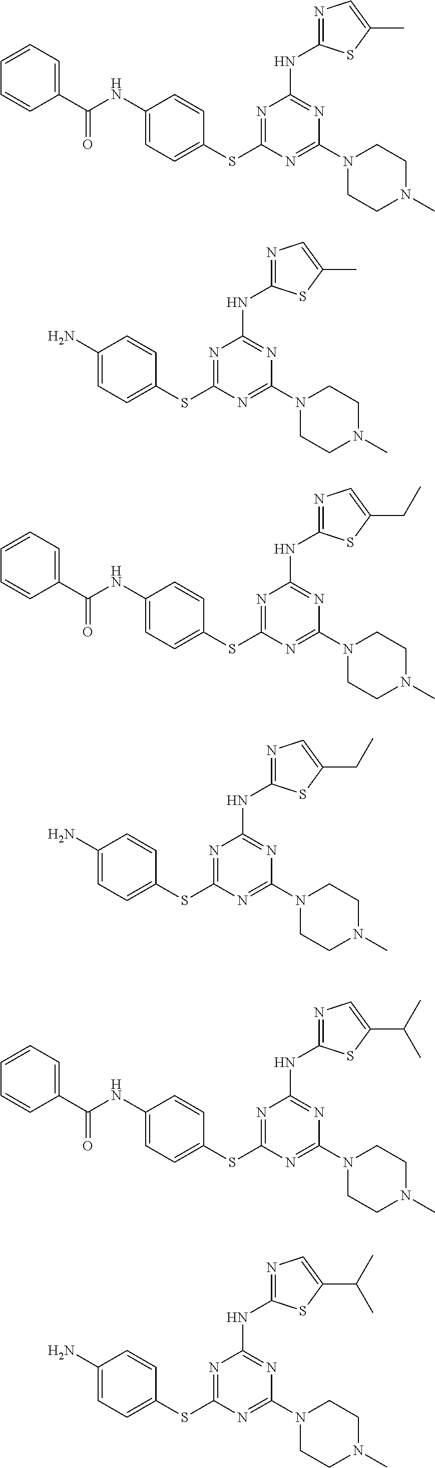

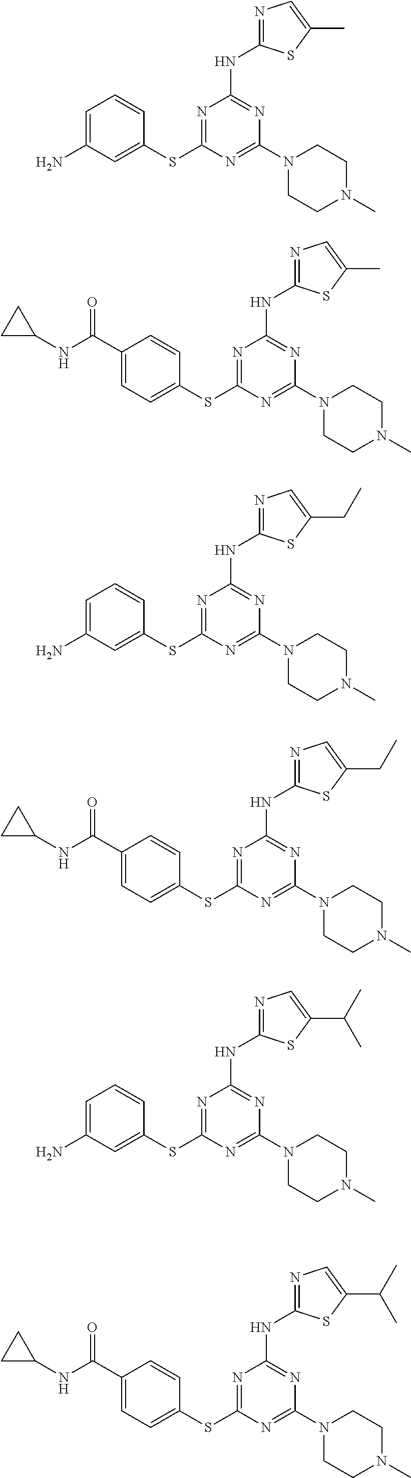

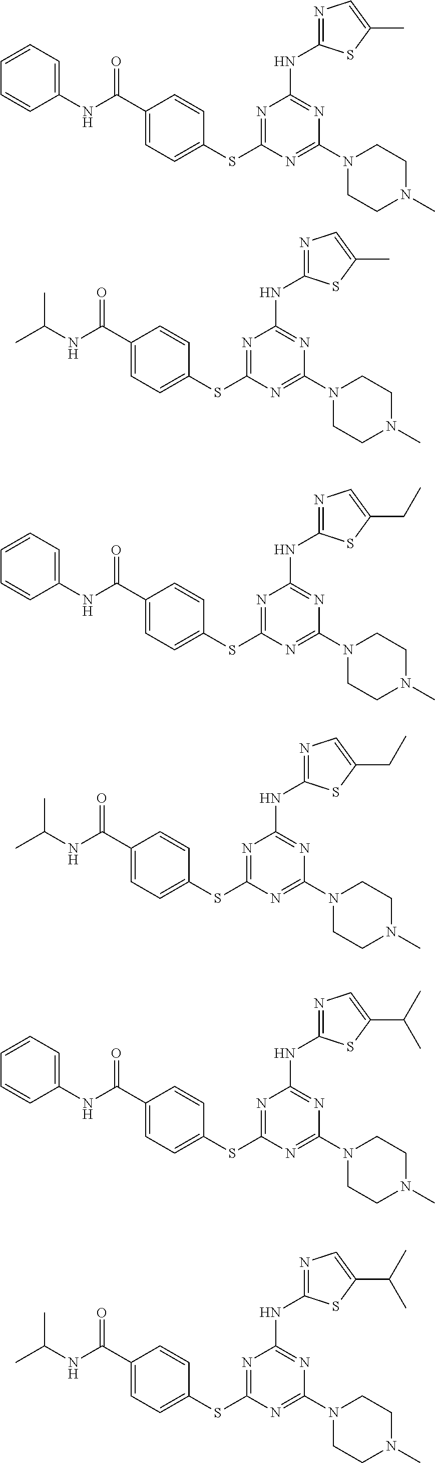

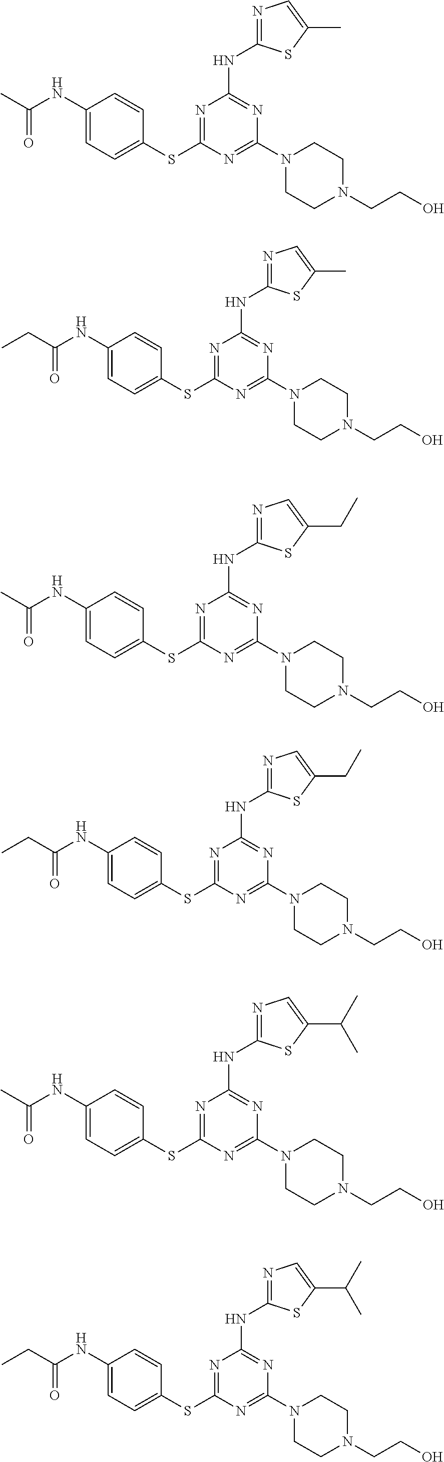

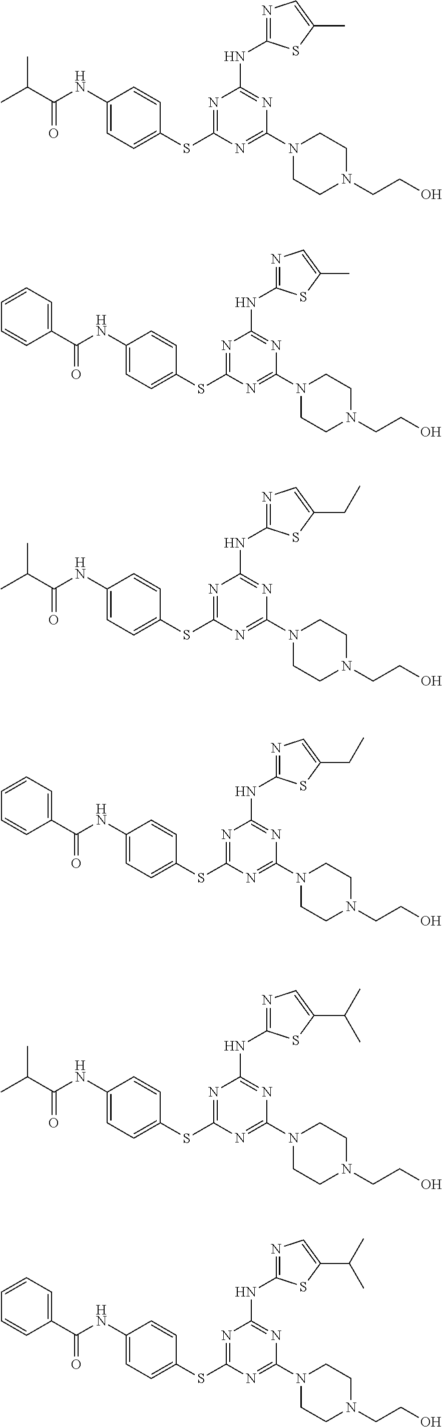

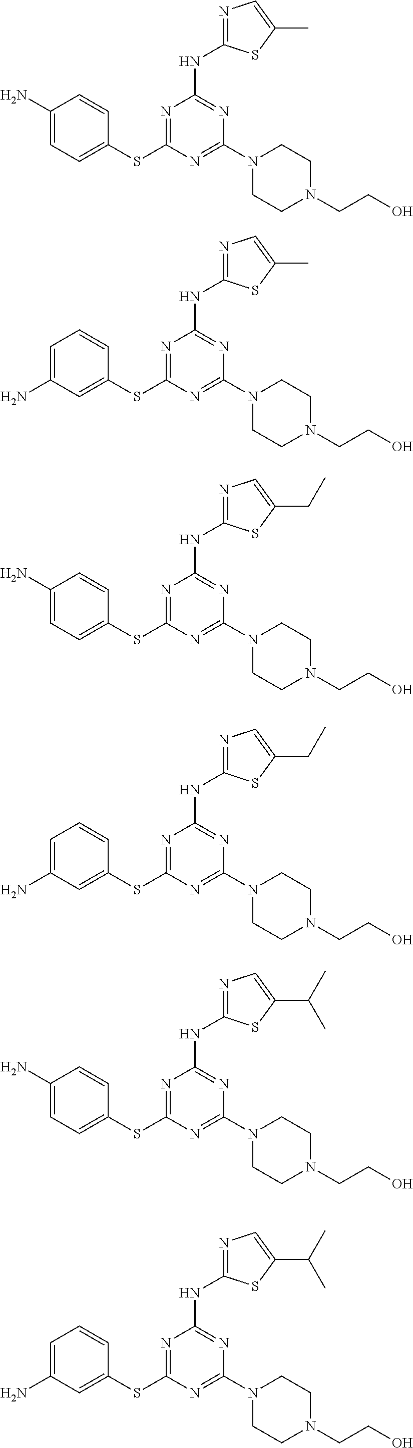

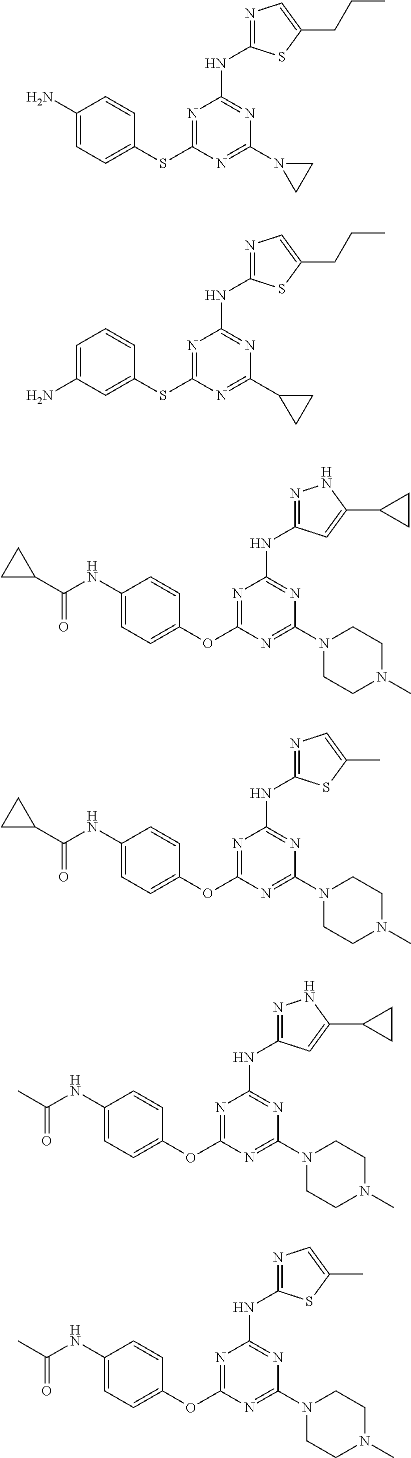

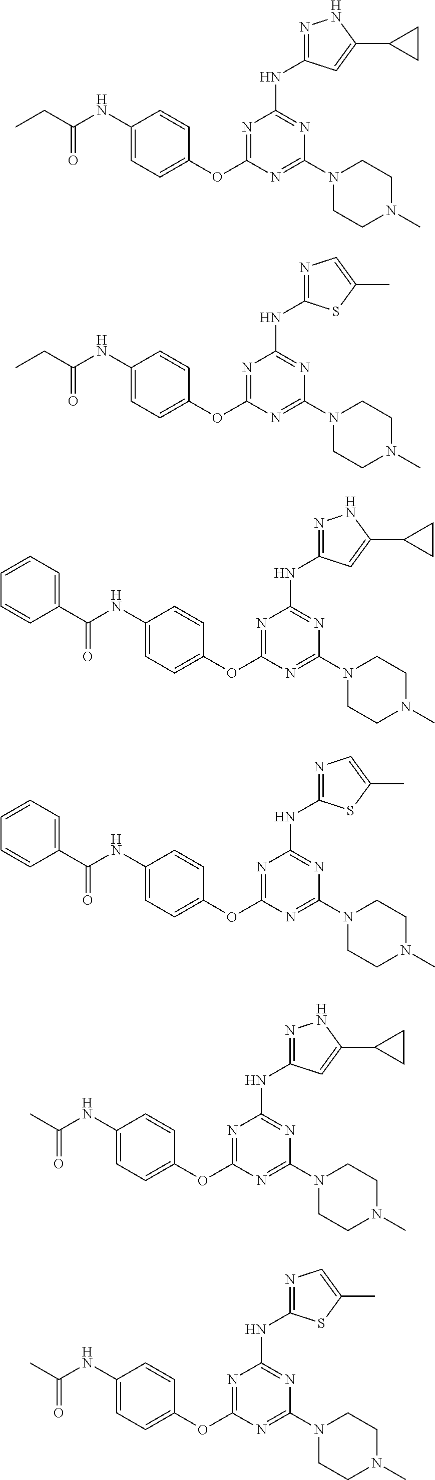

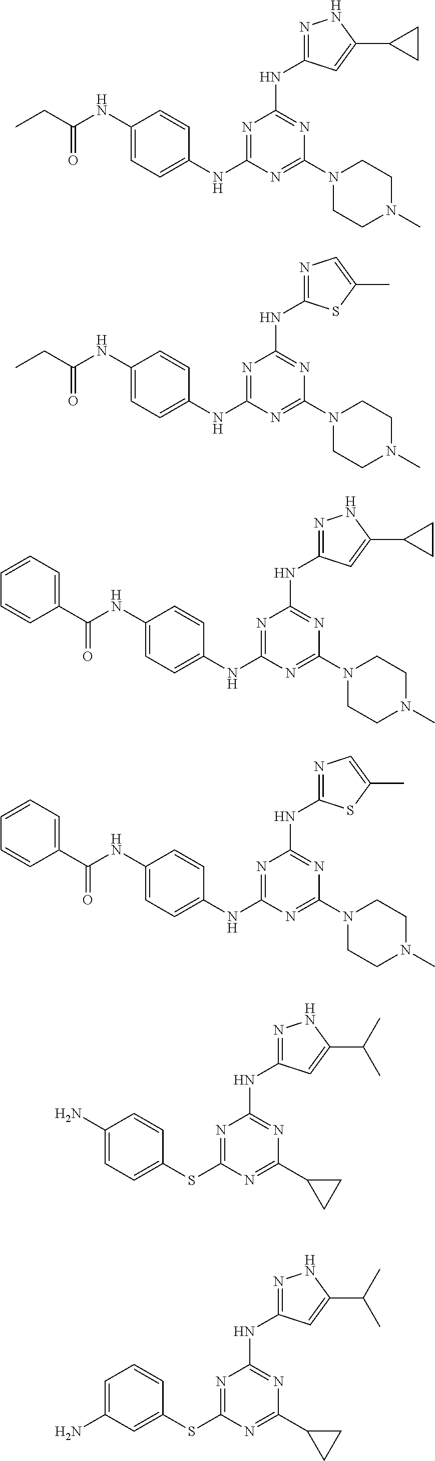

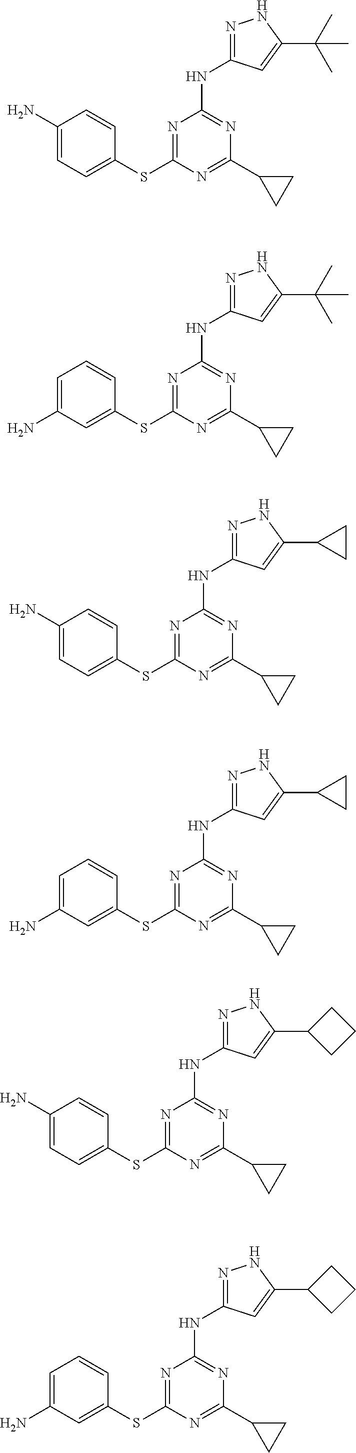

In another embodiment, a method of preparing the inventive compounds is provided. The compounds of the present invention can be generally prepared using cyanuric chloride as a starting material. Compounds of formula (I) or formula (II) may contain various stereoisomers, geometric isomers, tautomeric isomers, and the like. All of possible isomers and their mixtures are included in the present invention, and the mixing ratio is not particularly limited.

The triazine derivative compounds of formula (I) or formula (II) in this invention can be prepared by known procedure in the prior art. The examples could be found in US Patent Application Publication No. 2005/0250945A1; US Patent Application Publication No. 2005/0227983A1; PCT WO 05/007646A1; PCT WO 05/007648A2; PCT WO 05/003103A2; PCT WO 05/011703 A1; and J. Med. Chem. (2004), 47(19), 4649-4652. Starting materials are commercially available from suppliers such as Sigma-Aldrich Corp. (St. Louis, Mo.), or may be synthesized from commercially available precursors using established protocols. By way of example, a synthetic route similar to that shown in any of the following Schemes may be used, together with synthetic methods known in the art of synthetic organic chemistry, or variations thereon as appreciated by those skilled in the art. Each variable in the following schemes refers to any group consistent with the description of the compounds provided herein.

In the Schemes that follow the term “reduction” refers to the process of reducing a nitro functionality to an amino functionality, or the process of transforming an ester functionality to an alcohol. The reduction of a nitro group can be carried out in a number of ways well known to those skilled in the art of organic synthesis including, but not limited to, catalytic hydrogenation, reduction with SnCl₂ and reduction with titanium bichloride. The reduction of an ester group is typically performed using metal hydride reagents including, but not limited to, diisobutyl-aluminum hydride (DIBAL), lithium aluminum hydride (LAH), and sodium borohydride. For an overview of reduction methods see: Hudlicky, M. Reductions in Organic Chemistry, ACS Monograph 188, 1996. In the Schemes that follow, the term “hydrolyze” refers to the reaction of a substrate or reactant with water. More specifically, “hydrolyze” refers to the conversion of an ester or nitrite functionality into a carboxylic acid. This process can be catalyzed by a variety of acids or bases well known to those skilled in the art of organic synthesis.

The compounds of formula (I) or formula (II) may be prepared by use of known chemical reactions and procedures. The following general preparative methods are presented to aid one of skill in the art in synthesizing the inhibitors, with more detailed examples being presented in the experimental section describing the working examples.

Heterocyclic amines are defined in formula (III). Some of heterocyclic amines are commercially available, others may be prepared by known procedure in the prior art (Katritzky, et al. Comprehensive Heterocyclic Chemistry; Permagon Press: Oxford, UK, 1984, March. Advanced Organic Chemistry, 3rd Ed.; John Wiley: New York, 1985), or by using common knowledge of organic chemistry.

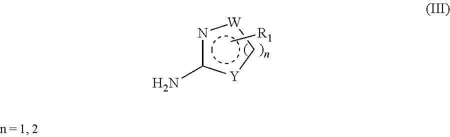

For example, substituted heterocyclic amines can be generated using standard methods (March, J. Advanced Organic Chemistry, 4th Ed.; John Wiley, New York (1992); Larock, R. C. Comprehensive Organic Transformations, 2nd Ed., John Wiley, New York (1999); PCT WO 99/32106). As shown in Scheme 1, heterocyclic amines can be commonly synthesized by reduction of nitroheteros using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroheteros may also be directly reduced using a strong hydride source, such as LAH, (Seyden-Penne. Reductions by the Alumino- and Borohydrides in Organic Synthesis; VCH Publishers: New York (1991)), or using a zero valent metal, such as Fe, Sn or Ca, often in acidic media. Many methods exist for the synthesis of nitroaryls (March, J. Advanced Organic Chemistry, 4th Ed.; John Wiley, New York (1992); Larock, R. C. Comprehensive Organic Transformations, 2nd Ed., John Wiley, New York (1999))).

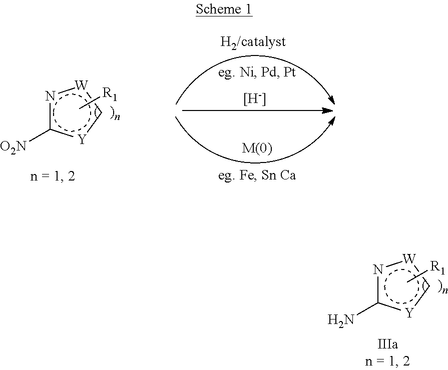

As illustrated in Scheme 2, thiazole amine with a substituent (IIIb) can be prepared from commercial compounds as illustrated in Scheme 2. By Route 1, a substituted aldehyde, which may be commercially available or prepared by oxidizing an alcohole, can be brominated by broming or NBS (N-Bromosuccinimide); after bromination, the aldehyde can be converted to the corresponding thiazole amine (IIIb) by reacting with thiourea. For the oxidation step, a variety of oxidizing reagent can be used, such as pyridinium chlorochromate (PCC) activated dimethyl sulfoxide (DMSO), hypervalent iodide compounds, Tetrapropylammonium perruthenate (TPAP) or 2,2,6,6-Tetramethylpiperidine-1-oxyl (TEMPO). A lot of thiazole amines can be prepared by this way.

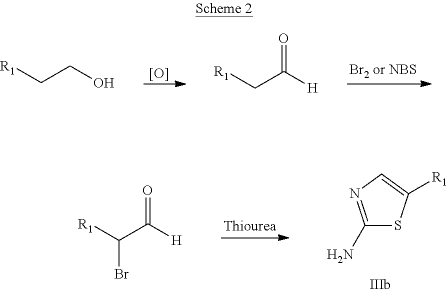

A lot of substituted pyrazole amines are commercially available and can be used directly. In some special case, pyrazole amines with a substituent (IIIc) can be prepared by known procedure in the prior art, such as U.S. Pat. No. 6,407,238; F. Gabrera Escribano, et al. Tetrahedron Letters, Vol. 29, No. 46, pp. 6001-6004, 1988; Org. Biomol. Chem., 2006, 4, 4158-4164; WO 2003/026666.

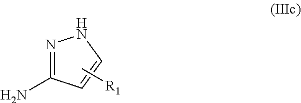

Precursors R₃H can be purchased from suppliers as exampled earlier, or synthesized from commercially available precursors using established protocols. For example, as illustrated in Scheme 3, substituted N-(mercaptophenyl)carboxamide (IVa) are readily available from the reaction of an aminobenzenethiol with a carboxylic acid or its derivatives such as acyl chloride, acid anhydride or ester.

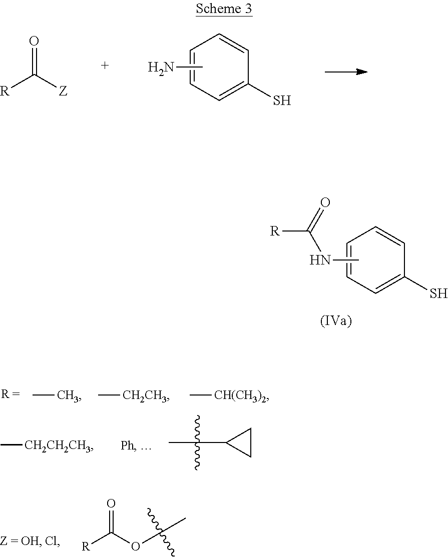

Alternatively, substituted mercapto-N-benzamide (IVb) can be prepared by mercaptobenzoic acid, which is protected by appropriate group, with the corresponding amines as was shown in Scheme 4.

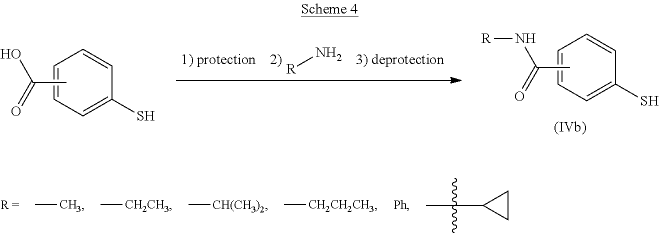

The preparation of the compounds of formula (I) or formula (II) in this invention can be carried out by methods known in the art (e.g., J. Med. Chem. 1996, 39, 4354-4357; J. Med. Chem. 2004, 47, 600-611; J. Med. Chem. 2004, 47, 6283-6291; J. Med. Chem. 2005, 48, 1717-1720; J. Med. Chem. 2005, 48, 5570-5579; U.S. Pat. No. 6,340,683 B1; JOC, 2004, 29, 7809, 7815.)

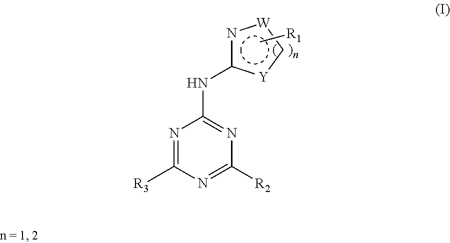

Scheme 5 illustrated the synthesis method for compounds with alkyl or aryl as R₂. The 6-alkyl or aryl substituted dichloro-triazine (b) may be synthesized by the methods known in the art (e.g., J. Med. Chem. 1999, 42, 805-818 and J. Med. Chem. 2004, 47, 600-611) from cyanuric chloride (a) and Grignard reagents. The monochloro-triazine (c) can be formed from the reaction of a 6-alkyl or aryl substituted dichloro-triazine (b) with heterocyclic amine, which can be converted to triazine derivatives of formula (I) or formula (II) by reaction with HR₃. Alternatively, the dichloro-triazine (b) can be converted to monochloro-triazine (d) by reacting with HR₂, which also can be converted to triazine derivatives of formula (I) or formula (II) by reacting with a heterocyclic amine.

Similarly compounds with alkyl or aryl as R₃ can be prepared eith the same method as illustrated in Scheme 6.

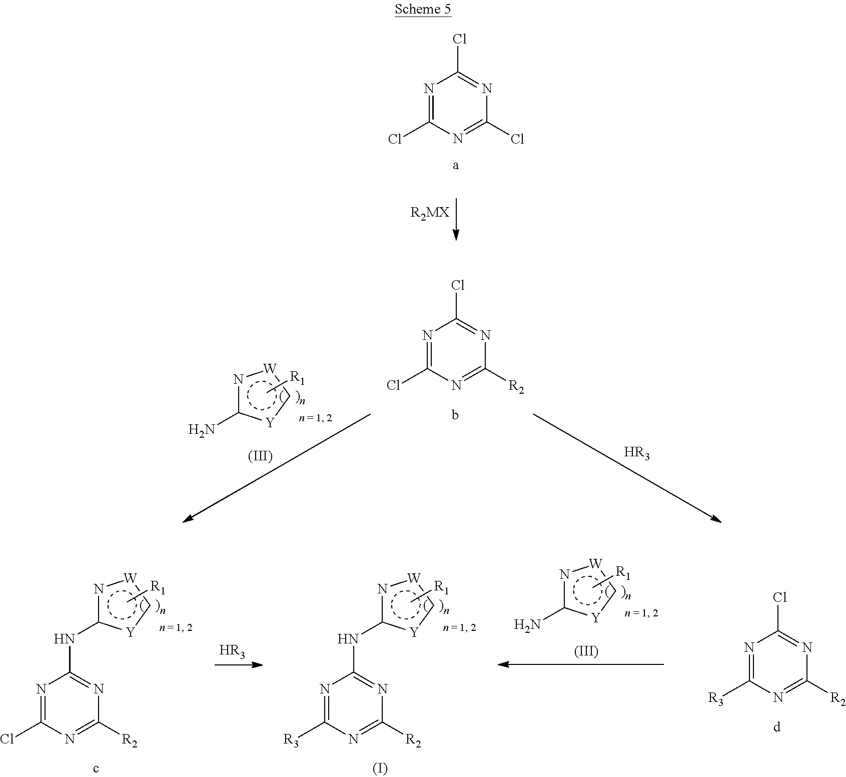

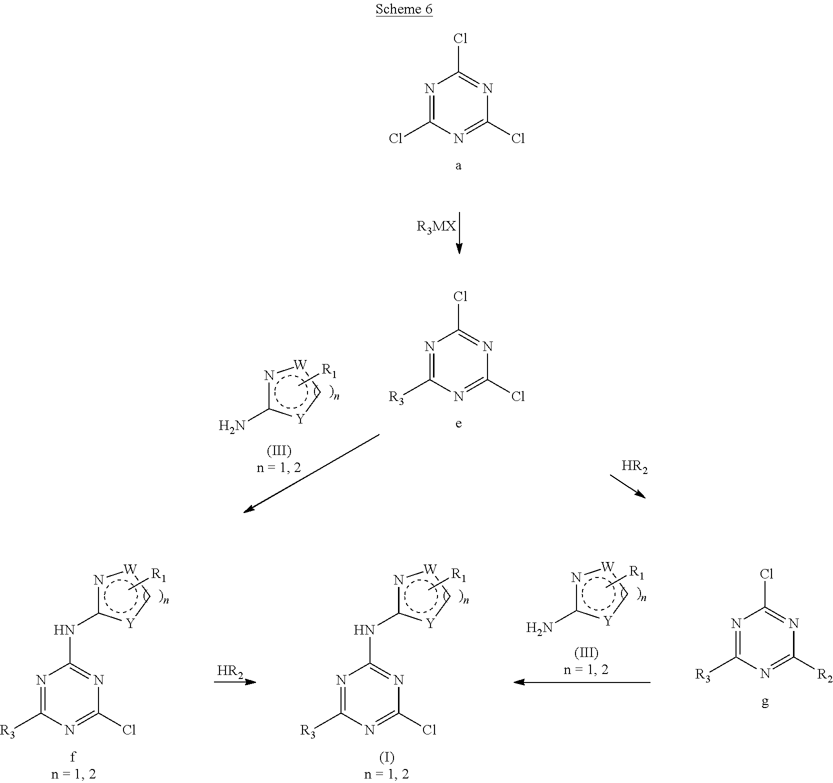

As shown in Scheme 7, the triazine derivative can also be synthesized by the reaction of cyanuric chloride with a sequence of heterocyclic amines and HR₂ to give 2,4-disubstituted-6-chloro-1,3,5-triazines. The displacement of the last chlorine by HR₃ can be achieved by increasing the temperature, affording the trisubstituted-1,3,5-triazines of formula (I) or formula (II). Alternative sequence can also be used to make triazine derivatives as illustrated in Scheme 7.

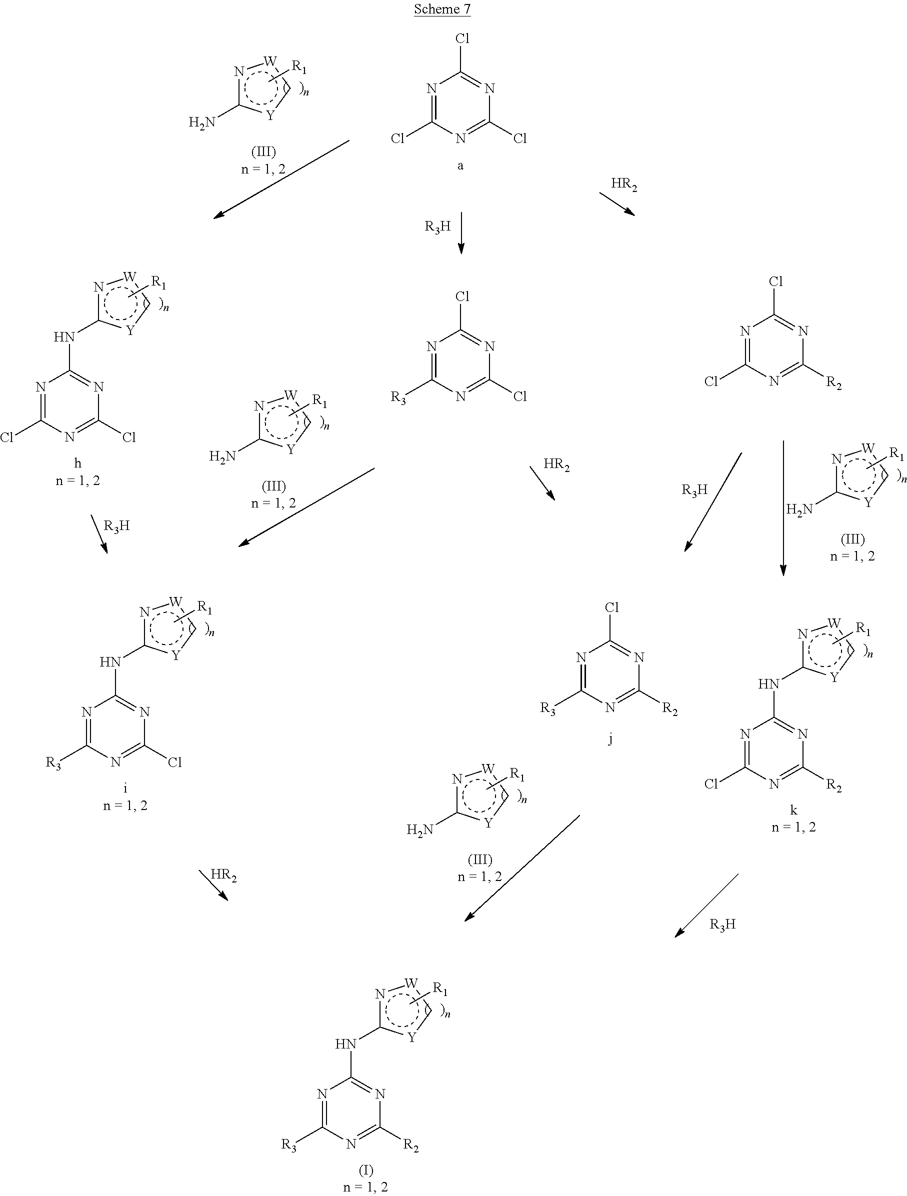

Furthermore, the triazine derivative can be modified to added or remove substituents. For example, a substituted thio-N-benzamide (Ic) can be prepared for the corresponding acid (Ib) by known methods as shown in Scheme 8.

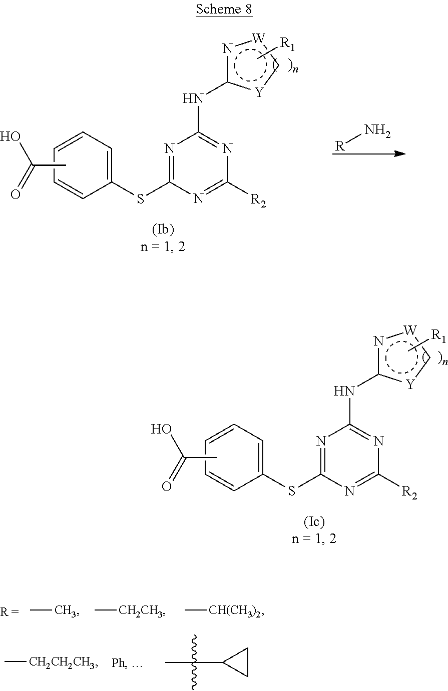

The reaction is preferably conducted in the presence of an inert solvent. There is no particular restriction on the nature of the solvent to be employed, provided that it has no adverse effect on the reaction or on the reagents involved and that it can dissolve the reagents, at least to some extent. Examples of suitable solvents include: aliphatic hydrocarbons, such as hexane, heptane, ligroin and petroleum ether; aromatic hydrocarbons, such as benzene, toluene and xylene; halogenated hydrocarbons, especially aromatic and aliphatic hydrocarbons, such as methylene chloride, chloroform, carbon tetrachloride, dichloroethane, chlorobenzene and the dichlorobenzenes; esters, such as ethyl formate, ethyl acetate, propyl acetate, butyl acetate and diethyl carbonate; ethers, such as diethyl ether, diisopropyl ether, tetrahydrofuran, dioxane. dimethoxyethane and diethylene glycol dimethyl ether; ketones, such as acetone, methyl ethyl ketone, methyl isobutyl ketone, isophorone and cyclohexanone; nitro compounds, which may be nitroalkanes or nitroaranes, such as nitroethane and nitrobenzene; nitriles, such as acetonitrile and isobutyronitrile; amides, which may be fatty acid amides, such as formamide, dimethylformamide, dimethylacetamide and hexamethylphosphoric triamide; and sulphoxides, such as dimethyl sulphoxide and sulpholane.

The reaction can take place over a wide range of temperatures, and the precise reaction temperature is not critical to the invention. In general, we find it convenient to carry out the reaction at a temperature of from −50° C. to 100° C.

The present invention provides compositions of matter that are formulations of one or more active drugs and a pharmaceutically-acceptable carrier. In this regard, the invention provides a composition for administration to a mammalian subject, which may include a compound of formula (I) or formula (II), or its pharmaceutically acceptable salts.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutically acceptable compositions of this invention may be orally administered in any orally acceptable dosage form including, but not limited to, capsules, tablets, troches, elixirs, suspensions, syrups, wafers, chewing gums, aqueous suspensions or solutions.

The oral compositions may contain additional ingredients such as: a binder such as microcrystalline cellulose, gum tragacanth or gelatin; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavoring agent such as peppermint, methyl salicylate, or orange flavoring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day dosing or dosing once every several days may also be utilized.

Sterile, injectable solutions may be prepared by incorporating a compound in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

In accordance with the invention, there are provided compositions containing triazine derivatives and methods useful for the in vivo delivery of triazine derivatives in the form of nanoparticles, which are suitable for any of the aforesaid routes of administration.

U.S. Pat. Nos. 5,916,596, 6,506,405 and 6,537,579 teach the preparation of nanoparticles from the biocompatible polymers, such as albumin. Thus, in accordance with the present invention, there are provided methods for the formation of nanoparticles of the present invention by a solvent evaporation technique from an oil-in-water emulsion prepared under conditions of high shear forces (e.g., sonication, high pressure homogenization, or the like).

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

In accordance with the invention, the compounds of the invention may be used to treat diseases associated with cellular proliferation or hyperproliferation, such as cancers which include but are not limited to tumors of the nasal cavity, paranasal sinuses, nasopharynx, oral cavity, oropharynx, larynx, hypopharynx, salivary glands, and paragangliomas. The compounds of the invention may also be used to treat cancers of the liver and biliary tree (particularly hepatocellular carcinoma), intestinal cancers, particularly colorectal cancer, ovarian cancer, small cell and non-small cell lung cancer, breast cancer, sarcomas (including fibrosarcoma, malignant fibrous histiocytoma, embryonal rhabdomysocarcoma, leiomysosarcoma, neuro-fibrosarcoma, osteosarcoma, synovial sarcoma, liposarcoma, and alveolar soft part sarcoma), neoplasms of the central nervous systems (particularly brain cancer), and lymphomas (including Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma).

The compounds and methods of the present invention, either when administered alone or in combination with other agents (e.g., chemotherapeutic agents or protein therapeutic agents described below) are also useful in treating a variety of disorders, including but not limited to, for example: stroke, cardiovascular disease, myocardial infarction, congestive heart failure, cardiomyopathy, myocarditis, ischemic heart disease, coronary artery disease, cardiogenic shock, vascular shock, pulmonary hypertension, pulmonary edema (including cardiogenic pulmonary edema), pleural effusions, rheumatoid arthritis, diabetic retinopathy, retinitis pigmentosa, and retinopathies, including diabetic retinopathy and retinopathy of prematurity, inflammatory diseases, restenosis, asthma, acute or adult respiratory distress syndrome (ARDS), lupus, vascular leakage, protection from ischemic or reperfusion injury such as ischemic or reperfusion injury incurred during organ transplantation, transplantation tolerance induction; ischemic or reperfusion injury following angioplasty; arthritis (such as rheumatoid arthritis, psoriatic arthritis or osteoarthritis); multiple sclerosis; inflammatory bowel disease, including ulcerative colitis and Crohn's disease; lupus (systemic lupus crythematosis); graft vs. host diseases; T-cell mediated hypersensitivity diseases, including contact hypersensitivity, delayed-type hypersensitivity, and gluten-sensitive enteropathy (Celiac disease); Type 1 diabetes; psoriasis; contact dermatitis (including that due to poison ivy); Hashimoto's thyroiditis; Sjogren's syndrome; Autoimmune Hyperthyroidism, such as Graves' disease; Addison's disease (autoimmune disease of the adrenal glands); autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; cancers, including those where kineses such as Src-family kineses are activated or overexpressed, such as colon carcinoma and thymoma, or cancers where kinase activity facilitates tumor growth or survival; glomerulonephritis, serum sickness; uticaria; allergic diseases such as respiratory allergies (asthma, hayfever, allergic rhinitis) or skin allergies; mycosis fungoides; acute inflammatory responses (such as acute or adult respiratory distress syndrome and ischemialreperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic schlerosis; morphea; peripheral limb ischemia and ischemic limb disease; bone disease such as osteoporosis, osteomalacia, hyperparathyroidism, Paget's disease, and renal osteodystrophy; vascular leak syndromes, including vascular leak syndromes induced by chemotherapies or immunomodulators such as IL-2; spinal cord and brain injury or trauma; glaucoma; retinal diseases, including macular degeneration; vitreoretinal disease; pancreatitis; vasculatides, including vasculitis, Kawasaki disease, thromboangiitis obliterans, Wegener s granulomatosis, and Behcet's disease; scleroderma; preeclampsia; thalassemia; Kaposi's sarcoma; von Hippel Lindau disease; and the like.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula 1, wherein the disease or condition is associated with a kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula (I) or formula (II), wherein the disease or condition is associated with a tyrosine kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula (I) or formula (II), wherein the disease or condition is associated with the kinase that is a serine kinase or a threonine kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula (I) or formula (II), wherein the disease or condition is associated with the kinase that is a Src family kinase.

The invention also provides methods of treating a mammal afflicted with the above diseases and conditions. The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

In one aspect, the invention compounds are administered in combination with chemotherapeutic agent, an anti-inflammatory agent, antihistamines, chemotherapeutic agent, immunomodulator, therapeutic antibody or a protein kinase inhibitor, e.g., a tyrosine kinase inhibitor, to a subject in need of such treatment.

The method includes administering one or more of the inventive compounds to the afflicted mammal. The method may further include the administration of a second active agent, such as a cytotoxic agent, including alkylating agents, tumor necrosis factors, intercalators, microtubulin inhibitors, and topoisomerase inhibitors. The second active agent may be co-administered in the same composition or in a second composition. Examples of suitable second active agents include, but are not limited to, a cytotoxic drug such as Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Cannustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-□a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

In accordance with the invention, the compounds and compositions may be used at sub-cytotoxic levels in combination with other agents in order to achieve highly selective activity in the treatment of non-neoplastic disorders, such as heart disease, stroke and neurodegenerative diseases (Whitesell et al., Curr Cancer Drug Targets (2003), 3(5), 349-58).

The exemplary therapeutical agents that may be administered in combination with invention compounds include EGFR inhibitors, such as gefitinib, erlotinib, and cetuximab. Her2 inhibitors include canertinib, EKB-569, and GW-572016. Also included are Src inhibitors, dasatinib, as well as Casodex (bicalutamide), Tamoxifen, MEK-1 kinase inhibitors, MARK kinase inhibitors, PI3 inhibitors, and PDGF inhibitors, such as imatinib, Hsp90 inhibitors, such as 17-AAG and 17-DMAG. Also included are anti-angiogenic and antivascular agents which, by interrupting blood flow to solid tumors, render cancer cells quiescent by depriving them of nutrition. Castration, which also renders androgen dependent carcinomas non-proliferative, may also be utilized. Also included are IGF1R inhibitors, inhibitors of non-receptor and receptor tyrosine kineses, and inhibitors of integrin.

The pharmaceutical composition and method of the present invention may further combine other protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay. The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

Other therapeutic agents for the combinatory therapy include cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and for gpn39 (i.e., CD154), fusion proteins constructed from CD40 and gp39 (CD401g and CD8gp39), inhibitors, such as nuclear translocation inhibitors, of NF-kappa B function, such as deoxyspergualin (DSG), cholesterol biosynthesis inhibitors such as HM:G CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-a inhibitors such as tenidap, anti-TNF antibodies or soluble TNF receptor, and rapamycin (sirolimus or Rapamune) or derivatives thereof.

When other therapeutic agents are employed in combination with the compounds of the present invention they may be used for example in amounts as noted in the Physician Desk Reference (PDR) or as otherwise determined by one having ordinary skill in the art.

### EXAMPLES

The following examples are provided to further illustrate the present invention but, of course, should not be construed as in any way limiting its scope.

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated.

Analytical thin layer chromatography (TLC) was carried out on Merck Kiesel gel 60 F254 plates with visualization by ultraviolet and/or anisaldehyde, potassium permanganate or phosphomolybdic acid dips.

NMR spectra: 1H Nuclear magnetic resonance spectra were recorded at 400 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.

Low resolution mass spectra: Electrospray (ES+) ionization was used. The protonated parent ion (M+H) or parent sodium ion (M+Na) or fragment of highest mass is quoted. Analytical gradient consisted of 10% ACN in water ramping up to 100% ACN over 5 minutes unless otherwise stated.

High performance liquid chromatography (HPLC) was use to analyze the purity of triazine derivatives. HPLC was performed on a Phenomenex Synergi Polar-RP, 4u, 80A, 150×4.6 mm column using a vShimadzusystem equipted with SPD-M10A Phosphodiode Array Detector. Mobile phase A was water and mobile phase B was acetonitrile with a gradient from 20% to 80% B over 60 minutes and re-equilibrate at A/B (80:20) for 10 minutes. UV detection was at 220 and 54 nm.

### Example 1

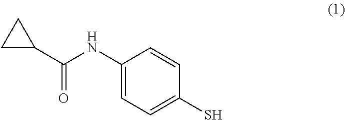

To a solution of 4-aminothiophenol (6.00 g, 47.93 mmol) and pyridine (5.3 mL, 65.53 mmol) in THF (100 mL) at −5° C. was added a solution of cyclopropanecarbonyl chloride (3.00 mL, 32.77 mmol) in THF (100 mL) drop wise. The reaction was stirred from 0° C. to room temperature for overnight, diluted with EtOAc (100 mL), washed with 1 N HCl (100 mL×5), dried over Na₂SO₄, concentrated, and dried under vacuum to yield the compound 1 as an off-white solid (6.01 g, 95%). Rf 0.50 (50% EtOAc/hexane); 1H NMR (400 MHz, DMSO-d6) δ 10.12 (s, 1H), 7.45 (d, J=8.8 Hz, 2H), 7.18 (d, J=8.8 Hz, 2H), 5.18 (s, 1H), 1.72 (m, 1H), 0.78 (m, 4H); ESI-MS: calcd for (C₁₀H₁₁NOS) 193. found 194 (MH+).

### Example 2

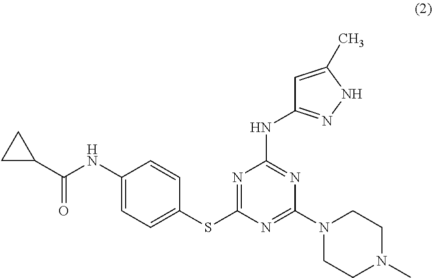

To a solution of cyanuric chloride (300 mg, 1.63 mmol) in THF (20 mL) was added a solution of 3-amino-5-methylpyrazole (158 mg, 1.63 mmol) and DIPEA (0.28 mL, 1.63 mmol) in THF (15 mL) dropwise at −10° C. After addition, the mixture was stirred at −10° C. for 30 more minutes. TLC was checked and the starting materials were consumed. In a separate flask, compound 1 (315 mg, 1.63 mmol) and DIPEA (0.28 mL, 1.63 mmol) was dissolved in THF (15 mL) and added to the above reaction flak drowise at 0° C. The mixture was stirred at room temperature overnight. Methyl piperazine (0.70 mL, 6.30 mmol) and DIPEA (0.57 mL, 3.26 mmol) was added to the reaction flask and the mixture was stirred at 60° C. for 2 hours. After cool to room temperature, saturated NaHCO₃ in water was added to the flask and the mixture was extracted by ethyl acetate two times. The combined organic was washed by brine, dried over sodium sulfate and concentrated. The resulting crude product was purified by flash column chromatography on silica gel using EtOAc/DCM/MeOH (7N NH3): 100/25/7 v/v/v as eluent to provide compound 2 as white solids (120 mg, 16%). 1H NMR (500 MHz, DMSO-d6) δ 11.71 (br, 1H), 10.43 (br, 1H), 9.50 (br, 1H), 7.72 (br, 2H), 7.48 (d, J=8.5 Hz, 2H), 5.30 (br, 1H), 3.67 (br, 4H), 2.30 (br, 4H), 2.18-1.95 (s, s, 6H), 1.80 (t, J=6.2 Hz, 1H), 0.81 (d, J=6.2 Hz, 4H); ESI-MS: calcd for (C₂₂H₂₇N₉OS) 465. found 466 (MH+). HPLC: retention time: 37.35 min. purity: 98%.

### Example 3

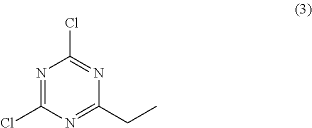

A solution of ethylmagnesium bromide in ether (3M, 15 ml, 45 mmole) was added dropwise to a stirred solution of cyanuric chloride (5.64 g, 30.58 mmole) in anhydrous dichloromethane at −10° C. After the addition was complete, the reaction mixture was stirred at −5° C. for 1 h, after which time water was added dropwise at a rate such that the temperature of the reaction stayed below 10° C. After warming to room temperature, the reaction mixture was diluted with additional water and methylene chloride and passed through a pad of cilite. The organic layer was dried and evaporated to give 2,4-dichloro-6-ethyl-1,3,5-triazine of compound 3 as yellow liquid, which solidified after storied in the refrigerator (5.20 g, 96%). 1H NMR (500 MHz, CDCl₃) δ 2.95 (q, J=7.5 Hz. 2H), 1.38 (t, J=7.5 Hz. 3H).

### Example 4

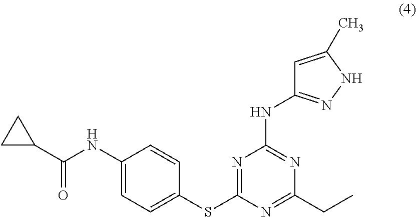

To a solution of compound 3 (163 mg, 0.90 mmol) in THF (10 mL) was added a solution of 3-amino-5-methylpyrazole (87 mg, 0.90 mmol) and DIPEA (0.16 mL, 0.90 mmol) in THF (5 mL) dropwise at 0° C. After addition, the mixture was stirred at 0° C. for additional 60 minutes. TLC was checked and the starting materials were consumed. A solution of compound 1 (303 mg, 1.56 mmol) and DIPEA (0.26 mL, 1.50 mmol) in THF (5 mL) was added to the above reaction flak at room temperature. The mixture was stirred at 70° C. for overnight. After cooling to room temperature, saturated NaHCO3 in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (7N NH₃): 00/3 v/v as eluent to provide compound 4 as white solids (150 mg, 42%). 1H NMR (400 MHz, DMSO-d6) δ 11.80 (br, 1H), 10.45 (br, 1H), 10.18 (br, 1H), 7.75 (d, J=9.2 Hz, 2H), 7.50 (d, J=8.8 Hz, 2H), 5.24 (br, 1H), 2.51 (q, J=7.6 Hz, 2H), 1.93 (s, 3H), 1.80 (m, 1H), 1.16 (t, J=7.6 Hz, 3H), 0.80 (d, J=6.0 Hz, 4H); ESI-MS: calcd for (C₁₉H₂₁N₇OS) 395. found 396 (MH+). HPLC: retention time: 21.97 min. purity: 98%.

### Example 5

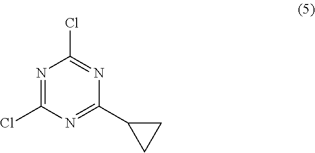

A solution of cyclopropylmagnesium bromide in THF (0.5 M, 25 ml, 12.5 mmol) was added dropwise to a stirred solution of cyanuric chloride (1.8 g, 10.00 mmol) in anhydrous dichloromethane at −10 to 0° C. After the addition was complete, the reaction mixture was stirred at 0° C. for 3 h, Water was added dropwise to the reaction mixture at a rate such that the temperature of the reaction stayed below 10° C. After warming to room temperature, the reaction mixture was diluted with additional water and methylene chloride and passed through a pad of cilite. The organic layer was dried and evaporated to give 2,4-dichloro-6-cyclopropyl-1,3,5-triazine of 5 as yellow liquid, which solidified after storied in the refrigerator (1.8 g, 95%). 1H NMR (400 MHz, CDCl₃) δ 2.20 (m, 1H), 1.38 (m, 2H), 1.32 (m, 2H).

### Example 6

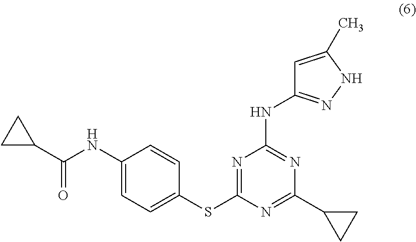

To a solution of compound 5 (195 mg, 1.03 mmol) in THF (10 mL) was added a solution of compound 1 (237 mg, 1.22 mmol) and DIPEA (0.17 mL, 1.00 mmol) in THF (5 mL) dropwise at 0° C. After addition, the mixture was stirred at room temperature for overnight. A solution 3-amino-5-methylpyrazole (146 mg, 1.50 mmol) and DIPEA (0.26 mL, 1.50 mmol) in THF (5 mL) was added to the above reaction flak at room temperature. The mixture was stirred at 60° C. for 2 hours. After cooling to room temperature, saturated NaHCO° in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (7N NH₃): 100/3 v/v as eluent to provide compound 6 as white solids (90 mg, 21%). 1H NMR (400 MHz, DMSO-d6) δ 11.80 (br, 1H), 10.43 (br, 1H), 10.06 (br, 1H), 7.75 (m, 2H), 7.48 (d, J=8.8 Hz, 2H), 5.25 (br, 1H), 1.93 (s, 3H), 1.80 (m, 2H), 0.96 (m, 4H), 0.80 (d, J=6.0 Hz, 4H); ESI-MS: calcd for (C₂₀H₂₁N₇OS) 407. found 408 (MH+). HPLC: retention time: 25.43 min. purity: 93%.

### Example 7

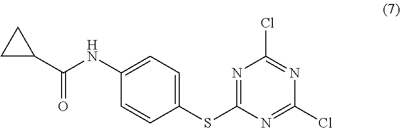

A solution of compound 1 (1.85 g, 9.57 mmol) and DIPEA (1.70 mL, 9.76 mmol) in THF (75 mL) was added dropwise to a stirred solution of cyanuric chloride (1.90 g, 10.30 mmol) in THF (50 mL) at 0° C. After the addition was complete, the reaction mixture was stirred at 10 to 20° C. for 30 more minute. Saturated ammonium chloride in water was added to the reaction mixture and the mixture was extracted with ethyl acetate (1×). The organic layer was washed by brine, dried (Na₂SO₄) and concentrated to give compound 7 as white solids (3.22 g, 99% yield). 1H NMR (400 MHz, DMSO-d6): δ 11.12 (s, 1H), 7.69 (d, t=8.4 Hz, 2H), 7.45 (d, t=8.4 Hz, 2H), 1.80 (m, 1H), 0.81 (m, 4H). ESI-MS: calcd for (C₁₃H₁₀Cl₂N₄OS) 340. found 341 (MH+).

### Example 8

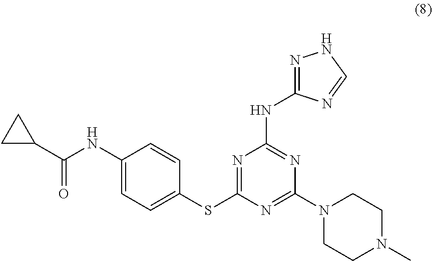

To a solution of compound 7 (180 mg, 0.53 mmol) in THF (10 mL) was added a solution of compound 3-amino-1,2,4-triazole (38 mg, 0.46 mmol) and DIPEA (0.08 mL, 0.46 mmol) in THF (5 mL) dropwise at 0° C. After addition, the mixture was stirred at 30° C. for overnight. A 1-methylpiperazine (0.10 ml, 0.90 mmol) and DIPEA (0.08 mL, 0.46 mmol) was added to the above reaction flak at room temperature. The mixture was stirred at 60° C. for 3 hours. After cooling to room temperature, saturated NaHCO₃ in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (2N NH3): 100/6 v/v as eluent to provide compound 8 as white solids (30 mg, 15%). 1H NMR (400 MHz, DMSO-d6) δ 10.41 (s, 1H), 9.50 (br, 1H), 7.74 (d, J=8.8 Hz, 2H), 7.53 (d, J=8.8 Hz, 2H), 7.10 (br, 1H), 4.60 (br, 2H), 3.60 (br, 2H), 3.00 (br, 4H), 2.80 (br, 3H), 1.80 (m, 1H), 0.81 (m, 4H); ESI-MS: calcd for (C₂₀H₂₄N₁₀OS) 452. found 453 (MH+). HPLC: retention time: 9.61 min. purity: 79%.

### Example 9

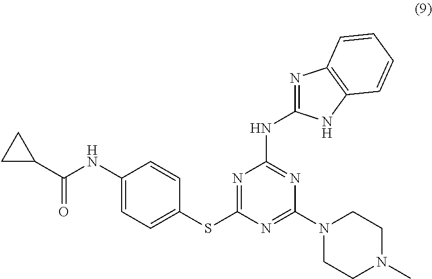

To a solution of compound 7 (180 mg, 0.53 mmol) in THF (10 mL) was added a solution of compound 2-amino-benzimidazole (61 mg, 0.46 mmol) and DIPEA (0.08 mL, 0.46 mmol) in THF (5 mL) dropwise at 0° C. After addition, the mixture was stirred at 30° C. for overnight. A 1-methylpiperazine (0.10 ml, 0.90 mmol) and DIPEA (0.08 mL, 0.46 mmol) was added to the above reaction flak at room temperature. The mixture was stirred at 60° C. for 3 hours. After cooling to room temperature, saturated NaHCO3 in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (2N NH₃): 100/3 v/v as eluent to provide compound 9 as white solids (60 mg, 26%). 1H NMR (400 MHz, DMSO-d6) δ 10.45 (s, 1H), 7.74 (d, J=8.8 Hz, 2H), 7.57 (m, 3H), 7.30 (br, 2H), 7.07 (d, J=9.2 Hz, 1H), 7.02 (t, J=8.4 Hz, 1H), 6.73 (t, J=8.4 Hz, 1H), 3.76 (br, 4H), 2.40 (br, 4H), 2.21 (br, 3H), 1.82 (m, 1H), 0.84 (m, 4H); ESI-MS: calcd for (C₂₅H₂₇N₉OS) 501. found 502 (MH+). HPLC: retention time: 10.56 min. purity: 92%.

### Example 10

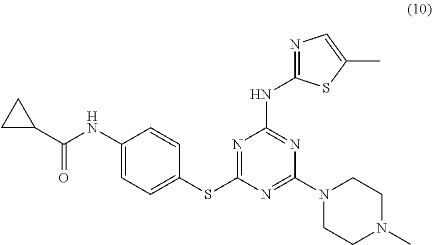

To a solution of compound 7 (180 mg, 0.53 mmol) in THF (10 mL) was added a solution of compound 2-amino-5-methylthiazole (52 mg, 0.46 mmol) and DIPEA (0.08 mL, 0.46 mmol) in THF (5 mL) dropwise at 0° C. After addition, the mixture was stirred at 30° C. for overnight. A 1-methylpiperazine (0.10 ml, 0.90 mmol) and DIPEA (0.08 mL, 0.46 mmol) was added to the above reaction flak at room temperature. The mixture was stirred at 60° C. for 3 hours. After cooling to room temperature, saturated NaHCO₃ in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (2N NH₃): 100/5 v/v as eluent to provide compound 10 as white solids (25 mg, 11%). 1H NMR (400 MHz, DMSO-d6) δ 11.15 (br, 1H), 10.40 (s, 1H), 7.69 (d, J=8.8 Hz, 2H), 7.50 (m, 2H), 7.00 (br, 1H), 3.76 (br, 4H), 2.33 (br, 4H), 2.18-2.00 (multiple s, 6H), 1.78 (m, 1H), 0.81 (m, 4H); ESI-MS: calcd for (C₂₂H₂₆N₈OS₂) 482. found 483 (MH+). HPLC: retention time: 15.51 min. purity: 96%.

### Example 11

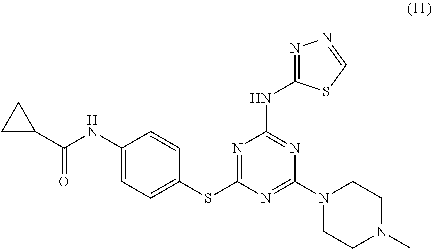

To a solution of compound 7 (180 mg, 0.53 mmol) in THF (10 mL) was added a solution of compound 2-amino-1,3,4-thiadiazole (46 mg, 0.46 mmol) and DIPEA (0.08 mL, 0.46 mmol) in THF (5 mL) dropwise at 0° C. After addition, the mixture was stirred at 30° C. for overnight. A 1-methylpiperazine (0.10 ml, 0.90 mmol) and DIPEA (0.08 mL, 0.46 mmol) was added to the above reaction flak at room temperature. The mixture was stirred at 60° C. for 3 hours. After cooling to room temperature, saturated NaHCO₃ in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (2N NH₃): 100/5 v/v as eluent to provide compound 11 as white solids (20 mg, 9%). 1H NMR (400 MHz, DMSO-d6) δ 12.00 (br, 1H), 10.37 (s, 1H), 9.00 (br, 1H), 7.65 (d, J=8.8 Hz, 2H), 7.50 (d, J=8.8 Hz, 2H), 3.76-3.50 (m, 4H), 2.40-2.20 (m, 4H), 2.16 (s, 3H), 1.78 (m, 1H), 0.81 (m, 4H); ESI-MS: calcd for (C₂₀H₂₃N₉OS₂) 469. found 470 (MH+). HPLC: retention time: 11.32 min. purity: 85%.

### Example 12

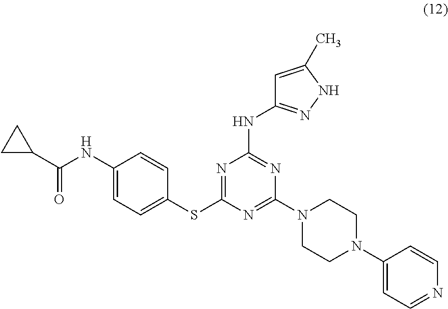

To a solution of compound 7 (200 mg, 0.59 mmol) in THF (10 mL) was added a solution of compound 3-amino-5-methylpyrazole (57 mg, 0.59 mmol) and DIPEA (0.10 mL, 0.59 mmol) in THF (3 mL) dropwise at 0° C. After addition, the mixture was stirred at 0° C. for 2 hours. A solution of 1-(4-pyridine)piperazine (110 ml, 0.67 mmol) and DIPEA (0.26 mL, 1.50 mmol) in THF (5 mL) was added to the above reaction flak at room temperature. The mixture was stirred at room temperature for overnight (white precipitation formed). Ethyl acetate and saturated NaHCO₃ in water was added to the flask. The solids was filtered and washed by ethyl acetate. The solids was dissolved in methanol and dichloromethane and mixed with silica gel. After removal of solvents, the sample was loaded on a silica gel column and eluted by DCM/MeOH (2N NH₃): 100/5 v/v to provide compound 12 as white solids (60 mg, 19%). 1H NMR (400 MHz, DMSO-d6) δ 11.85 (br, 1H), 10.41 (s, 1H), 9.59 (br, 1H), 8.15 (br, 2H), 7.75 (m, 2H), 7.49 (d, J=8.4 Hz, 2H), 6.83 (b, 2H), 5.25 (br, 1H), 3.78 (m, 4H), 3.39 (m, 4H), 1.94 (br, 3H), 1.78 (m, 1H), 0.81 (m, 4H); ESI-MS: calcd for (C₂₆H₂₈N₁₀OS) 528. found 529 (MH+). HPLC: retention time: 16.27 min. purity: 95%.

### Example 13

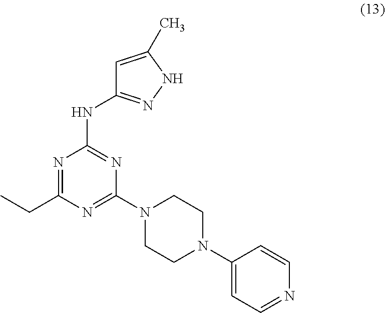

To a solution of compound 3 (163 mg, 0.90 mmol) in THF (10 mL) was added a solution of 3-amino-5-methylpyrazole (87 mg, 0.90 mmol) and DIPEA (0.16 mL, 0.90 mmol) in THF (5 mL) dropwise at 0° C. After addition, the mixture was stirred at 0° C. for additional 60 minutes. TLC was checked and the starting materials were consumed. A solution of 1-(4-pyridyl)piperazine (170 mg, 1.03 mmol) and DIPEA (0.26 mL, 1.50 mmol) in THF (5 mL) was added to the above reaction flak at room temperature. The mixture was stirred at 70° C. for 2 hours. After cooling to room temperature, saturated NaHCO₃ in water was added to the flask and the mixture was extracted by ethyl acetate (3×). The combined organic was washed by brine, dried over sodium sulfate and concentrated. The resulting crude product was purified by flash column chromatography on silica gel using DCM/MeOH (7N NH₃): 100/5 v/v as eluent to provide compound 13 as white solids (25 mg, 8%). 1H NMR (400 MHz, DMSO-d6) δ 11.80 (br, 1H), 9.50 (br, 1H), 8.16 (d, J=6.4 Hz, 2H), 6.83 (d, J=6.4 Hz, 2H), 6.30 (br, 1H), 3.85 (br, 4H), 3.40 (br, 4H), 2.51 (overlapped by solvent peak, 2H), 2.15 (s, 3H), 1.18 (t, J=7.6 Hz, 3H), ESI-MS: calcd for (C₁₈H₂₃N₉) 365. found 366 (MH+). HPLC: retention time: 3.43 min. purity: 79%.

### Example 14

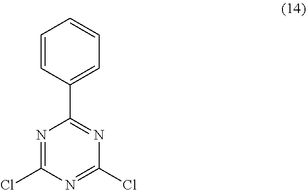

A solution of phenylmagnesium bromide in ether (3M, 16 ml, 48 mmole) was added dropwise to a stirred solution of cyanuric chloride (5.93 g, 32.16 mmole) in anhydrous dichloromethane at 5° C. After the addition was complete, the reaction mixture was stirred at 10-20° C. for 3 h. The mixture was cooled to 0° C. and added water dropwise at a rate such that the temperature of the reaction stayed below 10° C. After warming to room temperature, the reaction mixture was diluted with additional water and methylene chloride and passed through a pad of cilite, washed by saturated ammonium chloride, dried and concentrated to give 2,4-dichloro-6-phenyl-1,3,5-triazine (14) as yellow liquid, which solidified after storied in the refrigerator (1.8 g, 25%). 1H NMR (500 MHz, CDCl₃) δ 8.50 (d, J=8.0 Hz, 2H), 7.70 (t, J=8.0 Hz, 1H), 7.55 (t, J=8.0 Hz. 2H).

### Example 15

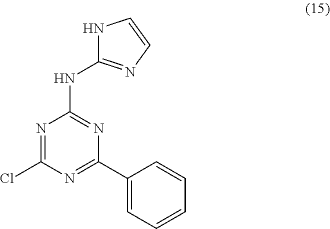

THF was added to a mixture of 2-amonoimidazole monosulfate (85 mg, 0.32 mmole) and sodium hydride (60%, 75 mg, 1.88 mmole) and the mixture was stirred for 2 hours. Compound 14 (183 mg, 0.81 mmole) was added and the mixture was stirred at 65° C. for 3 hours. Dilute NH₄Cl was added to the reaction mixture, followed by EtOAc. Light brown precipitate fanned, which was collected by filtration. The solids were washed by water, ethyl acetate and dried to give compound 15 (100 mg). The compound was used directly for further reaction without purification.

### Example 16

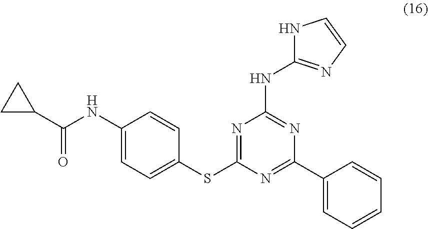

To a solution of compound 15 (80 mg, 0.29 mmol) in DMSO (5 mL) was added compound 1 (70 mg, 0.36 mmol) and DIPEA (0.20 mL, 1.15 mmol). The mixture was heated at 130° C. for 7 minutes using microwave initiator. After cooled to room temperature, saturated NaHCO₃ in water was added and the mixture was extracted by DCM/isopropal (90/10) (3×). The organic was dried (sodium sulfate) and concentrated. The crude product was purified on silica gel column and eluted by 3% MeOH in DCM to provide compound 16 as white solids (15 mg, 12%). 1H NMR (400 MHz, DMSO-d6) δ 10.45 (s, 1H), 8.36 (d, J=8.0 Hz, 2H), 7.77 (d, J=8.8 Hz, 2H), 7.60 (m, 5H), 7.35 (d, J=2.0 Hz, 1H), 56.58 (br, 1H), 6.55 (d, J=2.0 Hz, 1H), 1.82 (m, 1H), 0.83 (m, 4H); ESI-MS: calcd for (C₂₂H₁₉N₇OS) 429. found 430 (MH+). HPLC (two isomers were detected): retention time: 27.56 min, 23%; 31.25 min., 67%.

### Example 17

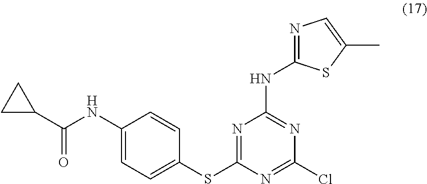

To a solution of compound 7 (1.00 g, 2.93 mmol) in THF (20 mL) was added DIPEA (0.45 mL, 2.60 mmol) and 2-amino-5-methyl-thiazole (285 mg, 2.50 mmol). The mixture was heated at 150° C. for 10 minutes using microwave initiator. After cooled to room temperature, 5 mL ethyl acetate was added and the orange solids on the wall of the tube were scratched off. The mixture was stirred at room temperature for 30 min and the solids were collected by filtration to give compound 17 (1.09 g, 88%). The crude product was used directly for the next step reaction without further purification.

### Example 18

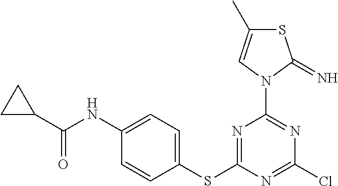

A solution of compound 7 (2.00 g, 5.86 mmol) in THF (80 mL) was cooled by using ice-NaCl batch (bath temperature −20° C.). A solution of DIPEA (1.00 mL, 5.75 mmol) and 2-amino-5-methyl-thiazole (570 mg, 5.00 mmol) was added to the above solution at −20° C. (batch temperature) dropwise. After the addition, the temperature was stirred 0° C. for 2 additional hours and then let it warmed to room temperature. The solids formed during the reaction was filtered off, washed with THF followed by ethyl acetate and dried to give light yellow solids of compound 18 (1.75 g, 83%) The crude product was used directly for the next step reaction without further purification.

### Example 19

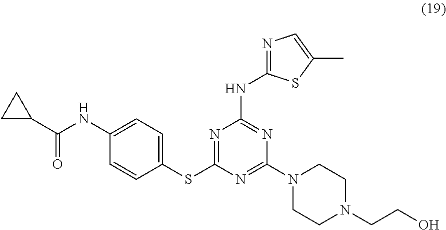

To a suspension of compound 17 (200 mg, 0.48 mmol) in isopropal (15 mL) was added 1-hydroxyethylpiperazine (130 mg, 1.00 mmol) and DIPEA (0.17 mL, 1.00 mmol) and the mixture was stirred at 60° C. for 5 minute using a micro wave initiator. After cooling to room temperature, saturated NaHCO₃ in water was added to the flask and the mixture was extracted by dichloromethane (3×). The combined organic was washed by brine, dried over sodium sulfate and concentrated. The resulting crude product was purified by flash column chromatography on silica gel using DCM/MeOH (2N NH₃): 100/5 v/v as eluent to provide compound 19 as white solids (50 mg, 20%). 1H NMR (400 MHz, DMSO-d6) δ 11.15 (br, 1H), 10.40 (s, 1H), 7.69 (br, 2H), 7.48 (d, J=8.0 Hz, 2H), 7.00 (br, 1H), 4.40 (br, 1H), 3.76 (br, 4H), 3.49 (m, 2H), 2.40-2.00 (m, 9H, 3×CH₂+CH₃), 1.78 (m, 1H), 0.78 (d, J=8.0 Hz, 4H); ESI-MS: calcd for (C₂₃H₂₈N₈O₂S₂) 512. found 513 (MH+). HPLC: retention time: 14.667 min. purity: 98%.

### Example 20

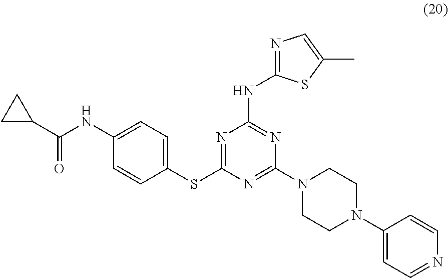

To a suspension of compound 17 (500 mg, 1.19 mmol) in DMSO/isopropal (1/1, 15 mL) was added 4-pyridylpiperazine (188 mg, 1.15 mmol) and DIPEA (0.50 mL, 2.87 mmol) and the mixture was stirred at 60° C. for 10 minute using a micro wave initiator. After cooling to room temperature, water was added to the flask and the solids were collected by filtration, washed by water, ethyl acetate. The yellow solids were suspended in MeOH/DCM and mixed with silica gel. After removal of the solvents, the sample was dry-loaded on silica gel column and purified by flash column chromatography (DCM/MeOH (2N NH₃): 100/5 v/v as eluent to provide compound 20 as white solids (50 mg, 8%). 1H NMR (400 MHz, DMSO-d6) δ 11.39 (br, 1H), 10.39 (s, 1H), 8.14 (d, J=8.0 Hz, 2H), 7.68 (br, 2H), 7.50 (d, J=8.0 Hz, 2H), 7.00 (br, 1H), 6.83 (d, J=8.0 Hz, 2H), 3.90-3.70 (m, 4H), 3.50-3.30 (m, 4H), 2.30 (br, 3H), 1.78 (m, 1H), 0.79 (d, J=8.0 Hz, 4H); ESI-MS: calcd for (C₂₆H₂₇N₉OS₂) 545. found 546 (MH+). HPLC: retention time: 20.757 min. purity: 90%.

### Example 21

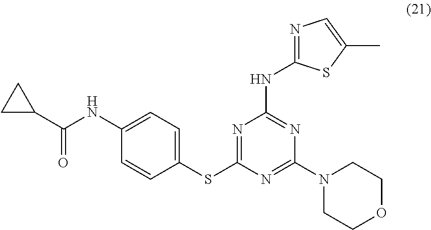

To a suspension of compound 17 (200 mg, 0.48 mmol) in isopropal (15 mL) was added morpholine (0.10 mL, 1.15 mmol) and DIPEA (0.17 mL, 1.00 mmol) and the mixture was stirred at 60° C. for 5 minute using a micro wave initiator. After cooling to room temperature, saturated NaHCO₃ in water was added to the flask and the mixture was extracted by dichloromethane (3×). The combined organic was washed by brine, dried over sodium sulfate and concentrated. The resulting crude product was purified by flash column chromatography on silica gel using DCM/MeOH (2N NH₃): 100/2 v/v as eluent to provide compound 21 as white solids (50 mg, 22%). 1H NMR (400 MHz, DMSO-d6) δ 11.15 (br, 1H), 10.36 (s, 1H), 7.69 (br, 2H), 7.48 (d, J=8.0 Hz, 2H), 7.00 (br, 1H), 4.00-3.50 (m 8H), 2.14 (m, 3H), 1.78 (m, 1H), 0.78 (br, 4H); ESI-MS: calcd for (C₂₁H₂₃N₇O₂S₂) 469. found 470 (MH+). HPLC: retention time: 29.216 min. purity: 95%.

### Example 22

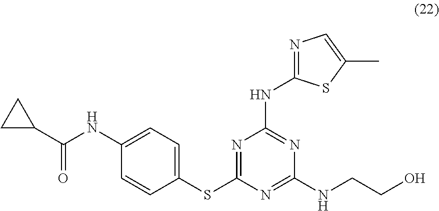

To a suspension of compound 17 (100 mg, 0.23 mmol) in isopropal (5 mL) was added ethanolamine (0.05 mL, 0.82 mmol) and DIPEA (0.10 mL, 0.50 mmol) and the mixture was stirred at 60° C. for 5 minute using a micro wave initiator. After cooling to room temperature, saturated NaHCO₃ in water was added to the flask and the mixture was extracted by dichloromethane (3×). The combined organic was washed by brine, dried over sodium sulfate and concentrated. The resulting crude product was purified by flash column chromatography on silica gel using DCM/MeOH (2N NH₃): 100/5 v/v as eluent to provide compound 22 as white solids (21 mg, 21%). ESI-MS: calcd for (C₁₉H₂₁N₇O₂S₂) 443. found 444 (MH+).

### Example 23

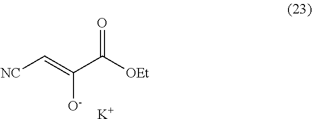

To a solution of Potassium ethoxide (24 wt % in ethanol, 100 mL, 253 mmol) was added dry Et₂O (50 mL) under Argon. The mixture was cooled to 0° C. in ice, diethyl oxalate (18.26 g, 125 mmol), dissolved in Et₂O (17 mL), was added dropwise, and the reaction mixture was stirred for 30 min. A solution of CH₃CN (5.18 g (6.57 mL), 125 mmol) in Et₂O (10 mL) was added, and the mixture was allowed to warm to room temperature and stirred for 1.5 h. The precipitated solid was collected by filtration to give compound 23 (17 g, 76% yield). The product was used without further purification.

### Example 24

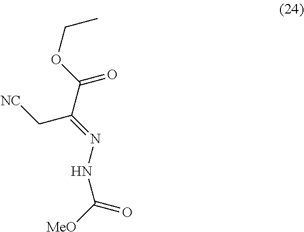

To a suspension of the potassium cyano pyruvate compound 23 (5.00 g, 25.35 mmol) in chloroform (250 mL) was added HCl (2M in ethyl ester, 20 mL, 40 mmol). Methyl hydrazino formate (2.28 g, 25.35 mmol) was added, the mixture was stirred for 24 h at room temperature, any precipitated solid was removed by filtration through a pad of celite, and the filtrate was concentrated to give an oil. The crude product was purified by column chromatography (30% ethyl acetate in hexane) to yield 24 (1.33 g, 25%) as a light-yellow oil; 1H NMR (400 MHz, CDCl₃) δ[ppm] 11.94 (br s, 1H, NH), 4.40 (q, 3J=7.1 Hz, 2H), 3.89 (s, 3H), 3.60 (s, 2H), 1.40 (t, 3H, 3J=7.1 Hz); ESI-MS: calcd for (C₈H₁₁N₃O₄) 213. found 236 (MNa+).

### Example 25

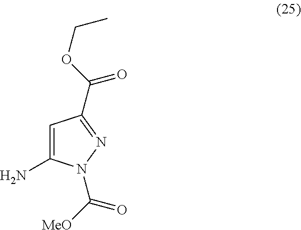

To a solution of 24 (1.15 g, 5.39 mmol) in CH₃CN (50 mL) was added triethylamine (1.5 mL, 10.71 mmol) and the mixture was stirred for 30 min at room temperature. The solvent was removed in vacuo and the solid residue was recrystallized from ethyl acetate to give N-methoxycarbonyl-3-aminopyrazole-5-carboxylic acid ethyl ester (compound 25) (613 mg, 53%) as colorless crystals. 1H NMR (400 MHz, CDCl₃) S[ppm] 5.90 (s, 1H), 5.42 (br s, 2H), 4.39 (q, 3J=7.1 Hz, 2H), 4.05 (s, 3H), 1.38 (t, 3J=7.1 Hz, 3 H); ESI-MS: calcd for (C₈H₁₁N₃O₄) 213. found 236 (MNa+).

### Example 26

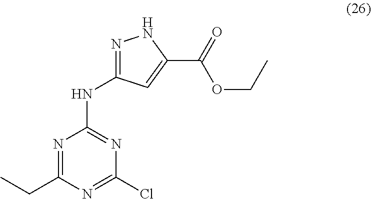

A mixture of compound 3 (420 mg, 2.36 mmol), compound 25 (320 mg, 1.50 mmol) and DIPEA (0.31 mL, 1.80 mmol) in THF/DCM (12 mL/3 mL) was heated at 180° C. for 20 minutes with microwave initiator. After cooling to room temperature, the mixture was mixed with silica gel and the solvents were removed in vacuo. The solids were dry-loaded on a silica gel column and purified by column chromatography (50% ethyl acetate in hexane) to give compound 26 as white solids (150 mg, 34%). 1H NMR (400 MHz, CDCl₃) δ: 13.68 (br, 1H), 11.17 (br, 1H), 7.16 (br, 1H), 4.31 (q, J=7.1 Hz, 2H), 2.62 (br, 2H), 1.28 (t, J=7.1 Hz, 3H), 1.21 (br, 3H); ESI-MS: calcd for (C₁₁H₁₃ClN₆O₂) 296. found 297 (MH+).

### Example 27

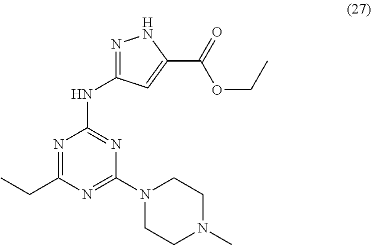

To a solution of compound 26 (120 mg, 0.40 mmol) in DMSO (5 mL) was added 1-methylpiperazine (0.22 ml, 1.98 mmol) and DIPEA (0.17 mL, 1.00 mmol) and the mixture was stirred at 60° C. for 10 minutes with micro wave initiator. After cooling to room temperature, water was added and yellow solids formed, which was collected by filtration. The resulting crude product was purified by flash column chromatography on silica gel using DCM/MeOH (2N NH₃): 100/3 v/v as eluent to provide compound 27 as light-yellow solids (115 mg, 80%). 1H NMR (400 MHz, DMSO-d6) δ 13.40 (br, 1H), 9.90 (br, 1H), 7.06 (br, 1H), 4.26 (q, J=8.0 Hz, 2H), 3.73 (br, 4H), 2.35 (br, 4H), 2.21 (br, 3H), 1.26 (t, J=8.0 Hz, 3H), 1.15 (t, J=8.0 Hz, 3H); ESI-MS: calcd for (C₁₆H₂₄N₈O₂) 360. found 361 (MH+). HPLC: retention time: 5.195 min. purity: 98%.

### Example 28

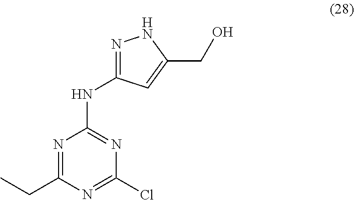

To a solution of compound 27 (90 mg, 0.25 mmol) in dichloromethane (15 mL) was added diisobutylaluminum hydride (1.-M solution in THF, 2.00 ml, 2.00 mmol) at 0° C. dropwise and the mixture was stirred at room temperature for 72 hours, upon which, the starting material was almost consumed. Rachelle salt (aq solution, 20 mL) was added and the mixture was stirred at room temperature for additional 3 hours. The mixture was extracted with DCM (3×) and the combined organic was washed by brine, dried with sodium sulfate and concentrated. The resulting crude product was purified by flash column chromatography on silica gel using DCM/MeOH: 100/20 v/v as eluent to provide compound 28 as colorless semi-solids (28 mg, 35%). 1H NMR (400 MHz, DMSO-d6) δ 12.00 (br, 1H), 9.52 (br, 1H), 6.356 (br, 1H), 5.13 (br, 1H), 4.39 (s, 1H), 3.71 (br, 4H), 3.28 (br, 4H), 2.42 (q, J=7.2 Hz, 2H), 32.17 (s, 1H), 1.16 (t, J=7.2 Hz, 3H); ESI-MS: calcd for (C₁₄H₂₂N₈O) 318. found 319 (MH+). HPLC: retention time: 1.835 min. purity: 99%.

### Example 29

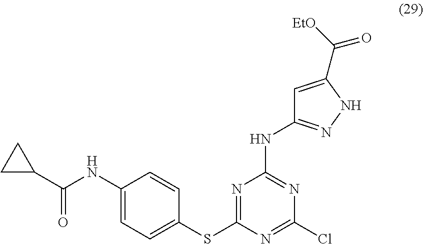

A mixture of compound 7 (210 mg, 0.62 mmol), compound 25 (88 mg, 0.41 mmol) and DIPEA (0.08 mL, 0.46 mmol) in THF/(15 mL) was heated at 180° C. for 40 minutes with micro wave initiator. After cooling to room temperature, the mixture was mixed with silica gel and the solvents were removed in vacuo. The solids were dry-loaded on a silica gel column and purified by pass a pad of silica gel (5% methanol in DCM) to give compound 29 which was used for the next step reaction without further purification.

### Example 30

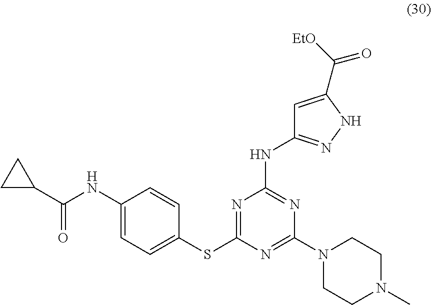

To a solution of compound 29 (obtained as described above)) in DMSO (10 mL) was added 1-methylpiperazine (0.15 ml, 1.35 mmol) and DIPEA (0.15 mL, 0.88 mmol) and the mixture was stirred at 60° C. for 10 minutes with micro wave initiator. After cooling to room temperature, water was added and yellow solids formed, which was collected by filtration. The resulting crude product was purified by flash column chromatography on silica gel using DCM/MeOH (7N NH₃): 100/2 v/v as eluent to provide compound 30 as light-yellow solids (45 mg, 21% for 2 steps). 1H NMR (400 MHz, DMSO-d6) δ 13.40 (br, 1H), 10.32 (s, 1H), 9.90 (br, 1H), 7.63 (d, J=8.4 Hz, 2H), 7.44 (d, d, J=8.4 Hz, 2H), 6.78 (br, 1H), 4.25 (q, J=7.2 Hz, 2H), 3.60 (br, 4H), 2.35 (br, 4H), 2.17 (s, 3H), 1.77 (m, 1H), 1.25 (t, J=7.2 Hz, 3H), 0.79 (m, 4H); ESI-MS: calcd for (C₂₄H₂₉N₉O₃S) 523. found 524 (MH+). HPLC: retention time: 19.595 min. purity: 94%.

### Example 31

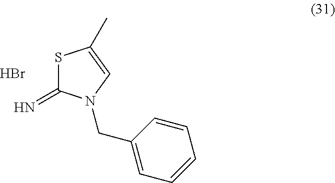

A mixture of 2-amino-5-methylthiazol (660 mg, 5.78 mmol) and benzyl bromide (0.76 mL, 6.36 mmol) in acetone (10 mL) was refluxed for 5 hours. After cooling to room temperature, the white solids formed during the reaction was collected by filtration, washed with acetone and dried under vac. To give compound 31 (350 mg, 22%). 1H NMR (400 MHz, DMSO-d6) δ 9.56 (s, 2H), 7.36-7.16 (m, 5H), 5.17 (s, 1H), 2.15 (s, 3H); ESI-MS: calcd for (free base) (C₁₁H₁₂N₂S) 204. found 205 (MH+).

### Example 32

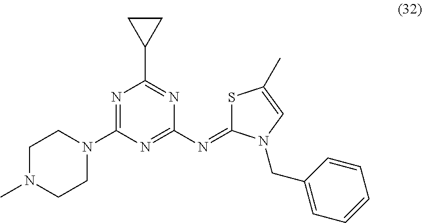

A mixture of compound 31 (25 mg, 0.088 mmol), compound 5 (21 mg, 0.11 mmol) and DIPEA (0.02 mL, 0.11 mmol) in THF (1 mL) was heated at 120° C. for 5 minutes with micro wave initiator. After cooling to room temperature, 1-methylpiperazine (0.1 mL, 1.00 mmol) and DIPEA (0.2 mL, 0.11 mmol) was added to the mixture and heated at 60° C. for 5 minutes with micro wave initiator. Saturated sodium bicarbonate in water was added and the mixture was extracted with dichloromethane (3×). the combined organic was dried over sodium sulfate and concentrated to give compound 32. The resulting crude product was not further purified (25 mg, 80%). 1H NMR (400 MHz, DMSO-d6) δ 7.33-7.26 (m, 5H), 6.41 (s, 1H), 5.37 (s, 2H), 3.95 (m, 4H), 2.45 (m, 4H), 2.35 (s, 3H), 2.19 (s, 3H), 2.05 (m, 1H), 1.22 (m, 2H), 0.95 (m, 2H); ESI-MS: calcd for (C₂₂H₂₇N₇S) 421. found 422 (MH+).

### Example 33

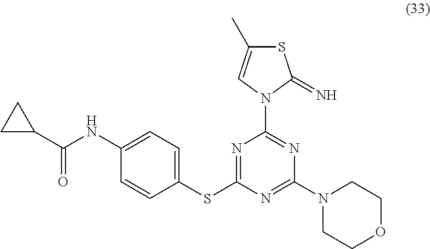

To a suspension of compound 18 (265 mg, 0.63 mmol) in DMSO/isopropal (15 mL/5 mL) was added morpholine (0.15 mL, 1.72 mmol) and DIPEA (0.15 mL, 0.86 mmol) and the mixture was stirred at 60° C. for 10 minute using a micro wave initiator. After cooling to room temperature, Water was added to the flask and the precipitate formed, which was collected by filtration, washed by water. The crude product was crystallized from methanol/DCM to give compound 33 as white solids (80 mg, 27%). 1H NMR (400 MHz, DMSO-d6) δ 10.39 (s, 1H), 8.93 (s, 1H), 7.68 (d, J=8.0 Hz, 2H), 7.46 (d, J=8.0 Hz, 2H), 7.17 (s, 1H), 3.70-3.50 (m 8H), 1.99 (s, 3H), 1.77 (m, 1H), 0.79 (br, 4H); ESI-MS: calcd for (C₂₁H₂₃N₇O₂S₂) 469. found 470 (MH+). HPLC: retention time: 23.125 min. purity: 99%.

### Example 34

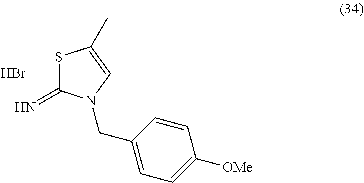

A mixture of 2-amino-5-methylthiazol (1.00 g, 8.76 mmol) and 4-methoxybenzyl bromide (1.53 mL, 10.51 mmol) in acetone (10 mL) was refluxed for 5 overnight. After cooling to room temperature, ethyl acetate (˜5 mL) was added and pink precipitate formed, which was collected by filtration, washed with acetone/ethyl acetate and dried under vac. To give compound 34 (250 mg, 11%). 1H NMR (400 MHz, DMSO-d6) δ 9.51 (s, 2H), 7.28 (d, J=8.86, 2H), 7.13 (s, 1H), 6.93 (d, J=8.86, 2H), 5.08 (s, 1H), 3.72 (s, 3H), 2.17 (s, 3H); ESI-MS: calcd for (free base) (C₁₂H₁₅BrN₂OS) 234. found 235 (MH+).

### Example 35

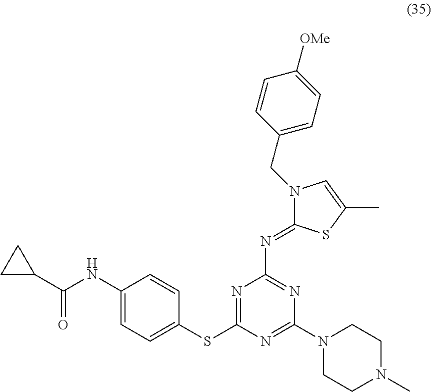

To a solution of compound 7 (170 mg, 0.50 mmol) in THF (5 mL) was added compound 34 (125 mg, 0.39 mmol) and DIPEA (0.10 mL, 0.53 mmol). After addition, the mixture was heated at 150° C. for 10 minutes using micro wave initiator. After cooling to room temperature, 1-methylpiperazine (0.10 ml, 0.90 mmol) and DIPEA (0.20 mL, 1.06 mmol) was added to the above reaction tube. The mixture was stirred at 60° C. for 10 minutes using micro wave initiator. After cooling to room temperature, saturated NaHCO₃ in water was added and the mixture was extracted by DCM (3×). The combined organic was washed by brine, dried over sodium sulfate and concentrated. The resulting crude product was purified by flash column chromatography on silica gel using DCM/MeOH (2N NH₃): 100/2 v/v as eluent to provide compound 35 as white solids (160 mg, 76%). 1H NMR (400 MHz, DMSO-d6) δ 10.36 (s, 1H), 7.67 (d, J=8.8 Hz, 2H), 7.48 (d, J=8.8 Hz, 2H), 7.17 (d, J=8.8 Hz, 2H), 6.99 (s, 1H), 6.85 (d, J=8.8 Hz, 2H), 5.11 (s, 2H), 3.67 (s, 1H), 3.65 (br, 4H), 2.25 (br, 4H), 2.18 (s, 3H), 1.99 (s, 3H), 1.78 (m, 1H), 0.78 (m, 4H); ESI-MS: calcd for (C₃₀H₃₄N₈O₂S₂) 602. found 603 (MH+).

### Example 36

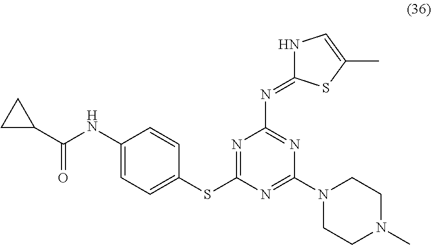

To a solution of compound 35 (˜150 mg, 0.25 mmol) in TFA (10 mL) was heated at 100° C. for 45 minutes using micro wave initiator. After cooling to room temperature, the solvent was removed under reduced pressure. Saturated NaHCO₃ was added and the mixture was extracted by DCM/isopropal (3×). The combined organic was dried over sodium sulfate and concentrated. The resulting crude product was purified by flash column chromatography on silica gel using DCM/MeOH (2N NH₃): 100/5 v/v as eluent to provide compound 36 as white solids (70 mg, 58%). 1H NMR (400 MHz, DMSO-d6) δ 11.15 (br, 1H), 10.40 (s, 1H), 7.69 (d, J=8.8 Hz, 2H), 7.50 (m, 2H), 7.00 (br, 1H), 3.76 (m, 4H), 2.33 (m, 4H), 2.18-2.00 (multiple s, 6H), 1.78 (m, 1H), 0.81 (m, 4H); ESI-MS: calcd for (C₂₂H₂₆N₈OS₂) 482. found 483 (MH+). HPLC: retention time: 16.26 min. purity: 98%.

### Example 37

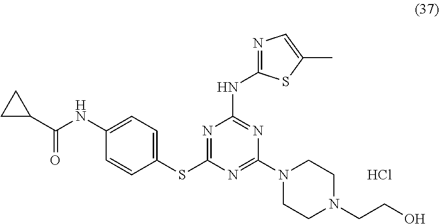

To a suspension of compound 19 (400 mg, 0.78 mmol) in imeOH/DCM (65 mL/15 mL) was added a solution of HCl in ethyl ether (1 M, 1.00 mL, 1.00 mmol) dropwise and the mixture was stirred at room temperature for 3 hours. The solvents were removed under reduced pressure, coevaperated with acetonitrile (3×) and further dried on vacuum line (<50 mmtor) to provide compound 37 as white solids (405 mg, 95%). ESI-MS: calcd for (free base) (C₂₃H₂₈N₈O₂S₂) 512. found 513 (MH+). HPLC: retention time: 21.899 min. purity: 98%.

### Example 38

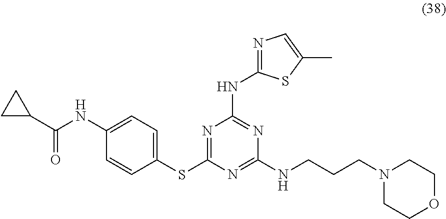

To a suspension of compound 17 (200 mg, 0.49 mmol) in DMSO (5 mL) was added 3-morpholinopropan-1-amine (0.20 mL, 1.37 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product formed. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/: 90/10/v/v/ as eluent to provide compound 38 as white solids (80 mg, 31%). 1H NMR (400 MHz, DMSO-d6) δ 11.15 (br, 1H), 10.40 (s, 1H), 7.69-7.20 (m, 4H), 7.00 (br, 1H), 3.60-3.20 (m, 6H), 2.40-2.00 (m, 9H, 3×CH₂+CH₃), 1.80-1.20 (m, 3H), 0.78 (d, J=8.0 Hz, 4H); ESI-MS: calcd for (C₂₄H₃₀N₈O₂S₂) 526. found 527 (MH+). HPLC: retention time: 16.011 min. purity: 95%.

### Example 39

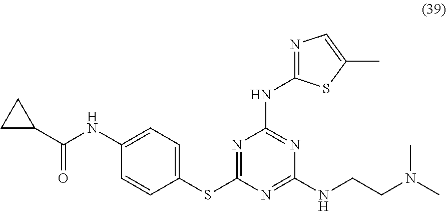

To a suspension of compound 17 (200 mg, 0.49 mmol) in DMSO (5 mL) was added N′,N′-dimethylethane-1,2-diamine (0.20 mL, 2.27 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product formed. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/TEA: 90/10/1 v/v/v as eluent to provide compound 39 as white solids (66 mg, 29%). 1H NMR (400 MHz, DMSO-d6) δ 11.15 (br, 1H), 10.52 (s, 1H), 7.69-7.20 (m, 4H), 7.00 (br, 1H), 3.28 (s, 6H), 2.80 (m, 4H), 2.20 (b, 3H), 1.80 (m, 1H), 0.78 (d, J=8.0 Hz, 4H); ESI-MS: calcd for (C₂₁H₂₆N₈OS₂) 470. found 471 (MH+). HPLC: retention time: 15.040 min. purity: 84%.

### Example 40

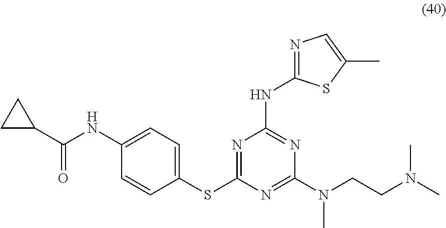

To a suspension of compound 17 (200 mg, 0.49 mmol) in DMSO (5 mL) was added N1,N1,N2-trimethylethane-1,2-diamine (0.20 mL, 1.55 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product formed. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/TEA: 90/10/1 v/v/v as eluent to provide compound 40 as white solids (110 mg, 46%). 1H NMR (400 MHz, DMSO-d6) δ 11.25 (br, 1H), 10.34 (br, 1H), 7.69-7.30 (m, 4H), 7.00 (br, 1H), 3.80-1.99 (m, 16H), 1.80 (m, 1H), 0.78 (d, J=8.0 Hz, 4H); ESI-MS: calcd for (C₂₂H₂₈N₈OS₂) 484. found 485 (MH+). HPLC: retention time: 18.283 min. purity: 100%.

### Example 41

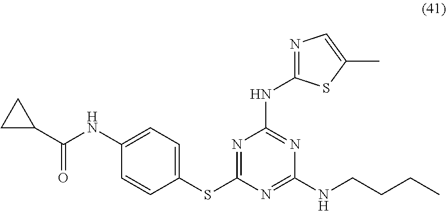

To a suspension of compound 17 (200 mg, 0.49 mmol) in DMSO (5 mL) was added butan-1-amine (0.20 mL, 2.02 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product Ruined. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/TEA: 90/10/1 v/v/v as eluent to provide compound 41 as white solids (11 mg, 5%). 1H NMR (400 MHz, DMSO-d6) δ 11.25 (br, 1H), 10.34 (br, 1H), 7.69-7.30 (m, 4H), 7.00 (br, 1H), 3.80-1.20 (m, 13H), 0.78 (d, J=8.0 Hz, 4H); ESI-MS: calcd for (C₂₁H₂₅N₇OS₂) 455. found 456 (MH+). HPLC: retention time: 32.437 min. purity: 90%.

### Example 42

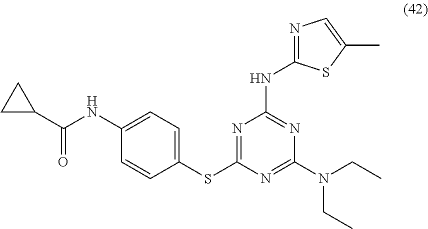

To a suspension of compound 17 (200 mg, 0.49 mmol) in DMSO (5 mL) was added diethylamine (0.20 mL, 1.94 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product formed. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/TEA: 90/10/1 v/v/v as eluent to provide compound 42 as white solids (58 mg, 26%). 1H NMR (400 MHz, DMSO-d6) δ 11.25 (br, 1H), 10.34 (br, 1H), 7.65 (d, J=8.4 Hz, 2H), 7.47 (d, J=8.4 Hz, 2H), 7.00 (br, 1H), 3.59-3.20 (m, 4H), 2.30 (br, 3H), 1.78 (m, 1H), 1.20 (br, 3H), 1.00 (br, 3H), 0.78 (m, 4H); ESI-MS: calcd for (C₂₁H₂₅N₇OS₂) 455. found 456 (MH+). HPLC: retention time: 35.371 min. purity: 99%.

### Example 43

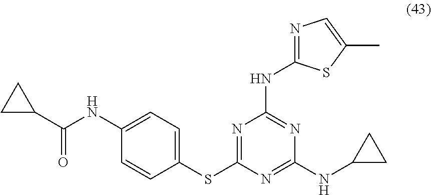

To a suspension of compound 17 (200 mg, 0.49 mmol) in DMSO (5 mL) was added cyclopropanamine (0.20 mL, 3.50 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product formed. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/TEA: 90/10/1 v/v/v as eluent to provide compound 43 as white solids (15 mg, 7%). ESI-MS: calcd for (C₂₀H₂₁N₇OS₂) 439. found 440 (MH+).

### Example 44

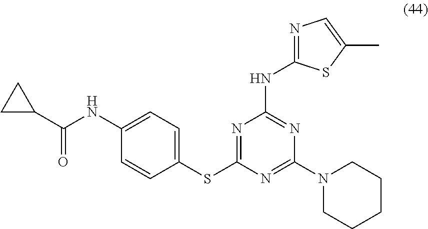

To a suspension of compound 17 (200 mg, 0.49 mmol) in DMSO (5 mL) was added diethylamine (0.20 mL, 2.35 mmol) and DIPEA (0.17 mL, 0.97 mmol) and the mixture was stirred at 60° C. for 15 minute using a micro wave initiator. After cooling to room temperature, water (˜15 mL) was added and the mixture was cooled to 4° C. overnight, during which, yellow solids of the crude product formed. The solids were collected by filtration, washed by water, hexanes and then suspended in MeOH/DCM and mixed with silica gel. After removal of the solvents, the sample was dry-loaded on silica gel column and purified by flash column chromatography (DCM/MeOH/TEA: 90/10/1 v/v/v as eluent to provide compound 44 as white solids (32 mg, 14%). 1H NMR (400 MHz, DMSO-d6) δ 11.25 (br, 1H), 10.34 (br, 1H), 7.65 (br, 2H), 7.47 (d, J=8.4 Hz, 2H), 7.00 (br, 1H), 3.80-3.20 (m, 4H), 2.30 (br, 3H), 1.78 (m, 1H), 1.60-1.20 (m, 6H), 0.78 (m, 4H); ESI-MS: calcd for (C₂₂H₂₅N₇OS₂) 467. found 468 (MH+). HPLC: retention time: 36.683 min. purity: 98%.

### Example 45

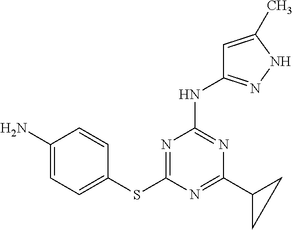

To a solution of compound 5 (230 mg, 1.21 mmol) in THF (15 mL) was added a solution of 3-amino-5-methylpyrazole (118 mg, 1.21 mmol) and DIPEA (0.21 mL, 1.21 mmol) in THF (15 mL) dropwise at 0° C. After addition, the mixture was stirred at 0° C. for 2 hours. A solution of 4-aminothiophenol (212 mg, 1.69 mmol) and sodium hydride (110 mg, 4.58 mmol) in DMF (3 mL) was added to the above reaction flak at room temperature. The mixture was stirred at room temperature for 5 hours. Saturated NH₄Cl in water was added to the flask and the mixture was extracted by DCM (3×). The combined organic was dried over sodium sulfate and concentrated. The resulting crude product was purified by flash column chromatography on silica gel using Hexanes/EtOAc: 40/60 v/v as eluent to provide compound 45 as white solids (180 mg, 44%). 1H NMR (400 MHz, DMSO-d6) δ 11.80 (br, 1H), 9.98 (s, 1H), 7.14 (d, J=8.4 Hz, 2H), 6.60 (d, J=8.4 Hz, 2H), 5.50 (br, 2H), 5.46 (s, 1H), 2.05 (br, 3H), 1.80 (m, 2H), 0.94 (m, 4H); ESI-MS: calcd for (C₁₆H₁₇N₇S) 339. found 340 (MH+). HPLC: retention time: 20.533 min. purity: 99%.

### Example 46

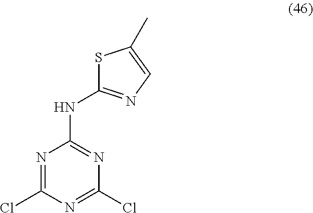

A solution of 2-amino-5-methylthiazol (1.30 g, 13.56 mmol) and DIPEA (2.00 mL, 11.48 mmol) in THF (55 ml) was added dropwise to a stirred solution of cyanuric chloride (2.50 g, 13.56 mmol) in THF (70 mL) at −5° C. After the addition was complete, the reaction mixture was stirred at −5° C. for 15 more minute. During the stirring, large amount of yellow precipitate formed, which was collected by filtration, washed with THF (3×20 mL), ethyl acetate (3×20 mL) and hexanes (1×10 mL). The compound 46 (2.72 g, 91%) was used directly for further reaction without purification.

### Example 47

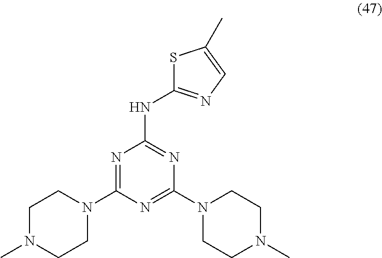

To a solution of compound 46 (50 mg, 0.19 mmol) in DMF (5 mL) was added 1-methylpiperazine (0.10 mL, 0.90 mmol) and the mixture was stirred at room temperature for 1 hours then at 60° C. for 10 minutes using microwave initiator. After cooling to room temperature, water was added and the solids were collected by filtration, washed with water, then hexanes to provide compound 47 as white solids (20 mg, 27%). 1H NMR (400 MHz, DMSO-d6) δ 10.89 (s, 1H), 7.00 (s, 1H), 3.78 (br, 8H), 2.35 (m, 11H), 2.18 (s, 6H); ESI-MS: calcd for (C₁₇H₂₇N₉S) 389. found 390 (MH+). HPLC: retention time: 1.813 min. purity: 93%.

### Example 48

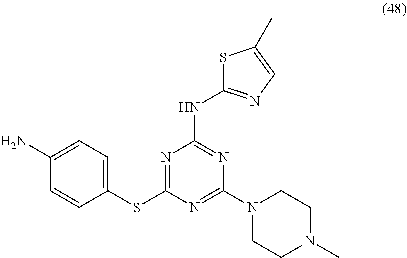

To a solution of compound 46 (565 mg, 2.16 mmol) in DMF (60 mL) was added a solution of 1-methylpiperazine (0.20 mL, 1.80 mmol) and DIPEA (0.35 mL, 1.80 mmol) in DMF (30 mL) dropwise at −15° C. After addition, the mixture was stirred at 0° C. for 30 minutes. A solution of 4-aminothiophenol (700 mg, 5.60 mmol) and sodium hydride (60%, 260 mg, 6.50 mmol) in DMF (7 mL) was added to the above reaction flak at room temperature. The mixture was stirred at room temperature for overnight. Saturated NH₄Cl in water was added to the flask and the mixture was extracted by DCM/isopropal (v/v: 97/3, 3×). The combined organic was washed with water, dried over sodium sulfate and concentrated. The resulting crude product was purified by flash column chromatography on silica gel using methanol/DCM: 10/90 v/v as eluent to provide compound 48 as white solids (320 mg, 43%). 1H NMR (400 MHz, DMSO-d6) δ 11.20 (br, 1H), 7.14 (d, J=8.4 Hz, 2H), 7.00 (br, 1H), 6.60 (d, J=8.4 Hz, 2H), 5.60 (br, 2H), 3.80 (m, 4H), 2.25 (m, 10H); ESI-MS: calcd for (C₁₈H₂₂N₈S₂) 414. found 415 (MH+). HPLC: retention time: 11.648 min. purity: 97%.

### Example 49

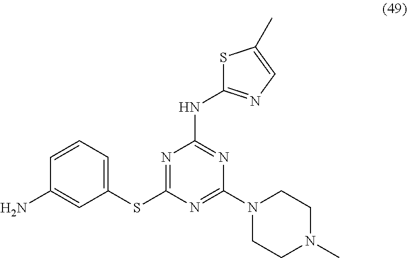

To a solution of compound 46 (1.30, 4.96 mmol) in DMF (60 mL) was added a solution of 1-methylpiperazine (0.42 mL, 3.81 mmol) and DIPEA (0.66 mL, 3.81 mmol) in DMF (50 mL) dropwise at −15° C. After addition, the mixture was stirred at 0° C. for 30 minutes. A solution of 3-aminothiophenol (700 mg, 5.60 mmol) and sodium hydride (60%, 260 mg, 6.50 mmol) in DMF (7 mL) was added to the above reaction flak at room temperature. The mixture was stirred at room temperature for overnight. Saturated NH₄Cl in water (20 mL) was added to the flask and the mixture was concentrated. The residue was washed by water, decanted and suspended in DCM. The resulting crude product was purified by flash column chromatography on silica gel using methanol/DCM: 15/85 v/v as eluent to provide compound 49 as white solids (210 mg, 13%). 1H NMR (400 MHz, DMSO-d6) δ 11.80 (br, 1H), 7.20-6.80 (m, 5H), 5.20 (br, 2H), 3.80 (m, 4H), 3.00 (m, 4H), 2.25 (m, 6H); ESI-MS: calcd for (C₁₈H₂₂N₈S₂) 414. found 415 (MH+).

### Example 50

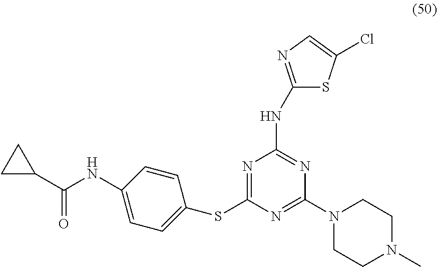

To a solution of compound 7 (150 mg, 0.49 mmol) in THF (15 mL) was added a solution of 2-amino-5-chlorothiozole (54 mg, 0.40 mmol) and DIPEA (0.09 mL, 0.49 mmol) in 10-20 mL microwave vial. Vial was sealed with a cap and the mixture was allowed to stir at 150° C. for 5 min. in the microwave synthesizer. Next, compound 1-methy piperazine (0.07 mL, 0.59 mmol) and DIPEA (0.10 mL, 0.59 mmol) were added to the above mixture and allowed to stir at 60° C. for 10 min. in the microwave synthesizer. Saturated NaHCO₃ in water was added and the mixture was extracted by ethyl acetate (3×50 mL). The combined organic was washed by brine, dried over sodium sulfate and concentrated. The residue was chromatographed on a silica gel column eluted with 0-5% MeOH/DCM afforded 50 white solid (30 mg, 14%). 1H NMR (400 MHz, DMSO-d6) δ 11.70 (bs, 1H, NH), 10.42 (s, 1H, NH), 7.85-7.17 (m, 5H, Ar—H), 3.83-3.51 (m, 4H, 2CH₂), 2.46-2.28 (m, 4H, 2CH₂), 2.20 (s, 3H, CH₃), 1.84-1.78 (m, 1H, CH), 0.81-0.80 (m, 4H, Ar—H); ESI-MS: calcd for (C₂₁H₂₃ClN₈OS₂) 502. found 503 [M+H]+. HPLC: retention time: 20.70 min. purity: 91%.

### Example 51

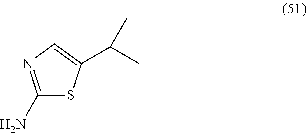

To a solution of 3-methylbutyl aldehyde (0.9 mL, 7.18 mmol) in Et₂O (15 mL) was added 5,5-dibromobarbituric acid (1.0 g, 3.59 mmol). Reaction was stirred at room temperature for 18 h. Mixture was filtered, washed with ether and concentrated. Residue was washed with hexane, filtered and concentrated. Residue was dissolved in EtOH (20 mL) and thiourea was added. Mixture was refluxed for 1 d. Reaction was neutralized with 7N ammonia and concentrated. Residue was chromatographed on a silica gel column eluted with 1-10% MeOH/DCM afforded 51. 1H NMR (400 MHz, DMSO-d6) δ 6.72 (d, J=1.2 Hz, H, Ar—H), 4.96 (bs, 2H, NH2), 3.03-2.96 (m, 1H, CH), 1.27 (s, 3H, CH₃), 1.25 (s, 3H, CH₃); ESI-MS: calcd for (C₆H₁₀N₂S) 142. found 143 [M+H]+.

### Example 52

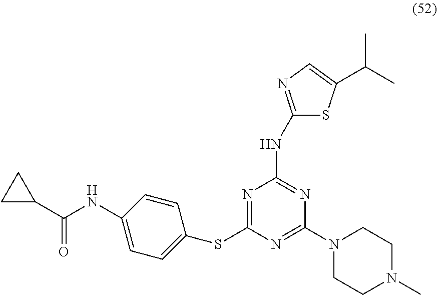

Compound 7 (200 mg, 0.59 mmol) was reacted with compound 51 and treated as described for preparation of compound 50. After purification, compound 52 was obtained as light yellow solid (50 mg, 17%). 1H NMR (400 MHz, DMSO-d6) δ 11.25 (bs, 1H, NH), 10.41 (s, 1H, NH), 7.74-7.72 (m, 2H, Ar—H), 7.52 (d, J=9.3 Hz, 2H, Ar—H), 6.98 (bs, 1H, Ar—H), 3.86-3.54 (m, 4H, 2CH₂), 2.98-2.80 (m, 1H, CH), 2.42-2.28 (m, 4H, 2CH₂), 2.20 (s, 3H, CH₃), 1.84-1.78 (m, 1H, CH), 1.15 (bs, 6H, 2CH₃), 0.83-0.81 (m, 4H, Ar—H); ESI-MS: calcd for (C₂₄H₃₀N₈OS₂) 510. found 511 [M+H]+. HPLC: retention time: 19.86 min. purity: 95%.

### Example 53

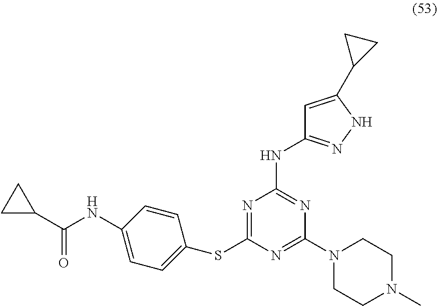

Compound 7 (300 mg, 0.88 mmol) was reacted syquencely with 5-cyclopropyl-1H-pyrazol-3-amine and 1-methylpiperazine as described for preparation of compound 50. compound 53 was obtained as light yellow solid (10 mg, 3%). 1H NMR (400 MHz, DMSO-d6) δ 11.66 (bs, 1H, NH), 10.40 (s, 1H, NH), 9.49 (bs, 1H, NH), 7.78-7.68 (m, 2H, Ar—H), 7.45 (d, J=8.4 Hz, 2H, Ar—H), 5.33 (bs, 1H, Ar—H), 3.65-3.56 (m, 4H, 2CH₂), 3.10-3.08 (m, 1H, CH), 2.39-2.31 (m, 4H, 2CH₂), 2.21 (s, 3H, CH₃), 1.81-1.75 (m, 1H, CH), 1.15 (bs, 6H, 2CH₃), 1.26-1.22 (m, 4H, Ar—H), 0.77-0.76 (m, 4H, Ar—H); ESI-MS: calcd for (C₂₄H₂₉N₈OS) 491. found 492 [M+H]+. HPLC: retention time: 15.02 min. purity: 97%.

### Example 54

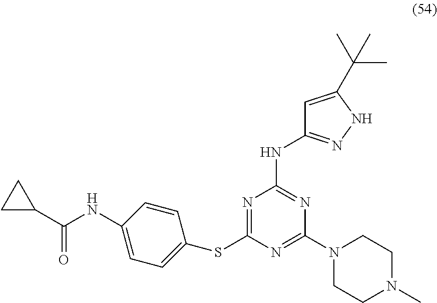

To a solution of compound 7 (300 mg, 0.88 mmol) in THF (15 mL) was added a solution of 2-amino-5-tert-butylpyrazole (98 mg, 0.70 mmol) and DIPEA (0.16 mL, 0.88 mmol). The reaction mixture was let to stir at room temperature for 3 h. Then, 1-methyl piperazine (0.15 mL, 1.32 mmol) and DIPEA (0.23 mL, 1.32 mmol) was added to the above reaction flak at room temperature. The mixture was stirred at room temperature for overnight. Saturated NaHCO₃ in water was added to the flask and the mixture was extracted by ethyl acetate (3×50 mL). The combined organic was washed by brine, dried over sodium sulfate and concentrated. The residue was chromatographed on a silica gel column eluted with 0-5% MeOH/DCM afforded 54 as off white solid (250 mg, 56%). 1H NMR (400 MHz, DMSO-d6) δ 11.88 (s, 1H, NH), 10.37 (s, 1H, NH), 9.59 (s, 1H, NH), 7.70-7.47 (m, 4H, Ar—H), 5.60 (s, 1H, Ar—H), 3.69-3.67 (m, 2H, CH₂), 2.33-2.31 (m, 2H, CH₂), 2.20 (s, 3H, CH₃), 1.84-1.78 (m, 1H, CH), 1.20 (bs, 10H, CH, 3CH₃), 0.82-0.80 (m, 4H, Ar—H); ESI-MS: calcd for (C₂₅H₃₃N₉OS) 507. found 508 [M+H]+. HPLC: retention time: 17.06 min. purity: 100%.

### Example 55

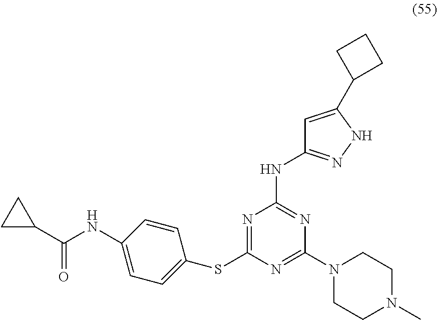

Compound 7 (300 mg, 0.88 mmol) was reacted syquencely with 5-cyclobutyl-1H-pyrazol-3-amine and 1-methylpiperazine as described for preparation of compound 50. compound 55 was obtained as light yellow solid (140 mg, 31%). 1H NMR (400 MHz, DMSO-d6) δ 11.88 (bs, 1H, NH), 10.46 (s, 1H, NH), 9.52 (bs, 1H, NH), 7.77-7.48 (m, 4H, Ar—H), 5.38 (s, 1H, Ar—H), 3.67 (bs, 2H, CH₂), 2.31 (bs, 2H, CH₂), 2.20 (s, 3H, CH₃), 2.12-1.75 (m, 7H, CH, 3CH₂), 1.24-1.16 (m, 1H, CH), 0.84-0.82 (m, 4H, Ar—H); ESI-MS: calcd for (C₂₅H₃₁N₉OS) 505. found 506 [M+H]+. HPLC: retention time: 16.75 min. purity: 100%.

### Example 56

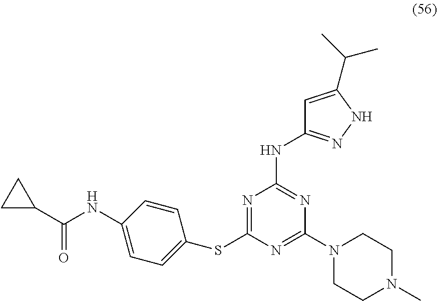

To a solution of cyanuric chloride (300 mg, 1.63 mmol) in THF (16 mL) was added 2-amino-5-isopropylpyrazole (263 mg, 1.63 mmol) and DIPEA (0.28 mL, 1.63 mmol) at 0° C. The reaction mixture was let to stir at 0° C. to room temperature for 2 h. Then, 1-methylpiperazine (0.18 mL, 1.63 mmol) and DIPEA (0.28 mL, 1.90 mmol) were added to the above mixture and allowed to stir at room temperature for 3 hours. Next, compound (I) (628 mg, 3.25 mmol) and DIPEA (0.57 mL, 2.25 mmol) were added to the mixture and stirred at room temperature overnight. Saturated NaHCO₃ in water was added and the mixture was extracted by ethyl acetate (3×50 mL). The combined organic was washed by brine, dried over sodium sulfate and concentrated. The residue was chromatographed on a silica gel column eluted with 0-5% MeOH/DCM afforded 56 as light yellow solid (110 mg, 37%). ¹H NMR (400 MHz, DMSO-d6) δ 11.77 (bs, 1H, NH), 10.42 (s, 1H, NH), 9.50 (bs, 1H, NH), 7.73 (bs, 2H, Ar—H), 7.48 (d, J=8.4 Hz, 2H, Ar—H), 5.40 (bs, 1H, Ar—H), 3.68 (bs, 4H, 2CH₂), 2.33 (bs, 4H, 2CH₂), 2.21 (s, 3H, CH₃), 1.83-1.81 (m, 1H, CH), 1.24 (bs, 3H, CH₃), 1.05 (bs, 3H, CH₃), 0.84-0.82 (m, 4H, Ar—H); ESI-MS: calcd for (C₂₄H₃₁N₉OS) 493. found 494 [M+H]+. HPLC: retention time: 15.38 min. purity: 99%.

### Example 57

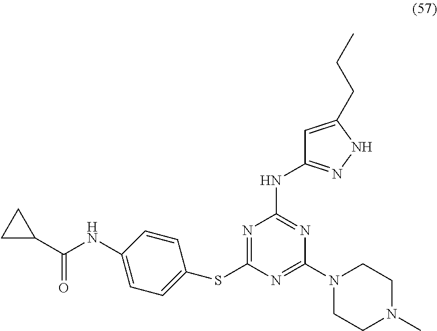

Compound 2 (300 mg, 0.88 mmol) was reacted syquencely with 5-propyl-1H-pyrazol-3-amine and 1-methylpiperazine as described for preparation of compound 54. compound 57 was obtained obtained as light yellow solid (35 mg, 8%). 1H NMR (400 MHz, DMSO-d6) δ 11.02 (bs, 1H, NH), 10.34 (s, 2H, NH), 7.63 (d, J=8.8 Hz, 2H, Ar—H), 7.42 (d, J=8.4 Hz, 2H, Ar—H), 5.17 (bs, 1H, Ar—H), 4.32 (bs, 2H, CH₂), 3.44-3.42 (m, 4H, 2CH₂), 2.39-2.35 (m, 2H, CH₂), 2.21-2.13 (m, 4H, 2CH₂), 2.13 (s, 3H, CH₃), 1.81-1.76 (m, 2H, CH₂), 1.55-1.49 (m, 2H, CH₂), 1.87 (t, J=7.6 Hz, 3H, CH₃), 0.83-0.80 (m, 4H, Ar—H); ESI-MS: calcd for (C₂₄H₃₁N₉OS) 493. found 494 [M+H]+. HPLC: retention time: 43.26 min. purity: 98%.

### Example 58

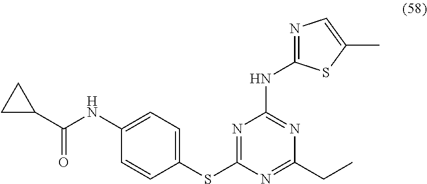

To a solution of compound 3 (180 mg, 0.95 mmol) in THF (10 mL) was added a solution of 2-amino-5-methylthiozole (110 mg, 0.95 mmol) and DIPEA (0.17 mL, 0.95 mmol) in THF (5 mL) dropwise at 0° C. The reaction mixture was let to stir at 0° C. to room temperature for 3 h. Then, a solution of compound 1 (280 mg, 1.50 mmol) and DIPEA (0.33 mL, 1.90 mmol) in THF (5 mL) was added to the above reaction flask at room temperature. The mixture was stirred at room temperature for overnight. Saturated NaHCO₃ in water was added to the flask and the mixture was extracted by ethyl acetate (3×50 mL). The combined organic was washed by brine, dried over sodium sulfate and concentrated. The residue was chromatographed on a silica gel column eluted with DCM/MeOH (30:1) afforded 58 as light yellow solid (25 mg, 6%). 1H NMR (400 MHz, DMSO-d6) δ 11.83 (s, 1H, NH), 10.48 (s, 1H, NH), 7.79-7.76 (m, 2H, Ar—H), 7.56 (d, J=8.5 Hz, 2H, Ar—H), 6.98 (s, 1H, Ar—H), 2.63 (dd, J=15.1 Hz, 2H, CH₂), 2.11 (bs, 3H, CH₃), 1.81 (m, 1H, CH), 1.21 (t, J=7.5 Hz, 3H, CH₃); ESI-MS: calcd for (C₁₉H₂₀N₆OS₂) 412. found 413 [M+H]+. HPLC: retention time: 26.59 min. purity: 96%.

### Example 59

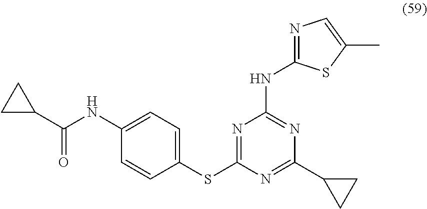

Compound 5 (50 mg, 0.26 mmol) was reacted sequencely with 2-amino-5-methylthiozole and compound 1 as described for preparation of 58. Compound 59 was obtained as white solid (5 mg, 4%). 1H NMR (400 MHz, DMSO-d6) δ 11.72 (bs, 1H, NH), 10.45 (s, 1H, NH), 7.74 (bs, 2H, Ar—H), 7.53 (d, J=8.3 Hz, 2H, Ar—H), 6.98 (bs, 1H, Ar—H), 2.11 (bs, 3H, CH₃), 1.90 (m, 1H, CH), 1.81 (m, 1H, CH), 1.06 (d, J=6.4 Hz, 4H, Ar—H), 0.81 (d, J=6.2 Hz, 4H, Ar—H); ESI-MS: calcd for (C₂₀H₂₀N₆OS₂) 424. found 425 [M+H]+. HPLC: retention time: 30.64 min. purity: 94%

### Example 60

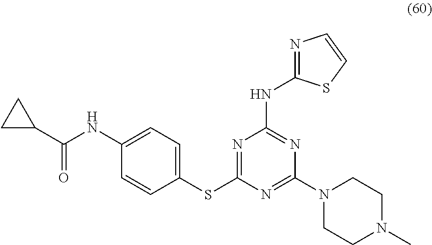

Compound 7 (300 mg, 0.88 mmol) was reacted sequencely with thiazol-2-amine and compound 1 as described for the preparation of compound 58. Compound 60 was obtained as white solid (80 mg, 19%). 1H NMR (400 MHz, DMSO-d6) δ 11.59 (bs, 1H, NH), 10.38 (s, 1H, NH), 7.67 (d, J=8.8 Hz, 2H, Ar—H), 7.51 (d, J=8.7 Hz, 2H, Ar—H), 7.38 (s, 1H, Ar—H), 7.15 (bs, 1H, Ar—H), 3.84-3.53 (m, 4H, 2CH₂), 2.36-2.25 (m, 4H, 2CH₂), 2.18 (s, 3H, CH₃), 1.82-1.78 (m, 1H, CH), 0.82 (m, 4H, Ar—H); ESI-MS: calcd for (C₂₁H₂₄N₈OS₂) 468. found 469 [M+H]+. HPLC: retention time: 15.59 min. purity: 76%.

### Example 61

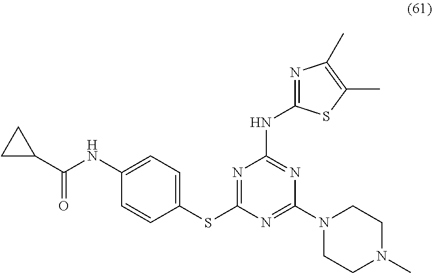

Compound 7 (300 mg, 0.88 mmol) was reacted sequencely with 4,5-dimethylthiazol-2-amine and compound 1 as described for the preparation of compound 58. Compound 61 was obtained as white solid (47 mg, 11%). 1H NMR (400 MHz, DMSO-d6) δ 11.22 (bs, 1H, NH), 10.41 (s, 1H, NH), 7.72-7.69 (m, 2H, Ar—H), 7.50 (d, J=8.4 Hz, 2H, Ar—H), 3.79-3.55 (m, 4H, 2CH₂), 2.34-2.25 (m, 4H, 2CH₂), 2.18 (s, 3H, CH₃), 2.07 (s, 6H, 2CH₃), 1.82-1.79 (m, 1H, CH), 0.81 (m, 4H, Ar—H); ESI-MS: calcd for (C₂₃H₂₈N₈OS₂) 496. found 497 [M+H]+. HPLC: retention time: 17.23 min. purity: 97%.

### Example 62

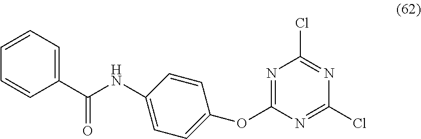

A solution of 3-hydroxybenzalinidine (2.00 g, 9.38 mmol) and DIPEA (1.70 mL, 9.38 mmol) in THF (15 mL) was added dropwise to a stirred solution of (1.73 g, 9.38 mmol) in THF (30 mL) at −10° C. Reaction mixture was let to stir for 1 h at this temperature. Saturated ammonium chloride was added to the reaction mixture and the mixture was extracted with ethyl acetate (1×100 mL). The organic layer was washed by brine, dried (Na₂SO₄) and concentrated to give compound 62 as white solid (2.60 g, 77% yield).

### Example 63

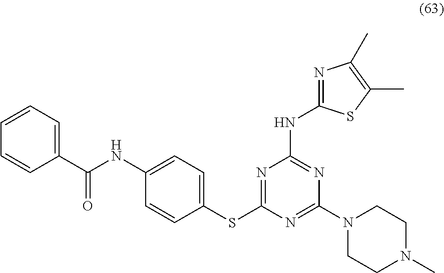

Compound 62 (300 mg, 0.83 mmol) was reacted sequencely with 2-amino-5-methylthiozole and 1-methylpiperazine as described for preparation of compound 58. Compound 63 was obtained as white solid (33 mg, 8%). 1H NMR (400 MHz, DMSO-d6) δ 11.45 (bs, 1H, NH), 10.20 (s, 1H, NH), 7.88-7.72 (m, 4H, Ar—H), 7.57 (t, J=8.0 Hz, 1H, Ar—H), 7.47-7.44 (m, 1H, Ar—H), 7.33 (t, J=8.4 Hz, 2H, Ar—H), 7.08 (t, J=7.2 Hz, 1H, Ar—H), 7.00 (bs, 1H, Ar—H), 3.88-3.63 (m, 4H, 2CH₂), 2.40-2.30 (m, 4H, 2CH₂), 2.17 (s, 3H, CH₃); ESI-MS: calcd for (C₂₅H₂₆N₈O₂S) 502. found 503 [M+H]+. HPLC: retention time: 18.96 min. purity: 90%.

### Example 64

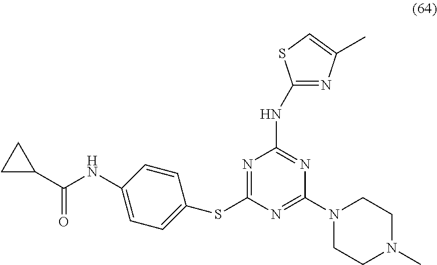

Compound 7 (300 mg, 0.88 mmol) was reacted sequencely with 4-methylthiazol-2-amine and 1-methylpiperazine as described for preparation of 58. Compound 64 was obtained as white solid (60 mg, 14%). 1H NMR (400 MHz, DMSO-d6) δ 11.49 (bs, 1H, NH), 10.37 (s, 1H, NH), 7.66 (d, J=8.8 Hz, 2H, Ar—H), 7.50 (d, J=8.8 Hz, 2H, Ar—H), 6.68 (bs, 1H, Ar—H), 3.80 (bs, 2H, CH₂), 3.50 (bs, 2H, CH₂), 2.34 (bs, 2H, CH₂), 2.25-2.21 (m, 5H, CH₂, CH₃), 2.17 (s, 3H, CH₃), 1.83-1.77 (m, 1H, CH), 0.82 (m, 4H, Ar—H); ESI-MS: calcd for (C₂₂H₂₆N₈OS₂) 482. found 483 [M+H]+. HPLC: retention time: 16.72 min. purity: 97%.

### Example 65

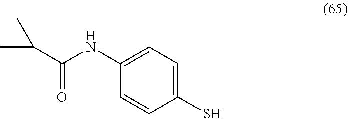

To a solution of 4-aminothiophenol (1.00 g, 7.99 mmol) and pyridine (0.97 mL, 11.99 mmol) in THF (30 mL) at 0° C. was added a solution of isobytric anhydride (1.33 mL, 7.99 mmol) in THF (40 mL) dropwise. The reaction was stirred from 0° C. to room temperature for overnight, diluted with EtOAc (100 mL), washed with 1 N HCl (100 mL×5), dried over Na₂SO₄, concentrated, and dried under vacuum to yield the compound 65 as a yellow solid, which was used for further reaction without purification.

### Example 66

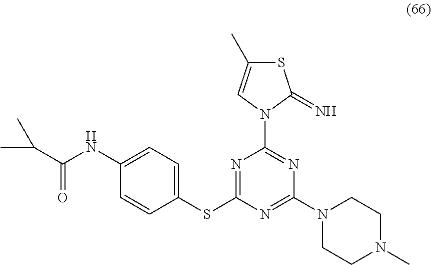

Cyanuric chloride (300 mg, 1.63 mmol) was reacted sequentially with 2-amino-5-methylthiozole, compound 65 and 1-methylpiperazine as described for preparation of 2. compound 66 was obtained as white solid (60 mg, 8%). 1H NMR (400 MHz, DMSO-d6) δ 10.05 (s, 1H, NH), 9.00 (s, 1H, NH), 7.74-7.72 (m, 2H, Ar—H), 7.51-7.49 (m, 2H, Ar—H), 7.18 (s, 1H, Ar—H), 3.73-3.67 (m, 4H, 2CH₂), 2.67-2.59 (m, 1H, CH), 2.37-2.28 (m, 4H, 2CH₂), 2.20 (s, 3H, CH₃), 2.02 (s, 3H, CH₃), 1.12 (s, 3H, CH₃), 1.11 (s, 3H, CH₃); ESI-MS: calcd for (C₂₂H₂₈N₈OS₂) 484. found 485 [M+H]+. HPLC: retention time: 7.42 min. purity: 94%.

### Example 67

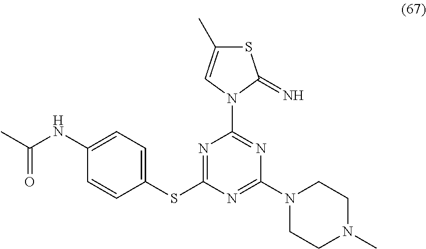

Cyanuric chloride (300 mg, 1.63 mmol) was reacted sequentially with 2-amino-5-methylthiozole, N-(4-mercaptophenyl)acetamide and 1-methylpiperazine as described for preparation of 2. Compound 67 was obtained as white solid (63 mg, 10%). 1H NMR (400 MHz, DMSO-d6) δ 10.17 (s, 1H, NH), 8.97 (s, 1H, NH), 7.71-7.68 (m, 2H, Ar—H), 7.51-7.49 (m, 2H, Ar—H), 7.18 (s, 1H, Ar—H), 3.72-3.66 (m, 4H, 2 CH₂), 2.67-2.59 (m, 1H, CH), 2.35-2.32 (m, 4H, 2CH₂), 2.20 (s, 3H, CH₃), 2.08 (s, 3H, CH₃), 2.02 (s, 3H, CH₃); ESI-MS: calcd for (C₂₀H₂₂N₈OS₂) 456. found 457 [M+H]+. HPLC: retention time: 4.12 min. purity: 80%.

### Example 68

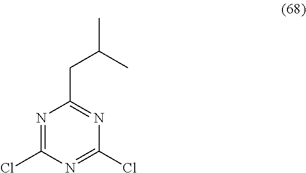

A solution of iso-butylmagnesium bromide in ether (2M, 35 ml, 70.0 mmole) was added dropwise to a stirred solution of cyanuric chloride (5.28 g, 28.63 mmole) in anhydrous dichloromethane at −5° C. After the reaction was completed as indicated by TLC, water was added water dropwise at a rate such that the temperature of the reaction stayed below 10° C. After warming to room temperature, the reaction mixture was diluted with additional water and methylene chloride and passed through a pad of cilite, washed by saturated ammonium chloride, dried and concentrated to give 2,4-dichloro-6-iso-butyl-1,3,5-triazine as yellow slurry liquid residue. The crude product was passed through a pad of silica gel eluted with 10% ethyl acetate in hexanes to give 68 as the light yellow liquid (3.0 g, 51%). 1H NMR (500 MHz, CDCl₃) δ 2.75 (d, J=7.0 Hz, 2H), 2.29 (m, 1H), 0.97 (d, J=7.0 Hz. 6H).

### Example 69

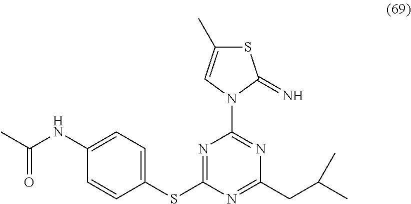

Compound 68 (300 mg, 1.63 mmol) was reacted sequentially with 2-amino-5-methylthiozole and N-(4-mercaptophenyl)acetamide as described for preparation of 58. Compound 69 was obtained as white solid (53 mg, 9%). 1H NMR (400 MHz, DMSO-d6) δ 10.18 (s, 1H, NH), 9.03 (s, 1H, NH), 7.72-7.70 (m, 2H, Ar—H), 7.55-7.50 (m, 2H, Ar—H), 7.10 (s, 1H, Ar—H), 2.54 (d, J=7.2 Hz, 2H, CH₂), 2.16-2.12 (m, 1H, CH), 2.06 (s, 3H, CH₃), 2.02 (s, 3H, CH₃), 0.91 (s, 3H, CH₃), 0.89 (s, 3H, CH₃); ESI-MS: calcd for (C₁₉H₂₂N₆OS₂) 414. found 415 [M+H]+. HPLC: retention time: 23.79 min. purity: 99%.

### Example 70

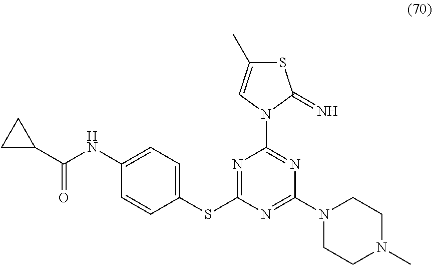

Compound 7 (500 mg, 1.47 mmol) was reacted sequentially with 2-amino-5-methylthiozole and 1-methylpiperazine as described for preparation of 58. Compound 70 was obtained as white solid (70 mg, 6%). 1H NMR (400 MHz, DMSO-d6) δ 10.42 (s, 1H, NH), 8.96 (s, 1H, NH), 7.72-7.70 (m, 2H, Ar—H), 7.51-7.49 (m, 2H, Ar—H), 7.19 (s, 1H, Ar—H), 3.72-2.66 (m, 4H, 2CH₂), 2.35-2.32 (m, 4H, 2CH₂), 2.20 (s, 3H, CH₃), 2.02 (s, 3H, CH₃), 1.83-1.79 (m, 1H, CH), 0.83-0.81 (m, 4H, Ar—H); ESI-MS: calcd for (C₂₂H₂₆N₈OS₂) 482. found 483 [M+H]+. HPLC: retention time: 7.42 min. purity: 99%.

### Example 71

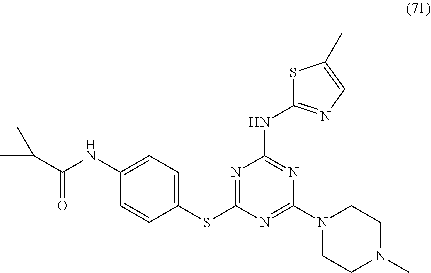

To a solution of cyanuric chloride (300 mg, 1.63 mmol) in THF (10 mL) was added dropwise a solution of compound 65 (320 mg, 1.63 mmol) and DIPEA (0.28 mL, 1.63 mmol) in THF (5 mL) at 0° C. The reaction mixture was let to stir in 10-20 mL microwave vial at 0° C. to room temperature for 2 h. Then, 2-amino-5-methylthiazole (150 mg, 1.30 mmol) and DIPEA (0.25 mL, 1.30 mmol) were added to the mixture and vial was sealed with a cap. The mixture was allowed to stir at 150° C. for 5 min. in the microwave synthesizer (Biotage, Initiator 2.0). Next, 1-methylpiperazine (0.18 mL, 1.63 mmol) and DIPEA (0.28 mL, 1.90 mmol) were added to the above mixture and allowed to stir at 60° C. for 10 min. in the microwave synthesizer. Saturated NaHCO₃ in water was added and the mixture was extracted by ethyl acetate (3×50 mL). The combined organic was washed by brine, dried over sodium sulfate and concentrated. The residue was recrystallized with DCM/MeOH mixture to give 71 as white solid (110 mg, 14%). 1H NMR (400 MHz, DMSO-d6) δ 11.28 (s, 1H, NH), 10.53 (s, 1H, NH), 7.75-7.73 (m, 2H, Ar—H), 7.51 (d, J=8.8 Hz, 2H, Ar—H), 6.97 (s, 1H, Ar—H), 3.78-3.61 (m, 4H, CH₂), 2.65-2.58 (m, 1H, CH), 2.35-2.27 (m, 4H, CH₂), 2.19 (s, 6H, CH₃), 1.12 (s, 3H, CH₃), 1.10 (s, 3H, CH₃); ESI-MS: calcd for (C₂₂H₂₈N₈OS₂) 484. found 485 [M+H]+. HPLC: retention time: 17.69 min. purity: 96%.

### Example 72

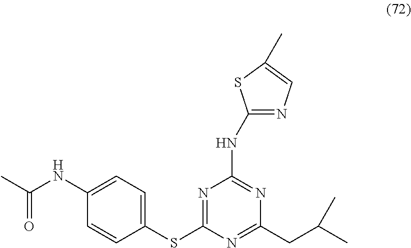

Compound 68 (300 mg, 1.46 mmol) was reacted sequentially with 2-amino-5-methylthiozole and N-(4-mercaptophenyl)acetamide using procedure similar to the preparation of 71. Compound 72 was obtained as white solid (300 mg, 50%). 1H NMR (400 MHz, DMSO-d6) δ 11.84 (s, 1H, NH), 10.22 (s, 1H, NH), 7.76-7.74 (m, 2H, Ar—H), 7.56 (d, J=8.4 Hz, 2H, Ar—H), 6.99 (s, 1H, Ar—H), 2.51-2.47 (m, 3H), 2.21-2.08 (m, 6H), 0.92 (s, 3H, CH₃), 0.91 (s, 3H, CH₃); ESI-MS: calcd for (C₁₉H₂₂N₆OS₂) 414. found 415 [M+H]+. HPLC: retention time: 26.43 min. purity: 96%.

### Example 73

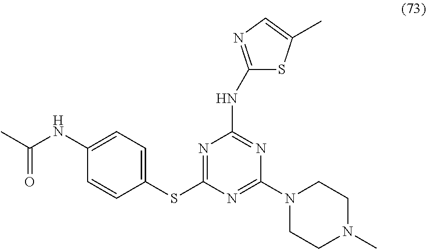

Cyanuric chloride (300 mg, 1.63 mmol) was reacted sequentially with 2-amino-5-methylthiozole, N-(4-mercaptophenyl)acetamide and 1-methylpiperazine as described for preparation of 71. Compound 73 was obtained as white solid (270 mg, 36%). 1H NMR (400 MHz, DMSO-d6) δ 11.31 (s, 1H, NH), 10.16 (s, 1H, NH), 7.71-7.69 (m, 2H, Ar—H), 7.51 (d, J=8.4 Hz, 2H, Ar—H), 6.99 (s, 1H, Ar—H), 3.77-3.51 (m, 4H, 2CH₂), 2.51-2.20 (m, 10H, 2CH₂, 2CH₃), 2.07 (s, 3H, CH₃); ESI-MS: calcd for (C₂₀H₂₄N₈OS₂) 456. found 457 [M+H]+. HPLC: retention time: 12.32 min. purity: 96%.

### Example 74

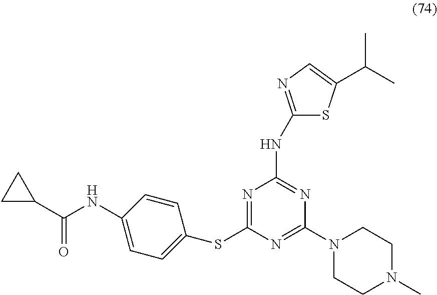

Compound 7 (200 mg, 0.59 mmol) was reacted sequentially with 2-amino-5-ieopropylthiozole (compound 51) and 1-methylpiperazine using the procedure similar to the preparation of 71. Compound 74 was obtained as light yellow solid (50 mg, 17%). 1H NMR (400 MHz, DMSO-d6) δ 11.25 (bs, 1H, NH), 10.41 (s, 1H, NH), 7.74-7.72 (m, 2H, Ar—H), 7.52 (d, J=9.3 Hz, 2H, Ar—H), 6.98 (bs, 1H, Ar—H), 3.86-3.54 (m, 4H, 2CH₂), 2.98-2.80 (m, 1H, CH), 2.42-2.28 (m, 4H, 2CH₂), 2.20 (s, 3H, CH₃), 1.84-1.78 (m, 1H, CH), 1.15 (bs, 6H, 2CH₃), 0.83-0.81 (m, 4H, Ar—H); ESI-MS: calcd for (C₂₄H₃₀N₈OS₂) 510. found 511 [M+H]+. HPLC: retention time: 19.86 min. purity: 95%.

### Example 75

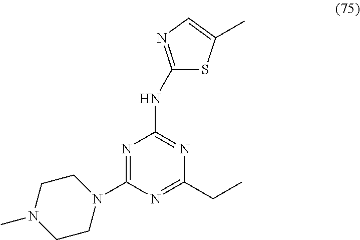

Compound 3 (300 mg, 1.69 mmol) was reacted sequentially with 2-amino-5-methylthiozole and methyl piperazine using procedure similar to the preparation of 71. Compound 75 was obtained as yellow solid (140 mg, 26%). 1H NMR (400 MHz, DMSO-d6) δ 11.28 (s, 1H, NH), 7.04 (s, 1H, Ar—H), 3.80-3.79 (bs, 4H, 2CH₂), 2.53-2.46 (m, 2H, CH₂), 2.34-2.30 (m, 4H, 2CH₂), 2.18 (s, 3H, CH₃), 1.18 (t, J=7.6 Hz, 1H, CH₃); ESI-MS: calcd for (C₁₄H₂₁N₇S) 319. found 320 [M+H]+. HPLC: retention time: 2.62 min. purity: 97%.

### Example 76

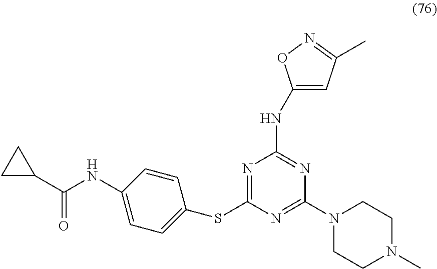

Compound 7 (300 mg, 0.88 mmol) was reacted sequentially sequentially with 3-methylisoxazol-5-amine and 1-methylpiperazine using procedure similar to the preparation of 71. Compound 76 was obtained as light yellow solid (20 mg, 5%). 1H NMR (400 MHz, DMSO-d6) δ 11.23 (bs, 1H, NH), 10.56 (s, 1H, NH), 7.79-7.51 (m, 4H, Ar—H), 4.61-4.51 (m, 2H, CH₂), 3.44-3.33 (m, 4H, 2CH₂), 3.07-3.01 (m, 2H, CH₂), 2.75 (s, 3H, CH₃), 2.00-1.81 (m, 4H, CH₃, CH), 0.82 (m, 4H, Ar—H); ESI-MS: calcd for (C₂₂H₂₆N₈O₂S) 466. found 467 [M+H]+. HPLC: retention time: 15.36 min. purity: 100%.

### Example 77

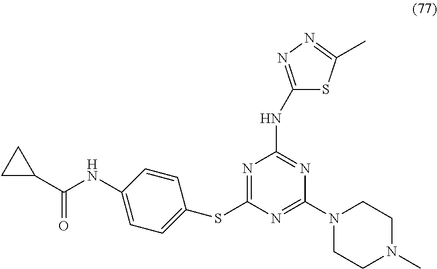

Compound 7 (300 mg, 0.88 mmol) was reacted sequentially sequentially with 5-methyl-1,3,4-thiadiazol-2-amine and 1-methylpiperazine using procedure similar to the preparation of 71. Compound 77 was obtained as white solid (70 mg, 15%). 1H NMR (400 MHz, DMSO-d6) δ 11.83 (bs, 1H, NH), 10.54 (s, 1H, NH), 7.77-7.75 (m, 2H, Ar—H), 7.52 (d, J=8.4 Hz, 1H, Ar—H), 3.97-3.63 (m, 4H, 2CH₂), 2.90-2.83 (m, 4H, 2CH₂), 2.41 (s, 3H, CH₃), 1.87-1.81 (m, 4H, Ar—H); ESI-MS: calcd for (C₂₁H₂₅N₉OS₂) 483. found 484 [M+H]+. HPLC: retention time: 13.07 min. purity: 94%.

### Example 78

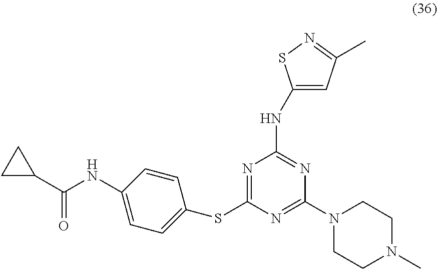

Compound 7 (300 mg, 0.88 mmol) was reacted sequentially sequentially with 3-methylisothiazol-5-amine and 1-methylpiperazine using procedure similar to the preparation of 71. Compound 78 was obtained as yellow solid (10 mg, 2%). 1H NMR (400 MHz, DMSO-d6) δ 11.48 (bs, 1H, NH), 10.50 (s, 1H, NH), 7.70 (d, J=8.8 Hz, 1H, Ar—H), 7.50 (d, J=8.4 Hz, 1H, Ar—H), 6.69 (s, 1H, Ar—H), 4.76-4.32 (m, 2H, CH₂), 4.58-4.37 (m, 2H, CH₂), 3.11-3.00 (m, 2H, CH₂), 2.76 (s, 3H, CH₃), 2.28 (s, 3H, CH₃), 2.87-2.81 (m, 1H, CH), 1.83-1.81 (m, 4H, Ar—H); ESI-MS: calcd for (C₂₂H₂₆N₈OS₂) 482. found 483 [M+H]+. HPLC: retention time: 15.39 min. purity: 99%.

### Example 79

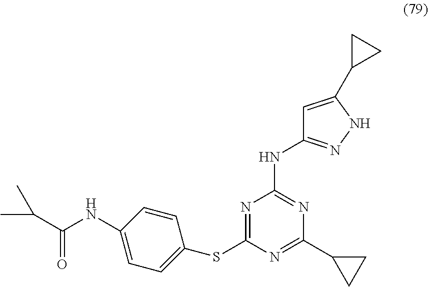

Compound 5 (65 mg, 0.34 mmol) was reacted sequentially with 5-cyclopropyl-1H-pyrazol-3-amine and compound 65 using procedure similar to the preparation of 71. Compound 79 was obtained as light yellow solid (30 mg, 20%). 1H NMR (400 MHz, DMSO-d6) δ 11.81 (s, 1H, NH), 10.07 (s, 2H, NH), 7.80-7.50 (m, 4H, Ar—H), 6.36 (s, 1H, Ar—H), 2.65-2.58 (m, 1H, CH), 1.85-1.80 (m, H, 1CH), 1.62-1.58 (m, 1H, CH), 1.11 (s, 3H, CH₃), 1.09 (s, 3H, CH₃), 0.99-0.44 (m, 8H, Ar—H); ESI-MS: calcd for (C₂₂H₂₅N₇OS) 436. found 436 [M+H]+. HPLC: retention time: 28.99 min. purity: 95%.

### Example 80

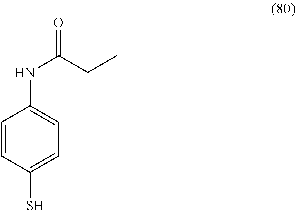

To the 4-aminothiophenol (1.0 g, 7.98 mMol) in 30 mL of CH₂Cl₂ at −10° C. was added pyridine (966 μL, 947 mg, 11.97 mMol) followed by dropwise addition of propionyl chloride (690 μL, 738 mg, 7.98 mMol). Reaction mixture was stirred overnight to room temperature. Reaction mixture was washed with 1N HCl and solvent was removed under reduced pressure. Crude material was dissolved in 25 mL of MeOH and 10 mL of H₂O. K₂CO₃ (1.1 g, 7.98 mMol) was added and reaction mixture was stirred at room temperature for 1 hr. After adjusting pH to 1 using 1N HCl, MeOH was evaporated and resulting aqueous solution was extracted with CH₂Cl₂. Organic fractions were combined, washed with brine, dried over Na₂SO₄, filtered and solvent was evaporated to give compound 80 as off-yellow solid (980 mg, 68%). 1H NMR (400 MHz, CDCl₃) δ 7.40 (d, J=8.40 Hz, 2H), 7.24 (d, J=8.40 Hz, 2H), 7.11 (bs, 1H), 3.41 (s, 1H), 2.37 (q, J=7.6 Hz, 2H), 1.24 (t, J=7.6 Hz, 3H). MS (ESI) m/z 182 [M+H]+

### Example 81

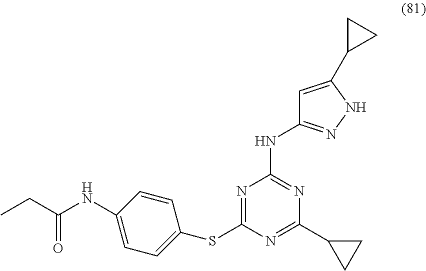

Compound 5 (300 mg, 1.58 mmol) was reacted sequentially with 5-cyclopropyl-1H-pyrazol-3-amine and compound 80 using procedure similar to the preparation of 71. Compound 81 was obtained as off white solid (185 mg, 28%). 1H NMR (400 MHz, DMSO-d6) δ 11.81 (s, 1H, NH), 10.10 (s, 2H, NH), 7.78-7.76 (m, 2H, Ar—H), 7.51 (d, J=8.4 Hz, 1H, Ar—H), 6.36 (s, 1H, Ar—H), 2.36-2.31 (m, 2H, CH₂), 1.84-1.80 (m, 1H, CH), 1.65-1.56 (m, 1H, CH), 1.09 (t, J=7.6 Hz, 3H, CH₃), 0.99-0.45 (m, 8H, Ar—H); ESI-MS: calcd for (C₂₁H₂₃N₇OS) 421. found 422 [M+H]+. HPLC: retention time: 25.92 min. purity: 99%.

### Example 82

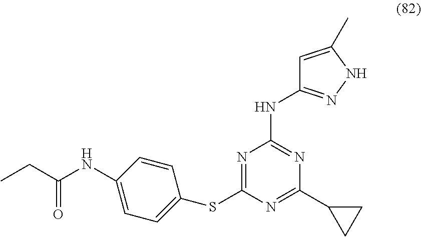

Compound 5 (300 mg, 1.58 mmol) was reacted sequentially with 5-methyl-1H-pyrazol-3-amine and compound 80 using procedure similar to the preparation of 71. Compound 82 was obtained as white solid (200 mg, 32%). 1H NMR (400 MHz, DMSO-d6) δ 11.82 (s, 1H, NH), 10.16 (s, 1H, NH), 10.09 (s, 1H, NH), 7.78-7.76 (m, 2H, Ar—H), 7.51 (d, J=8.4 Hz, 1H, Ar—H), 5.27 (s, 1H, Ar—H), 2.38-2.32 (m, 2H, CH₂), 1.95 (s, 3H, CH₃), 1.84-1.80 (m, 1H, CH), 1.10 (t, J=7.6 Hz, 3H, CH₃), 0.97 (bs, 4H, Ar—H); ESI-MS: calcd for (C₁₉H₂₁N₇OS) 395. found 396 [M+H]+. HPLC: retention time: 23.26 min. purity: 100%.

### Example 83

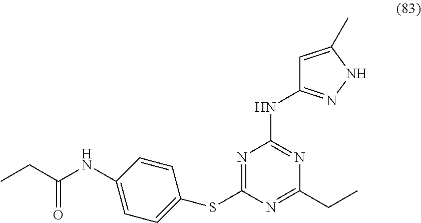

Compound 3 (300 mg, 1.69 mmol) was reacted sequentially with 5-methyl-1H-pyrazol-3-amine and compound 80 using procedure similar to the preparation of 71. Compound 83 was obtained as white solid (36 mg, 6%). 1H NMR (400 MHz, DMSO-d6) δ 11.83 (s, 1H, NH), 10.20 (s, 1H, NH), 10.15 (s, 1H, NH), 7.77 (d, J=8.8 Hz, 2H, Ar—H), 7.52 (d, J=8.8 Hz, 2H, Ar—H), 5.26 (s, 1H, Ar—H), 2.55-2.50 (m, 2H, CH₂), 2.35 (dd, J=15.2 Hz, 2H, CH₂), 1.94 (s, 3H, CH₃), 1.18 (t, J=7.6 Hz, 3H, CH₃), 1.10 (t, J=7.6 Hz, 3H, CH₃); ESI-MS: calcd for (C₁₈H₂₁N₇OS) 383. found 384 [M+H]+. HPLC: retention time: 19.29 min. purity: 99%.

### Example 84

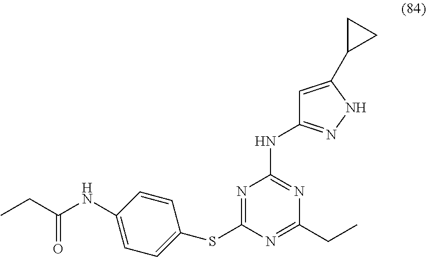

Compound 3 (100 mg, 0.56 mmol) was reacted sequentially with 5-cyclopropyl-1H-pyrazol-3-amine and compound 80 using procedure similar to the preparation of 71. Compound 84 was obtained as off white solid (150 mg, 65%). 1H NMR (400 MHz, DMSO-d6) δ 11.82 (s, 1H, NH), 10.20 (s, 1H, NH), 10.12 (s, 1H, NH), 7.79 (d, J=8.4 Hz, 2H, Ar—H), 7.53 (d, J=8.4 Hz, 2H, Ar—H), 5.35 (s, 1H, Ar—H), 2.55-2.50 (m, 2H, CH₂), 2.35 (dd, J=14.8 Hz, 2H, CH₂), 1.62-1.60 (m, 1H, CH), 1.18 (t, J=7.2 Hz, 3H, CH₃), 1.09 (t, J=7.6 Hz, 3H, CH₃), 0.78-0.76 (m, 2H, CH₂), 0.45-0.44 (m, 2H, CH₂); ESI-MS: calcd for (C₂₀H₂₃N₇OS) 409. found 410 [M+H]+. HPLC: retention time: 22.46 min. purity: 99%.

### Example 85

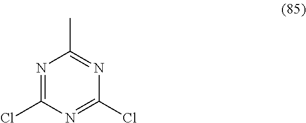

A solution of methylmagnesium bromide in ether (3M, 30 ml, 90 mmole) was added dropwise to a stirred solution of cyanuric chloride (3.91 g, 21.20 mmole) in anhydrous dichloromethane at −10° C. After the addition was complete, the reaction mixture was stirred at −5° C. for 4 h, after which time water was added dropwise at a rate such that the temperature of the reaction stayed below 10° C. After warming to room temperature, the reaction mixture was diluted with additional water and methylene chloride and passed through a pad of cilite. The organic layer was dried and evaporated to give 2,4-dichloro-6-methyl-1,3,5-triazine of 85 as yellow solids (3.02 g, 87%). 1H NMR (CDCl₃) δ 2.70 (s, 3H)

### Example 86

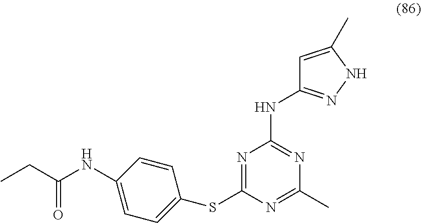

Compound 85 (300 mg, 1.82 mmol) was reacted sequentially with 5-methyl-1H-pyrazol-3-amine and compound 80 using procedure similar to the preparation of 71. Compound 86 was obtained as white solid (350 mg, 52%). 1H NMR (400 MHz, DMSO-d6) δ 11.84 (s, 1H, NH), 10.24 (s, 1H, NH), 10.16 (s, 1H, NH), 7.77 (d, J=8.4 Hz, 2H, Ar—H), 7.52 (dd, J=6.8 Hz, 2H, Ar—H), 5.25 (s, 1H, Ar—H), 2.36 (dd, J=14.8 Hz, 2H, CH₂), 1.94 (s, 3H, CH₃), 1.18 (t, J=7.6 Hz, 3H, CH₃); ESI-MS: calcd for (C₁₈H₂₁N₇OS) 369. found 370 [M+H]+. HPLC: retention time: 16.00 min. purity: 96%.

### Example 87

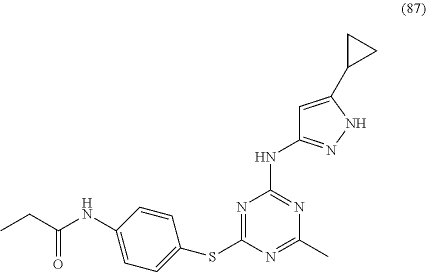

Compound 85 (300 mg, 1.82 mmol) was reacted sequentially with 5-cyclopropyl-1H-pyrazol-3-amine and compound 80 using procedure similar to the preparation of 71. Compound 87 was obtained as off white solid (150 mg, 21%). 1H NMR (400 MHz, DMSO-d6) δ 11.84 (s, 1H, NH), 10.22 (s, 1H, NH), 10.14 (s, 1H, NH), 7.78 (d, J=8.4 Hz, 2H, Ar—H), 7.52 (dd, J=8.4 Hz, 2H, Ar—H), 5.34 (s, 1H, Ar—H), 2.33 (dd, J=15.2 Hz, 2H, CH₂), 2.27 (s, 3H, CH₃), 1.63-1.59 (m, 1H, CH), 1.08 (t, J=7.6 Hz, 3H, CH₃), 0.78-0.76 (m, 2H, CH₂), 0.45-0.44 (m, 2H, CH₂); ESI-MS: calcd for (C₁₉H₂₁N₇OS) 395. found 396 [M+H]+. HPLC: retention time: 19.19 min. purity: 99%.

### Example 88

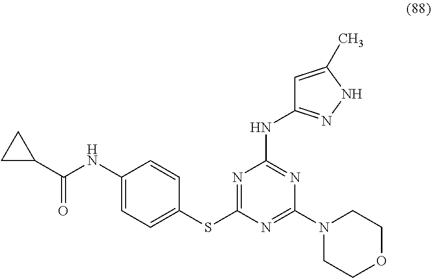

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of morpholine (204 mg, 2.35 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×25 ml) and washed by brine, dried over sodium sulfate and concentrated. The resulting crude product was purified by Teledyne-Isco flash system by using DCM/MeOH, 0 to 5% of Methanol in dichloromethane to provide compound 88 as white solids (20 mg, 7.5%). 1H NMR (400 MHz, DMSO-d6) δ 11.75 (br, 1H), 10.36 (s, 1H), 9.65 (br s, 1H), 7.69 (m, 2H), 7.48 (d, J=8.8 Hz, 2H), 5.23 (br s, 1H), 3.62-3.52 (m 8H), 2.14 (m, 3H), 1.78 (m, 1H), 0.78 (m, 4H); ESI-MS: calcd for (C₂₁H₂₄N₈O₂S) 452. found 453 (MH+). HPLC: retention time: 29.35 min. purity: 98%.

### Example 89

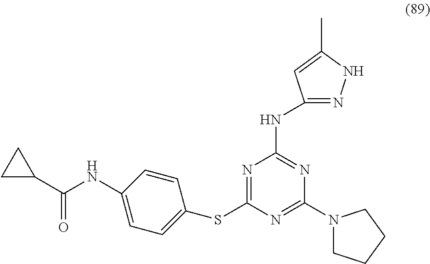

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of pyrrolidine (128 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using DCM/MeOH, 0 to 5% of Methanol in dichloromethane to provide compound 89 as white solids (76 mg, 30%). 1H NMR (400 MHz, DMSO-d6) δ 10.39 (br s, 1H), 9.50 (br s, 1H), 7.69 (m, 2H), 7.48 (d, J=8.8 Hz, 2H), 5.23 (br s, 1H), 3.55-3.12 (m 6H), 2.12 (m, 6H), 0.78 (m, 4H); ESI-MS: calcd for (C₂₁H₂₄N₈OS) 436. found 437 (MH+). HPLC: retention time: 26.73 min. purity: 100%.

### Example 90

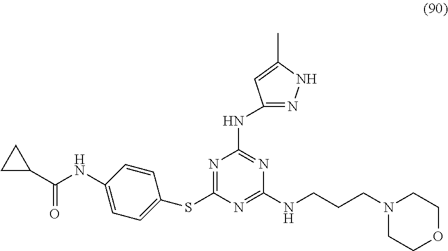

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of 3-morpholinopropan-1-amine (212 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using 0 to 7% of 7N NH₃ in Methanol/dichloromethane to provide compound 90 as white solids (95 mg, 40%). 1H NMR (400 MHz, DMSO-d6) δ 11.45 (br s, 1H), 10.39 (br s, 1H), 9.40 (br s, 1H), 7.69 (m, 2H), 7.48 (d, J=8.6 Hz, 2H), 5.23 (br s, 1H), 3.60-3.20 (m, 6H), 2.40-2.00 (m, 9H, 3×CH₂+CH₃), 1.80-1.20 (m, 3H), 0.78 (d, J=8.0 Hz, 4H); ESI-MS: calcd for (C₂₄H₃₁N₉O₂S) 509. found 510 (MH+). HPLC: retention time: 11.21 min. purity: 92%.

### Example 91

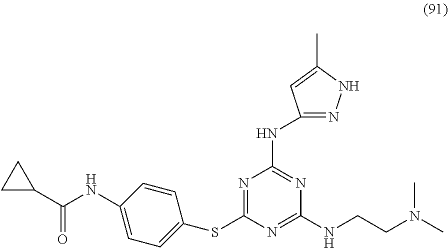

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of N,N-dimethylethane-1,2-diamine (129 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using 0 to 7% of 7N NH₃ in Methanol/dichloromethane to provide compound 91 as white solids (25 mg, 9.5%). 1H NMR (400 MHz, DMSO-d6) δ 11.80 (br s, 1H), 10.45 (br s, 1H), 9.60 (br s, 1H), 7.69 (m, 2H), 7.48 (d, J=8.6 Hz, 2H), 5.23 (br s, 1H), 3.4 (brs, 6H), 2.80 (m, 4H), 2.20 (m, 3H), 1.80 (m, 1H), 0.78 (d, J=8.0 Hz, 4H); ESI-MS: calcd for (C₂₁H₂₇N₉OS) 453. found 454 (MH+). HPLC: retention time: 10.16 min. purity: 95%.

### Example 92

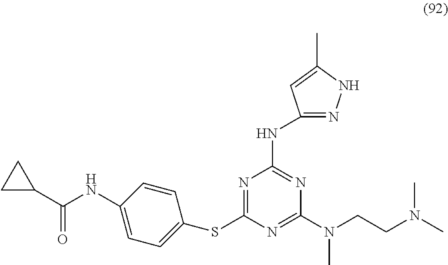

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of N1,N1,N2-trimethylethane-1,2-diamine (150 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using DCM/MeOH/TEA: (90/10/1) to provide compound 92 as white solids (25 mg, 9%). 1H NMR (400 MHz, DMSO-d6) δ 11.90 (br s, 1H), 10.40 (br s, 1H), 9.60 (br s, 1H), 7.70 (m, 2H), 7.45 (d, J=8.6 Hz, 2H), 5.23 (br s, 1H), 3.80-1.99 (m, 16H), 1.80 (m, 1H), 0.78 (d, J=8.0 Hz, 4H); ESI-MS: calcd for (C₂₂H₂₉N₉OS) 467. found 468 (MH+). HPLC: retention time: 13.08 min. purity: 84%.

### Example 93

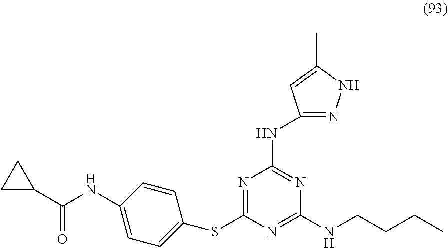

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of n-butyl amine (107 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using DCM/MeOH: (95/5) to provide compound 93 as white solids (22 mg, 8.5%). ¹H NMR (400 MHz, DMSO-d6) δ 11.90 (br s, 1H), 10.35 (br s, 1H), 9.60 (br s, 1H), 7.70 (m, 2H), 7.45 (d, J=8.6 Hz, 2H), 5.3 (br s, 1H), 3.80-1.20 (m, 13H), 0.78 (d, J=8.0 Hz, 4H); ESI-MS: calcd for (C₂₁H₂₆N₈OS) 438. found 439 (MH+). HPLC: retention time: 27.3 min. purity: 97%.

### Example 94

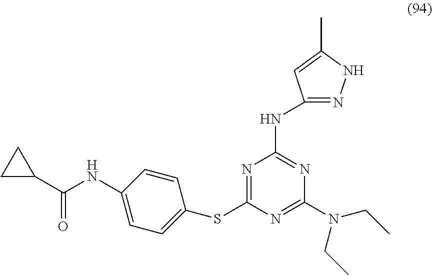

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of diethylamine (107 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using DCM/MeOH: (95/5) to provide compound 94 as white solids (35 mg, 14%). 1H NMR (400 MHz, DMSO-d6) δ 11.90 (br s, 1H), 10.38 (br s, 1H), 9.28 (br s, 1H), 7.70 (m, 2H), 7.45 (d, J=8.6 Hz, 2H), 5.28 (br s, 1H), 3.4-3.20 (m, 4H), 2.30 (m, 3H), 1.78 (m, 1H), 1.20 (m, 3H), 1.00 (m, 3H), 0.78 (m, 4H); ESI-MS: calcd for (C₂₁H₂₆N₈OS) 438. found 439 (MH+). HPLC: retention time: 29.9 min. purity: 98%.

### Example 95

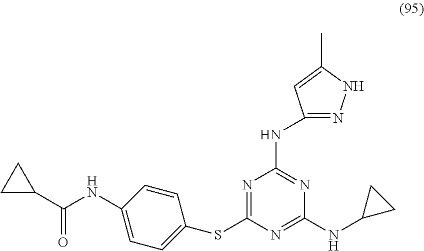

To a suspension of compound 2 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of cyclopropylamine (83 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using DCM/MeOH: (95/5) to provide compound 95 as white solids (40 mg, 16%). ESI-MS: calcd for (C₂₀H₂₂N₈OS) 422. found 423 (MH+). HPLC: retention time: 21.42 min. purity: 83%.

### Example 96

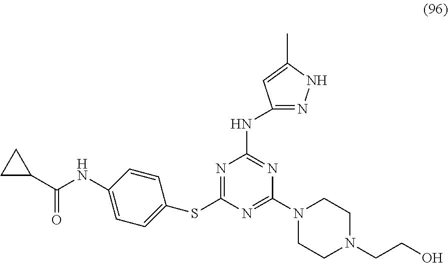

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. A solution of 2-(piperazin-1-yl) ethanol (191 mg, 1.47 mmol) and DIPEA (0.21 mL, 1.17 mmol) in THF (5 mL) was added to the above vial at room temperature. The mixture was heated at 60° C. for 0.5 h. After cooling to room temperature, saturated NaHCO₃ in water was added to the flask and the mixture was extracted by dichloromethane (3×20 ml) and washed by brine, dried over sodium sulfate and concentrated. The resulting crude product was purified by Teledyne-Isco flash system by using DCM/MeOH: (90/10) to provide compound 96 as white solids (45 mg, 15%). 1H NMR (400 MHz, DMSO-d6) δ 11.90 (br s, 1H), 10.38 (br s, 1H), 9.90 (br s, 1H), 7.70 (m, 2H), 7.45 (d, J=8.6 Hz, 2H), 5.3 (br s, 1H), 4.40 (br s, 1H), 3.76-3.2 (br, 6H), 3.49 (m, 2H), 2.40-2.00 (m, 9H), 1.78 (m, 1H), 0.78 (d, J=8.0 Hz, 4H); ESI-MS: calcd for (C₂₃H₂₉N₉O₂S) 495. found 496 (MH+). HPLC: retention time: 21.42 min. purity: 99%.

### Example 97

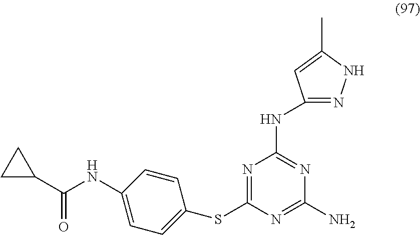

To a suspension of compound 7 (0.2 g, 0.588 mmol) in THF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. Added 10 ml of THF, 5 ml of DMSO and 10 ml NH₃OH to the above reaction mixture and heated at 80° C. for 20 min in microwave. The resulting precipitate was filtered and washed with cold water. The resulting solids were vacuum dried to provide compound 97 as white solids (45 mg, 20%). 1H NMR (400 MHz, DMSO-d6) δ 11.90 (br s, 1H), 10.38 (br s, 1H), 9.35 (br s, 1H), 7.70 (m, 2H), 7.45 (d, J=8.6 Hz, 2H), 6.95 (br s, 2H), 5.3 (br s, 1H), 4.40 (br s, 1H), 3.76-3.2 (br, 6H), 3.49 (m, 2H), 1.90 (br s, 3H) (m, 9H), 1.78 (m, 1H), 0.78 (d, J=7.5.0 Hz, 4H); ESI-MS: calcd for (C₁₇H₁₈N₈OS) 382. found 383 (MH+). HPLC: retention time: 14 min. purity: 81%.

### Example 98

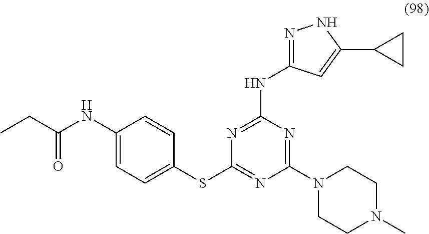

To the cyanuric chloride (300 mg, 1.62 mMol) in 15 mL of THF at −15° C. was dropwise added thiol 80 (295 mg, 1.63 mMol) and DIPEA (0.312 μL, 1.79 mMol) in 10 mL of THF. Reaction mixture was stirred for 90 minutes at −15° C. 5-cyclopropyl-1H-pyrazol-3-amine was added (198 mg, 1.63 mMol) followed by DIPEA (312 μL, 1.79 mMol) and reaction mixture was microwaved at 150° C. for 10 minutes. 1-Methylpiparezine (181 μL, 1.63 mMol) and DIPEA (312 μL, 1.79 mMol) was added and reaction mixture was stirred for 36 h. 30 mL of EtOAC was added and reaction mixture was washed with saturated NaHCO₃, brine, dried over Na₂SO₄, filtered and solvent was removed under reduced pressure. Flash column chromatography (silica, CH₂Cl₂/MeOH 95/5 to 90/10) afforded 152 mg (20%) of desired product 98. 1H NMR (400 MHz, DMSO) δ11.25 (bs, 1H), 10.09 (s, 1H), 7.70 (m, 2H), 7.51 (m, 2H), 6.99 (bs, 1H), 3.90-3.50 (m, 4H), 2.45-2.21 (m, 9H), 2.19 (s, 3H), 1.09 (t, J=7.6 Hz, 3H). MS (ESI) m/z 471 [M+H]+.

### Example 99

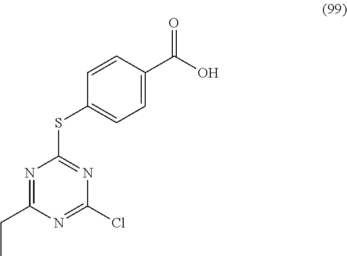

To the 4-mercaptobenzoic acid (318 mg of 90% acid, 1.85 mMol) in 10 mL of THF at 0° C. was added DIPEA (645 μL, 478 mg, 3.7 mMol) followed by dichloroethyltriazine (compound 3) (300 mg, 1.69 mMol) in 5 mL of THF. Reaction mixture was stirred at 0° C. for 30 minutes followed by 2 hours at room temperature. Disappearance of starting material was confirmed by TLC (CH₂Cl₂/MeOH 95/5). 5 mL of 1N HCl was added, organic layer was separated and aqueous fraction was extracted with EtOAc (3×50 mL). Organic fractions were combined, washed with brine, dried over Na₂SO₄, filtered and solvent was removed under reduced pressure. Flash column chromatography (silica, CH₂Cl₂/MeOH 95/5) yielded 360 mg (72%) of 99 as off-white solid. 1H NMR (400 MHz, CDCl₃) δ 8.18 (d, J=8.4 Hz, 2H), 7.72 (d, J=8.8 Hz, 2H), 2.78 (q, J=7.2 Hz, 2H), 1.24 (t, J=7.2 Hz, 3H). MS (ESI) m/z 296 [M+H]+.

### Example 100

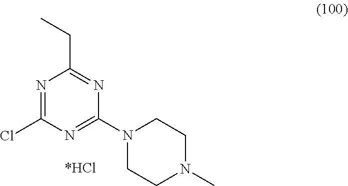

To the dichloroethyltriazine (compound 3) (4 g, 22.4 mMol) in THF/Acetone/Water (200 mL/50 mL/50 mL) was added 5% aq. NaHCO₃ (40 mL) followed by 1-methylpiparezine (2.26 mL, 2.04 g, 20.4 mMol). Reaction mixture was stirred overnight at room temperature. 200 mL of water was added, organic layer was separated and aqueous layer was extracted with EtOAc (4×50 mL). Organic fractions were combined, washed with brine, dried over Na₂SO₄, filtered and solvent was removed under reduced pressure. After flash column chromatography (silica, CH₂Cl₂/MeOH 95/5 0.1% TEA) fractions containing product were combined, solvent was removed under reduced pressure to give yellow oil that was re-dissolved in 20 mL of CH₂Cl₂ and 20 mL of MeOH and cooled to 0° C. Addition of 2N HCl in Et₂O (20 mL, 40 mMol of HCl) followed by removal of solvent under reduced pressure yielded 2.1 g (34%) of 100. 1H NMR (400 MHz, DMSO) δ 3.47 (bs, 6H), 3.08 (bs, 2H), 2.77 (s, 3H), 2.65 (q, J=7.6 Hz, 2H), 1.20 (t, J=7.6 Hz), 3H). MS (ESI) m/z 242 [M+H]+.

### Example 101

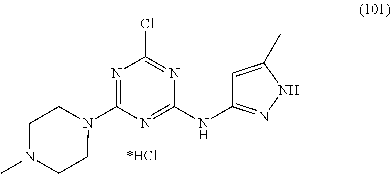

5-methyl-1H-pyrazol-3-amine (526 mg, 5.42 mMol) and DIPEA (942 μL, 700 mg, 5.42 mMol) in 50 mL of THF was added dropwise to the cyanuric chloride (1 g, 5.42 mMol) in 50 mL of THF at −10° C. After 30 minutes TLC confirmed disappearance of starting material (CH₂Cl₂/MeOH 95/5). Reaction mixture was warmed to 0° C. followed by dropwise addition of 1-methylpiparezine (602 μL, 543 mg, 5.42 mMol) and DIPEA (942 μL, 700 mg, 5.42 mMol) in 50 mL of THF. After overnight stirring at room temperature 150 mL of water was added, organic layer was separated and aqueous layer was extracted with EtOAc (3×100 mL). Organic fractions were combined, washed with brine, dried over Na₂SO₄, filtered and solvent was removed under reduced pressure. After flash column chromatography (silica, CH₂Cl₂/MeOH 90/10 to 85/15 0.1% TEA) fractions containing product were combined, solvent was removed under reduced pressure to give white semi solid that was re-dissolved in 20 mL of CH₂Cl₂ and 20 mL of MeOH and cooled to 0° C. Addition of 2N HCl in Et₂O (5 mL, 10 mMol of HCl) followed by removal of solvent under reduced pressure yielded 550 mg (30%) of 101. 1H NMR (400 MHz, CDCl₃) δ 10.98 (bs, 1H), 10.22 (bs, 1H), 5.74 (s, 1H), 2.89 (bs, 4H), 2.18 (s, 3H), 1.93 (bs, 4H), 1.70 (s, 3H). MS (ESI) m/z 309 [M+H]+.

### Example 102

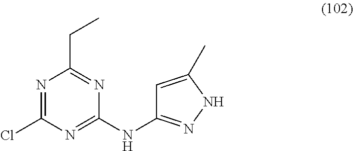

To the 5-methyl-1H-pyrazol-3-amine (86 mg, 0.86 mMol) in 2 mL of THF at 0° C. was added DIPEA (165 μL, 123 mg, 0.95 mMol). Reaction mixture was stirred at 0° C. for 5 minutes followed by addition of dichloroethyltriazine (compound 3) (200 mg, 1.12 mMol) in 1 mL of THF. Reaction mixture was stirred at 0° C. for 2 hours. 25 mL of water was added, reaction mixture was extracted with EtOAc (4×10 mL). Organic fractions were combined, washed with brine, dried over Na₂SO₄, filtered and solvent was removed over reduced pressure to yield 185 mg (90%) of unstable, crude pyrazoletriazine 102.

### Example 103

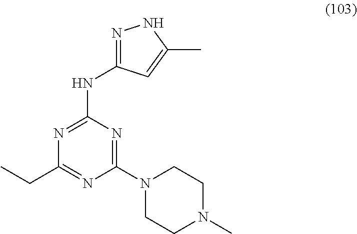

To the 1-methylpiperazine (55 μL, 50 mg, 0.5 mMol) in 1 mL of THF at room temperature was added DIPEA (103 μL, 76 mg, 0.59 mMol). Reaction mixture was stirred for 5 minutes at room temperature and the pyrazoletriazine 102 (92 mg—crude, 0.39 mMol) in 1 mL of THF was added at room temperature. Reaction mixture was stirred for 3 days. 5 mL of water was added, reaction mixture was extracted with EtOAc (3×5 mL). Organic fractions were combined, washed with brine, dried over Na₂SO₄, filtered and solvent was evaporated. Column (silica, CH₂Cl₂/MeOH 97/3) yielded 80 mg (68%) of 103 as white solid. 1H NMR (400 MHz, CDCl₃) δ 6.22 (s, 1H), 3.87 (bs, 4H), 2.59 (q, J=7.4 Hz, 2H), 2.43 (bs, 4H), 2.31 (s, 3H), 2.24 (s, 3H), 1.25 (t, J=7.4 Hz, 3H). MS (ESI) m/z 303 [M+H]+.

### Example 104

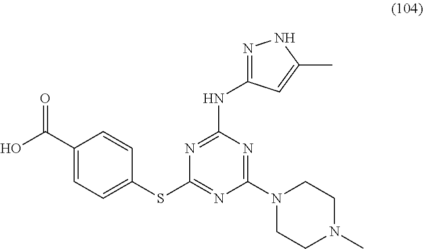

To the cyanuric chloride (300 mg, 1.63 mMol) in 15 mL of THF at −10° C. was dropwise added a solution of 5-methyl-1H-pyrazol-3-amine (158 mg, 1.63 mMol) and DIPEA (298 μL, 221 mg, 1.71 mMol) in 10 mL of THF. Reaction mixture was stirred for 30 minutes at −10° C. 4-mercaptobenzoic acid (280 mg of 90% acid, 1.63 mMol) in 10 mL of THF was added at −10° C. followed by DIPEA (596 μL, 442 mg, 3.42 mMol). Reaction mixture was stirred for 1 hour at 0° C. and 3 hours at room temperature. 1-methylpiparezine (181 μL, 163 mg, 1.63 mMol) in 10 mL of THF was added at room temperature followed by DIPEA (298 μL, 221 mg, 1.71 mMol). After overnight stirring at room temperature 100 mL of H₂O was added, reaction mixture was acidified with 2N HCl (0.8 mL, 1.6 mMol of HCl) and extracted with CHCl₃/i-PrOH (3/1) mixture (10×75 mL). Organic fractions were combined and solvent was removed under reduced pressure. Flash column chromatography (silica, CH₂Cl₂/MeOH/H₂O 80/18/2) yielded 250 mg (36%) of 104 as a white solid. 1H NMR (400 MHz, DMSO) δ 9.68 (bs, 1H), 8.00 (bs, 2H), 7.25 (d, J=8.4 Hz, 2H), 6.15 (bs, 1H), 5.27 (s, 1H), 3.73 (bs, 4H), 2.48 (bs, 4H), 2.29 (s, 3H). MS (ESI) m/z 427 [M+H]+.

### Example 105

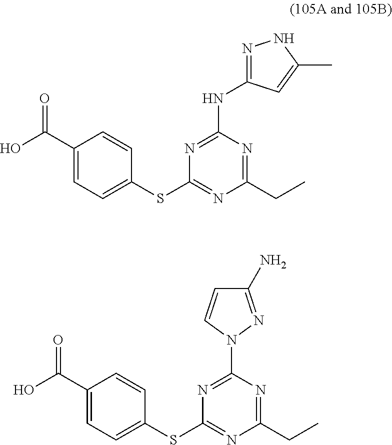

To the 5-methyl-1H-pyrazol-3-amine (66 mg, 0.68 mMol) in 2.5 mL of THF at room temperature was added DIPEA (261 μL, 193 mg, 1.5 mMol). After 5 minutes, compound 99 (200 mg, 0.68 mMol) in 2.5 mL of THF was added. Reaction mixture was stirred at room temperature for 2 days. 20 mL of H₂O was added followed by 350 μL of 2N HCl. Reaction mixture was extracted with EtOAc (4×20 mL). Organic fractions were combined, washed with brine, dried over Na₂SO₄, filtered and solvent was removed under reduced pressure. Flash column chromatography (silica CH₂Cl₂/MeOH/H₂O 80/20/0 gradient to 80/18/2) yielded top Rf product 105B (35 mg, 15%) 1H NMR (400 MHz, DMSO) δ 8.02 (d, J=8.0 Hz, 2H), 7.73 (d, J=8.0 Hz, 2H), 6.15 (s, 2H), 5.15 (s, 1H), 2.74 (q, J=7.6 Hz, 2H), 2.03 (s, 3H), 1.20 (t, J=7.2 Hz, 3H). MS (ESI) m/z 357 [M+H]+ and low Rf product 105A (55 mg, 23%). 1H NMR (400 MHz, DMSO) δ 10.27 (s, 1H), 8.03 (d, J=8.0 Hz, 2H), 7.70 (d, J=8.0 Hz, 2H), 5.21 (s, 1H), 2.55 (q, J=7.6 Hz, 2H), 1.94 (s, 3H), 1.18 (t, J=7.2 Hz, 3H). MS (ESI) m/z 357 [M+H]+.

### Example 106

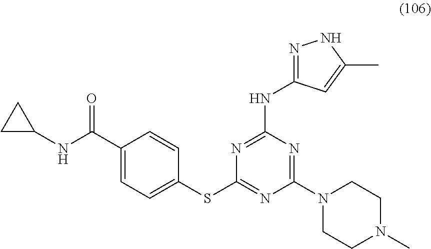

To the carboxylic acid 104 (50 mg, 0.12 mMol) in 3 mL of DMF at room temperature was added HBTU (55 mg, 0.14 mMol) followed by DIPEA (52 μL, 39 mg, 0.3 mMol). Reaction mixture was stirred at room temperature for 5 minutes and cyclopropyl amine (21 μL, 17 mg, 0.3 mMol) was added. After overnight stirring at room temperature 30 mL of H₂O was added and reaction mixture was extracted with EtOAc (4×25 mL). Organic fractions were combined, washed with water, brine, dried over Na₂SO4, filtered and solvent was removed under reduced pressure. Flash column chromatography (silica, CH₂Cl₂/MeOH 85/15) yielded 106 as white solid (52 mg, 95%). 1H NMR (400 MHz, DMSO) δ 11.73 (bs, 1H), 9.56 (bs, 1H), 8.56 (bs, 1H), 7.92 (bs, 2H), 7.67 (d, J=8.8 Hz, 2H), 5.23 (s, 1H), 3.68 (bs, 4H), 2.85 (o, J=4 Hz, 1H), 2.32 (bs. 4H), 2.20 (s, 3H), 0.71 (m, 2H), 0.58 (m, 2H). MS (ESI) m/z 466 [M+H]+.

### Example 107

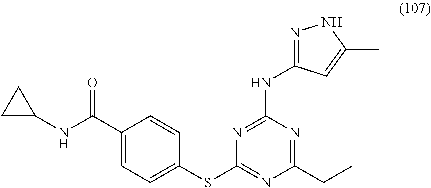

To the carboxylic acid 105A (40 mg, 0.11 mMol) in 3 mL of DMF at room temperature was added HBTU (49 mg, 0.13 mMol) followed by DIPEA (49 μL, 36 mg, 0.28 mMol). Reaction mixture was stirred at room temperature for 5 minutes and cyclopropyl amine (20 μL, 16 mg, 0.28 mMol) was added. After overnight stirring at room temperature 30 mL of H₂O was added and reaction mixture was extracted with EtOAc (4×25 mL). Organic fractions were combined, washed with water, brine, dried over Na₂SO₄, filtered and solvent was removed under reduced pressure. Flash column chromatography (silica, CH₂Cl₂/MeOH 95/5) yielded 107 as white solid (30 mg, 69%). 1H NMR (400 MHz, DMSO) δ 11.85 (bs, 1H), 10.26 (bs, 1H), 8.60 (bs, 1H), 7.97 (d, J=8.4 Hz, 2H), 7.71 (d, J=8.4 Hz, 2H), 5.16 (s, 1H), 2.86 (o, J=4 Hz, 1H), 2. 55 (q, J=7.6 Hz, 2H), 1.90 (s, 3H), 1.18 (t, J=7.6 Hz, 3H), 0.71 (m, 2H), 0.57 (m, 2H). MS (ESI) m/z 396 [M+H]+.

### Example 108

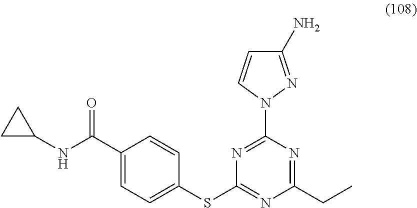

To the carboxylic acid 105B (30 mg, 0.084 mMol) and cyclopropyl amine (15 μL, 12 mg, 0.21 mMol) in 3 mL of DMF at room temperature was added HBTU (38 mg, 0.1 mMol) followed by DIPEA (37 μL, 27 mg, 0.21 mMol). After overnight stirring at room temperature 20 mL of H₂O was added and reaction mixture was extracted with EtOAc (4×25 mL). Organic fractions were combined, washed with water, brine, dried over Na₂SO₄, filtered and solvent was removed under reduced pressure. Flash column chromatography (silica, CH₂Cl₂/MeOH 95/5) yielded 108 as white solid (4 mg, 12%). 1H NMR (400 MHz, DMSO) δ 7.79 (d, J=8.4 Hz, 2H), 7.68 (d, J=8.4 Hz), 6.26 (bs. 1H), 5.60 (s, 1H), 4.00 (bs. 2H), 2.94 (o, J=3.6 Hz, 1H), 2.83 (q, J=7.6 Hz, 2H), 2.00 (s, 3H), 1.29 (t, J=7.6 Hz, 3H), 0.89 (m, 2H), 0.66 (m, 2H). MS (ESI) m/z 396 [M+H]+.

### Example 109

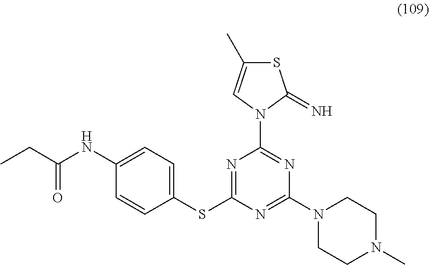

To the cyanuric chloride (300 mg, 1.63 mMol) in 15 mL of THF at −20° C. was dropwise added amide compound 80 (295 mg, 1.63 mMol) and DIPEA (312 mL, 232 mg, 1.79 mMol) in 10 mL of THF. Reaction mixture was stirred at −20° C. for 1 hr and 2-amino-5-methylthiazole (186 mg, 1.63 mMol) and DIPEA (312 μL, 232 mg, 1.79 mMol) in 10 mL of THF was added dropwise. Reaction mixture was warmed to 0° C. and stirred for 3 hours at 0° C. and 2 hours at room temperature. Methylpiperazine (181 μL, 163 mg, 1.63 mMol) and DIPEA (312 μL, 232 mg, 1.79 mMol) in 10 mL of THF was added dropwise. Reaction mixture was stirred overnight. 100 mL of H₂O was added and reaction mixture was extracted with EtOAc (3×) and CH₂Cl₂ (3×). Organic layers were combined, washed with brine, dried over Na₂SO₄, filtered and solvent was evaporated to give crude solid. Addition of a small amount of CH₂Cl₂ resulted in formation of solid product that was filtered to give 80 mg (10%) of desired triazine 109. 1H NMR (400 MHz, DMSO) δ 10.10 (bs, 1H), 8.97 (bs. 1H), 7.71 (d, J=8.8 Hz, 2H), 7.49 (d, J=8.8 Hz, 2H), 7.19 (s, 1H), 3.75-3.60 (m, 4H), 2.36 (q, J=7.6 Hz, 2H), 2.33 (m, 4H), 2.20 (s, 3H), 2.02 (d, J=1.6 Hz, 3H), 1.09 (t, J=7.6 Hz, 3H). MS (ESI) m/z 236 [M+2H]2+, 471 [M+H]+.

### Example 110

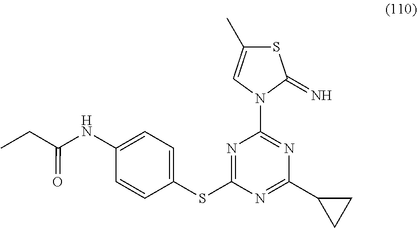

To the cyclopropyldichlorotriazine (compound 5) (200 mg, 1.05 mMol) in 10 mL of THF at 0° C. was dropwise added 2-amino-5-methylthiazole (120 mg, 1.05 mMol) and DIPEA (200 μL, 148 mg, 1.15 mMol) in 10 mL of THF. Reaction mixture was stirred for 3 hours at 0° C., 2 hours at room temperature. Amide 80 (190 mg, 1.05 mMol) and DIPEA (200 μL, 148 mg, 1.15 mMol) in 10 mL of THF was added and reaction mixture was stirred at 60° C. overnight. 50 mL of H₂O was added, organic layer was separated and aqueous layer was extracted with EtOAc. Combined organic layers were washed with brine, dried over Na₂SO₄ and filtered. Removal of solvent yielded crude material. Addition of small amount of CH₂Cl₂ resulted in formation of solid product that was filtered to give 120 mg (28%) of compound 110. 1H NMR (400 MHz, DMSO) δ 10.12 (bs, 1H), 9.03 (bs. 1H), 7.74 (d, J=8.8 Hz, 2H), 7.52 (d, J=8.8 Hz, 2H), 7.13 (d, J=1.6 Hz, 1H), 2.36 (q, J=7.6 Hz, 2H), 2.03 (d, J=1.6 Hz, 3H), 2.01 (m, 1H), 1.20-1.00 (m, 4H), 1.10 (t, J=7.6 Hz, 3H). m/z 413 [M+H]+.

### Example 111

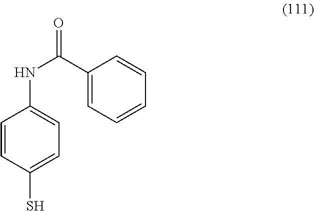

To the 4-aminothiophenol (1.0 g, 7.98 mMol) in 30 mL of CH₂Cl₂ at −10° C. was added pyridine (966 μL, 947 mg, 11.97 mMol) followed by dropwise addition of benzoyl chloride (930 μL, 1.12 g, 7.98 mMol). Reaction mixture was stirred overnight to room temperature. Reaction mixture was washed with 1N HCl and solvent was removed under reduced pressure. Crude material was dissolved in 25 mL of MeOH and 10 mL of H₂O. K₂CO₃ (1.1 g, 7.98 mMol) was added and reaction mixture was stirred at room temperature for 1 hr. After adjusting pH to 1 using 1N HCl, MeOH was evaporated and resulting aqueous solution was extracted with CH₂Cl₂. Organic fractions were combined, washed with brine, dried over Na₂SO₄, filtered and solvent was evaporated to give compound III as off-yellow solid (940 mg, 51%). 1H NMR (400 MHz, CDCl₃) δ 7.92-7.82 (m, 2H), 7.60-7.46 (m, 5H), 7.34-7.28 (m, 2H), 3.46 (s, 1H). MS (ESI) m/z 230 [M+H]+.

### Example 112

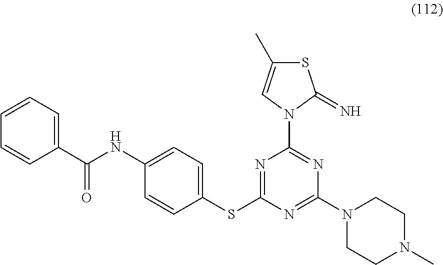

To the cyanuric chloride (300 mg, 1.63 mMol) in 15 mL of THF at −20° C. was dropwise added amide 111 (374 mg, 1.63 mMol) and DIPEA (312 μL, 232 mg, 1.79 mMol) in 10 mL of THF. Reaction mixture was stirred at −20° C. for 1 hr, and 2-amino-5-methylthiazole (186 mg, 1.63 mMol) and DIPEA (312 μL, 232 mg, 1.79 mMol) in 10 mL of THF was added dropwise. Reaction mixture was warmed to 0° C. and stirred for 3 hours at 0° C. and 2 hours at room temperature. Methylpiperazine (181 μL, 163 mg, 1.63 mMol) and DIPEA (312 μL, 232 mg, 1.79 mMol) in 10 mL of THF was added dropwise. Reaction mixture was stirred overnight. 100 mL of H₂O was added and reaction mixture was extracted with EtOAc (3×) and CH₂Cl₂ (3×). Organic layers were combined, washed with brine, dried over Na₂SO₄, filtered and solvent was evaporated to give crude solid. Addition of a small amount of CH₂Cl₂ resulted in formation of solid product 112 that was filtered to give 90 mg (11%) of desired triazine. 1H NMR (400 MHz, DMSO) δ 10.47 (bs, 1H), 9.04 (bs. 1H), 7.95 (m, 4H), 7.56 (m, 5H), 7.20 (d, J=1.6 Hz, 1H), 3.78-3.60 (m, 4H), 2.38 (m, 4H), 2.20 (s, 3H), 2.03 (d, J=1.6 Hz, 3H). MS (ESI) m/z 260 [M+2H]2+, 519 [M+H]+.

### Example 113

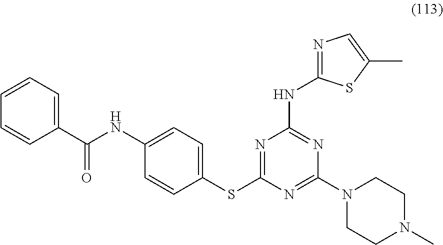

To the cyanuric chloride (300 mg, 1.62 mMol) in 10 mL of THF at −15° C. was dropwise added thiol 111 (374 mg, 1.63 mMol) and DIPEA (312 μL, 232 mg, 1.79 mMol) in 10 mL of THF. Reaction mixture was stirred for 90 minutes at −15° C. 2-amino-5-methylthiazole was added (186 mg, 1.63 mMol) followed by DIPEA (312 μL, 232 mg, 1.79 mMol) and reaction mixture was microwaved at 150° C. for 5 minutes. 1-Methylpiparezine (181 μL, 163 mg, 1.63 mMol) and DIPEA (312 μL, 232 mg, 1.79 mMol) was added and reaction mixture was microwaved at 60° C. for 15 minutes. 30 mL of EtOAC was added and reaction mixture was washed with saturated NaHCO₃, brine, dried over Na₂SO₄, filtered and solvent was removed under reduced pressure. Flash column chromatography (silica, CH₂Cl₂/MeOH 95/5 to 90/10) afforded 134 mg (16%) of desired product 113. 1H NMR (400 MHz, DMSO) δ11.25 (bs, 1H), 10.46 (s, 1H), 7.95 (m, 4H), 7.57 (m, 5H), 6.99 (bs, 1H), 3.90-3.50 (m, 4H), 2.45-2.21 (m, 7H), 2.20 (s, 3H). MS (ESI) m/z 519 [M+H]+.

### Example 114

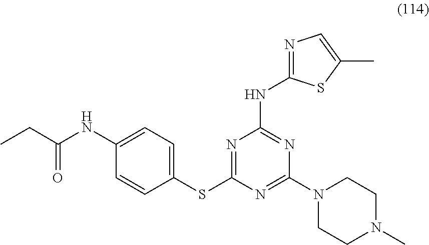

To the cyanuric chloride (300 mg, 1.62 mMol) in 10 mL of THF at −15° C. was dropwise added thiol 80 (295 mg, 1.63 mMol) and DIPEA (312 μL, 232 mg, 1.79 mMol) in 10 mL of THF. Reaction mixture was stirred for 90 minutes at −15° C. 2-amino-5-methylthiazole was added (186 mg, 1.63 mMol) followed by DIPEA (312 μL, 232 mg, 1.79 mMol) and reaction mixture was microwaved at 150° C. for 5 minutes. 1-Methylpiparezine (181 μL, 163 mg, 1.63 mMol) and DIPEA (312 μL, 232 mg, 1.79 mMol) was added and reaction mixture was microwaved at 60° C. for 15 minutes. 30 mL of EtOAC was added and reaction mixture was washed with saturated NaHCO₃, brine, dried over Na₂SO₄, filtered and solvent was removed under reduced pressure. Flash column chromatography (silica, CH₂Cl₂/MeOH 95/5 to 90/10) afforded 152 mg (20%) of desired product 114. 1H NMR (400 MHz, DMSO) 811.25 (bs, 1H), 10.09 (s, 1H), 7.70 (m, 2H), 7.51 (m, 2H), 6.99 (bs, 1H), 3.90-3.50 (m, 4H), 2.45-2.21 (m, 9H), 2.19 (s, 3H), 1.09 (t, J=7.6 Hz, 3H). MS (ESI) m/z 471 [M+H]+.

### Example 115

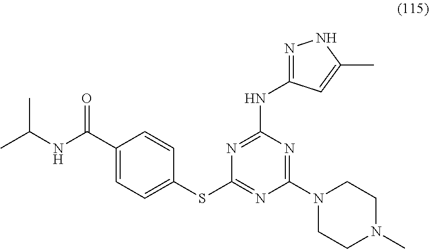

To the acid 104 (350 mg, 0.82 mMol) in 20 mL of DMF at room temperature was added DIPEA (357 μL, 265 mg, 2.05 mMol) and HBTU (374 mg, 0.99 mMol). Reaction mixture was stirred at room temperature for 120 minutes, and diisopropylamine (174 μL, 121 mg, 2.05 mMol) was added. After overnight stirring, 50 mL of water was added, and reaction mixture was extracted with EtOAc. Organic fractions were combined, washed with water (2×), brine (2×), dried over Na₂SO₄, filtered and solvent was evaporated. Flash column chromatography (silica, CH₂Cl₂/MeOH 95/95 to 85/15) yielded 220 mg (57%) of desired amide compound 115. 1H NMR (400 MHz, DMSO) δ 11.73 (bs, 1H), 9.57 (bs, 1H), 8.34 (bs, 1H), 7.96 (bs, 2H), 7.67 (d, J=7.2 Hz, 2H), 5.21 (s, 1H), 4.10 (h, J=6.8 Hz, 1H), 3.80-3.40 (m, 4H), 2.31 (m, 4H), 2.19 (s, 3H), 1.91 (s, 3H), 1.17 (d, J=6.8 Hz, 6H). MS (ESI) m/z 468 [M+H]+.

### Example 116

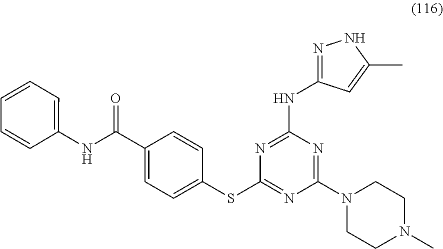

To the acid 104 (350 mg, 0.82 mMol) in 20 mL of DMF at room temperature was added DIPEA (357 μL, 265 mg, 2.05 mMol) and HBTU (374 mg, 0.99 mMol). Reaction mixture was stirred at room temperature for 120 minutes, and aniline (187 μL, 191 mg, 2.05 mMol) was added. After overnight stirring, 50 mL of water was added, and reaction mixture was extracted with EtOAc. Organic fractions were combined, washed with water (2×), brine (2×), dried over Na₂SO₄, filtered and solvent was evaporated. Flash column chromatography (silica, CH₂Cl₂/MeOH 95/95 to 85/15) yielded 151 mg (37%) of desired amide compound 116. 1H NMR (400 MHz, DMSO) δ 11.74 (bs, 1H), 10.34 (bs, 1H), 9.58 (bs, 1H), 8.07 (bs, 2H), 7.76 (m, 4H), 7.37 (m, 2H), 7.12 (m, 1H), 5.31 (s, 1H), 3.80-3.40 (m, 4H), 2.31 (m, 4H), 2.19 (s, 3H), 1.94 (s, 3H). MS (ESI) m/z 502 [M+H]+.

### Example 117

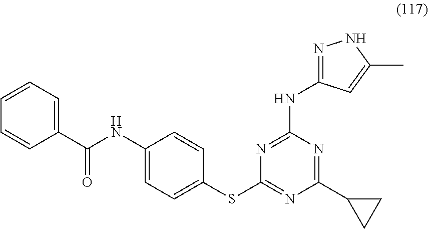

To the compound 5 (300 mg, 1.58 mMol) in 10 mL of THF at 0° C. was added 3-amino-5-methylpyrrazole (153 mg, 1.58 mMol) and DIPEA (303 μL, 225 mg, 1.74 mMol) in 5 mL of THF. Reaction mixture was stirred at 0° C. for 2 hours. 4-aminobenzanilide (335 mg, 1.58 mMol) and DIPEA (303 μL, 225 mg, 1.74 mmol) was added and reaction mixture was stirred at 60° C. overnight. 30 mL of EtOAc was added and reaction mixture was washed with saturated NaHCO₃, brine, dried over Na₂SO₄, filtered and solvent was evaporated. Flash column chromatography (silica, CH₂Cl₂/MeOH 95/5 to 90/10) yielded 230 mg (34%) of desired product compound 117. 1H NMR (400 MHz, DMSO) δ 11.93 (s, 1H), 10.17 (s, 1H), 9.48 (bs, 2H), 8.00-7.45 (m, 9H), 6.36 (bs, 1H), 2.22 (s, 3H), 1.85 (m, 1H), 1.00 (m, 4H). MS (ESI) m/z 427 [M+H]+.

### Example 118

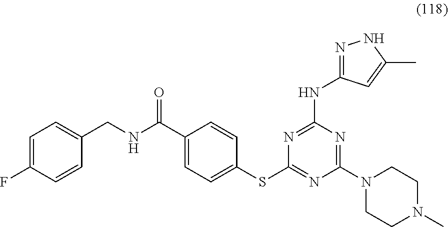

To the acid 104 (200 mg, 0.47 mMol) in 10 mL of DMF at room temperature was added DIPEA (204 μL, 151 mg, 1.17 mMol) and HBTU (212 mg, 0.56 mMol). Reaction mixture was stirred at room temperature for 120 minutes, and 4-fluoroaniline (132 μL, 146 mg, 1.17 mMol) was added. After overnight stirring, 50 mL of water was added, and reaction mixture was extracted with EtOAc. Organic fractions were combined, washed with water (2×), brine (2×), dried over Na₂SO₄, filtered and solvent was evaporated. Flash column chromatography (silica, CH₂Cl₂/MeOH 95/95 to 85/15) yielded 195 mg (78%) of desired amide compound 118. 1H NMR (400 MHz, DMSO) δ 11.70 (bs, 1H), 9.56 (bs, 1H), 9.19 (s, 1H), 7.98 (bs, 2H), 7.70 (m, 2H), 7.37 (m, 2H), 7.13 (m, 2H), 5.23 (s, 1H), 4.47 (d, J=6.0 Hz, 2H), 3.80-3.60 (m, 4H), 2.30 (m, 4H), 2.19 (s, 3H), 1.77 (s, 3H). MS (ESI) m/z 534 [M+H]+.

### Example 119

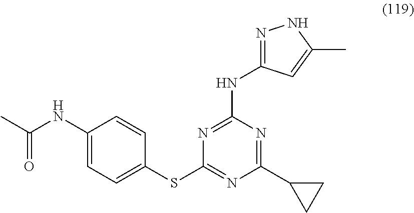

To the compound 5 (200 mg, 1.05 mMol) in 10 mL of THF at 0° C. was added 3-amino-5-methylpyrrazole (102 mg, 1.05 mMol) and DIPEA (201 μL, 150 mg, 1.15 mMol) in 5 mL of THF. Reaction mixture was stirred at room temperature for 2 hours. 4-aminoacetanilide (158 mg, 1.05 mMol) and DIPEA (201 μL, 150 mg, 1.15 mmol) was added and reaction mixture was stirred at 60° C. overnight. 30 mL of EtOAc was added and reaction mixture was washed with saturated NaHCO₃, brine, dried over Na₂SO₄, filtered and solvent was evaporated. Flash column chromatography (silica, CH₂Cl₂/MeOH 95/5 to 90/10) yielded 60 mg (16%) of desired product compound 119. 1H NMR (400 MHz, DMSO) δ 11.92 (s, 1H), 9.81 (s, 1H), 9.40 (bs, 2H), 7.60 (bs, 2H), 7.48 (m, 2H), 6.36 (bs, 1H), 2.21 (s, 3H), 2.02 (s, 3H), 1.82 (m, 1H), 1.00 (m, 4H). MS (ESI) m/z 365 [M+H]+.

### Example 120

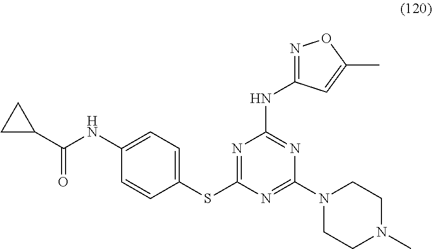

To the compound 7 (200 mg, 0.59 mMol) in 3 mL of DMF was added 3-amino-5-methylisoxazole (58 mg, 0.59 mMol) and DIPEA (112 μL, 83 mg, 0.65 mMol) in 1 mL of DMF. Reaction was stirred for 3 hours at room temperature. 1-methylpiparezine (66 μL, 59 mg, 0.59 mMol) and DIPEA (112 μL, 83 mg, 0.65 mMol) was added and reaction was stirred overnight at room temperature. Added 10 mL of water, reaction mixture was extracted with EtOAc. Organic fractions were combined, washed with brine, dried over Na₂SO₄, filtered and solvent was evaporated. Flash column chromatography (silica, CH₂Cl₂/MeOH 98/2 to 95/5) yielded 57 mg (21%) of desired product compound 120. 1H NMR (400 MHz, DMSO) δ 10.45 (s, 1H), 10.28 (s, 1H), 7.73 (d, J=8.8 Hz, 2H), 7.51 (d, J=8.8 Hz, 2H), 5.75 (bs, 1H), 3.69 (bs, 4H), 2.31 (bs, 4H), 2.19 (s, 3H), 2.15 (s, 3H), 1.81 (p, J=6.4 Hz, 1H), 0.81 (m, 4H). MS (ESI) m/z 467 [M+H]+.

### Example 121

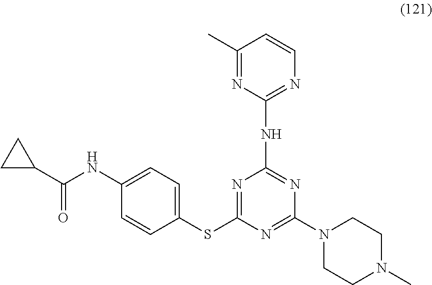

To the compound 7 (200 mg, 0.59 mMol) in 3 mL of DMF was added 2-amino-4-methylpyridine (64 mg, 0.59 mMol) and DIPEA (112 μL, 83 mg, 0.65 mMol) in 1 mL of DMF. Reaction was stirred for 3 hours at room temperature. 1-methylpiparezine (66 μL, 59 mg, 0.59 mMol) and DIPEA (112 μL, 83 mg, 0.65 mMol) was added and reaction was stirred overnight at room temperature. Added 10 mL of water, reaction mixture was extracted with EtOAc. Organic fractions were combined, washed with brine, dried over Na₂SO₄, filtered and solvent was evaporated. Flash column chromatography (silica, CH₂Cl₂/MeOH 95/5 to 90/10) yielded 5 mg (2%) of desired product compound 121. 1H NMR (400 MHz, DMSO) δ 10.34 (s, 1H), 10.03 (s, 1H), 8.44 (d, J=5.2 Hz, 1H), 7.63 (m, 2H), 7.49 (m, 2H), 6.98 (d, J=5.2 Hz, 1H), 3.80-3.40 (bs, 4H), 2.36 (s, 3H), 2.30 (bs, 4H), 2.17 (s, 3H), 1.79 (m, 1H), 0.82 (m, 4H). MS (ESI) m/z 478 [M+H]+.

### Example 122

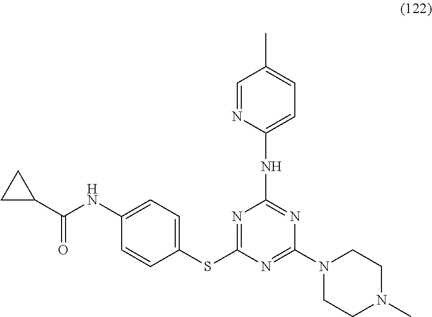

To the compound 7 (200 mg, 0.59 mMol) in 3 mL of DMF was added 2-amino-5-methylpicoline (63 mg, 0.59 mMol) and DIPEA (112 μL, 83 mg, 0.65 mMol) in 1 mL of DMF. Reaction was stirred for 3 hours at room temperature. 1-methylpiparezine (66 μL, 59 mg, 0.59 mMol) and DIPEA (112 μL, 83 mg, 0.65 mMol) was added and reaction was stirred overnight at room temperature. Added 10 mL of water, reaction mixture was extracted with EtOAc. Organic fractions were combined, washed with brine, dried over Na₂SO₄, filtered and solvent was evaporated. Flash column chromatography (silica, CH₂Cl₂/MeOH 95/5 to 90/10) yielded 51 mg (20%) of desired product compound 122. 1H NMR (400 MHz, DMSO) δ 10.46 (s, 1H), 9.51 (s, 1H), 8.02 (m, 1H), 7.70 (d, J=8.8 Hz, 2H), 7.51 (d, J=8.8 Hz, 2H), 7.35 (m, 1H), 7.20 (m, 1H), 3.63 (m, 4H), 2.29 (m, 4H), 2.19 (s, 3H), 2.16 (s, 3H), 1.85 (m, 1H), 0.86 (m, 4H). MS (ESI) m/z 477 [M+H]+.

### Example 123

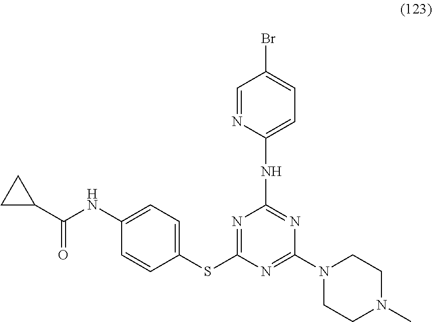

To the compound 7 (200 mg, 0.59 mMol) in 3 mL of DMF was added 2-amino-5-bromopyridine (102 mg, 0.59 mMol) and DIPEA (112 μL, 83 mg, 0.65 mMol) in 1 mL of DMF. Reaction was stirred for 3 hours at room temperature. 1-methylpiparezine (66 μL, 59 mg, 0.59 mMol) and DIPEA (112 μL, 83 mg, 0.65 mMol) was added and reaction was stirred overnight at room temperature. Added 10 mL of water, reaction mixture was extracted with EtOAc. Organic fractions were combined, washed with brine, dried over Na₂SO₄, filtered and solvent was evaporated. Flash column chromatography (silica, CH₂Cl₂/MeOH 98/2 to 95/5) yielded 54 mg (17%) of desired product compound 123. 1H NMR (400 MHz, DMSO) δ 10.48 (s, 1H), 9.90 (s, 1H), 8.29 (m, 1H), 7.71 (d, J=8.8 Hz, 2H), 7.52 (d, J=8.8 Hz, 2H), 7.45 (m, 1H), 7.40 (m, 1H), 3.80-3.55 (m, 4H), 2.31 (m, 4H), 2.19 (s, 3H), 2.16 (s, 3H), 1.85 (m, 1H), 0.88 (m, 4H). MS (ESI) m/z 541 and 543 [M+H]+.

### Example 124

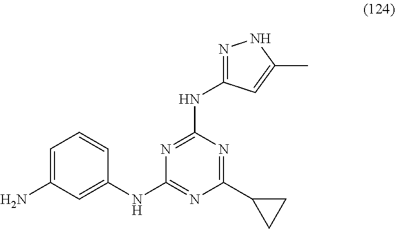

To the compound 5 (200 mg, 1.05 mMol) in 5 mL of THF at room temperature was added 3-amino-5-methylpyrazole (102 mg, 1.05 mMol) and DIPEA (201 μL, 150 mg, 1.15 mMol) in 5 mL of THF. Reaction mixture was stirred at room temperature for 2 hours. 1,3-phenylenediamine (114 mg, 1.05 mMol) and DIPEA (201 μL, 150 mg, 1.15 mMol) was added in 5 mL of THF, and reaction mixture was stirred at 60° C. overnight. 30 mL of EtOAc was added, and reaction mixture was washed with saturated NaHCO₃, brine, dried over Na₂SO₄, filtered and solvent was evaporated. Flash column chromatography (silica, CH₂Cl₂/MeOH 98/2 to 95/5 to 90/10) yielded 140 mg (74%) of desired product compound 124. 1H NMR (400 MHz, DMSO) δ 11.89 (s, 1H), 9.40 (s, 1H), 9.19 (s, 1H), 6.88 (s, 2H), 6.42 (s, 1H), 6.24 (s, 1H), 5.05 (s, 1H), 4.87 (s, 2H), 2.21 (s, 3H), 1.82 (m, 1H), 1.00 (m, 4H).

### Example 125

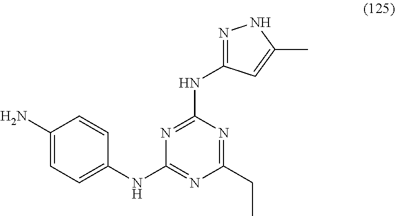

To the compound 3 (3 g, 16.9 mMol) in 10 mL of THF was carefully added 3-amino-5-methylpyrazole (1.64 g, 16.9 mMol) and DIPEA (3.24 mL, 2.41 g, 18.6 mMol) in 5 mL of THF. Reaction mixture was stirred for 2 hours at room temperature. 1,4-phenylenediamine (1.83 g, 16.9 mMol) and DIPEA (3.24 mL, 2.41 g, 18.6 mMol) was added, and reaction was microwaved at 100° C. for 60 minutes. Solvent was evaporated and flash column chromatography (silica, CH₂Cl₂/MeOH 95/5 to 90/10 0.1% Et₃N) yielded 2.7 g (51%) of desired product compound 125. 1H NMR (400 MHz, DMSO) δ 11.87 (s, 1H), 9.46 (s, 1H), 9.17 (s, 1H), 7.33 (m, 2H), 6.51 (d, J=8.4 Hz, 2H), 6.36 (bs, 1H), 4.82 (s, 2H), 2.47 (m, 2H), 2.20 (s, 3H), 1.20 (t, J=7.6 Hz, 3H). MS (ESI) m/z 311 [M+H]+.

### Example 126

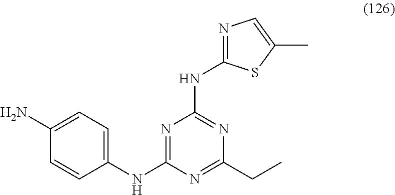

To the compound 3 (3 g, 16.9 mMol) in 10 mL of THF was carefully added 2-amino-5-methylthiazole (1.93 g, 16.9 mMol) and DIPEA (3.24 mL, 2.41 g, 18.6 mMol) in 5 mL of THF. Reaction mixture was stirred for 3 hours at room temperature. 1,4-phenylenediamine (1.83 g, 16.9 mMol) and DIPEA (3.24 mL, 2.41 g, 18.6 mMol) was added and reaction was microwaved at 150° C. for 120 minutes. Solvent was evaporated and flash column chromatography (silica, CH₂Cl₂/MeOH 95/5 to 90/10 0.1% Et₃N) yielded 1.9 g (34%) of desired product compound 126. 1H NMR (400 MHz, DMSO) δ 11.27 (s, 1H), 9.47 (s, 1H), 7.32 (m, 2H), 7.05 (s, 1H), 6.53 (d, J=8.4 Hz, 2H), 4.88 (s, 2H), 2.56 (q, J=7.2 Hz, 2H), 2.32 (s, 3H), 1.26 (m, 3H). MS (ESI) m/z 328 [M+H]+.

### Example 127

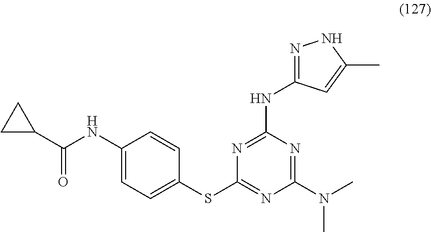

To a suspension of compound 2 (0.2 g, 0.588 mmol) in DMF (4 mL) was added DIPEA (0.13 mL, 0.65 mmol) and 3-amino-5-methylpyrazole (51 mg, 0.53 mmol). The mixture was heated at 150° C. for 15 minutes using microwave initiator. After cooling to room temperature, saturated NaHCO₃ in water was added to the flask and the mixture was extracted by dichloromethane (3×25 ml) and washed by brine, dried over sodium sulfate and concentrated. The resulting crude product was purified by Teledyne-Isco flash system by using DCM/MeOH, 0 to 5% of Methanol in dichloromethane to provide compound 127 as white solids (20 mg, 7.5%). 1H NMR (400 MHz, DMSO-d6) δ 11.75 (br, 1H), 10.38 (s, 1H), 9.52 (br s, 1H), 7.65 (m, 2H), 7.48 (d, J=8.8 Hz, 2H), 5.33 (br s, 1H), 3.05 (s, 6H), 2.14 (m, 3H), 1.78 (m, 1H), 0.78 (m, 4H); ESI-MS: calcd for (C₁₉H₂₂N₈OS) 410. found 411 (MH+). HPLC: retention time: 24.04 min. purity: 99%.

### Example 128

This example illustrated Aurora Kinase Assays of selected Compounds from this invention (referred to Daniele Fancelli et al, J. Med. Chem., 2006, 49 (24), pp 7247-7251). The KinaseProfiler™ Service Assay Protocols (Millipore) were used to test the kinase inhibiting activity of novel compounds from this invention. To do this, the buffer composition was as: 20 mM MOPS, 1 mM EDTA, 0.01% Brij-35, 5% Glycerol, 0.1% β-mercaptoethanol, 1 mg/mL BSA. Test compounds were initially dissolved in DMSO at the desired concentration, then serially diluted to the kinase assay buffer. In a final reaction volume of 25 μL, Aurora-A(h) (5-10 mU) is incubated with 8 mM MOPS pH 7.0, 0.2 mM EDTA, 200 μM LRRASLG (Kemptide), 10 mM MgAcetate and [γ33P-ATP]. The reaction was initiated by the addition of the MgATP mix. After incubation for 40 minute at room temperature, the reaction was stopped by addition of 5 μL of a 3% phosphoric acid solution. 10 μL of the reaction was then spotted onto a P30 filtermat and washed three times for 5 minutes in 50 mM phosphoric acid and once in methanol prior to drying and scintillation counting. Wells containing substrate but no kinase and wells containing a phosphopeptide control were used to set 0% and 100% phosphorylation value, respectively.

Also Kinase Hotspot SM kinase assay was used to test the compounds for IC₅₀ or % inhibitions (Reaction Biology Corp.). Inhibitor IC50 values were determined by titration of compound at the optimal kinase concentration (Kinase EC50).

Table 1 shows representative data for the inhibition of Aurora-A kinase by the compounds of this invention at a concentration of 1 μM.

TABLE 1

| Example No. | % Inhibition of aurora A @1 μM |
|---|---|
| 2 | >90 |
| 4 | >90 |
| 6 | >90 |
| 8 | <50 |
| 9 | >50 |
| 10 | >90 |
| 11 | <50 |
| 12 | >90 |
| 13 | >50 |
| 16 | <50 |
| 19 | >90 |
| 20 | >90 |
| 21 | >90 |
| 30 | >90 |
| 38 | >90 |
| 39 | >90 |
| 40 | >50 |
| 41 | <50 |
| 42 | >50 |
| 44 | >50 |
| 45 | >50 |
| 47 | <50 |
| 48 | >50 |
| 49 | >50 |
| 50 | >90 |
| 52 | >90 |
| 53 | >90 |
| 54 | >50 |
| 55 | >90 |
| 56 | >50 |
| 57 | <50 |
| 58 | <50 |
| 59 | >50 |
| 60 | <50 |
| 61 | <50 |
| 63 | <50 |
| 64 | <50 |
| 66 | <50 |
| 67 | <50 |
| 69 | <50 |
| 70 | <50 |
| 71 | >90 |
| 72 | <50 |
| 73 | >90 |
| 74 | >90 |
| 75 | <50 |
| 76 | >50 |
| 77 | <50 |
| 78 | <50 |
| 79 | >90 |
| 81 | >90 |
| 82 | >90 |
| 83 | >90 |
| 84 | >90 |
| 86 | >90 |
| 87 | >90 |
| 88 | >90 |
| 89 | >90 |
| 90 | >90 |
| 91 | >90 |
| 92 | >50 |
| 93 | >90 |
| 94 | >90 |
| 96 | >90 |
| 97 | >90 |
| 98 | >90 |
| 103 | <50 |
| 106 | >50 |
| 107 | <50 |
| 108 | <50 |
| 109 | <50 |
| 110 | >50 |
| 112 | >50 |
| 113 | >90 |
| 114 | >90 |
| 115 | >90 |
| 116 | >90 |
| 117 | >50 |
| 118 | >90 |
| 119 | >50 |
| 120 | <50 |
| 121 | <50 |
| 122 | >50 |
| 123 | >50 |
| 127 | >90 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound of the formula (I) 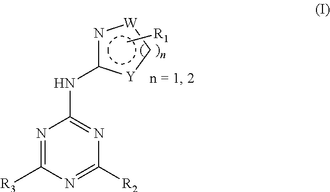
or a pharmaceutically acceptable salt thereof, wherein:
W and Y are independently selected from S, O, NR₄, CR₄ or CR₁;
R₄ is independently selected from hydrogen or an optionally substituted C₁₋₄ aliphatic group.
R₁ represents hydrogen, halogen, hydroxy, amino, cyano, alkyl, cycloalkyl, alkenyl, alkynyl, alkylthio, aryl, arylalkyl, heterocyclic, heteroaryl, heterocycloalkyl, alkylsulfonyl, alkoxycarbonyl and alkylcarbonyl.
R₂ is selected from:
(i) amino, alkyl amino, aryl amino, heteroaryl amino;
(ii) C₁-C₆ alkyl, C₂-C₆ alkenyl, C₂-C₆ alkynyl;
(iii) aryl, heterocyclic, heteroaryl; and
(iv) groups of the formula (Ia): 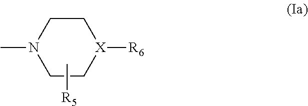
wherein:
R₅ represents hydrogen, C₁-C₁ alkyl, oxo;
X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl)sulfonamido, and mono- and di-(C₁-C₆alkyl)aminocarbonyl, each of which is substituted with from 0 to 4 substituents independently chosen from halogen, hydroxy, cyano, amino, —COOH and oxo;
R₃ is selected from:
(i) C₁-C₆ alkyl, C₂-C₆ alkenyl, C₂-C₆ alkynyl;
(ii) heterocyclic,
(iii) K—Ar;
Ar represents heteroaryl or aryl, each of which is substituted with from 0 to 4 substituents independently chosen from:
(1) halogen, hydroxy, amino, amide, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl)sulfonamido and mono- and di-(C₁-C₆alkyl)aminocarbonyl; phenylC₀-C₄alkyl and (4- to 7-membered heterocycle)-(C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.
K is selected from
i) absence;
ii) O, S, SO, SO₂;
iii) (CH₂)ₘ=0-3, —O(CH₂)ₚ, p=1-3, —S(CH₂)ₚ, p=1-3, —N(CH₂)ₚ, p=1-3, —(CH₂)ₚO, p=1-3;
iv) NR₇
R₇ represents hydrogen, alkyl, cycloalkyl, alkenyl, alkynyl, alkylthio, aryl, arylalkyl.

2. A process for making compound of claim 1 or its pharmaceutically acceptable salts, hydrates, solvates, crystal forms salts and individual diastereomers thereof.

3. A pharmaceutical composition comprising at least one compound of claim 1 or its pharmaceutically acceptable salts, hydrates, solvates, crystal forms salts and individual diastereomers thereof, and a pharmaceutically acceptable carrier.

4. A compound selected from the group consisting of:

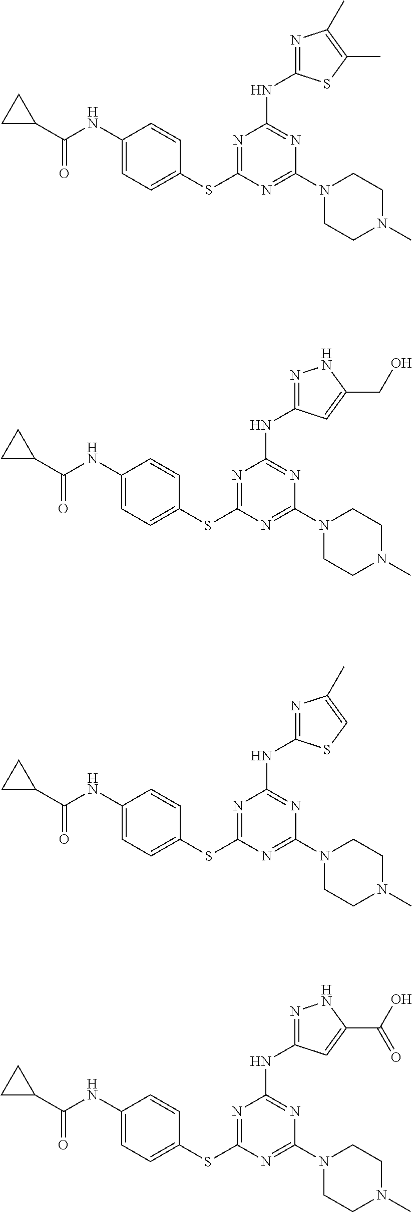

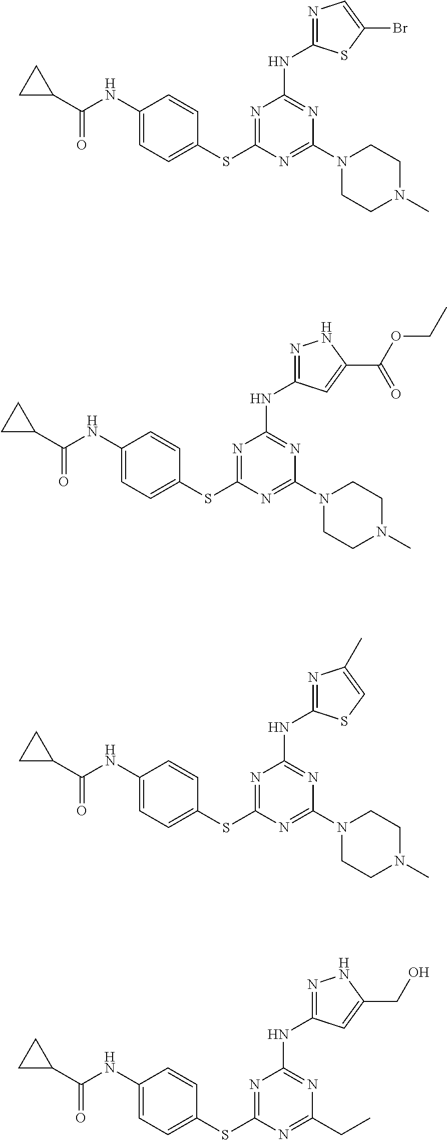

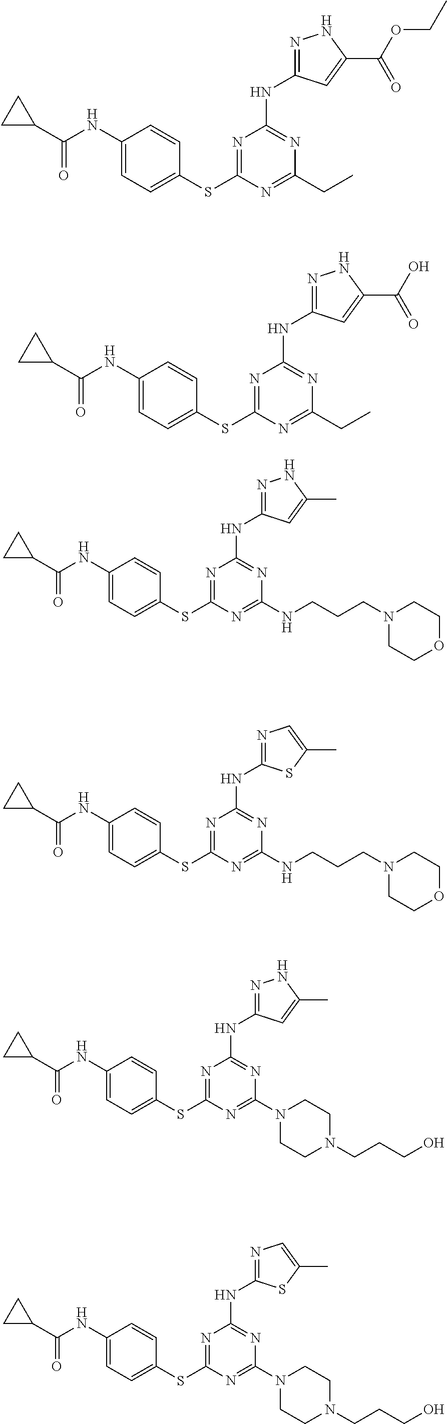

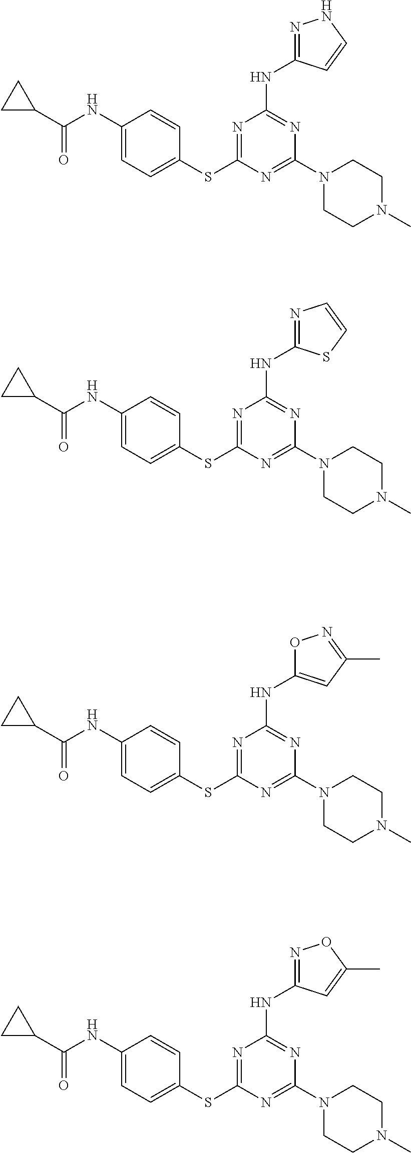

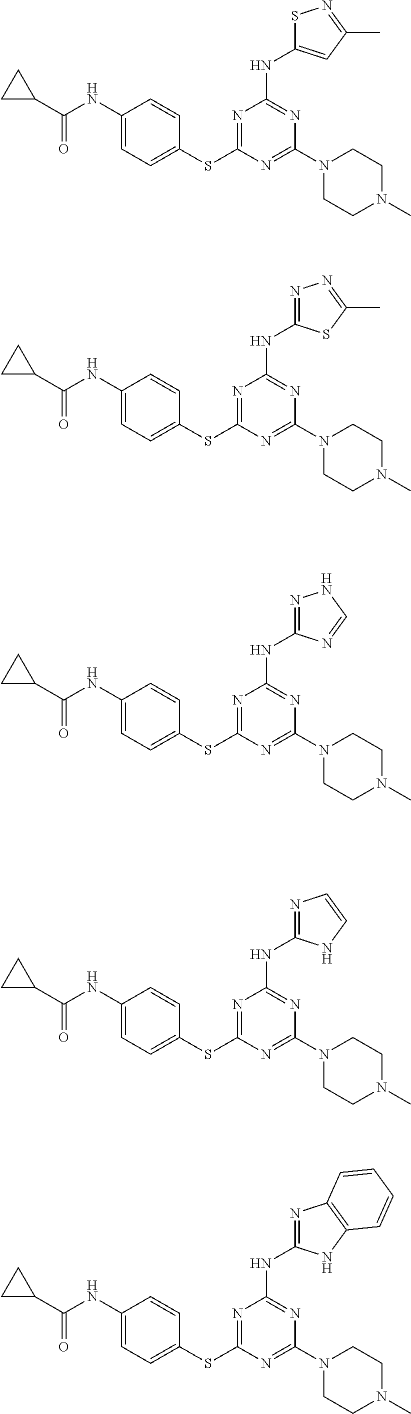

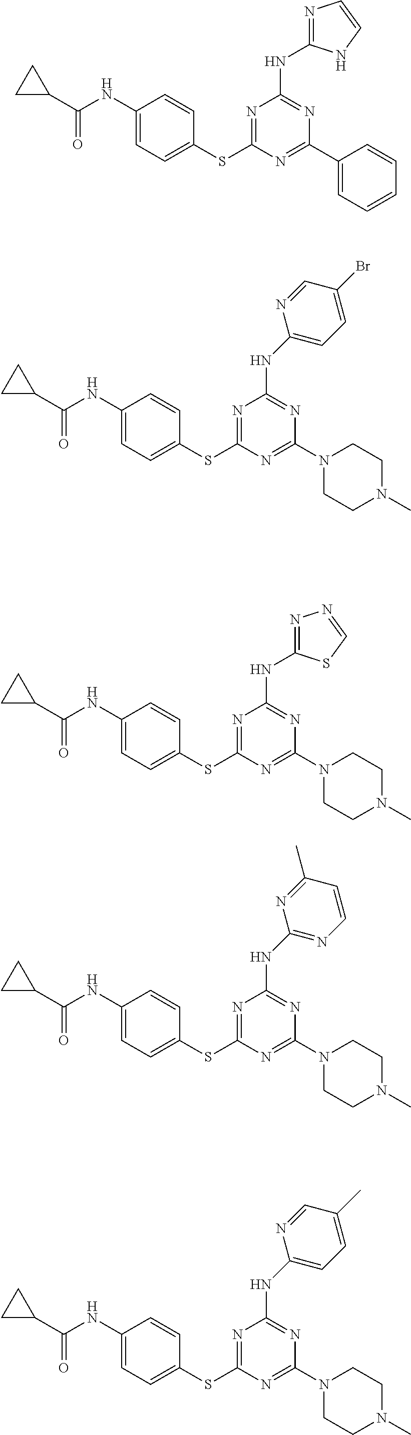

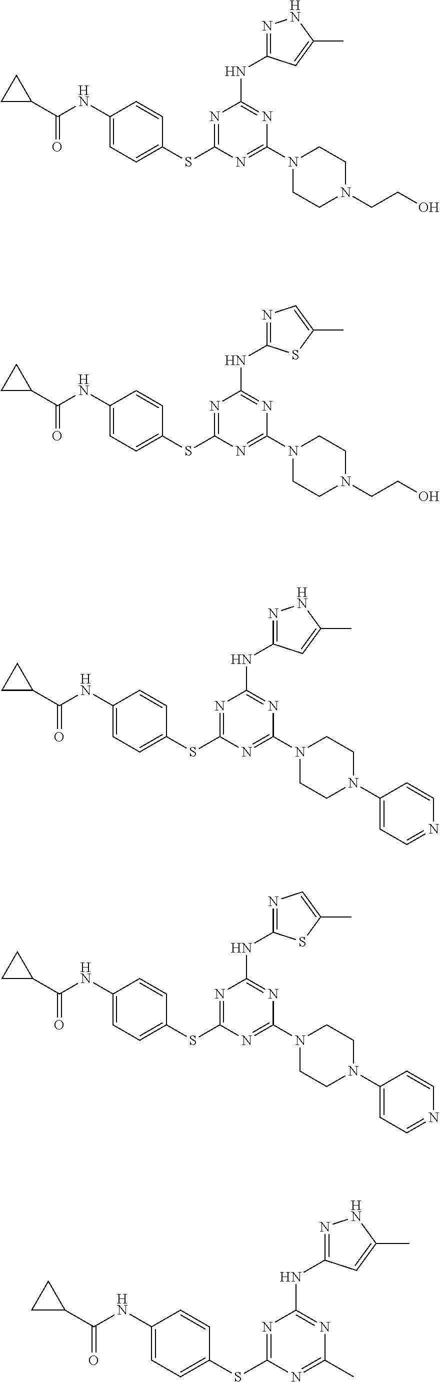

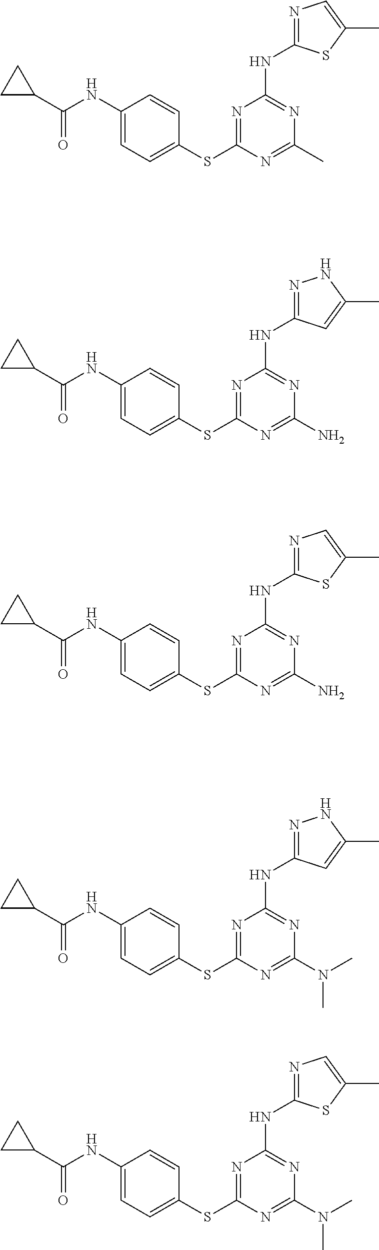

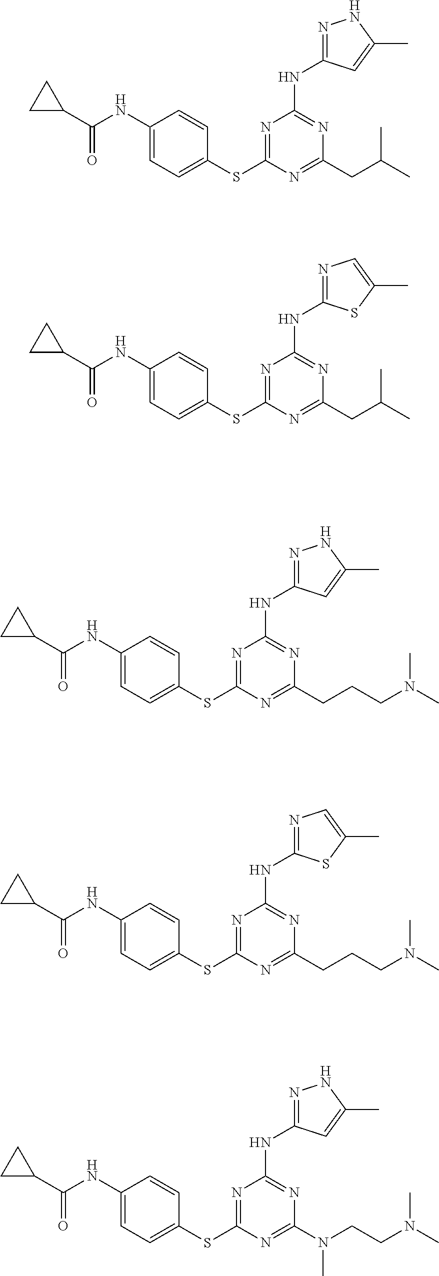

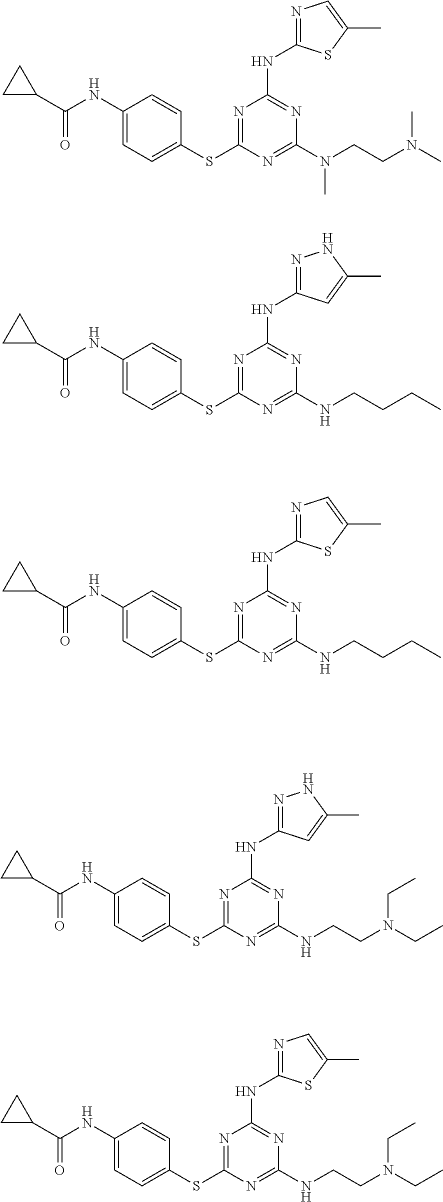

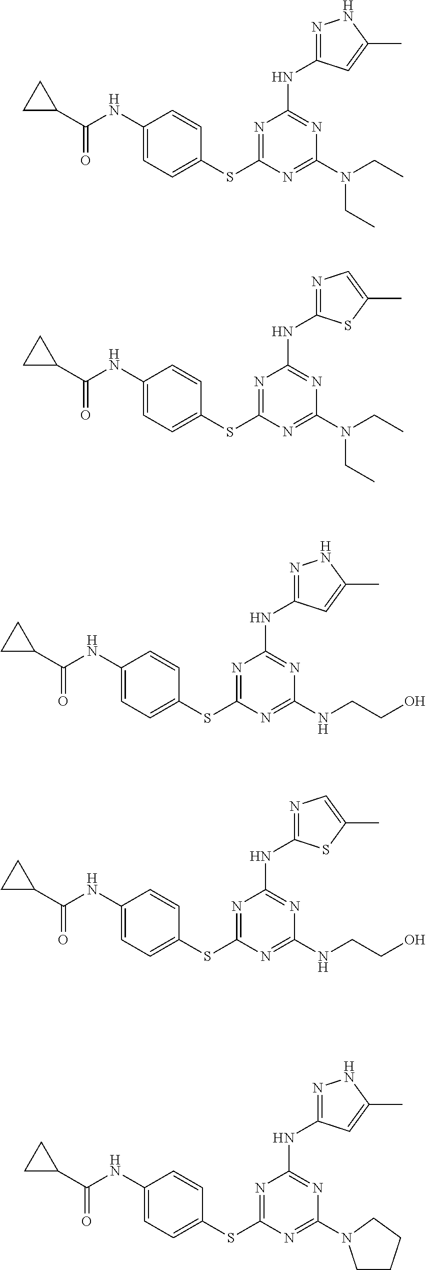

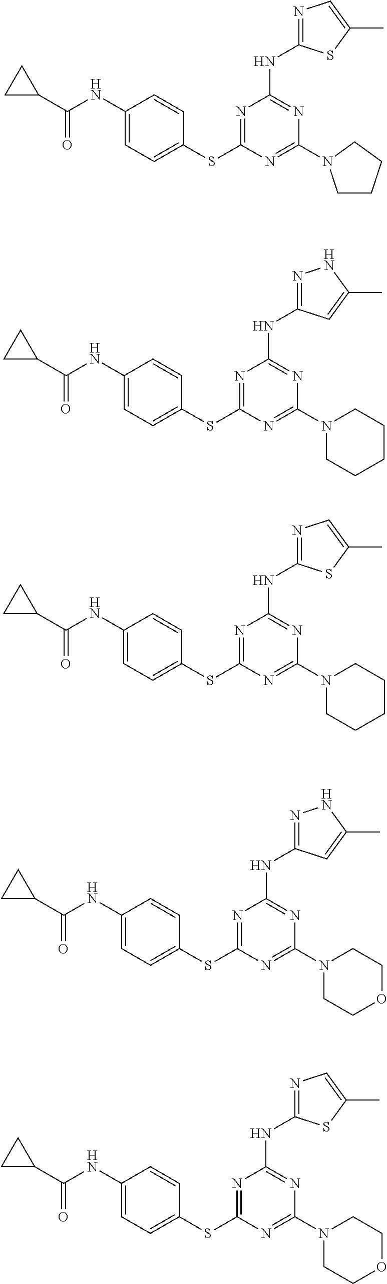

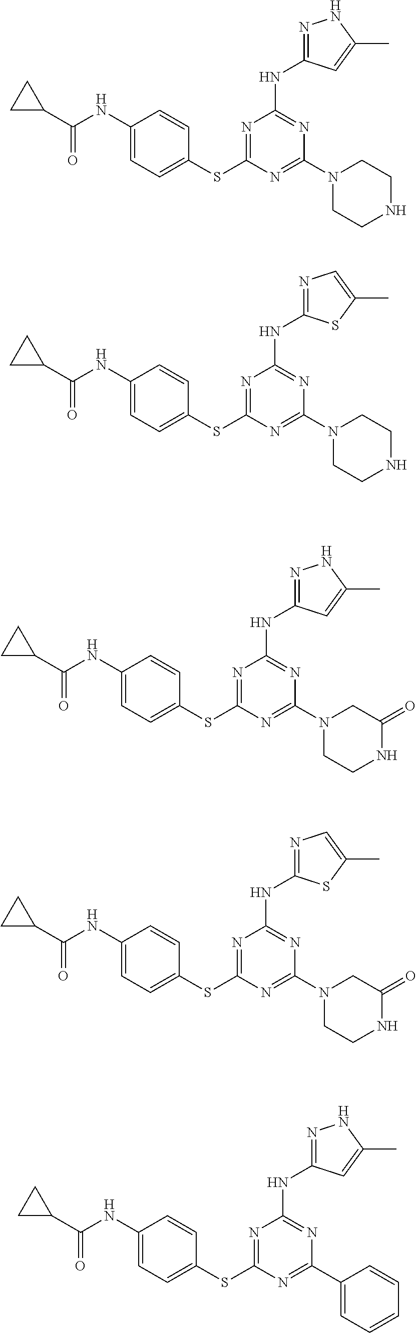

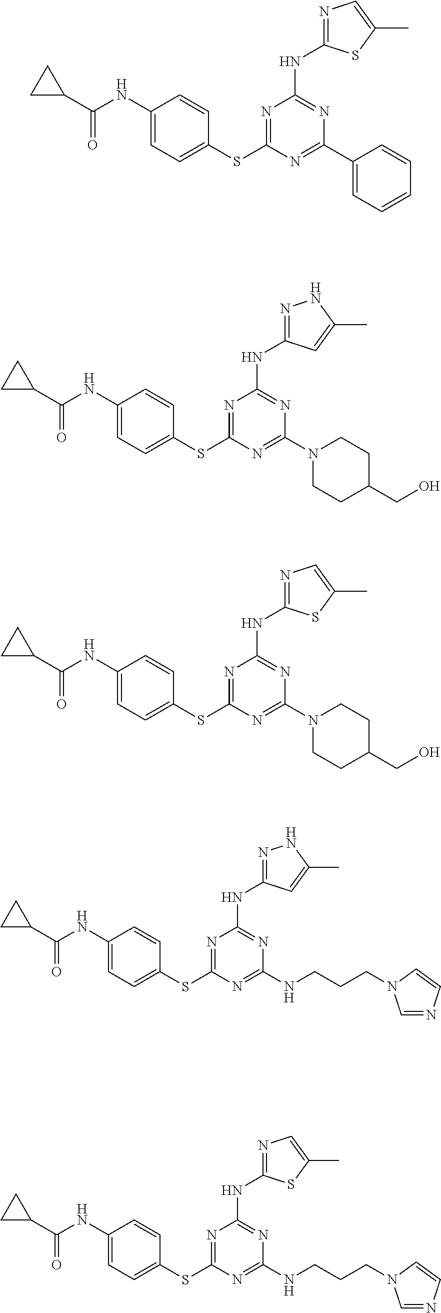

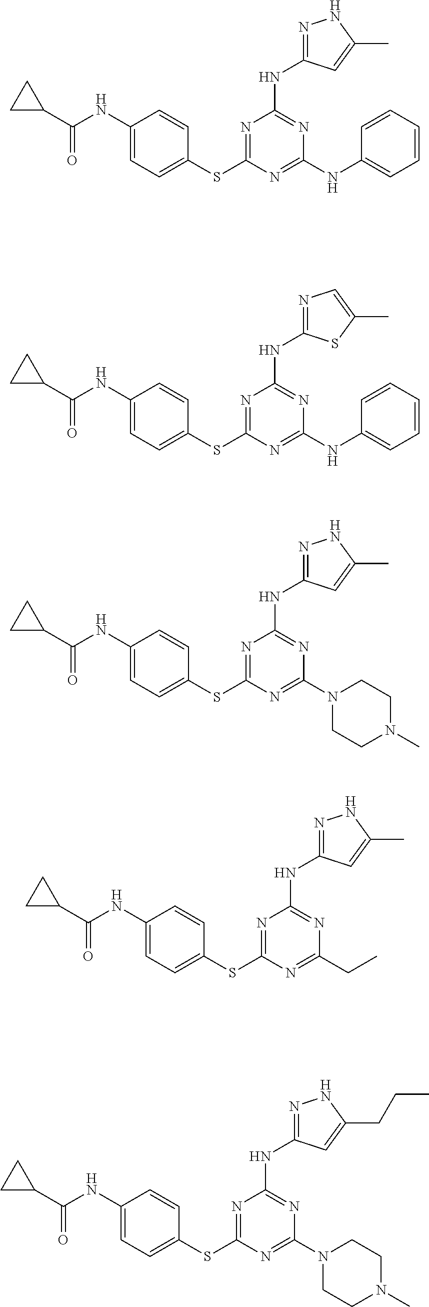

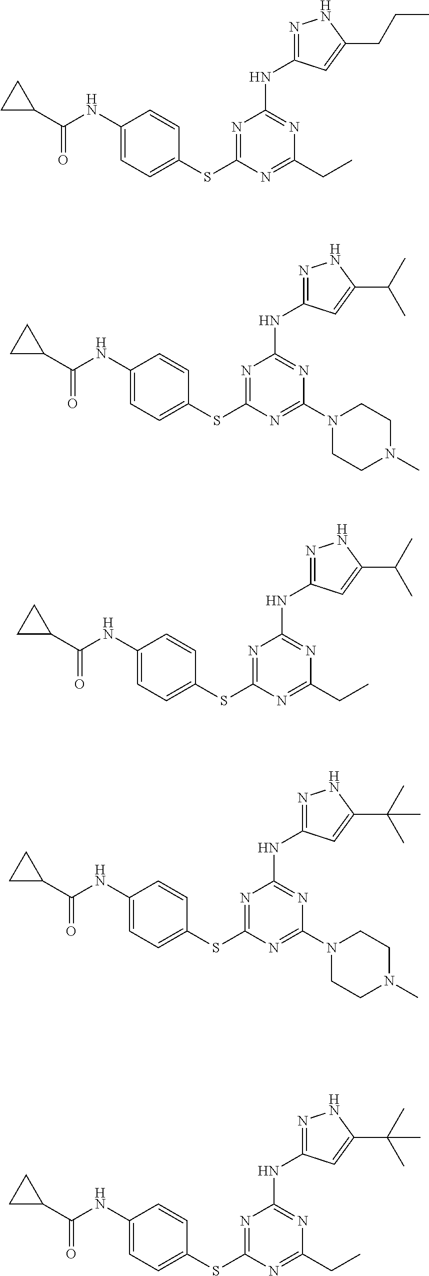

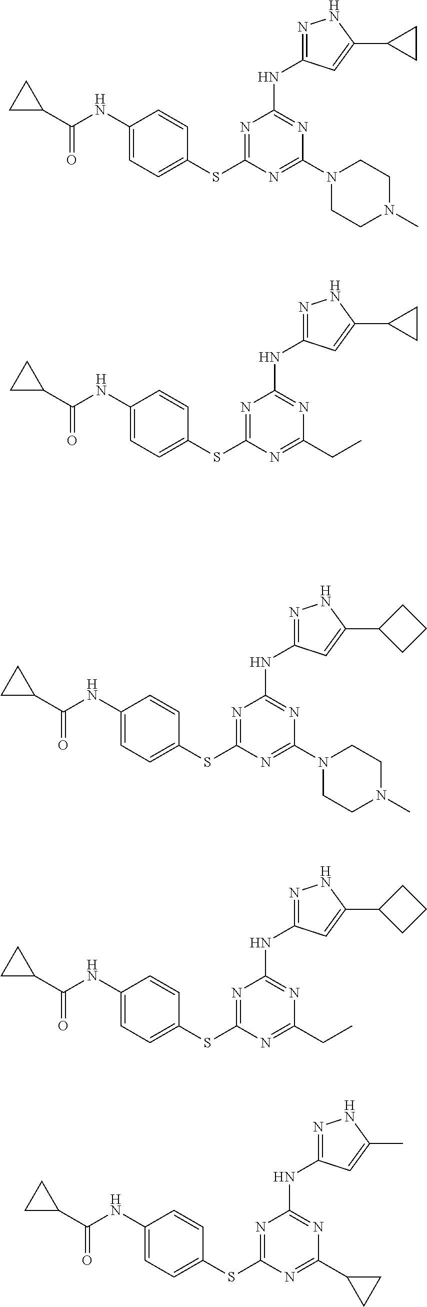

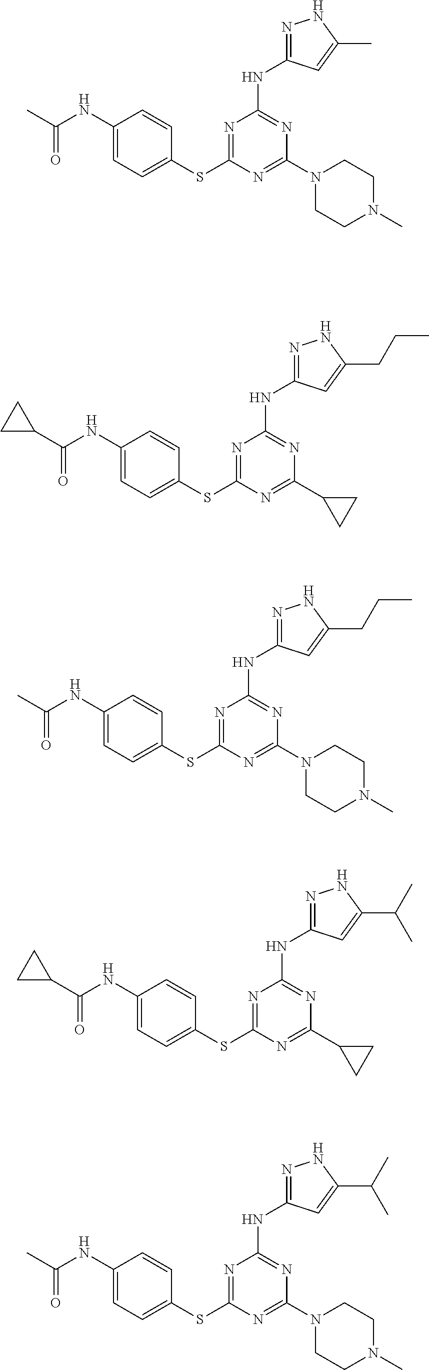

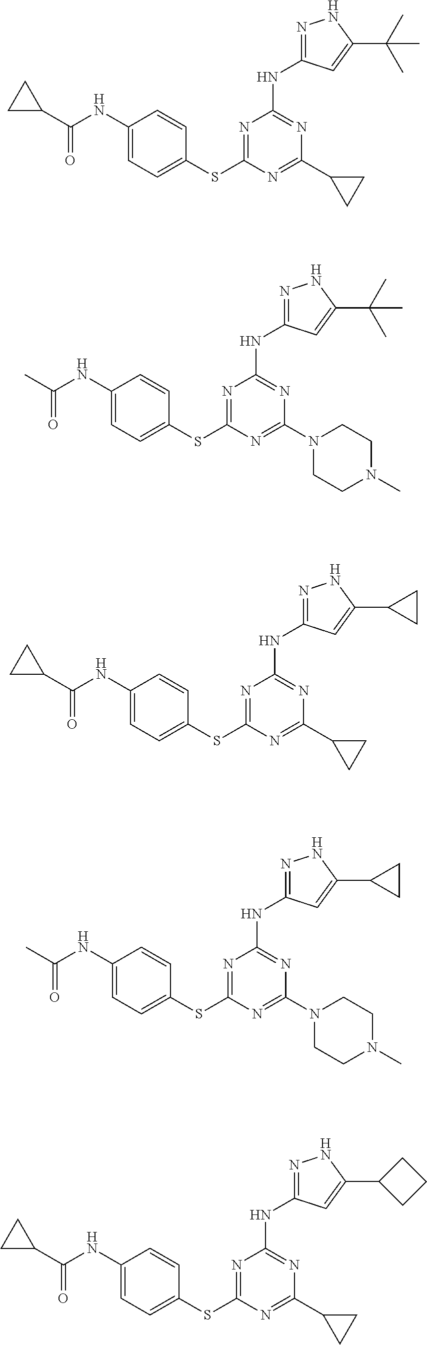

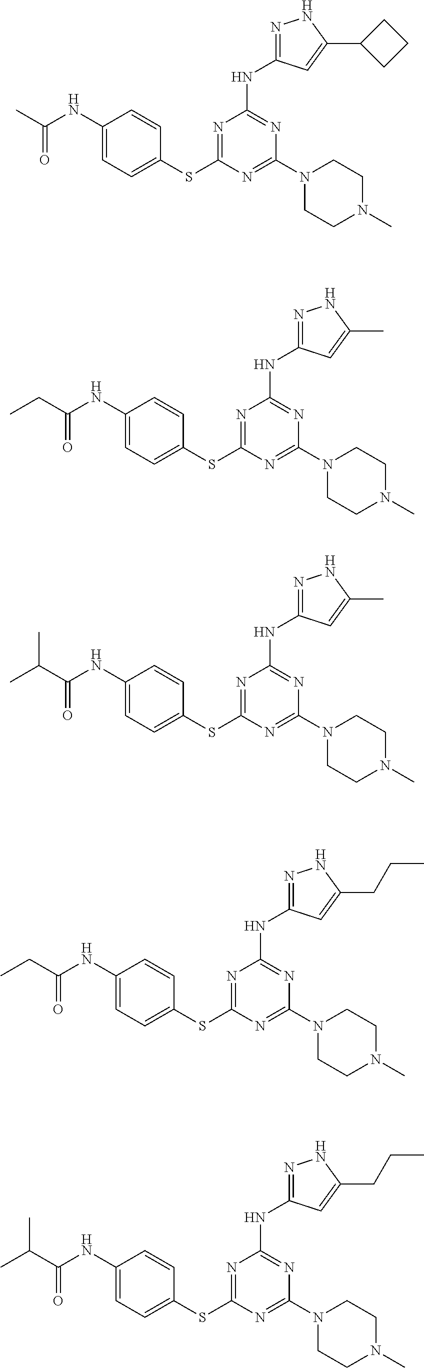

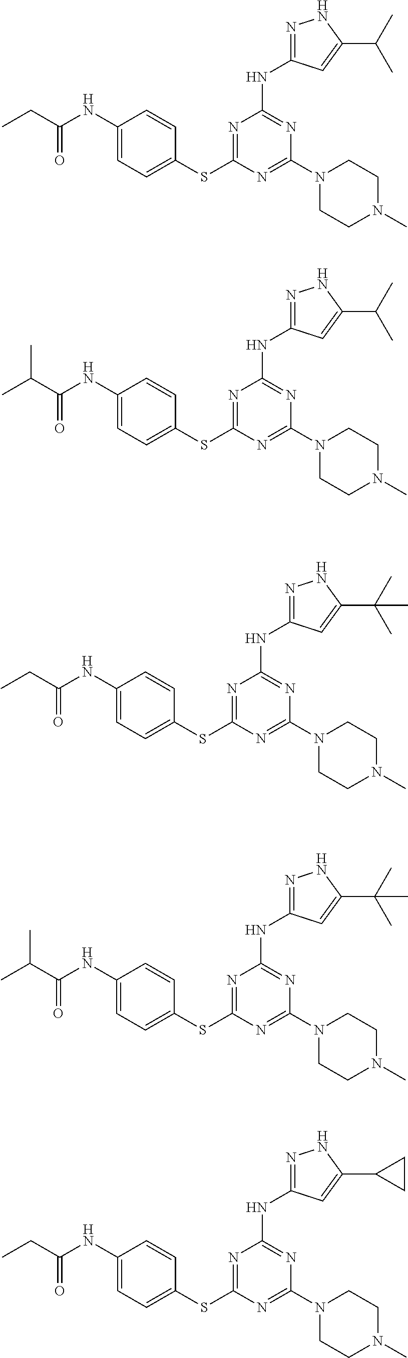

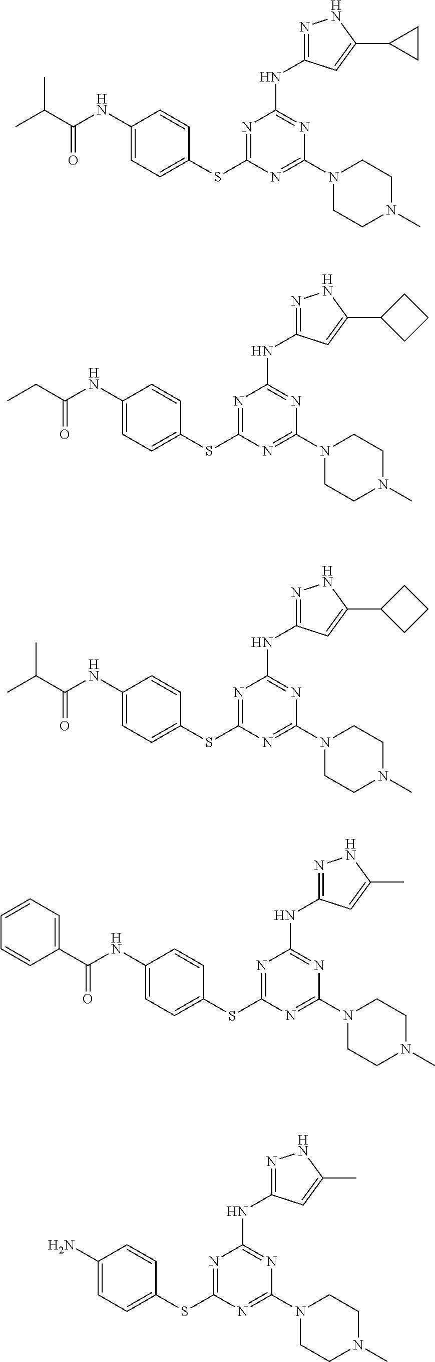

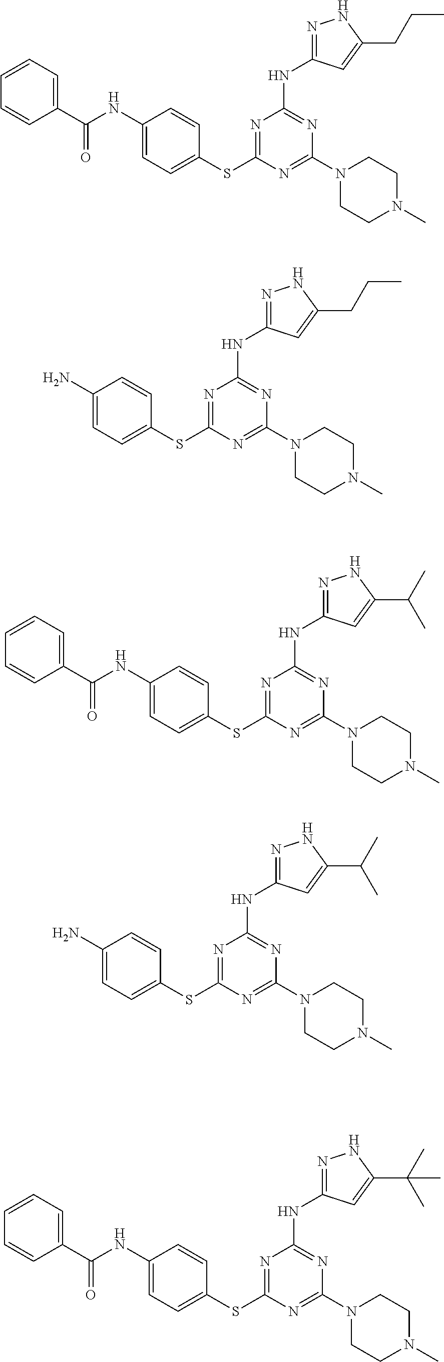

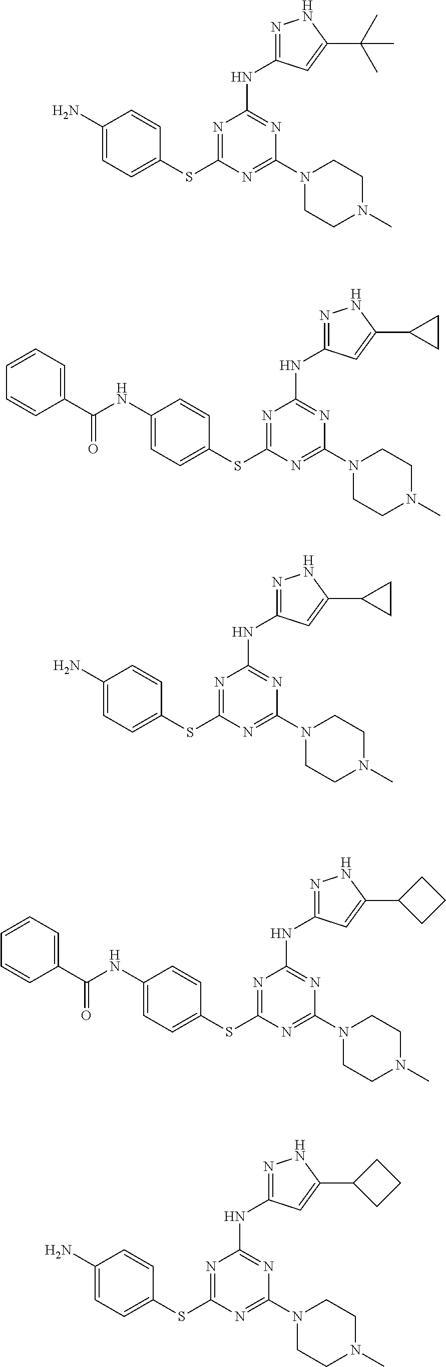

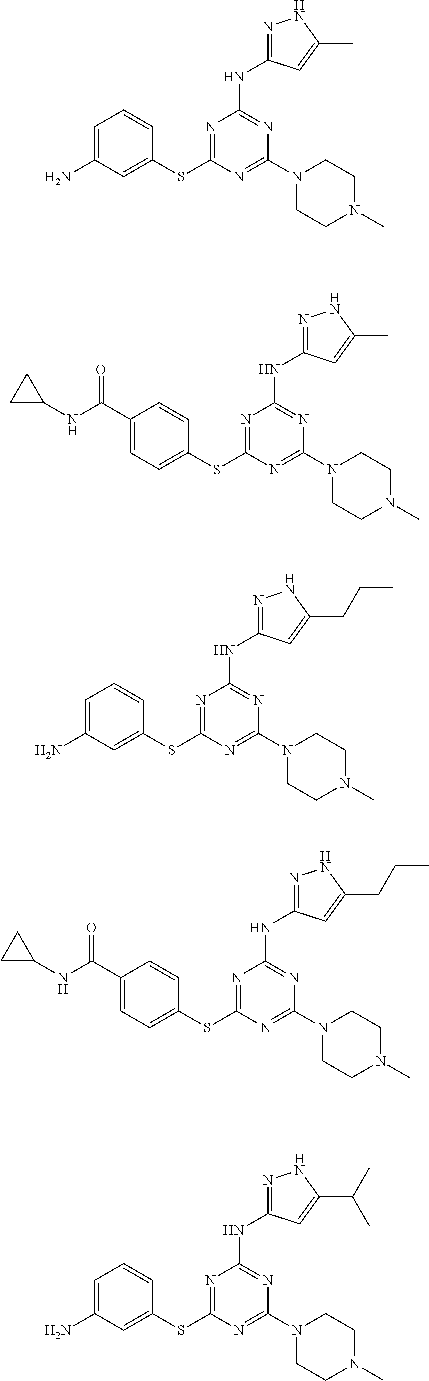

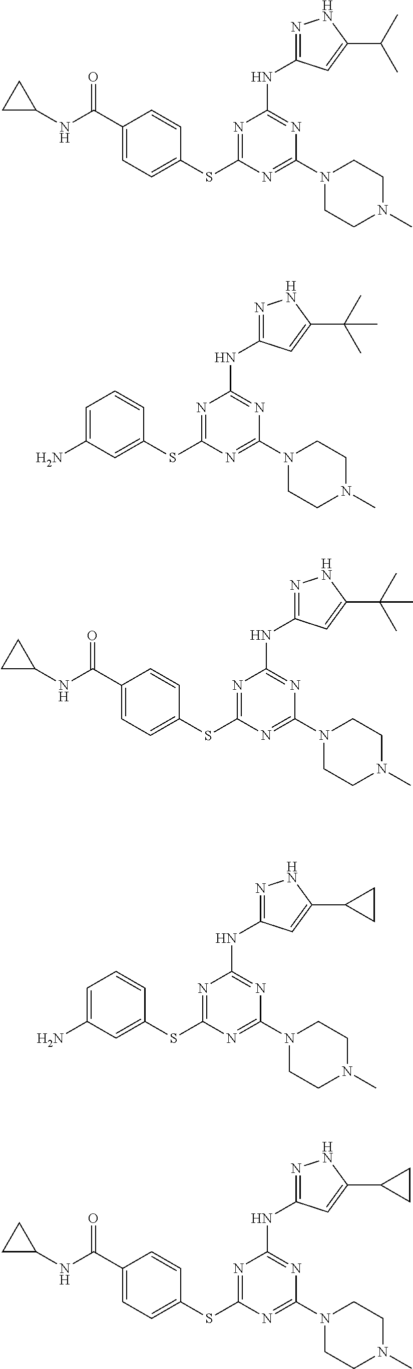

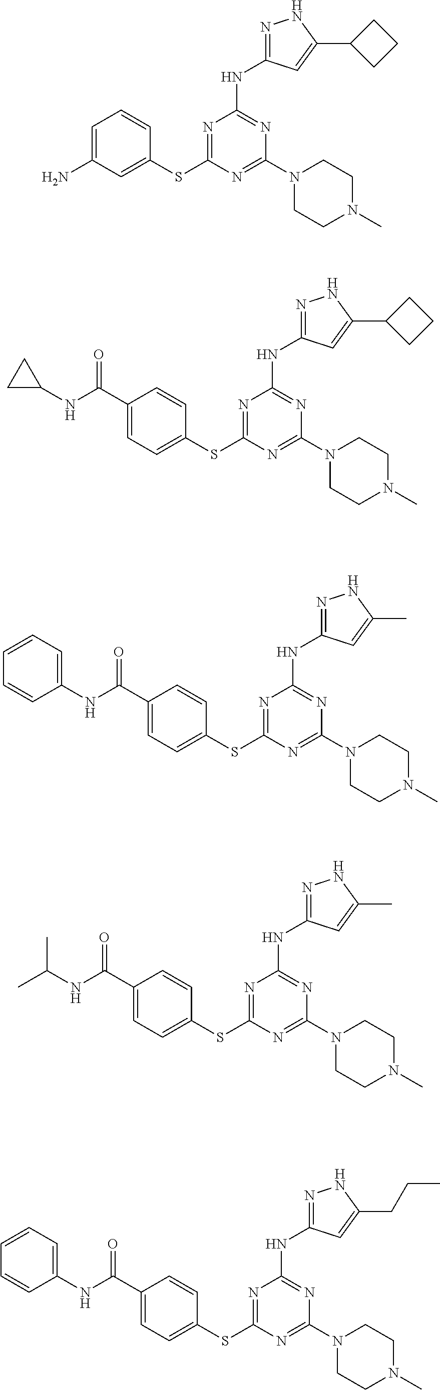

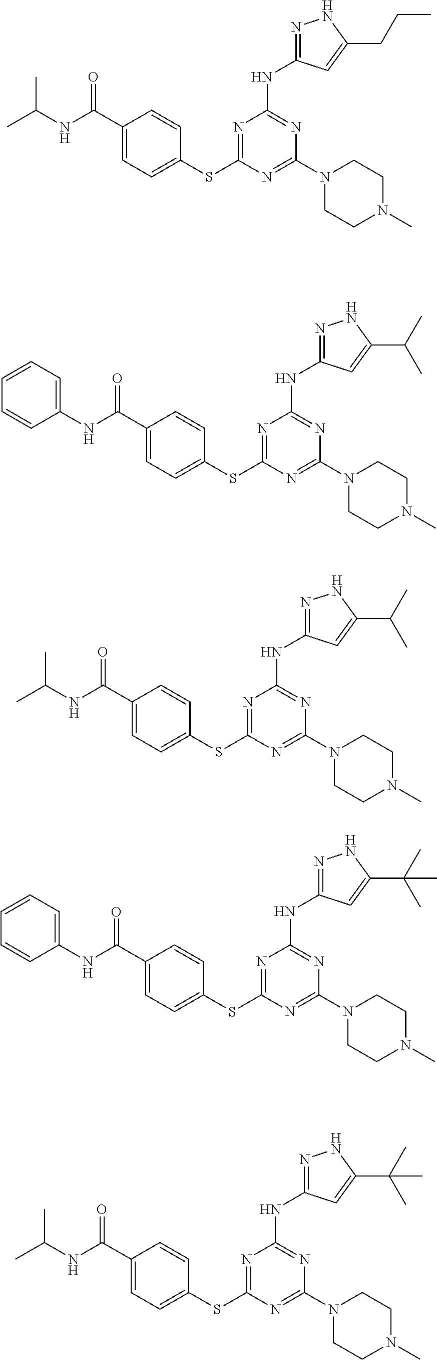

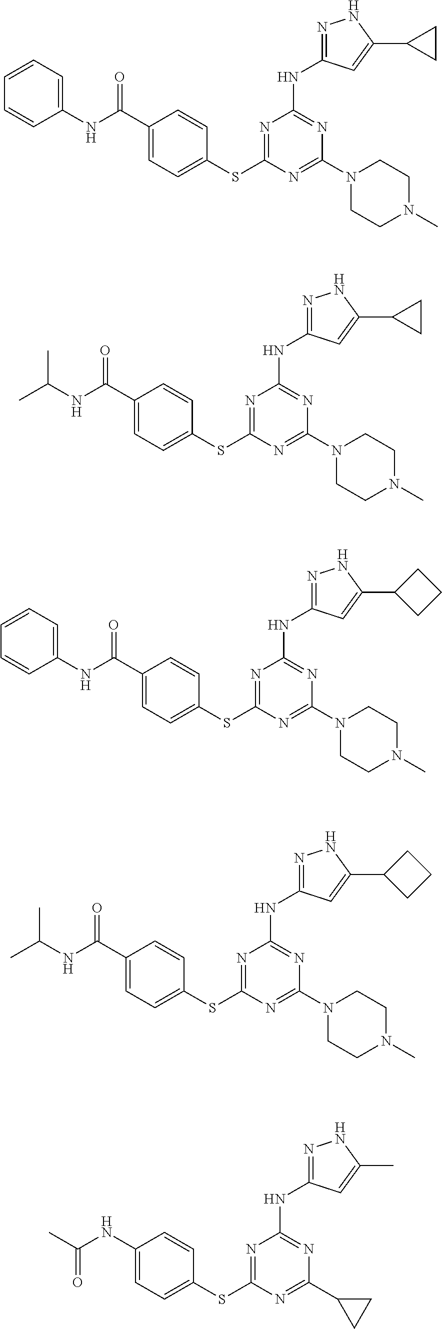

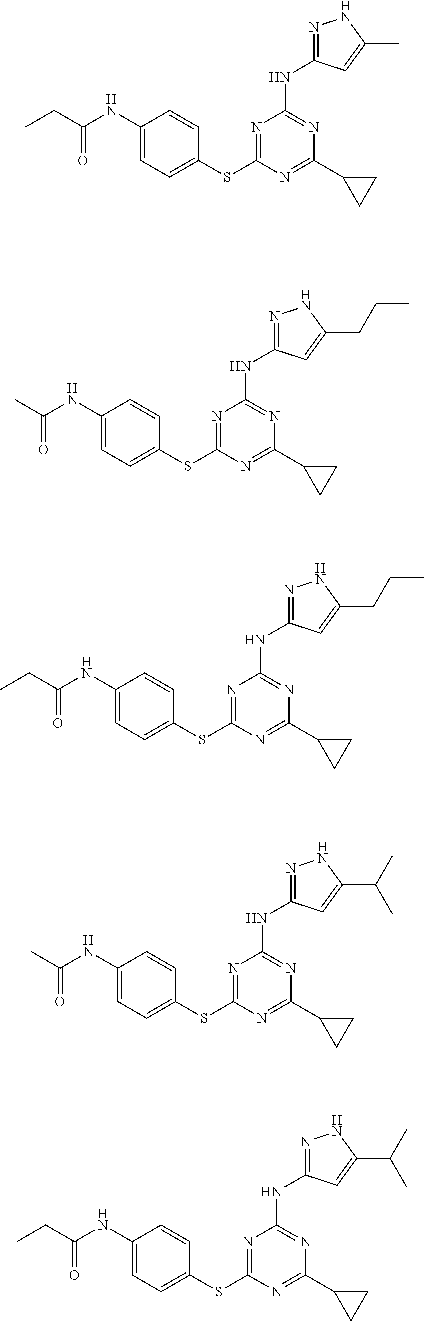

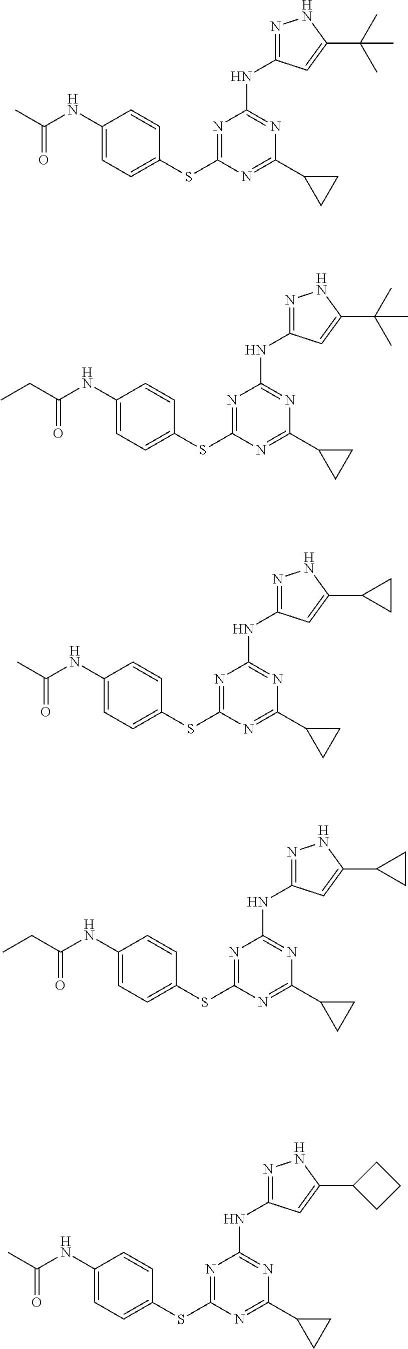

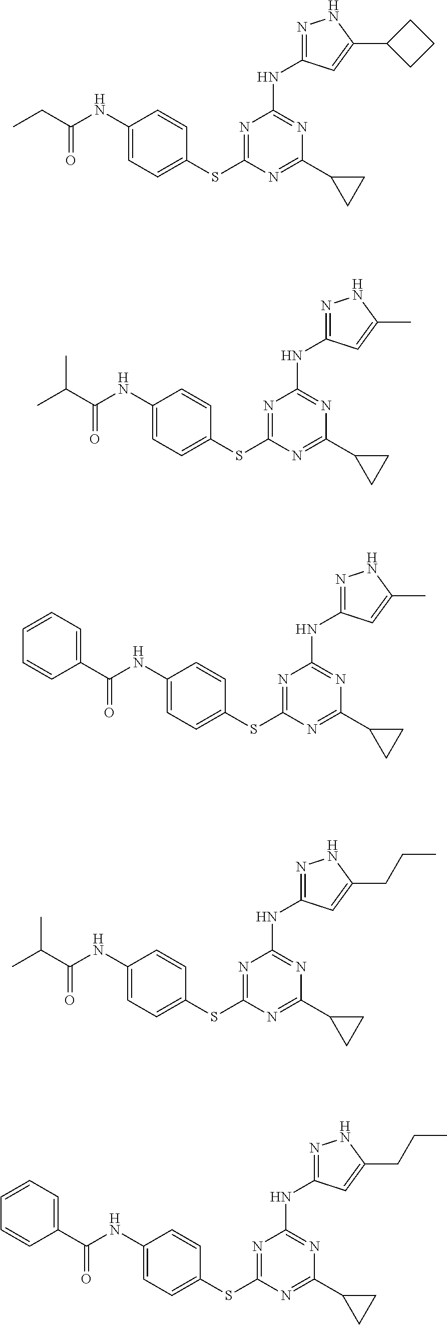

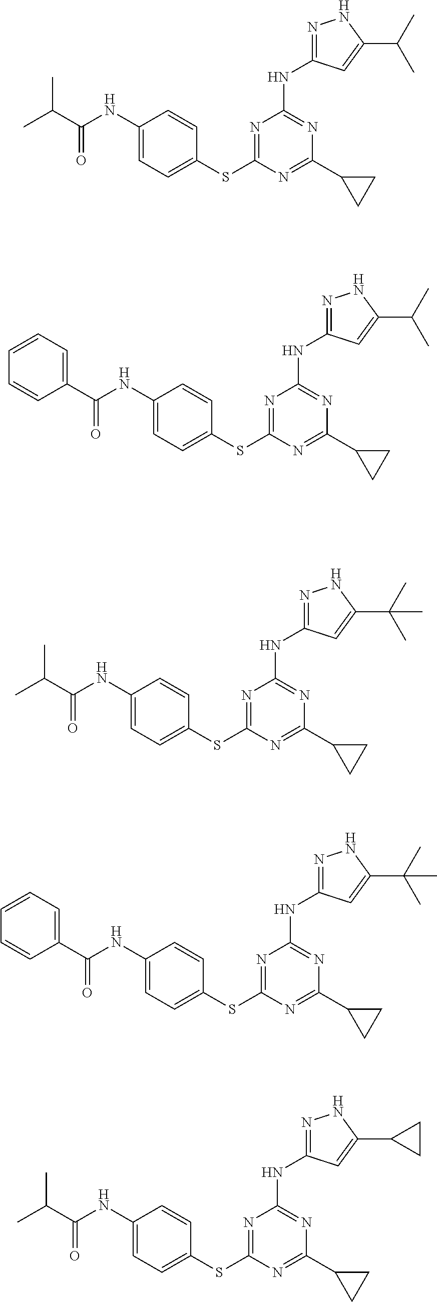

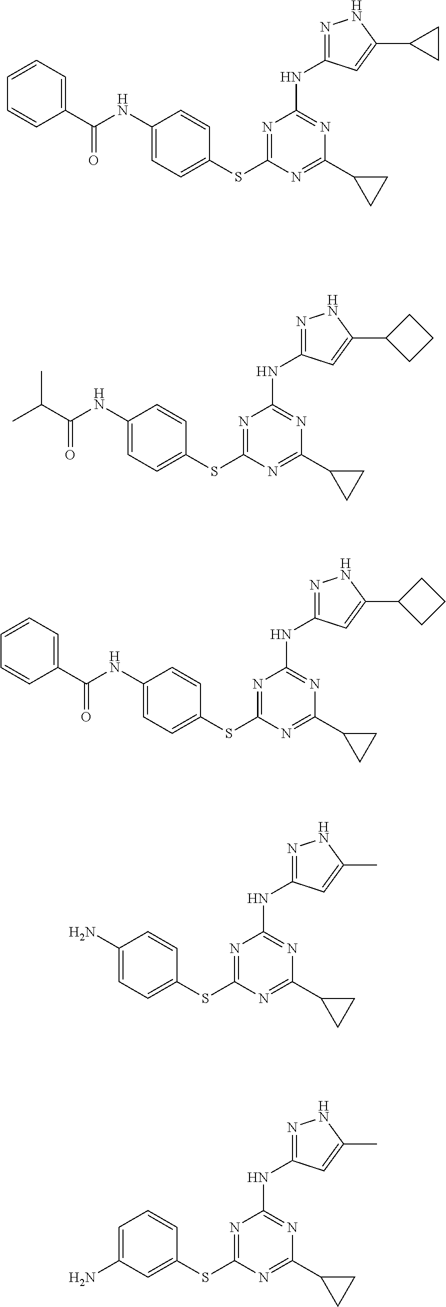

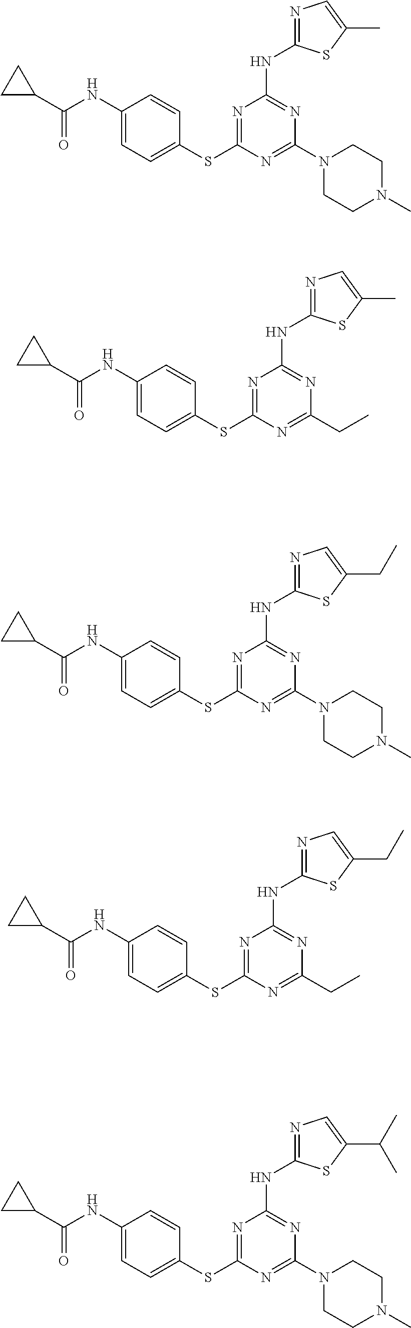

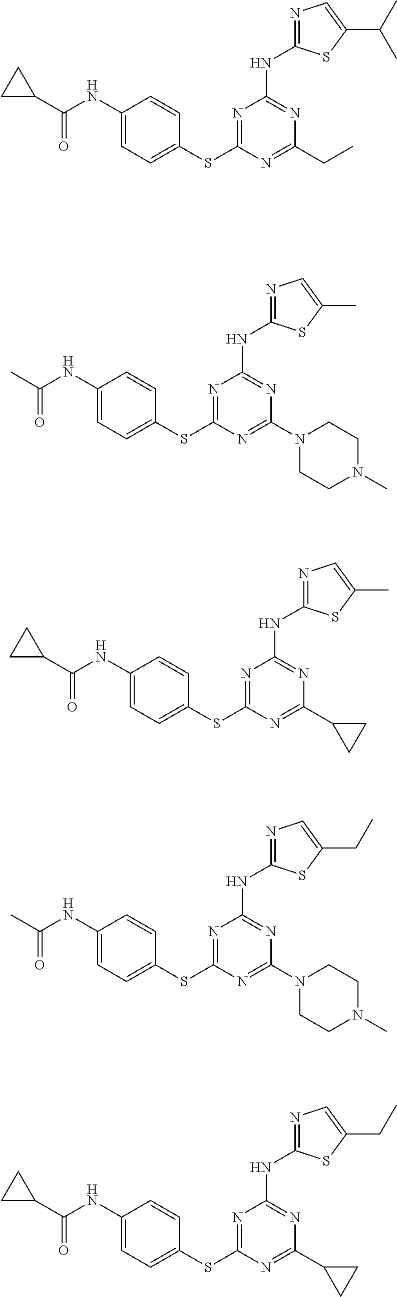

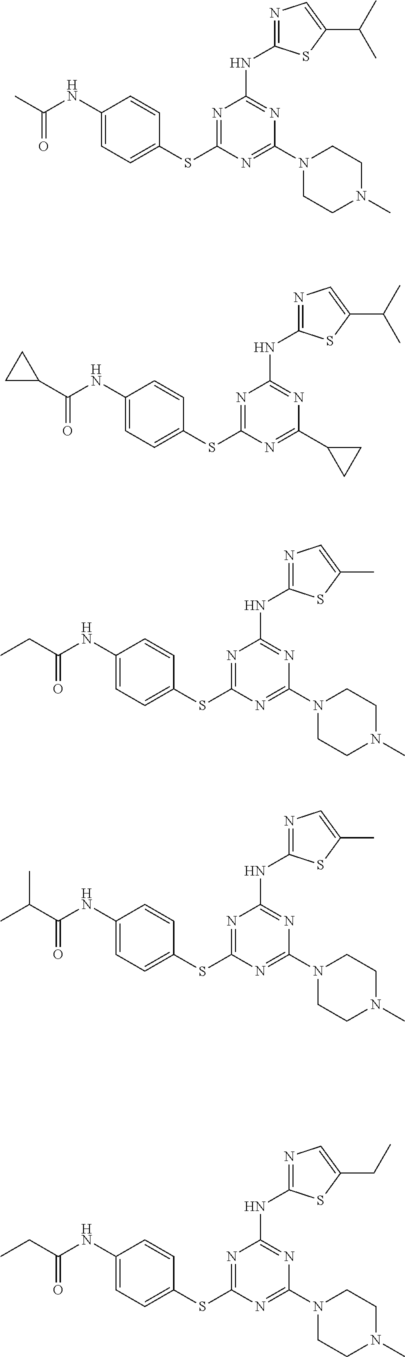

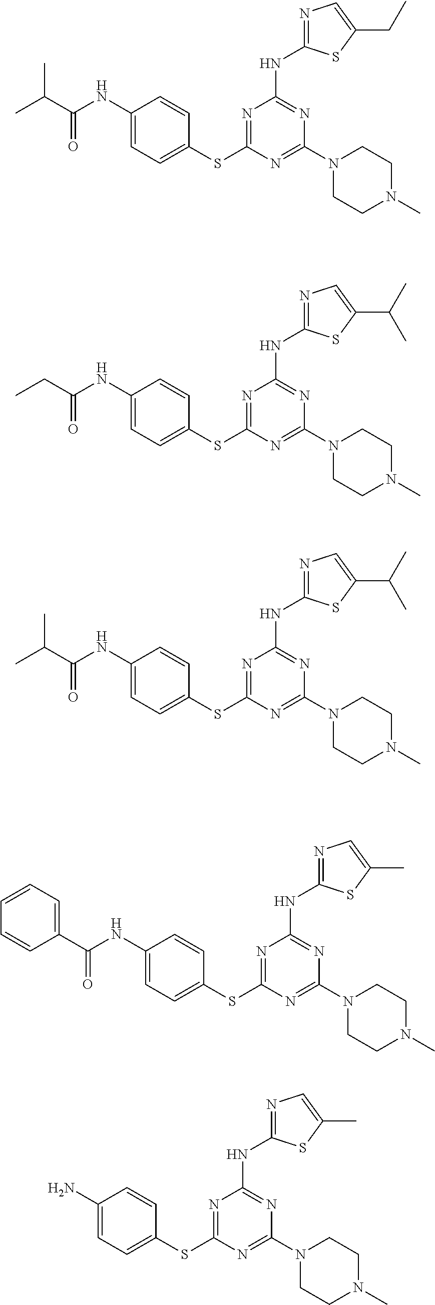

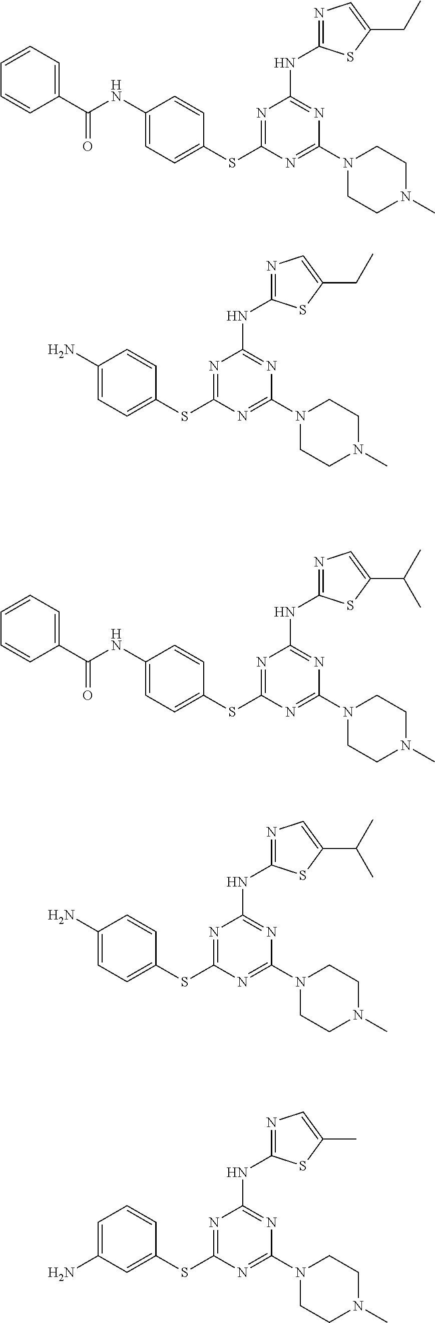

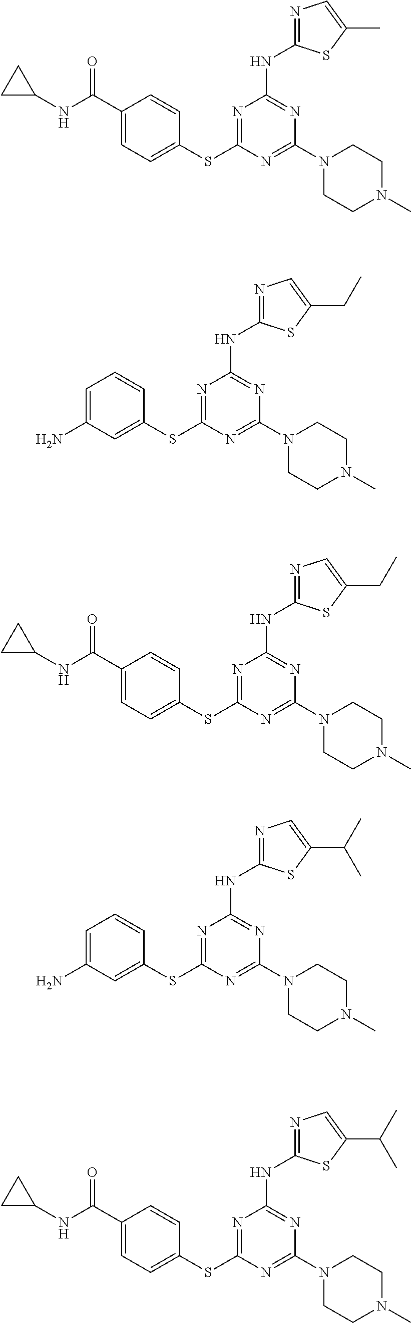

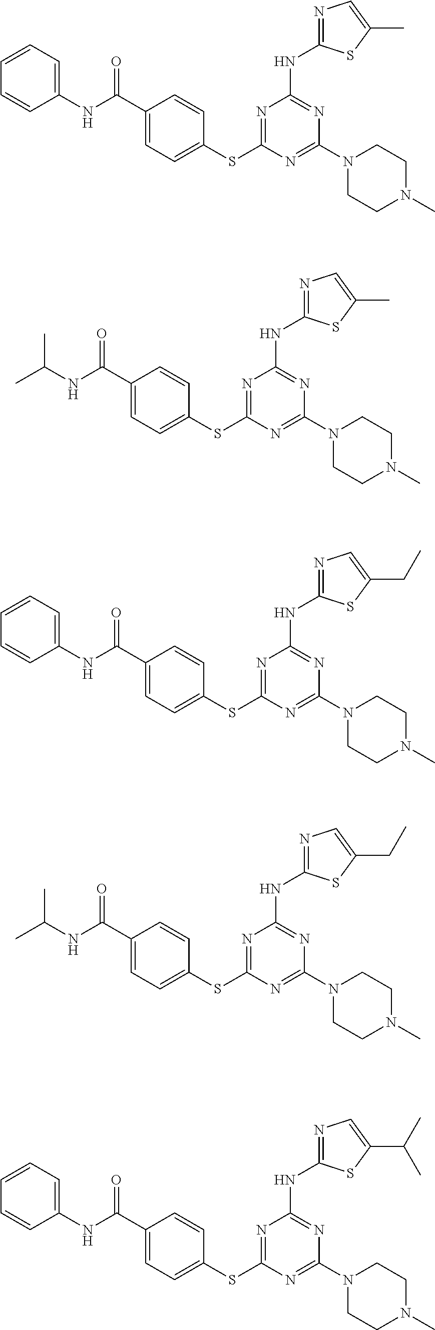

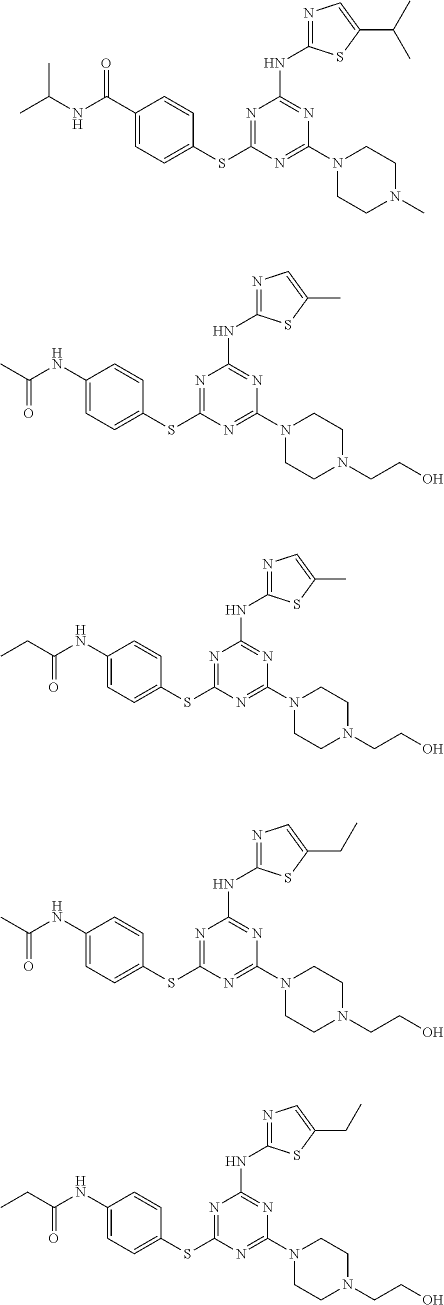

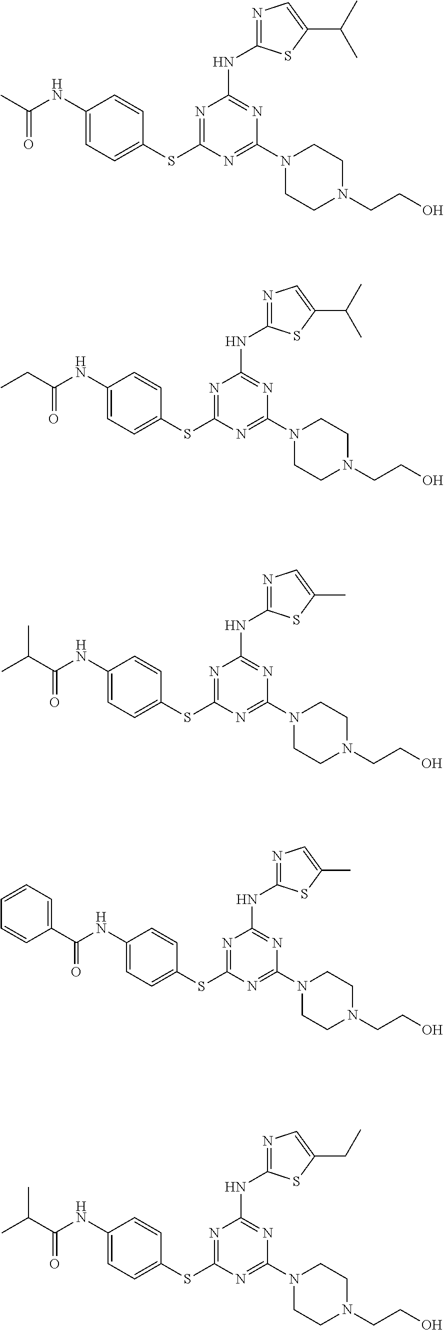

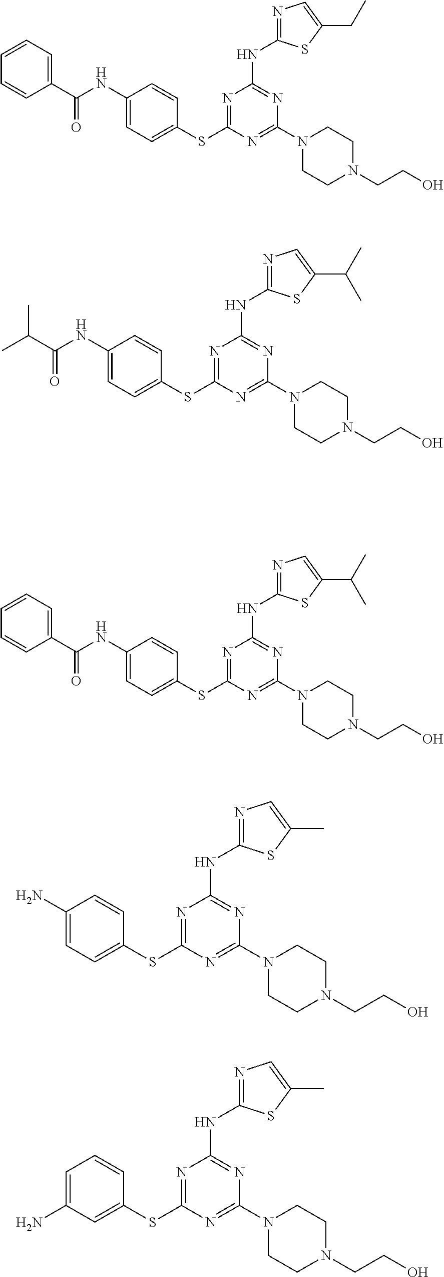

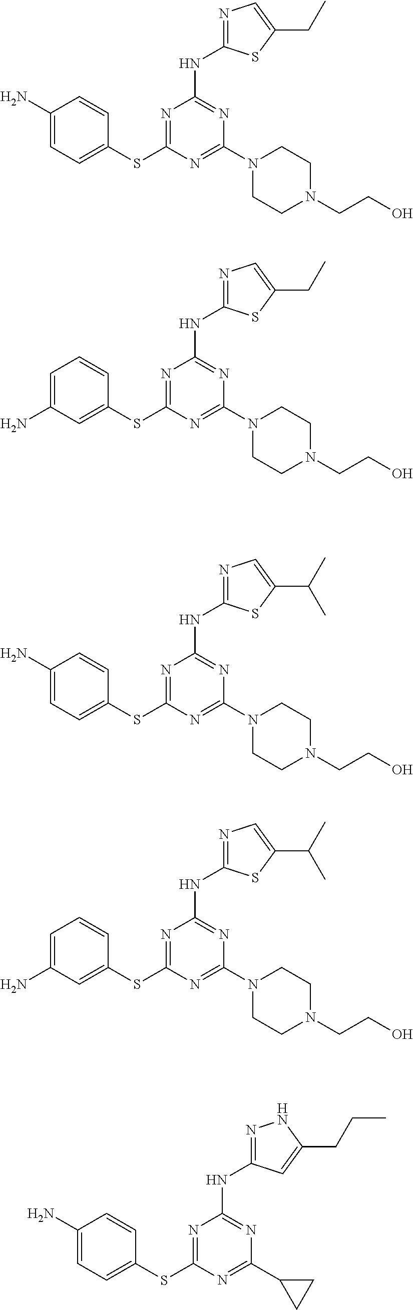

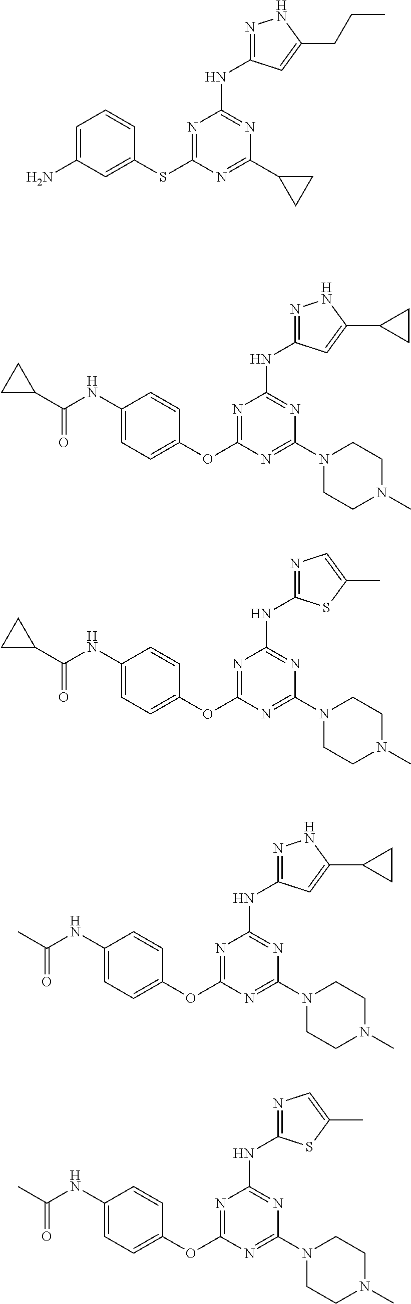

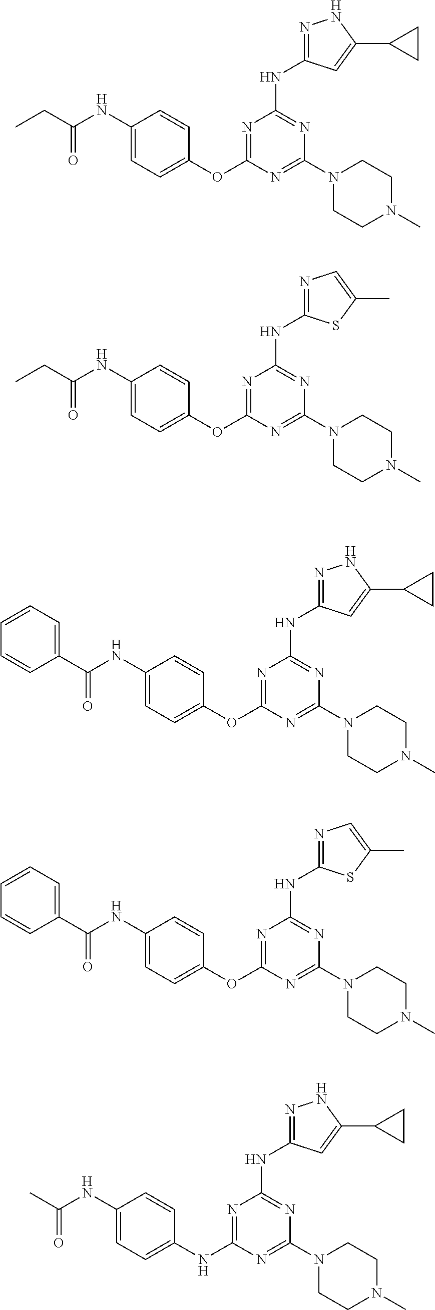

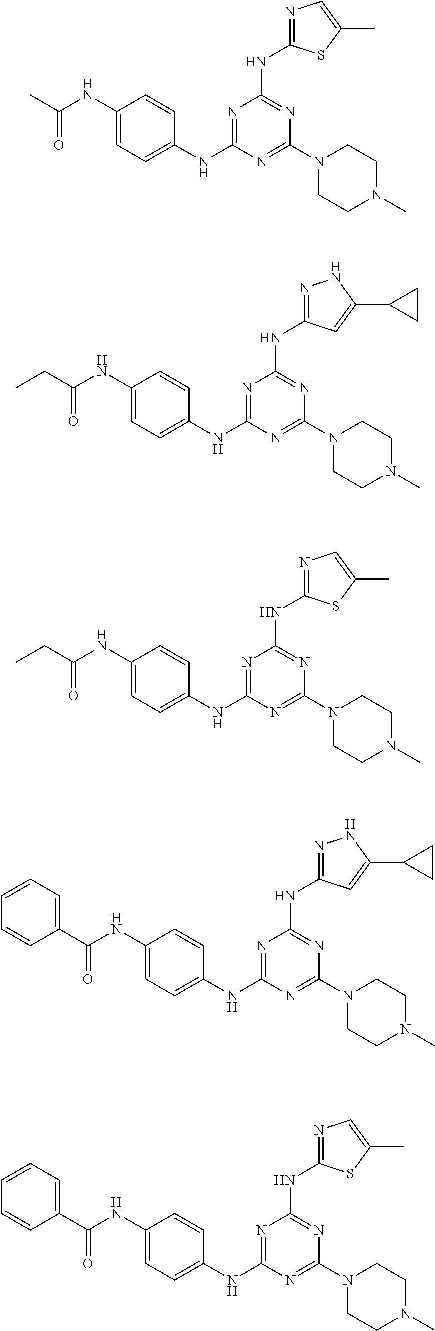

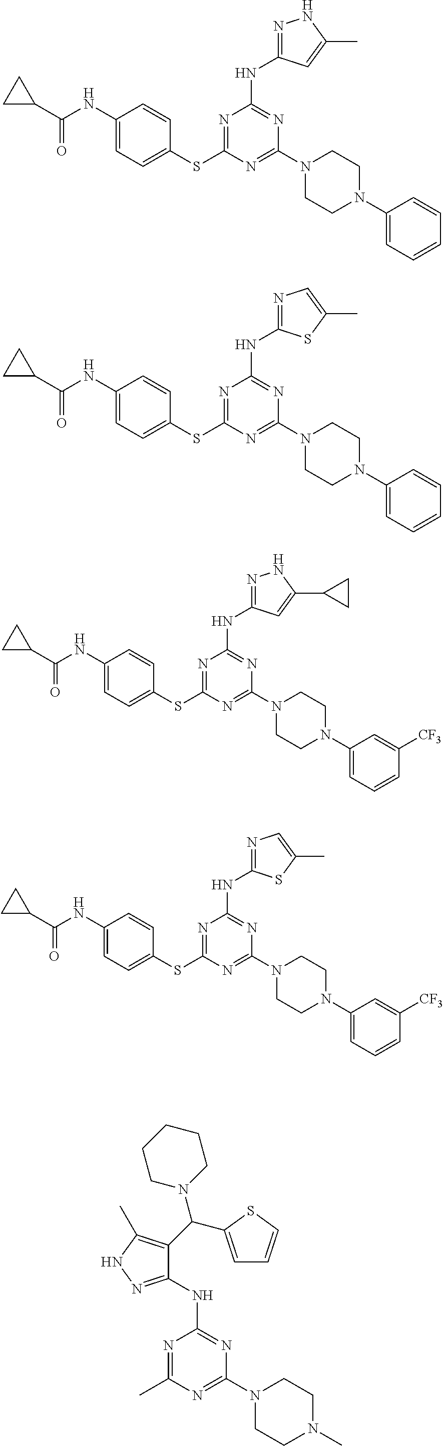

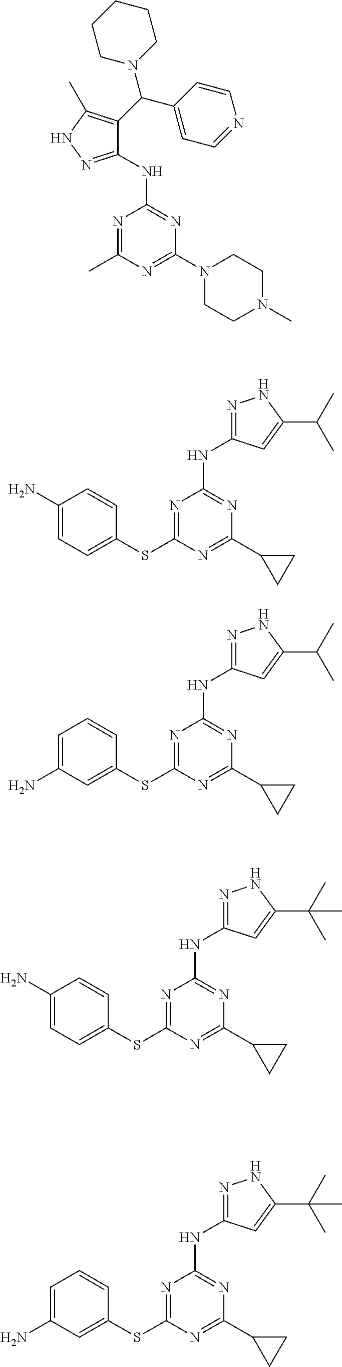

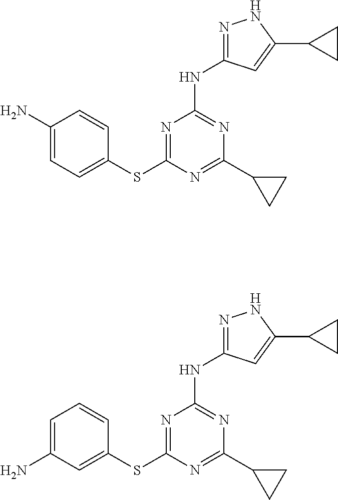

5. The composition according to claim 3, further comprising an additional therapeutic agent

6. A method for treating a disease or condition in a mammal characterized by undesired cellular proliferation or hyperproliferation comprising identifying the mammal afflicted with said disease or condition and administering to said afflicted mammal a composition comprising the compound of claim 1.

7. The method of claim 6, wherein the disease or condition is cancer, stroke, congestive heart failure, an ischemia or reperfusion injury, arthritis or other arthropathy, retinopathy or vitreoretinal disease, macular degeneration, autoimmune disease, vascular leakage syndrome, inflammatory disease, edema, transplant rejection, burn, or acute or adult respiratory distress syndrome.

8. The method of claim 7, wherein the disease or condition is cancer.

9. The method of claim 7, wherein the disease or condition is autoimmune disease.

10. The method of claim 7, wherein the disease or condition is stroke.

11. The method of claim 7, wherein the disease or condition is arthritis.

12. The method of claim 7, wherein the disease or condition is inflammatory disease.

13. The method of claim 7, wherein the disease or condition is associated with a kinase.

14. The method according to claim 7, wherein said method further comprises administering an additional therapeutic agent.

15. The method according to claim 7, wherein said additional therapeutic agent is a chemotherapeutic agent.

16. The method of claim 13, wherein the kinase is a tyrosine kinase.

17. The method of claim 13, wherein the kinase is a serine kinase or a threonine kinase.

18. The method of claim 16, wherein the kinase is an aurora family kinase.

19. The method of claim 8, wherein said cancer is selected from the group consisting of cancers of the liver and biliary tree, intestinal cancers, colorectal cancer, ovarian cancer, small cell and non-small cell lung cancer, breast cancer, sarcomas, fibrosarcoma, malignant fibrous histiocytoma, embryonal rhabdomysocarcoma, leiomysosarcoma, neuro-fibrosarcoma, osteosarcoma, synovial sarcoma, liposarcoma, alveolar soft part sarcoma, neoplasms of the central nervous systems, brain cancer, and lymphomas, including Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma, and combinations thereof.

20. A compound of the formula (II) 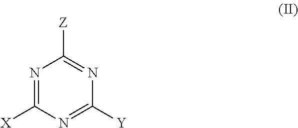
or a pharmaceutically acceptable salt thereof, wherein:
Y is selected from C₁-C₆ alkyl, C₂-C₆ alkenyl, C₂-C₆ alkynyl, —NR⁴R⁵, and -Q-R³;
Q is selected from aryl, heteroaryl, cycloalkyl, and heterocycloalkyl, each of which is optionally substituted with C₁-C₆ alkyl or oxo;
R³ is selected from H, C₁-C₆ alkyl, C₂-C₆ alkenyl, C₂-C₆ alkynyl, C₁-C₆ alkyl-R⁶, aryl, and heteroaryl;
R⁴ and R⁵ are each independently selected from H, C₁-C₆ alkyl, and C₁-C₆ alkyl-R⁶;
R⁶ is selected from hydroxy, —NH₂, mono(C₁-C₆ alkyl)amino, di(C₁-C₆ alkyl)amino, cycloalkyl, and heterocycloalkyl;
X is selected from —K—Ar¹—R¹, C₁-C₆ alkyl, cycloalkyl, and heterocycloalkyl, each of which is optionally substituted with C₁-C₆ alkyl, halogen, hydroxy, amino, cyano, —COOH, or oxo;
K is selected from O and S;
Ar¹ is selected from aryl and heteroaryl;
R¹ is selected from H, —NHC(O)W, —C(O)NHW, and —NH₂;
W is selected from C₁-C₆ alkyl, aryl, heteroaryl, and aryl(C₁-C₆)alkyl, each of which is optionally substituted with C₁-C₆ alkyl, halogen, hydroxy, amino, cyano, —COOH, or oxo;
Z is —(NH)ₙ—Ar²—R²;
n=0, 1;
Ar² is selected from aryl and heteroaryl, each of which is optionally substituted with C₁-C₆ alkyl, halogen, hydroxy, amino, cyano, —COON, or oxo;
R² is selected from H, C₁-C₆ alkyl, —NH₂, ═NH, C₁-C₆ alkoxycarbonyl, halo, and cycloalkyl.

21. A compound of the formula (II) 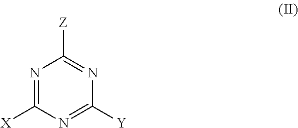
or a pharmaceutically acceptable salt thereof, wherein:
Y is selected from C₁-C₆ alkyl, phenyl, morpholinyl, piperidinyl, pyrrolidinyl, —NR⁴R⁵, and -Q-R³;
Q is piperazinyl;
R³ is selected from C₁-C₆ alkyl, hydroxy(C₁-C₆)alkyl, and pyridinyl;
R⁴ and R⁵ are each independently selected from H, C₁-C₆ alkyl, and C₁-C₆ alkyl-R⁶;
R⁶ is selected from morpholinyl and di(C₁-C₆ alkyl)amino;
X is selected from C₁-C₆ alkyl, methylpiperazinyl, and —K—Ar¹—R¹;
K is selected from O and S;
Ar¹ is phenyl;
R¹ is selected from —NHC(O)W, —C(O)NHW, and —NH₂;
W is selected from C₁-C₆ alkyl, phenyl, and halobenzyl;
Z is —(NH)ₙ—Ar²—R²;
n=0, 1;
Ar² is selected from methylthiazolyl, pyrazolyl, imidazolyl, triazolyl, benzimidazolyl, thiadiazolyl, thiazolyl, isoxazolyl, isothiazolyl, pyrimidinyl, and pyridinyl;
R² is selected from C₁-C₆ alkyl, —NH₂, ═NH, C₁-C₆ alkoxycarbonyl, and halo.

22. A process for making compound of claim 20 or its pharmaceutically acceptable salts, hydrates, solvates, crystal forms salts and individual diastereomers thereof.

23. A pharmaceutical composition comprising at least one compound of claim 20 or its pharmaceutically acceptable salts, hydrates, solvates, crystal forms salts and individual diastereomers thereof, and a pharmaceutically acceptable carrier.

24. A process for making compound of claim 21 or its pharmaceutically acceptable salts, hydrates, solvates, crystal forms salts and individual diastereomers thereof.

25. A pharmaceutical composition comprising at least one compound of claim 21 or its pharmaceutically acceptable salts, hydrates, solvates, crystal forms salts and individual diastereomers thereof, and a pharmaceutically acceptable carrier.
